SEQUENCE LISTING (4 sequences)

SEQ ID NO: 1 - protein, 509 aa, Homo sapiens
STQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVN

SEQ ID NO: 2 - protein, 501 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 3 - protein, 500 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLS

SEQ ID NO: 4 - protein, 502 aa, Homo sapiens
QPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLS

(19) United States
(12) Patent Application Publication
(10) Pub. No.: US 2007/0281365 A1
(43) Pub. Date: Dec. 6, 2007

(54) Crystal Structure of Erbb2 and Uses Thereof

(75) Inventors: Thomas Peter John Garrett, Victoria (AU); Thomas Charles Elleman, Victoria (AU); Timothy Edward Adams, Victoria (AU); Anthony Wilkes Burgess, Victoria (AU); Robert Nicholas Jorissen, Victoria (AU); Meizhen Lou, Victoria (AU); George Oscar Lovrecz, Victoria (AU); Neil Moreton McKern, Victoria (AU); Colin Wesley Ward, Victoria (AU)

Correspondence Address: FOLEY AND LARDNER LLP;SUITE 500, 3000 K STREET NW, WASHINGTON, DC, 20007, US

(73) Assignees: Commonwealth Scientific and Industrial Research Organisation; Ludwig Institute for Cancer Research; Walter & Eliza Hall Institute of Medical Research

(21) Appl. No.: 10/529,887

(22) PCT Filed: Oct. 6, 2003
(86) PCT No.: PCT/AU03/01310

§ 371 Date: Nov. 28, 2005

(30) Foreign Application Priority Data
Oct. 4, 2002 (AU) 2002951853

(51) Int. Cl. G01N 33/566; C07K 14/70; C07K 16/30

(52) U.S. Cl. 436/501; 530/350; 530/387.7

(57) ABSTRACT

More particularly, the present invention relates to the crystal structure of the ErbB2, in particular the crystal structure of an extracellular portion of ErbB2 and to methods of using the crystal and related structural information to screen for and design compounds that interact with ErbB2, or variants of thereof.

### FIELD OF THE INVENTION

The present invention relates generally to structural studies of ErbB2. More particularly, the present invention relates to the crystal structure of the ErbB2, in particular the crystal structure of an extracellular portion of ErbB2 and to methods of using the crystal and related structural information to screen for and design compounds that interact with or modulate ErbB2; or variants thereof.

### BACKGROUND TO THE INVENTION

ErbB2 was discovered as an oncogene (neu) in a rat brain tumor (Schecter et al., 1984, Nature 312, 513-516). ErbB2/HER2 is closely related to the EGF receptor and is the most oncogenic member of the EGFR family. It is amplified and/or overexpressed in approximately 30% of human breast cancers and in many other types of human malignancies and this overexpression is correlated with poor clinical prognosis (see Mendelsohn and Baselga, 2000, Oncogene 19, 6550-6565; Yu and Hung, 2000, Oncogene 19, 6115-6121). Overexpression of ErbB2 enhances metastasis-related properties such as invasion, angiogenesis and increased survival of cancer cells, and confers increased resistance to various cancer therapies including chemotherapy and gamma-radiation (see Mendelsohn and Baselga, 2000; Yu and Hung, 2000). Some forms of breast cancer are now treated with antibodies that recognise ErbB2 and improvements in anti-ErbB2 therapies are likely to flow from a better understanding of its 3D structure and its mechanism of action.

Considerable resources have been directed to the identification of an ErbB2 ligand. No ligand has been found, however the search led to the discovery of ErbB4 and considerable improvements in our biological understanding of the EGF receptor family (Harari and Yarden, 2000, Oncogene 19, 6102-6114; Yarden and Sliwkowski, 2001, Nat. Rev. Mol. Cell. Biol. 2, 127-137). It now seems certain that ErbB2 has no ligand. Instead it acts as a second receptor sub-unit in three EGF receptor family heterodimers: ErbB1-ErbB2, ErbB3-ErbB2 and ErbB4-ErbB2 (Daly et al., 1997, Cancer Res. 57, 3804-3811; Sundaresan et al., 1998, Endocrinol. 139, 4756-4764). There is definitive evidence that the EGF receptor homodimer signals differently to the EGF receptor-ErbB2 heterodimer. Unless ErbB2 carries an oncogenic mutation, as in c-neu, it signals only after activation of its heterodimer partner by EGF or other relevant ligand.

The human ErbB2 is a large (1234 residues), monomeric, modular glycoprotein with an extracellular domain, a single transmembrane region and an intracellular cytoplasmic tyrosine kinase, which is flanked by noncatalytic regulatory regions (Yamamoto et al., 1986, Nature 319, 230-234). The extracellular portion of human ErbB2 (residues 1-632), like the EGFR, consists of four sub-domains L1, CR1, L2 and CR2 (Bajaj et al., 1987, Biochim. Biophys. Acta 916, 220-226; Ward et al., 1995, Proteins: Struct. Funct. Genet. 22, 141-153) also referred to as domains I-IV (Lax et al., 1988, Mol. Cell. Biol. 8, 1970-1978).

### SUMMARY OF THE INVENTION

We have determined the three dimensional structure of a truncated form (residues 1-509) of the ectodomain of the tyrosine kinase receptor ErbB2 at 2.5 Å resolution and compared it with the recently solved structures of the EGFR ectodomain with TGFα or EGF and the unliganded ErbB3 ectodomain. Lack of ligand binding by ErbB2 appears to be caused by amino acid differences in the L1 and L2 domains of ErbB2. Furthermore, ligands would not be able to bind to the observed conformation of ErbB2 here as kinks in the first Cys-rich region (CR1) lead to a closer juxtaposition of the L domains, occluding the region of ErbB2 that is analogous to the EGFR ligand binding site. The L1/L2 buried surface area and the degree of complementarity in the L domain interface implies that this “closed” form is biologically relevant.

Accordingly, in one aspect, the present invention provides a method for identifying a potential modulator compound for ErbB2 which method comprises:

(a) providing a three-dimensional structure of

(i) amino acids 1-509 of ErbB2 polypeptide having the atomic coordinates shown in Appendix I, or atomic coordinates having a root mean square deviation of backbone atoms of not more than 1.5 Å when superimposed on the corresponding backbone atoms described by the atomic coordinates shown in Appendix I; or

(ii) a subset of said amino acids having a corresponding subset of the atomic coordinates shown in Appendix I, or atomic coordinates having a root mean square deviation of backbone atoms of not more than 1.5 Å when superimposed on the corresponding backbone atoms described by the atomic coordinates shown in Appendix I;

(b) providing the three-dimensional structure of a candidate compound;

(c) assessing the stereochemical complementarity between the three-dimensional structure of step (b) and a region of the three-dimensional structure of step (a); and

(d) selecting a compound on the basis of the stereochemical complementarity.

In a preferred embodiment, the method further comprises:

(e) synthesising or obtaining a candidate compound assessed in step (c) as possessing stereochemical complementarity with the three-dimensional structure of step (a);

(f) determining the ability of the candidate compound to interact with and/or modulate the activity of ErbB2.

In yet a further aspect the present invention provides a method for preparing a pharmaceutical composition for treating diseases associated with aberrant ErbB2 signalling, the method comprising:

(a) providing a three-dimensional structure of

(i) amino acids 1-509 of ErbB2 polypeptide having the atomic coordinates shown in Appendix I, or atomic coordinates having a root mean square deviation of backbone atoms of not more than 1.5 Å when superimposed on the corresponding backbone atoms described by the atomic coordinates shown in Appendix I; or

(ii) a subset of said amino acids having a corresponding subset of the atomic coordinates shown in Appendix I, or atomic coordinates having a root mean square deviation of backbone atoms of not more than 1.5 Å when superimposed on the corresponding backbone atoms described by the atomic coordinates shown in Appendix I;

(b) providing the three-dimensional structure of a candidate compound;

(c) assessing the stereochemical complementarity between the three-dimensional structure of step (b) and a region of the three-dimensional structure of step (a); and

(d) selecting a compound on the basis of the stereochemical complementarity;

(e) synthesising or obtaining a candidate compound assessed in step (c) as possessing stereochemical complementarity with the three-dimensional structure of step (a);

(f) determining the ability of the candidate compound to interact with and/or modulate the activity of ErbB2; and

(g) incorporating the compound into a pharmaceutical composition.

The method may be used for either targeted or broad screening. Targeted screening involves the design and synthesis of chemical compounds that are analogs of some active compounds or that can specifically act with the biological target under investigation. Broad screening involves the design and synthesis of a large array of maximally diverse chemical compounds, leading to diverse libraries that are tested against a variety of biological targets.

In a further aspect, the present invention provides a method of modulating ErbB2, the method comprising contacting the receptor with a compound that matches a region selected from at least one of the CR1 domain, the potential CR1 loop docking site between the L1, CR1 and L2 domains, the CR1-L2 hinge region, the regions of the L1 and L2 domains that contact each other in a closed conformation.

The compound may be a small molecule modulator. The term “small molecule” includes an organic compound either synthesized in the laboratory or found in nature. Typically, a small molecule is any organic molecule having a molecular weight of less than about 1500. Preferably the molecule has a molecular weight less that about 1000, more preferably less than about 500.

The term “ErbB2” as used herein includes wild-type ErbB2 and variants thereof including allelic variants and naturally occurring mutations and genetically engineered variants.

The present invention also provides a set of coordinates as shown in Appendix I, or a subset thereof, where said coordinates define a three dimensional structure of amino acids 1-509 of an ErbB2 polypeptide or a subset of said amino acids, or a set of coordinates having a root mean square deviation of backbone atoms of not more than 1.5 Å when superimposed on the corresponding backbone atoms described by the atomic coordinates shown in Appendix I, or a subset thereof.

In a related aspect, the present invention provides a computer for producing a three-dimensional representation of a molecule or molecular complex, wherein the computer comprises:
(a) a machine-readable data storage medium comprising a data storage material encoded with machine-readable data, wherein the machine readable data comprises (i) the atomic coordinates of amino acids 1-509 of an ErbB2 polypeptide as shown in Appendix I, or atomic coordinates having a root mean square deviation of backbone atoms of not more than 1.5 Å when superimposed on the corresponding backbone atoms described by the atomic coordinates shown in Appendix I; or (ii) the atomic coordinates of a subset of said amino acids having a corresponding subset of the atomic coordinates shown in Appendix I, or atomic coordinates having a root mean square deviation of backbone atoms of not more than 1.5 Å when superimposed on the corresponding backbone atoms described by the atomic coordinates shown in Appendix I;
(b) a working memory for storing instructions for processing the machine-readable data;
(c) a central-processing unit coupled to the working memory and to the machine-readable data storage medium, for processing the machine-readable data into the three dimensional representation; and
(d) an output hardware coupled to the central processing unit, for receiving the three-dimensional representation.

Preferably, said subsets of amino acids are selected from the CR1 domain and the potential CR1 loop docking site between the L1, CR1 and L2 domains equivalent to that seen in the TGFα:sEGFR dimer complex (Garrett et al., 2002, Cell 110, 763-773), or the CR1-L2 hinge region or the regions of the L1 and L2 domains that contact each other in this closed conformation.

More preferably the subset of amino acids defines a homodimerisation or heterodimerisation surface with other EGF receptor family members. Preferred heterodimerisation surfaces include (i) the N-terminal end of the CR1 domain (residues 200-203, 210-213, 216-218, 225-230), (ii) the CR1 domain dimerisation loop (residues 247-268) and adjacent residues (residues 244-246, 285-289) and (iii) the C-terminal end of the CR1 domain (residues 294-319).

In a further preferred embodiment, the subset of amino acids comprises the following residues: Gln 36, Gln 60, Arg 82, Thr 84, Gln 85, Phe 237, Thr 269, Phe 270, Gly 271, Ala 272, Tyr 282, Thr 285, Gly 288, Ser 289, Cys 290, Thr 291, Leu 292, Val 293, Cys 294, Pro 295 and Cys 310.

The three-dimensional structure of ErbB2 may be used to develop models useful for drug design and in silico screening of candidate compounds that modulate ErbB2 activity. Other physicochemical characteristics may also be used in developing the model, e.g. bonding, electrostatics etc.

Generally the term “in silico” refers to the creation in a computer memory, i.e., on a silicon or other like chip. Stated otherwise “in silico” means “virtual.” When used herein the term “in silico” is intended to refer to screening methods based on the use of computer models rather than in vitro or in vivo experiments.

By “modulate” we mean that the compound increases or decreases signal transduction via ErbB2. The phrase “decreases signal transduction” is intended to encompass partial or complete inhibition of signal transduction via ErbB2. The ability of a candidate compound to increase or decrease signal transduction via ErbB2 can be assessed by any one of the ErbB2 cell-based assays described herein.

The term “small molecule” includes a compound with a molecular weight of 1500 or less. Preferably, the small molecule has a molecular weight of less than 1000, particularly preferred is a molecule having a molecular weight of less than 500.

Accordingly, in yet a further aspect, the present invention provides a computer-based method of identifying a candidate modulator of ErbB2, which method comprises fitting the structure of
(i) amino acids 1-509 of an ErbB2 polypeptide having the atomic coordinates shown in Appendix I, or atomic coordinates having a root mean square deviation of backbone atoms of not more than 1.5 Å when superimposed on the corresponding backbone atoms described by the atomic coordinates shown in Appendix I; or
(ii) a subset of said amino acids having a corresponding subset of the atomic coordinates shown in Appendix 1, or atomic coordinates having a root mean square deviation of backbone atoms of not more than 1.5 Å when superimposed on the corresponding backbone atoms described by the atomic coordinates shown in Appendix I;
to the structure of a candidate modulator molecule.

In a further related aspect, the present invention provides a computer-assisted method for identifying candidate compounds able to interact with ErbB2 and thereby modulate an activity mediated by the receptor, using a programmed computer comprising a processor, an input device, and an output device, which method comprises the steps of:
(a) entering into the programmed computer, through the input device, data comprising the atomic coordinates of amino acids 1-509 of ErbB2 as shown in Appendix I, or atomic coordinates having a root mean square deviation of backbone atoms of not more than 1.5 Å when superimposed on the corresponding backbone atoms described by the atomic coordinates shown in Appendix I, or a subset of said coordinates;
(b) generating, using computer methods, a set of atomic coordinates of a structure that possesses stereochemical complementarity to the atomic coordinates entered in step (a), thereby generating a criteria data set;
(c) comparing, using the processor, the criteria data set to a computer database of chemical structures;
(d) selecting from the database, using computer methods, chemical structures which are similar to a portion of said criteria data set; and
(e) outputting, to the output device, the selected chemical structures which are complementary to or similar to a portion of the criteria data set.

In another related aspect, the present invention provides a method for evaluating the ability of a chemical entity to interact with an ErbB2, said method comprising the steps of:
(a) providing a computer model of at least one region of ErbB2 using structure coordinates wherein the root mean square deviation between said structure coordinates and the structure coordinates of amino acids 1-509 of ErbB2 as set forth in Appendix I is not more than 1.5 Å;
(b) employing computational means to perform a fitting operation between the chemical entity and said computer model of the binding surface; and
(c) analysing the results of said fitting operation to quantify the association between the chemical entity and the binding surface model.

The model may be adaptive in a sense that it allows for slight surface changes to improve the fit between the candidate compound and the protein, e.g. by small movements in side chains or main chain.

Preferably, the region of ErbB2 is defined by the CR1 domain and the potential CR1 loop docking site between the L1, CR1 and L2 domains equivalent to that seen in the TGFα:sEGFR dimer complex (Garrett et al., 2002), or the CR1-L2 hinge region or the regions of the L1 and L2 domains that contact each other in this closed conformation and combinations thereof.

More preferably the region defines a heterodimerisation surface with other EGF receptor family members. Preferred heterodimerisation surfaces include (i) the N-terminal end of the CR1 domain (residues 200-203, 210-213, 216-218, 225-230), (ii) the CR1 domain dimerisation loop (residues 247-268) and adjacent residues (residues 244-246, 285-289) and (iii) the C-terminal end of the CR1 domain (residues 294-319).

In a further preferred embodiment, the region comprises the following amino acid residues: Gln 36, Gln 60, Arg 82, Thr 84, Gln 85, Phe 237, Thr 269, Phe 270, Gly 271, Ala 272, Tyr 282, Thr 285, Gly 288, Ser 289, Cys 290, Thr 291, Leu 292, Val 293, Cys 294, Pro 295 and Cys 310.

The ErbB2 crystal structure provided herein may also be used to model/solve the structure of a new crystal using molecular replacement. Accordingly, in a further aspect the present invention provides a method of using molecular replacement to obtain structural information about a molecule or a molecular complex of unknown structure, comprising the steps of:
(i) crystallising said molecule or molecular complex;
(ii) generating an X-ray diffraction pattern from said crystallized molecule or molecular complex;
(iii) applying at least a portion of the structure coordinates set forth in Appendix I, or structure coordinates having a root mean square deviation of backbone atoms of not more than 1.5 Å when superimposed on the corresponding backbone atoms described by the structure coordinates set forth in Appendix I, to the X-ray diffraction pattern to generate a three-dimensional electron density map of at least a portion of the molecule or molecular complex whose structure is unknown.

Preferably the molecule of unknown structure is ErbB2 or variant thereof.

In one embodiment, the molecular complex of unknown structure is a complex of ErbB2, or variant thereof, and a ligand or candidate ligand.

In another embodiment the molecular complex of unknown structure is a complex of ErbB2 and an EGF receptor. The molecular complex of unknown structure may also be a complex of ErbB2, an ErbB1 (EGF receptor), ErbB3 or ErbB4 receptor and a ligand or candidate ligand.

The screening methods of the fourth aspect of the invention may be used to identify compounds that modulate ErbB2 signalling. Such compounds may be used to treat disorders associated with ErbB2 dysfunction.

Accordingly, in a further aspect, the present invention provides a method for preventing or treating a disease associated with signaling by ErbB2 which method comprises administering to a subject in need thereof a compound identified by the screening methods of the invention.

The present invention also provides a pharmaceutical composition comprising a compound identified by the screening methods of the invention, which compound is able to bind to the extracellular domain of ErbB2 and modulate an activity of said receptor, as well as a method of preventing or treating a disease associated with signalling by ErbB2 which method comprises administering to a subject in need thereof a composition of the invention.

In yet a further aspect, the present invention provides a crystal of an ErbB2 polypeptide. In particular the present invention provides a crystal of an ErbB2 polypeptide having a space group P2₁2₁2₁ with unit cell dimensions of a=75.96 Å, b=82.24 Å, and c=110.06 Å with up to about 1% variation in any cell dimension. Preferably said ErbB2 polypeptide is a truncated soluble extracellular domain of the full-length ErbB2.

The present invention also provides a crystalline composition comprising a crystal of ErbB2.

In a further aspect, the invention provides a computer system for identifying one or more candidate modulators of ErbB2, the system containing data representing the structure of

(i) amino acids 1-509 of ErbB2 polypeptide having the atomic coordinates shown in Appendix I, or atomic coordinates having a root mean square deviation of backbone atoms of not more than 1.5 Å when superimposed on the corresponding backbone atoms described by the atomic coordinates shown in Appendix I; or

(ii) a subset of said amino acids having a corresponding subset of the atomic coordinates shown in Appendix I, or atomic coordinates having a root mean square deviation of backbone atoms of not more than 1.5 Å when superimposed on the corresponding backbone atoms described by the atomic coordinates shown in Appendix I.

The present invention further provides a computer readable media having recorded thereon data representing a model and/or the atomic coordinates of a ErbB2 crystal. Also provided is a computer readable media having recorded thereon coordinate data according to Appendix I, or a subset thereof, where said coordinate data define a three dimensional structure of amino acids 1-509 of ErbB2 polypeptide or a subset of said amino acids, or coordinate data having a root mean square deviation of backbone atoms of not more than 1.5 Å when superimposed on the corresponding backbone atoms described by the atomic coordinate according to Appendix I, or a subset thereof.

Particular diseases associated with signalling by ErbB2 include cancerous conditions such as cancer of the brain, head and neck, prostate, testicular, ovary, breast, cervix, lung, pancreas and colon; and melanoma, rhabdomyosarcoma, mesothelioma, squamous carcinomas of the skin and glioblastoma.

The information provided in Appendix I shows that there are a number of loop structures that line the ErbB2 dimerisation surface. It is envisaged that antibodies directed against these loop structures would interfere with the formation of heterodimers with other members of the EGF receptor family.

Accordingly, in a further aspect the present invention provides an antibody that binds to ErbB2, the antibody being directed against a structure defined by (i) ErbB2 amino acid residues 200-203, (ii) ErbB2 amino acid residues 210-213, (iii) ErbB2 amino acid residues 216-218, (iv) ErbB2 amino acid residues 225-230, (v) ErbB2 amino acid residues 247-268 or a subset thereof; (vi) ErbB2 amino acid residues 244-246, (vii) ErbB2 amino acid residues 285-289, or (viii) ErbB2 amino acid residues 294-319 or a subset thereof.

In yet a further aspect the present invention provides an isolated conformationally constrained peptide or, peptidomimetic consisting essentially of (i) ErbB2 amino acid residues 200-203, (ii) ErbB2 amino acid residues 210-213, (iii) ErbB2 amino acid residues 216-218, (iv) ErbB2 amino acid residues 225-230, (v) ErbB2 amino acid residues 247-268 or a subset thereof; (vi) ErbB2 amino acid residues 244-246, (vii) ErbB2 amino acid residues 285-289, or (viii) ErbB2 amino acid residues 294-319 or a subset thereof.

In yet a further aspect the present invention provides a computer-assisted method for identifying potential mimetics of ErbB2, using a programmed computer comprising a processor, a data storage system, an input device, and an output device, comprising the steps of:

(a) inputting into the programmed computer through said input device data comprising the atomic coordinates of amino acids 200-203, 210-213, 216-218, 225-230, 247-268, 244-246, 285-289, or 294-319 of ErbB2 as shown in Appendix I, or atomic coordinates having a root mean square deviation of backbone atoms of not more than 1.5 Å when superimposed on the corresponding backbone atoms described by the atomic coordinates shown in Appendix I, thereby generating a criteria data set;

(b) comparing, using said processor, said criteria data set to a computer database of chemical structures stored in said computer data storage system;

(c) selecting from said database, using computer methods, chemical structures having a portion that is structurally similar to said criteria data set;

(d) outputting to said output device the selected chemical structures having a portion similar to said criteria data set.

In yet a further aspect the present invention provides a computer-assisted method for identifying potential mimetics of ErbB2, using a programmed computer comprising a processor, a data storage system, an input device, and an output device, comprising the steps of:

(a) inputting into the programmed computer through said input device data comprising the atomic coordinates of amino acids 200-203, 210-213, 216-218, 225-230, 247-268, 244-246, 285-289, or 294-319 of ErbB2 as shown in Appendix I, or atomic coordinates having a root mean square deviation of backbone atoms of not more than 1.5 Å when superimposed on the corresponding backbone atoms described by the atomic coordinates shown in Appendix I, thereby generating a criteria data set;

(b) constructing, using computer methods, a model of a chemical structure having a portion that is structurally similar to said criteria data set;

(c) outputting to said output device the constructed model.

In yet a further aspect the present invention provides a compound having a chemical structure selected using a method of the present invention, said compound being an ErbB2 mimetic. Preferably, the compound is a peptidomimetic that has fewer than 30 amino acids, more preferably fewer than 25 amino acids.

As will be readily understood by those skilled in this field the methods of the present invention provide a rational method for designing and selecting compounds including antibodies which interact with ErbB2. In the majority of cases these compounds will require further development in order to increase activity. Such further development is routine in this field and will be assisted by the structural information provided in this application. It is intended that in particular embodiments the methods of the present invention includes such further developmental steps.

It is also intended that embodiments of the present invention include manufacturing steps such as incorporating the compound into a pharmaceutical composition in the manufacture of a medicament.

Throughout this specification, preferred aspects and embodiments apply, as appropriate, separately, or in combination, to other aspects and embodiments, mutatis mutandis, whether or not explicitly stated as such.

### BRIEF DESCRIPTION OF THE FIGURES

FIG. 1. Structure-based sequence alignment of the human ErbB2 ectodomain with other members of the ErbB family.

(A) The receptor L1 and L2 domains plus the first module of the cys-rich regions, CR1 and CR2. Positions with conserved physicochemical properties of amino acids are boxed. Disulfide bond connections are shown are solid lines. Secondary structure elements are indicated above and below the sequences as cylinders for α-helices and arrows for β-strands. Residues buried at L1/L2 interface are denoted by R. Sequence sources are: EGFR (Ullrich et al., 1984, Nature 309, 418-425), ErbB2 (Yamamoto et al., 1986); ErbB3 (Kraus et al., 1989, Proc Natl Acad Sci USA. 86, 9193-9197; Plowman et al., 1990, Proc. Natl. Acad. Sci. USA. 87, 4905-4909); ErbB4 (Plowman et al., 1993, Proc. Natl. Acad. Sci. USA. 90, 1746-1750).

(B) Modules 2 to 8 of the ErbB family cys-rich region CR1 and modules 2 to 7 of CR2. Three types of disulfide bonded modules are indicated by bars below the sequences. The unfilled bars below parts of the cys-rich sequences indicate modules with 2 disulfide bonds (in a Cys 1-3 and 2-4 arrangement), the solid bars indicate modules which contain a single disulfide bond and have a β-finger motif, and the dashed bar indicates residues present in a disulfide-linked bend consisting of only five residues. Disulfide bonds are shown in solid lines and except for those that do not conform to the CR1 pattern which are indicated as dashed lines. The number in parentheses shows where amino acids have been omitted. Boxed residues and secondary structure elements are as in A.

FIG. 2. Polypeptide fold for residues 1-509 of ErbB2 and its comparison with EGFR (1-501) as seen in the 2:2 complex with TGFα, and the full length ectodomain of ErbB3.

FIG. 3. Percentage inhibition of thymidine incorporated in a cell line expressing erbB2 on EGFR-K721R (a kinase defective EGFR)+full length ErbB2 by compounds 39293, 94289, 19378 and 20697.

### DETAILED DESCRIPTION OF THE INVENTION

Unless defined otherwise, all technical and scientific terms used herein have the same meaning as commonly understood by one of ordinary skill in the art (e.g. in molecular biology, biochemistry, structural biology, and computational biology). Standard techniques are used for molecular and biochemical methods (see generally, Sambrook et al., Molecular Cloning: A Laboratory Manual, 3^{rd} ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

ErbB2 Crystals and Crystal Structures

The present invention provides a crystal comprising an ErbB2 polypeptide. Such crystals preferably are of the space group P2₁2₁2₁ with unit cell dimensions of a=75.96 Å, b=82.24 Å, and c=110.06 Å.

As used herein, the term “crystal” means a structure (such as a three dimensional (3D) solid aggregate) in which the plane faces intersect at definite angles and in which there is a regular structure (such as internal structure) of the constituent chemical species. The term “crystal” refers in particular to a solid physical crystal form such as an experimentally prepared crystal.

Crystals according to the invention may be prepared using full-length ErbB2 polypeptides. However, preferably the extracellular domain is employed in isolation. Thus, preferably the ErbB2 polypeptide is a truncated polypeptide containing the extracellular domain and lacking the transmembrane domain and the intracellular tyrosine kinase domain. Typically, the extracellular domain comprises residues 1 to 632 (mature receptor numbering) of human ErbB2, or the equivalent thereof, or a truncated version thereof, preferably comprising amino acids 1 to 509, or the equivalent residues in other ErbB2 polypeptides.

In a preferred embodiment the ErbB2 polypeptide is human ErbB2 (Accession No. A24571—mature protein begins at residue 22). However, the ErbB2 polypeptide may also be obtained from other species, such as other mammalian species.

Crystals may be constructed with wild-type ErbB2 polypeptide sequences or variants thereof, including allelic variants and naturally occurring mutations as well as genetically engineered variants. Typically, variants have at least 95 or 98% sequence identity with a corresponding wild-type ErbB2 polypeptide.

Optionally, the crystal of ErbB2 may comprise one or more molecules which bind to ErbB2, or otherwise soaked into the crystal or cocrystallise with ErbB2. Such molecules include ligands or small molecules, which may be candidate pharmaceutical agents intended to modulate the interaction between ErbB2 and its biological targets or dimer partners, such as other members of the EGF receptor family. The crystal of ErbB2 may also be a molecular complex with other receptors of the EGF receptor family such as ErbB1 (the EGF receptor), ErbB3 or ErbB4. The complex may also comprise additional molecules such as the ligands to these receptors.

The production of ErbB2 crystals is described below.

In a preferred embodiment, an ErbB2 crystal of the invention has the atomic coordinates set forth in Appendix I. It will be understood by those skilled in the art that atomic coordinates may be varied, without affecting significantly the accuracy of models derived therefrom; thus, although the invention provides a very precise definition of a preferred atomic structure, it will be understood that minor variations are envisaged and the claims are intended to encompass such variations. Preferred are variants in which the r.m.s. deviation of the x, y and z co-ordinates for all backbone atoms other than hydrogen is less than 1.5 Å (preferably less than 1 Å, 0.7 Å or less than 0.3 Å) compared with the coordinates given in Appendix I.

In a highly preferred embodiment, the crystal has the atomic coordinates as shown in Appendix I.

As used herein, the term “atomic co-ordinates” refer to a set of values which define the position of one or more atoms with reference to a system of axes.

The present invention also provides a crystal structure of an ErbB2 polypeptide, in particular a crystal structure of the extracellular domain of an ErbB2 polypeptide, or a region thereof.

The atomic coordinates obtained experimentally for amino acids 1 to 509 (mature receptor numbering) of human ErbB2 are shown in Appendix I. However, a person skilled in the art will appreciate that a set of atomic coordinates determined by X-ray crystallography is not without standard error. Accordingly, any set of structure coordinates for an ErbB2 polypeptide that has a root mean square deviation of protein backbone atoms of less than 0.75 Å when superimposed (using backbone atoms) on the atomic coordinates listed in Appendix I shall be considered identical.

The present invention also comprises the atomic coordinates of an ErbB2 polypeptide that substantially conform to the atomic coordinates listed in Appendix I.

A structure that “substantially conforms” to a given set of atomic coordinates is a structure wherein at least about 50% of such structure has an average root-mean-square deviation (RMSD) of less than about 1.5 Å for the backbone atoms in secondary structure elements in each domain, and more preferably, less than about 1.3 Å for the backbone atoms in secondary structure elements in each domain, and, in increasing preference, less than about 1.0 Å, less than about 0.7 Å, less than about 0.5 Å, and most preferably, less than about 0.3 Å for the backbone atoms in secondary structure elements in each domain.

In a more preferred embodiment, a structure that substantially conforms to a given set of atomic coordinates is a structure wherein at least about 75% of such structure has the recited average root-mean-square deviation (RMSD) value, and more preferably, at least about 90% of such structure has the recited average root-mean-square deviation (RMSD) value, and most preferably, about 100% of such structure has the recited average root-mean-square deviation (RMSD) value.

In an even more preferred embodiment, the above definition of “substantially conforms” can be extended to include atoms of amino acid side chains. As used herein, the phrase “common amino acid side chains” refers to amino acid side chains that are common to both the structure which substantially conforms to a given set of atomic coordinates and the structure that is actually represented by such atomic coordinates.

The present invention also provides subsets of said atomic coordinates listed in Appendix I and subsets that conform substantially thereto. Preferred subsets define one or more regions of the human ErbB2 extracellular domain selected from the CR1 domain and the potential CR1 loop docking site between the L1, CR1 and L2 domains equivalent to that seen in the TGFα:sEGFR dimer complex (Garrett et al., 2002), or the CR1-L2 hinge region or the regions of the L1 and L2 domains that contact each other in this closed conformation. A particularly preferred subset defines the heterodimerisation surface of ErbB2 with other members of the EGF receptor family, such as ErbB1, ErbB3 and/or ErbB4.

It will be appreciated that a set of structure coordinates for a polypeptide is a relative set of points that define a shape in three dimensions. Thus, it is possible that an entirely different set of coordinates could define a similar or identical shape. Moreover, slight variations in the individual coordinates will have little effect on overall shape.

The variations in coordinates may be generated due to mathematical manipulations of the structure coordinates. For example, the structure coordinates set forth in Appendix I could be manipulated by crystallographic permutations of the structure coordinates, fractionalisation of the structure coordinates, integer additions or subtractions to sets of the structure coordinates, inversion of the structure coordinates, or any combination thereof.

Alternatively, modification in the crystal structure due to mutations, additions, substitutions, and/or deletions of amino acids, or other changes in any of the components that make up the crystal could also account for variations in structure coordinates.

Various computational analyses are used to determine whether a molecular complex or a portion thereof is sufficiently similar to all or parts of the structure of the extracellular domain of ErbB2 described above. Such analyses may be carried out in current software applications, such as the Molecular Similarity program of QUANTA (Molecular Simulations Inc., San Diego, Calif.) version 4.1.

The Molecular Similarity program permits comparisons between different structures, different conformations of the same structure, and different parts of the same structure.

Comparisons typically involve calculation of the optimum translations and rotations required such that the root mean square difference of the fit over the specified pairs of equivalent atoms is an absolute minimum. This number is given in angstroms.

Accordingly, structural coordinates of an ErbB2 within the scope of the present invention include structural coordinates related to the atomic coordinates listed in Appendix I by whole body translations and/or rotations. Accordingly, r.m.s deviations listed above assume that at least the backbone atoms of the structures are optimally superimposed which may require translation and/or rotation to achieve the required optimal fit from which to calculate the r.m.s.d.

A three dimensional structure of an ErbB2 protein or region thereof which substantially conforms to a specified set of atomic coordinates can be modeled by a suitable modeling computer program such as MODELER (Sali and Blundell, 1993, J. Mol. Biol., vol. 234:779-815), as implemented in the Insight II Homology software package (Insight II (97.0), MSI, San Diego)), using information, for example, derived from the following data: (1) the amino acid sequence of the human ErbB2 protein; (2) the amino acid sequence of the related portion(s) of the protein represented by the specified set of atomic coordinates having a three dimensional configuration; and, (3) the atomic coordinates of the specified three dimensional configuration. A three dimensional structure of an ErbB2 protein which substantially conforms to a specified set of atomic coordinates can also be calculated by a method such as molecular replacement, which is described in detail below.

Structure coordinates/atomic coordinates are typically loaded onto a machine readable-medium for subsequent computational manipulation. Thus models and/or atomic coordinates are advantageously stored on machine-readable media, such as magnetic or optical media and random-access or read-only memory, including tapes, diskettes, hard disks, CD-ROMs and DVDs, flash memory cards or chips, servers and the internet. The machine is typically a computer.

The structure coordinates/atomic coordinates may be used in a computer to generate a representation, e.g. an image, of the three-dimensional structure of the ErbB2 crystal which can be displayed by the computer and/or represented in an electronic file.

The structure coordinates/atomic coordinates and models derived therefrom may also be used for a variety of purposes such as drug discovery and X-ray crystallographic analysis of other protein crystals.

Design/Selection of Chemical Entities That Bind ErbB2

Using a variety of known modelling techniques, the crystal structure of the present invention can be used to produce a model for at least part of ErbB2.

As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models.

Molecular modelling techniques can be applied to the atomic coordinates of the ErbB2 to derive a range of 3D models and to investigate the structure of binding sites, such as the binding sites of monoclonal antibodies and inhibitory peptides.

These techniques may also be used to screen for or design small and large chemical entities which are capable of binding ErbB2 and modulating the ability of ErbB2 to interact with extracellular biological targets, such as other members of the EGF receptor family e.g. which modulate the ability of ErbB2 to heterodimerise. The screen may employ a solid 3D screening system or a computational screening system.

Such modelling methods are to design or select chemical entities that possess stereochemical complementary to particular regions of ErbB2.

By “stereochemical complementarity” we mean that the compound or a portion thereof makes a sufficient number of energetically favourable contacts with the receptor as to have a net reduction of free energy on binding to the receptor.

Such stereochemical complementarity is characteristic of a molecule that matches intra-site surface residues lining the groove of the receptor site as enumerated by the coordinates set out in Appendix I. By “match” we mean that the identified portions interact with the surface residues, for example, via hydrogen bonding or by non-covalent Van der Waals and Coulomb interactions (with surface or residue) which promote desolvation of the molecule within the site, in such a way that retention of the molecule within the groove is favoured energetically.

It is preferred that the stereochemical complementarity is such that the compound has a K_{d} for the receptor site of less than 10⁻⁴M, more preferably less than 10⁻⁵M and more preferably 10⁻⁶M. In a most preferred embodiment, the K_{d} value is less than 10⁻⁸M and more preferably less than 10⁻⁹M.

Chemical entities which are complementary to the shape and electrostatics or chemistry of the receptor site characterised by amino acids positioned at atomic coordinates set out in Appendix I will be able to bind to the receptor, and when the binding is sufficiently strong, substantially prohibit the interaction of the ErbB2 with biological target molecules such as other EGF receptors.

It will be appreciated that it is not necessary that the complementarity between chemical entities and the receptor site extend over all residues lining the groove in order to inhibit binding of a molecule or complex that naturally interacts with ErbB2.

A number of methods may be used to identify chemical entities possessing stereo-complementarity to a region of the extracellular domain of ErbB2. For instance, the process may begin by visual inspection of potential binding sites, for example, the binding sites for anti-ErbB2 antibodies, on the computer screen based on the ErbB2 coordinates in Appendix I generated from the machine-readable storage medium. Alternatively, selected fragments or chemical entities may then be positioned in a variety of orientations, or docked, within an individual binding site of ErbB2, as defined supra. Modelling software that is well known and available in the art may be used (Guida, W. C. (1994). “Software For Structure-Based Drug Design.” Curr. Opin. Struct. Biology 4: 777-781). These include QUANTA and InsightII [Molecular Simulations, Inc., San Diego, Calif., a division of Pharmacopiea, Inc., Princeton, N.J., 1992], SYBYL [Molecular Modeling Software, Tripos Associates, Inc., St. Louis, Mo., 1992], This modelling step may be followed by energy minimization with standard molecular mechanics force fields such as AMBER [S. J. Weiner, P. A. Kollman, D. A. Case, U. C. Singh, C. Ghio, G. Alagona, and P. Weiner, J. Am. Chem. Soc., vol. 106, pp. 765-784 (1984)], and CHARMM [B. R. Brooks, R. E. Bruccoleri, B. D. Olafson, D. J. States, S Swaminathan, and M. Karplus, J. Comp. Chem. vol. 4, pp. 187-217 (1983)]. In addition, there are a number of more specialized computer programs to assist in the process of selecting the binding moieties of this invention.

Specialised computer programs may also assist in the process of selecting fragments or chemical entities. These include, inter alia:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method. “Proteins: Structure, Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound. In one embodiment, assembly may proceed by visual inspection of the relationship of the fragments to each other on the three-dimensional image displayed on a computer screen in relation to the structure coordinates of ErbB2. This is followed by manual model building using software such as Quanta or Sybyl. Alternatively, fragments may be joined to additional atoms using standard chemical geometry.

The above-described evaluation process for chemical entities may be performed in a similar fashion for chemical compounds.

Useful programs to aid one of skill in the art in connecting the individual chemical entities or fragments include:

1. CAVEAT (Bartlett et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area is reviewed in Martin, “3D Database Searching in Drug Design”, J. Med. Chem., 35, pp. 2145-2154 (1992)).

3. HOOK (available from Molecular Simulations, Burlington, Mass.).

Other molecular modeling techniques may also be employed in accordance with this invention. See, e.g., Cohen et al., “Molecular Modeling Software and Methods for Medicinal Chemistry”, J. Med. Chem., 33, pp. 883-894 (1990). See also Navia and Murcko, “The Use of Structural Information in Drug Design”, Current Opinions in Structural Biology, 2, pp. 202-210 (1992).

There are two preferred approaches to designing a molecule, according to the present invention, that complement the stereochemistry of ErbB2. The first approach is to in silico directly dock molecules from a three-dimensional structural database, to the receptor site, using mostly, but not exclusively, geometric criteria to assess the goodness-of-fit of a particular molecule to the site. In this approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule).

This approach is illustrated by Kuntz et al., 1982, J. Mol. Biol. 161: 269, and Ewing et al., 2001, J. Comput-Aid. Mol. Design 15: 411, the contents of which are hereby incorporated by reference, whose algorithm for ligand design is implemented in a commercial software package, DOCK version 4.0, distributed by the Regents of the University of California and further described in a document, provided by the distributor, which is entitled “Overview of the DOCK program suite” the contents of which are hereby incorporated by reference. Pursuant to the Kuntz algorithm, the shape of the cavity represented by a site on ErbB2 is defined as a series of overlapping spheres of different radii. One or more extant databases of crystallographic data, such as the Cambridge Structural Database System maintained by Cambridge University (University Chemical Laboratory, Lensfield Road, Cambridge, U.K.), the Protein Data Bank maintained by the Research Collaboratory for Structural Bioinformatics (Rutgers University, N.J., U.S.A.), LeadQuest (Tripos Associates, Inc., St. Louis, Mo.), Available Chemicals Directory (Molecular Design Ltd., San Leandro, Calif.), and the NCI database (National Cancer Institute, U.S.A) is then searched for molecules which approximate the shape thus defined.

Molecules identified on the basis of geometric parameters, can then be modified to satisfy criteria associated with chemical complementarity, such as hydrogen bonding, ionic interactions and Van der Waals interactions. Different scoring functions can be employed to rank and select the best molecule from a database. See for example Bohm and Stahl, 1999, M. Med. Chem. Res. 9: 445. The software package FlexX, marketed by Tripos Associates, Inc. (St. Louis, Mo.) is another program that can be used in this direct docking approach (see Rarey, M. et al., J. Mol. Biol. 1996, 261: 470).

The second preferred approach entails an assessment of the interaction of respective chemical groups (“probes”) with the active site at sample positions within and around the site, resulting in an array of energy values from which three-dimensional contour surfaces at selected energy levels can be generated. The chemical-probe approach to ligand design is described, for example, by Goodford, 1985, J. Med. Chem. 28:849, the contents of which are hereby incorporated by reference, and is implemented in several commercial software packages, such as GRID (product of Molecular Discovery Ltd., West Way House, Elms Parade, Oxford OX2 9LL, U.K.).

Pursuant to this approach, the chemical prerequisites for a site-complementing molecule are identified at the outset, by probing the active site with different chemical probes, e.g., water, a methyl group, an amine nitrogen, a carboxyl oxygen, or a hydroxyl. Favoured sites for interaction between the active site and each probe are thus determined, and from the resulting three-dimensional pattern of such sites a putative complementary molecule can be generated. This may be done either by programs that can search three-dimensional databases to identify molecules incorporating desired pharmacophore patterns or by programs which using the favoured sites and probes as input to perform de novo design. Suitable programs for determining and designing pharmacophores include CATALYST (including HypoGen or HipHop) (Molecular Simulations, Inc), and CERIUS2, DISCO (Abbott Laboratories, Abbott Park, Ill.) and ChemDBS-3D (Chemical Design Ltd., Oxford, U.K.).

The pharmacophore can be used to screen in silico compound libraries/three-dimensional databases, using a program such as CATALYST (Molecular Simulations, Inc); MACCS-3D and ISIS/3D (Molecular Design Ltd., San Leandro, Calif.), ChemDBS-3D (Chemical Design Ltd., Oxford, U.K.), and Sybyl/3DB Unity (Tripos Associates, Inc., St. Louis, Mo.).

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), Chemical Abstracts Service (Columbus, Ohio), the Available Chemical Directory (MDL Inc), the Derwent World Drug Index (WDI), BioByteMasterFile, the National Cancer Institute database (NCI), and the Maybridge catalogue.

De novo design programs include LUDI (Biosym Technologies Inc., San Diego, Calif.), Leapfrog (Tripos Associates, Inc.), Aladdin (Daylight Chemical Information Systems, Irvine, Calif.), and LigBuilder (Peking University, China).

Once an entity or compound has been designed or selected by the above methods, the efficiency with which that entity or compound may bind to ErbB2 can be tested and optimised by computational evaluation. For example, a compound that has been designed or selected to function as an ErbB2 binding compound must also preferably traverse a volume not overlapping that occupied by the binding site when it is bound to the native ErbB2. An effective ErbB2 binding compound must preferably demonstrate a relatively small difference in energy between its bound and free states (i.e., a small deformation energy of binding). Thus, the most efficient ErbB2 binding compound should preferably be designed with a deformation energy of binding of not greater than about 10 kcal/mole, preferably, not greater than 7 kcal/mole. ErbB2 binding compounds may interact with ErbB2 in more than one conformation that is similar in overall binding energy. In those cases, the deformation energy of binding is taken to be the difference between the energy of the free compound and the average energy of the conformations observed when the compound binds to the protein.

A compound designed or selected as binding to ErbB2 may be further computationally optimised so that in its bound state it would preferably lack repulsive electrostatic interaction with the target protein.

Such non-complementary (e.g., electrostatic) interactions include repulsive charge-charge, dipole-dipole and charge-dipole interactions. Specifically, the sum of all electrostatic interactions between the compound and the protein when the compound is bound to ErbB2, preferably make a neutral or favourable contribution to the enthalpy of binding.

Once an ErbB2-binding compound has been optimally selected or designed, as described above, substitutions may then be made in some of its atoms or side groups to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analysed for efficiency of fit to ErbB2 by the same computer methods described in detail above.

Specific computer software is available in the art to evaluate compound deformation energy and electrostatic interaction. Examples of programs designed for such uses include: Gaussian 92, revision C (Frisch, Gaussian, Inc., Pittsburgh, Pa.); AMBER, version 4.0 (Kollman, University of California at San Francisco); QUANTA/CHARMM (Molecular Simulations, Inc., Burlington, Mass.); and Insight II/Discover (Biosysm Technologies Inc., San Diego, Calif.).

The screening/design methods may be implemented in hardware or software, or a combination of both. However, preferably, the methods are implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object-oriented programming language to communicate with a computer system. However, the programs can be implemented in assembly or machine language, if desired. In any case, the language may be compiled or interpreted language.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Compounds identified by, or designed by the methods of the invention can be synthetic or naturally occurring, preferably synthetic. In one embodiment, a synthetic compound selected or designed by the methods of the invention preferably has a molecular weight equal to or less than about 5000 or 1000 daltons. A compound selected or designed by methods of this invention is preferably soluble under physiological conditions.

Confirmation of Binding and Biological Activity

Compounds selected or designed in accordance with the in silico methods of the invention may be subjected to further confirmation of binding to ErbB2 by cocrystallization of the compound with ErbB2 and structural determination, as described herein.

Compounds designed or selected according to the methods of the present invention are preferably assessed by a number of in vitro and in vivo assays of ErbB2 function to confirm their ability to interact with and modulate ErbB2 activity. For example, compounds may be tested for their ability to bind to ErbB2 and/or for their ability to modulate e.g. disrupt, heterodimerisation of ErbB2 to other members of the EGF receptor family such as ErbB1, ErbB3 or ErbB4.

Suitable assays include in vitro binding assays and ErbB2-dependent proliferation assays, such as described by Deb et al., 2001, J Biol Chem 276:15554-15560 or Berezov et al., 2001, J. Med. Chem. 44: 2565-2574.

Particular examples of suitable assays are described below.

Inhibition of Heterodimer Formation Between ErbB2 and Other ErbB Family Members

Rationale: While ErB2 is a major oncogenic therapeutic target in its own right, it is now clear that part of the tumour-promoting activity associated with ErbB2 often depends on ligand-induced heterodimer formation with other ErbB family members. There is no known ligand for ErbB2, however ligand binding to other ErbB family members (ErbB1, ErbB3 and ErbB4) causes their heterodimerization with ErbB2. Thus reagents that block this association, for example the ErbB2-specific antibody 2C4, inhibit ligand-stimulated growth in vitro and tumour xenograft in vivo (Agus, D. B. et. al. Cancer Cell 2:127-137). Heterodimerization results in cross-phosphorylation by the ErbB2 kinase of the dimerization partner. In particular, ErbB3 mediated signalling requires heterodimer formation as this particular ErbB family member lacks a functional kinase. Thus, while it is not possible to directly ligand-activate the ErbB2 kinase, it is possible to monitor its activity in cells co-expressing ErbB2 with one or more members of the EGFR family by adding ligands specific to the heterodimerization partners.

Methods: a number of readouts can be used to assess the efficacy, and specificity, of ErbB2 compounds/antibodies in cell-based assays of ligand-induced heterodimer formation. Activity can be assessed by one or more of the following:

(i) Inhibition of ligand-induced heterodimerisation of ErbB2 with other ErbB family members in a target cell line, for example MCF-7 breast cancer cells. Immunoprecipitation of ErbB2 complexes from cell lysates can be performed with a receptor-specific antibody, and the absence/presence of other ErbB receptors and their biologically relevant ligands within the complex can be analysed following electrophoresis/Western transfer by probing with antibodies to other ErbB receptors.

(ii) Inhibition of the activation of signalling pathways by ligand-activated heterodimers. Association with ErbB2 appears critical for other members of the ErbB family of receptors to elicit maximal cellular response following ligand binding. In the case of the kinase-defective ErbB3, ErbB2 provides a functional tyrosine kinase domain to enable signalling to occur following binding of growth factor ligands. Thus, cells co-expressing ErbB2 and ErbB3 can be treated with ligand, for example heregulin, in the absence and presence of inhibitor and the effect on ErbB3 tyrosine phosphorylation monitored by a number of ways including immunoprecipitation of ErbB3 from treated cell lysates and subsequent Western blotting using anti-phosphotyrosine antibodies (see Agus op. cit. for details). Alternatively, a high-throughput assay can be developed by trapping ErbB3 from solubilized lysates onto the wells of a 96-well plate coated with an anti-ErbB3 receptor antibody, and the level of tyrosine phosphorylation measured using, for example, europium-labelled anti-phosphotyrosine antibodies, as embodied by Waddleton, D. et. al. Anal. Biochem. 309:150-157, 2002.

In a broader extension of this approach, effector molecules known to be activated downstream of activated receptor heterodimers, such as mitogen-activated protein kinases (MAPK) and Akt, may be analysed directly, by immunoprecipitation from treated lysates and blotting with antibodies that detect the activated forms of these proteins, or by analysing the ability of these proteins to modify/activate specific substrates.

(iii) Inhibition of ligand-induced cellular proliferation. A variety of cell lines are known to co-express combinations of ErbB receptors, for example many breast and prostate cancer cell lines. Assays may be performed in 24/48/96-well formats with the readout based around DNA synthesis (tritiated thymidine incorporation), increase in cell number (crystal violet staining) etc. However, co-expression of ErbB1 or ErbB4 in such cell lines will mean that it is difficult to determine whether ErbB1 or ErbB4 homodimer signalling is responsible for the proliferative response to ligand.

A new, semi-automated assay system to monitor ErbB2 signalling activity that may be used to confirm the ability of candidate compounds to interact with and modulate ErbB2 activity has been developed. This assay exploits the heterodimerization characteristic of the ErbB family of receptor. We have created a BaF/3 cell line, which normally does not express any members of the ErbB family and is IL-3 dependent, that co-expresses wild-type ErbB2 and a kinase defective (but ligand responsive) ErbB-1 mutant (EGFR-K721R). Upon exposure of the cells to EGF (or other ErbB1 ligand), heterodimer formation occurs leading to phosphorylation of the kinase-defective ErbB1 by the ErbB2 kinase, initiation of the signal transduction pathways downstream of the receptors and ultimately to DNA synthesis. In this experimental system signalling is strictly ligand-dependent but is entirely mediated by the ErbB2 kinase, providing a specific and sensitive assay for inhibitors of ErbB2 heterodimerization. Non-specific toxicity of the test samples is assessed in parallel by testing the cells' responsiveness to IL-3 in the absence of EGF.

Method: BaF/3 cells co-expressing EGFR-K721R and full length wild type ErbB2 are routinely grown in RPMI/10% FCS containing IL-3. Before assay, cells are washed three times to remove residual IL-3 and resuspended in RPMI 1640+10% FCS. Cells are seeded into 96 well plates using a Biomek 2000 (Beckman) at 2×10⁴ cells per 200 μl and incubated for 4 hours at 37° C. in 10% CO₂. Putative ErbB2 inhibitors are added to the first titration point and titrated in two-fold dilutions across the 96 well plate in duplicate with or without a constant amount of EGF (1 nM) or IL-3 (1 μl). ³H-Thymidine (0.5 μCi/well) is added and the plates incubated for 20 hours at 37° C. in 5% CO₂. At the end of the incubation the cells are lysed in 0.5M NaOH at room temperature for 30 minutes then harvested onto nitrocellulose filter mats using an automatic harvester (Tomtec, Conn., USA). The mats are dried, placed in a plastic counting bag and scintillant (10 ml) added. Incorporated 3H-Thymidine is determined using a beta counter (1205 Betaplate, Wallac, Finland).

(iv) Inhibition of growth in soft-agar. This is the benchmark-type assay undertaken to assess anti-tumour activity prior to xenograft studies in animals. Cells are seeded into liquid soft agar cultures, the agar allowed to set, and the appearance of cell colonies monitored over the next 14-21 days. The appearance of colonies in semi-solid media is known as anchorage-independent growth, and is characteristic of the tumour phenotype. Cultures of tumour cell lines can be set up in the presence of both ligand and candidate antagonists of receptor heterodimerisation, and colony growth monitored.

(v) Ability of candidate compounds to block in vivo growth of tumour xenografts of human tumour cell lines whose tumorigenic phenotype is known to be at least partly dependent on ligand activation of ErbB2 heterodimer cell signalling e.g. MCF7 breast cancer cells, LNCap prostate cancer cells etc. This can be assessed in immunocompromised mice either alone or in combination with an appropriate cytotoxic agent for the cell line in question.

Modulation of Ligand-Receptor Interaction

Rationale & method: ErbB2 has no identified ligand of its own, yet in association with other ErbB family members can markedly influence the interaction of its heterodimer partner with ligand.

(i) Heterodimers of ErbB2/3, either on the cell-surface or generated as recombinant fusion proteins using an immunoglobulin Fc domain, bind heregulin with 2-3 orders of magnitude higher affinity than the equivalent ErbB3 homodimers (Jones, J. T. et. al. FEBS Lett. 447:227-231, 1999). Similarly, ErbB4 homodimers do not bind EGF, whereas ErbB2/4 heterodimers do (Jones op.cit.). The heterodimer antagonist antibody 2C4 blocks heregulin binding to cell-surface and Fc fusion heterodimers very efficiently, possibly as a result of steric hindrance through the ligand-binding site, although this remains to be established. This observation suggests that candidate inhibitors of heterodimer association, in particular the ErbB2 CR1 loop-specific antibodies can be tested for activity in this manner. Hence, it is possible to assay in a 96-well format the ability of lead entities (which may or may not be antibodies) to block the binding of tagged ligand, for example europium-labelled EGF, to immobilised ErbB2 hetrodimer combinations, in one example ErbB2/4 Fc fusion proteins, using time-resolved fluorescence as a readout.

(ii) Berezov, A. et. al. J. Biol. Chem. 277: 28330-28339 (2002) describe a screen using the BIAcore whereby small ErbB2 peptide mimetics are used to inhibit heterodimer formation between immobilised ErbB1, 2 or 3 ectodomains and a solution containing ErbB3 ectodomain and ligand (heregulin). The peptides are derived from the C-terminal region of the second cysteine-rich domain of ErbB2.

Molecular Replacement/Binding

The structure coordinates of ErbB2, such as those set forth in Appendix I, can also be used for determining at least a portion of the three-dimensional structure of a molecular complex which contains at least some structural features similar to at least a portion of ErbB2. In particular, structural information about another crystallised molecular complex may be obtained. This may be achieved by any of a number of well-known techniques, including molecular replacement.

Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; Bentley, Meth. Enzym., vol. 276, pp. 611-619, 1997); Lattman, “Use of the Rotation and Translation Functions”, in Meth. Enzymol., 115, pp. 55-77 (1985); and Rossmann, ed., “The Molecular Replacement Method”, Int. Sci. Rev. Ser., No. 13, Gordon & Breach, New York (1972)).

Generally, X-ray diffraction data are collected from the crystal of a crystallised target structure. The X-ray diffraction data is transformed to calculate a Patterson function. The Patterson function of the crystallised target structure is compared with a Patterson function calculated from a known structure (referred to herein as a search structure). The Patterson function of the crystallised target structure is rotated on the search structure Patterson function to determine the correct orientation of the crystallised target structure in the crystal. The translation function is then calculated to determine the location of the target structure with respect to the crystal axes. Once the crystallised target structure has been correctly positioned in the unit cell, initial phases for the experimental data can be calculated. These phases are necessary for calculation of an electron density map from which structural differences can be observed and for refinement of the structure. Preferably, the structural features (e.g., amino acid sequence, conserved di-sulphide bonds, and beta-strands or beta-sheets) of the search molecule are related to the crystallised target structure.

The electron density map can, in turn, be subjected to any well-known model building and structure refinement techniques to provide a final, accurate structure of the unknown crystallised molecular complex (eg see Jones, T. A., Zou, J. Y., Cowan, S. W., and Kjeldgaard (1991). Improved methods for binding protein models in electron density maps and the location of errors in these models. Acta Crystallogr. A 47, 110-119; Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography and NMR system: a new software suite for macromolecular structure determination. Acta Crystallogr. D Biol. Crystallogr. 54, 905-921).

Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

By using molecular replacement, all or part of the structure coordinates of ErbB2 provided herein (and set forth in Appendix) can be used to determine the structure of a crystallised molecular complex whose structure is unknown more rapidly and efficiently than attempting to determine such information ab initio. This method is especially useful in determining the structure of ErbB2 mutants and homologues.

The structure of any portion of any crystallised molecular complex that is sufficiently homologous to any portion of the extracellular domain of ErbB2 can be solved by this method.

Such structure coordinates are also particularly useful to solve the structure of crystals of ErbB2 co-complexed with a variety of molecules, such as other EGF receptor family receptors to which ErbB2 dimerises, or chemical entities. For example, this approach enables the determination of the optimal sites for the interaction between chemical entities, and the interaction of candidate ErbB2 agonists or antagonists.

All of the complexes referred to above may be studied using well-known X-ray diffraction techniques and may be refined versus 1.5-3.5 A resolution X-ray data to an R value of about 0.25 or less using computer software, such as X-PLOR (Yale University, distributed by Molecular Simulations, Inc.; see Blundell & Johnson, supra; Meth. Enzymol., vol. 114 & 115, H. W. Wyckoff et al., eds., Academic Press (1985)). This information may thus be used to optimize known ErbB2 agonist/antagonists, such as anti-ErbB2 antibodies, and more importantly, to design new or improved ErbB2 agonists/antagonists.

Production of ErbB2 Crystals

The crystals of the present invention may be prepared by expressing a nucleotide sequence encoding ErbB2 or a variant thereof in a suitable host cell, and then crystallising the purified protein(s). Preferably the ErbB2 polypeptide contains the extracellular domain (amino acids 1 to 632 of the mature human polypeptide or a truncated version thereof, preferably comprising amino acids 1 to 509, or the equivalent residues in other ErbB2 polypeptides) but lacks the transmembrane and intracellular domains. Preferred host cells are those that provide for reduced glycosylation of recombinant polypeptides, such as a glycosylation-defective mammalian cell line e.g. the Lec8 Chinese hamster cell line, a derivative of CHO-K1 fibroblasts (ATCC CRC:1737) (Stanley, 1989, Mol. Cell Biol. 9: 377-383).

ErbB2 polypeptides may also be produced as fusion proteins, for example to aid in extraction and purification. Examples of fusion protein partners include glutathione-S-transferase (GST), hexahistidine, GAL4 (DNA binding and/or transcriptional activation domains) and beta-galactosidase. It may also be convenient to include a proteolytic cleavage site between the fusion protein partner and the protein sequence of interest to allow removal of fusion protein sequences.

After expression, the proteins may be purified and/or concentrated, for example by immobilised metal affinity chromatography, ion-exchange chromatography, and/or gel filtration.

The protein(s) may be crystallised using techniques described herein. Usually, in a crystallisation process, a crystallisation buffer is prepared with a lower concentration of a precipitating agent necessary for crystal formation. For crystal formation, the concentration of the precipitating agent has to be increased, by addition of precipitating agent or by diffusion of the precipitating agent between the crystallisation buffer and a reservoir buffer. Diffusion may be achieved by known techniques such as the “hanging drop” or the “sitting drop” method. In these methods, a drop of crystallisation buffer containing the protein (s) is hanging above or sitting beside a much larger pool of reservoir buffer. Alternatively, the balancing of the precipitating agent can be achieved through a semi-permeable membrane that separates the crystallisation buffer and prevents dilution of the protein into the reservoir buffer.

We have found that the inclusion of about 15% PEG 1500 provides optimal crystallization conditions for the extracellular domain of human ErbB2.

Generating the Crystal Structure

Once the crystals have been obtained, the structure may be solved by known X-ray diffraction techniques. Many techniques use chemically modified crystals, such as those modified by heavy atom derivatization. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can then be determined by X-ray diffraction analysis of the soaked crystal. The patterns obtained on diffraction of a monochromatic beam of X-rays by the atoms (scattering centres) of the crystal can be solved by mathematical equations to give mathematical coordinates. The diffraction data are used to calculate an electron density map of the repeating unit of the crystal. The electron density maps are used to establish the positions of the individual atoms within the unit cell of the crystal (Blundel, T. L. and N. L. Johnson, Protein Crystallography, Academic Press (1976)).

Target Binding Sites for Modulators of ErbB2

The three-dimensional structure of ErbB2 provided herein allows the identification of target binding sites for potential ErbB2 modulators.

Preferred target binding sites are those involved in heterodimerisation of ErbB2 with other members of the EGF receptor family, such as ErbB1, ErbB3 and/or ErbB4.

One preferred binding site involved in heterodimerisation is the CR1 dimerisation loop (residues 247-268) and adjacent residues (residues 244-246, 285-289). Other suitable binding sites include the N-terminal end of the CR1 domain (residues 200-203, 210-213, 216-218, 225-230), and the C-terminal end of the CR1 domain (residues 294-319).

In a further preferred embodiment, the binding site is the docking site on ErbB2 for the CR1 dimerisation loop of heterodimer partners. This docking site is located on ErbB2 between the L1, CR1 and L2 domains. Preferably, the docking site comprises the following ErbB2 residues: Gln 36, Gln 60, Arg 82, Thr 84, Gln 85, Phe 237, Thr 269, Phe 270, Gly 271, Ala 272, Tyr 282, Thr 285, Gly 288, Ser 289, Cys 290, Thr 291, Leu 292, Val 293, Cys 294, Pro 295 and Cys 310.

In yet another preferred embodiment, the target binding site is located on the L1 or L2 domains. Unlike the unligated structure of ErbB3 (Cho, H. S. & Leahy, D. J. Science 297, 1330-1333 (2002)) or the pseudo-unligated structure of EGFR (Ferguson et al., Molecular Cell, Vol. 11, 507-517, (2003)), the structure of ErbB2 exists in a conformation similar to that of the 2:2 ligand-receptor dimer. This is in large part maintained by the L1:L2 contact, as described in Garrett, et al., Molecular Cell, Vol. 11, 495-505. Thus a small molecule or antibody which binds to either the L1 or L2 domain or intercalates between them can modulate receptor dimer formation by either preventing the domains from binding to each other or by modifying the relative positions of the domains. Thus binding of a chemical entity to the L1 and/or L2 domain may cause the protein to adopt a conformation similar to that of its unligated relatives (EGFR or ErbB3) and thereby inhibit dimerisation. Alternatively, binding of a chemical entity to the L1 and/or L2 domain may cause modifications in the CR1 (dimerisation domain) as described in Garrett, et al., Molecular Cell, Vol. 11, 495-505 to inhibit receptor dimer formation. The relevant binding sites of the L1 or L2 domain consist of the atoms of either one of these domains that lie within about 4.5 Angstroms of the other domain.

Antibodies

The term “antibody” as used in this invention includes intact molecules as well as fragments thereof, such as Fab, F(ab′)2, and Fv which are capable of binding the epitopic determinant. These antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

Antibodies of the present invention may be produced, for example, by immunizing mice with purified ErbB2 fragment 1-509. After determining that the mice are producing anti-ErbB2 antibodies, hybridomas may be prepared and antibody specificity assayed by ELISA or Flow Cytometry using two cell lines: Baf/wt-EGFR cells and Baf/EGFR-“mutation x” cells. These mouse cell lines express either the wild type ErbB2 or the ErbB2 containing an amino acid substitution, for example an Ala substitution (ie mutation x), within the specific site against which the antibody is to be directed. When hybridomas secreting antibodies which recognize Baf/wt-ErbB2, but not Baf/ErbB2-“mutant x” are identified, the corresponding hybridoma may be cloned and the monoclonal antibody purified.

Alternatively, in raising antibodies of the invention, it may be desirable to use derivatives of the peptides or loop structures which are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure. For example, amino acids adjacent to or flanking the ErbB2 loop structures may be included in the construct to maintain conformation of the peptide used to raise antibodies.

In addition, the active conformation of the peptide may be stabilized by a covalent modification, such as cyclization or by incorporation of gamma-lactam or other types of bridges. For example, side chains can be cyclized to the backbone so as create a L-gamma-lactam moiety on each side of the interaction site. See, generally, Hruby et al., “Applications of Synthetic Peptides,” in Synthetic Peptides: A User's Guide: 259-345 (W.H. Freeman & Co. 1992). Cyclization also can be achieved, for example, by formation of cystine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken. See Wood and Wetzel, 1992, Int'l J. Peptide Protein Res. 39: 533-39.

Further the conformation of the peptide analogues may be stabilised by including amino acids modified at the alpha carbon atom (eg. α-amino-150-butyric acid) (Burgess and Leach, 1973, Biopolymers 12(12):2691-2712; Burgess and Leach, 1973, Biopolymers 12(11):2599-2605) or amino acids which lead to modifications on the peptide nitrogen atom (eg. sarcosine or N-methylalanine) (O'Donohue et al, 1995, Protein Sci. 4(10):2191-2202).

Another approach described in U.S. Pat. No. 5,891,418 is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred.

Peptides and Peptidomimetics

In yet a further aspect the present invention provides an isolated conformationally constrained peptide or peptidomimetic consisting essentially of (i) ErbB2 amino acid residues 200-203, (ii) ErbB2-amino acid residues 210-213, (iii) ErbB2 amino acid residues 216-218, (iv) ErbB2 amino acid residues 225-230, (v) ErbB2 amino acid residues 247-268 or a subset thereof; (vi) ErbB2 amino acid residues 244-246, (vii) ErbB2 amino acid residues 285-289, or (viii) ErbB2 amino acid residues 294-319 or a subset thereof.

The term “conformationally constrained molecules” means conformationally constrained peptides and conformationally constrained peptide analogues and derivatives.

The term “analogues” refers to molecules having a chemically analogous structure to the naturally occurring alpha-amino acids present in ErbB2. Examples include molecules containing gem-diaminoalkyl groups or alklylmalonyl groups.

The term “derivatives” includes alpha amino acids wherein one or more side groups found in the naturally occurring alpha-amino acids present in ErbB2 have been modified. Thus, for example the naturally-occurring amino acids present in ErbB2 may be replaced with a variety of uncoded or modified amino acids such as the corresponding D-amino acid or N-methyl amino acid. Other modifications include substitution of hydroxyl, thiol, amino and carboxyl functional groups with chemically similar groups.

The present invention encompasses the use of conformationally constrained peptidomimetics of fragments of ErbB2 (such as amino acid residues 247-268), i.e. analogues and derivatives which mimic the activity of ErbB2 and are therefore capable of modulating ErbB2 activity in vivo. These peptidomimetics are preferably substantially similar in three-dimensional shape to the peptide structures (for example, loop structures) as they exist on the native ErbB2. Substantial similarity means that the geometric relationship of groups in the ErbB2 peptide fragment is preserved such that the peptidomimetic will mimic the activity of ErbB2 in vivo.

A “peptidomimetic” is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Whether completely or partially non-peptide, peptidomimetics for use in the methods of the invention provide a spatial arrangement of reactive chemical moieties that closely resembles the three-dimensional arrangement of active groups in the peptide on which the peptidomimetic is based. As a result of this similar active-site geometry, the peptidomimetic has effects on biological systems which are similar to the biological activity of the peptide.

There are clear advantages for using a mimetic of a given peptide rather than the peptide itself, because peptides commonly exhibit two undesirable properties: (1) poor bioavailability; and (2) short duration of action. Peptide mimetics offer an obvious route around these two major obstacles, since the molecules concerned are small enough to be both orally active and have a long duration of action. There are also considerable cost savings and improved patient compliance associated with peptide mimetics, since they can be administered orally compared with parenteral administration for peptides. Furthermore, peptide mimetics are much cheaper to produce than peptides.

Suitable peptidomimetics based on, for example, residues 247-268, can be developed using readily available techniques. Thus, for example, peptide bonds can be replaced by non-peptide bonds that allow the peptidomimetic to adopt a similar structure, and therefore biological activity, to the original peptide. Further modifications can also be made by replacing chemical groups of the amino acids with other chemical groups of similar structure. The development of peptidomimetics derived from ErbB2 peptides based on residues 247-268 can be aided by reference to the three dimensional structure of these residues as provided in Appendix I. This structural information can be used to search three-dimensional databases to identify molecules having a similar structure, using programs such as MACCS-3D and ISIS/3D (Molecular Design Ltd., San Leandro, Calif.), ChemDBS-3D (Chemical Design Ltd., Oxford, U.K), and Sybyl/3DB Unity (Tripos Associates, St. Louis, Mo.).

Those skilled in the art will recognize that the design of a peptidomimetic may require slight structural alteration or adjustment of a chemical structure designed or identified using the methods of the invention. In general, chemical compounds identified or designed using the methods of the invention can be synthesized chemically and then tested for ability to modulate ErbB2 activity using any of the methods described herein. The methods of the invention are particularly useful because they can be used to greatly decrease the number potential mimetics which must be screened ability to modulate ErbB2 activity.

The peptides or peptidomimetics of the present invention can be used in assays to screening for candidate compounds which bind to regions of ErbB2 and potentially interfere with the hereodimerisation of ErbB2 with another member of the EGF receptor family.

Standard solid-phase ELISA assay formats are particularly useful for identifying inhibitors of dimerisation. In accordance with this embodiment, the peptide or peptidomimetic of the invention is immobilized on a solid matrix, such as, for example an array of polymeric pins or a glass support. Conveniently, the immobilized peptide or peptidomimetic is a fusion polypeptide comprising Glutathione-S-transferase (GST; e.g. a CAP-ERK fusion), wherein the GST moiety facilitates immobilization of the protein to the solid phase support. This assay format can then be used to screen for candidate compounds that bind to the immobilised peptide or peptidomimetic and/or interefere with binding of a natural binding partner of ErbB2 to the immobilised peptide or peptidomimetic.

Uses of Modulators of ErbB2

Compounds/chemical entities designed or selected by the methods of the invention described above may be used to modulate ErbB2 activity in cells, i.e. activate or inhibit ErbB2 activity. In particular, they may be used to modulate the interaction between ErbB2 and other heterodimerisation partners of the EGF receptor family, such as ErbB1, ErbB2 and ErbB4.

Modulation of heterodimerisation between ErbB2 and other members of the EGF receptor family may be achieved by direct binding of the chemical entity to a heterodimerisation surface of ErbB2 and/or by an allosteric interaction elsewhere in the ErbB2 extracellular domain.

Given that aberrant EGF/ErbB2 activity is implicated in a range of disorders, the compounds described above may also be used to treat, ameliorate or prevent disorders characterised by abnormal ErbB2 signalling. Examples of such disorders include malignant conditions including tumours of the brain, head and neck, prostate, ovary, breast, cervix, lung, pancreas and colon; and melanoma, rhabdomyosarcoma, mesothelioma, squamous carcinomas of the skin and glioblastoma.

Administration

Compounds of the invention, i.e. antibodies of the invention or modulators of ErbB2 identified or identifiable by the screening methods of the invention, may preferably be combined with various components to produce compositions of the invention. Preferably the compositions are combined with a pharmaceutically acceptable carrier or diluent to produce a pharmaceutical composition (which may be for human or animal use).

The formulation will depend upon the nature of the compound and the route of administration but typically they can be formulated for topical, parenteral, intramuscular, oral, intravenous, intra-peritoneal, intranasal inhalation, lung inhalation, intradermal or intra-articular administration. The compound may be used in an injectable form. It may therefore be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The compounds of the present invention may be admixed with any suitable binder(s), lubricant(s), suspending agent(s), coating agent(s), solubilising agent(s). It is also preferred to formulate the compound in an orally active form.

In general, a therapeutically effective daily oral or intravenous dose of the compounds of the invention, including compounds of the invention and their salts, is likely to range from 0.01 to 50 mg/kg body weight of the subject to be treated, preferably 0.1 to 20 mg/kg. The compounds of the invention and their salts may also be administered by intravenous infusion, at a dose which is likely to range from 0.001-10 mg/kg/hr.

Tablets or capsules of the compounds may be administered singly or two or more at a time, as appropriate. It is also possible to administer the compounds in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

The compositions (as well as the compounds alone) can also be injected parenterally, for example intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the compounds of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time, as appropriate. As indicated above, the physician will determine the actual dosage which will be most suitable for an individual patient and it will vary with the age, weight and response of the particular patient.

The routes of administration and dosages described are intended only as a guide since a skilled practitioner will be able to determine readily the optimum route of administration and dosage for any particular patient depending on, for example, the age, weight and condition of the patient.

The present invention will now be described further with reference to the following examples, which are illustrative only and non-limiting. The examples refer to the figures:

### EXAMPLES

### Experimental Procedures

Construction of the ErbB2-509 Expression Vector

An ErbB2 cDNA clone encompassing the entire coding region in the expression vector pRc/CMV (Invitrogen) was a gift from Dr. Rod Fiddes (AMBRI Pty. Ltd.). A Hind III/EcoR 1 fragment spanning the 5′-non-coding region and nucleotides encoding amino acids 1-412 was isolated and cloned into pUC19 (Pharmacia). A 324 basepair EcoR1 fragment incorporating amino acids 413-509 of ErbB2 and a C-terminal FLAG epitope (Brizzard et al., 1994, Biotechniques 16, 730-735) was generated by the polymerase chain reaction (PCR) using the primers 5′-CGGACAGCCTGCCTGACCTC-3′ (upstream) and 5′-CCGGAATTCTAGACTACTTATCATCGTCATCTTTGTAATCGTTGACACA CTGGGTGGGC-3′, and cloned into the EcoR 1 site of this plasmid. This plasmid was further modified by replacement of the 5′ Hind III/BamH I of ErbB2 with a truncated Hind III/BamH I fragment, corresponding to nucleotides 171-1170 (GenBank accession number X03363), generated by PCR using the primers 5′-GGGGAAGCTTGCCACCATGGAGCTGGCGGCC-3′ (upstream) and 5′-GCTGCACTTCTCACACCGCTG-3′ (downstream). The fidelity of all amplification products was established by nucleotide sequencing. The modified ErbB2 cDNA insert was subsequently excised as a Hind III/Xba I fragment and cloned into the corresponding restriction sites of the mammalian expression vector pEE14 (Bebbington and Hentschel, 1987, In: DNA Cloning (Glover, D., ed.), Vol. 1, pp. 163-188, IRL Press, Oxford, U.K) to generate pESE.ErbB2-509.

Cell Culture and Transfection

The Lec8 Chinese hamster cell line, a derivative of CHO-K1 fibroblasts was obtained from the American Tissue Culture Collection (ATCC CRC:1737) and maintained in Glasgow's modified Eagle's medium (Life Technologies) supplemented with 10% fetal calf serum (FCS). Cells were transfected with pESE.ErbB2-509 that had been linearised by digestion with Fsp I, using FuGENE (Roche Molecular Biochemicals) according to the manufacturer's instructions. Stable transfectants were isolated by culturing cells in glutamine-free medium containing 10% dialysed FCS and 25 μM methionine sulfoximine. Supernatants were screened by dot-blotting onto nitrocellulose and probing with the anti-FLAG monoclonal antibody, M2 (Brizzard et al., 1994).

Protein Production and Purification

A positive polyclonal culture was used for scale-up protein production by growing the cells in roller bottles, during which time they were adapted to DMEM/F12 (JRH) media, supplemented with 10% dialysed FCS (Life Techologies) and 25 uM methionine sulfoximine. After verifying the yield and quality of the ErbB2-509 fragment, four 500 ml spinner flasks, each containing 10 g of FibraCell disks (New Brunswick Scientific), were inoculated with harvested cells from eight confluent roller bottles. Over a period of three weeks, spent media was collected daily from the spinner flasks and replaced with fresh media Undialysed serum (CSL) was used instead of dialysed serum after day three. Approximately 30 litres of media harvest was collected over three weeks.

ErbB2-509 FLAG-tagged protein was purified by immunoaffinity chromatography over a 50 ml column of M2 anti-FLAG antibody covalently coupled to Mini Leak Low (Kem-En-Tek Denmark) as per manufacturer's instructions. Batches of four to six litres of culture media at 4° C. were passed over the column at 100-200 ml/h and washed with ˜20 column volumes of 40 mM Tris-buffered saline at pH8/0.02% sodium azide (TBSA). FLAG-tagged protein was eluted from the column after 90 min of recirculating 50 ml of a 0.25 mg/ml solution of the FLAG peptide DYKDDDDK in TBSA, followed by elution with three to four column volumes of 0.1 mg/ml FLAG peptide in TBSA. The affinity column was regenerated with 0.1M sodium citrate pH 3 before re-equilibration at pH 8 with TBSA, ready for the next batch of harvest. Further purification was effected by passing a concentrated solution of the peptide-eluted product over a Superdex 200 column (Pharmacia 26/60) in TBSA at 5 ml/min. Greater than 90% of the 280 nm-absorbing material eluted as a single symmetrical peak of apparent mass ˜70 kDa, at a yield of 1-2 mg/L of the spinner-flask harvest. The peak fraction was buffer-exchanged into 10 mM HEPES pH7.5 and concentrated to 8 mg/ml.

Crystallization and Data Collection

Crystallization trials were performed with a factorial screen (Jancarik and Kim, 1991, J. Appl. Cryst. 24, 409-411) using the hanging drop method. Initially, rod-shaped crystals grew within 4 days, which diffracted to ˜3.5 Å. However, after further crystallization trials the best conditions were 15% PEG 1500 and the resolution extended to 2.5 Å. Crystals (space group P2₁2₁2₁, a=75.96, b=82.24, c=110.06 Å) were cryo-cooled to −170° C. in 20% PEG, 20% glycerol. Diffraction data were recorded as 192 1° exposures on a Rigaku RAXIS IV area detector using RU-300 Rigaku generator equipped with elliptical glass capillary optics (AXCO). Data were integrated to 2.5 Å and scaled using the DENZO/SCALEPACK (mosaic spread 0.8°, R_{sym}=0.103, multiplicity 7.2, completeness 97.2%)

Structure Solution and Refinement

The structure was solved by molecular replacement with AMORE using data 10-4 Å resolution and two fragments of EGFR (residues 4-238 and 310-500) as search models. In both rotation and translation functions the highest peaks corresponded to the correct solution. By inspection of electron density maps (10-3.5 Å resolution) with O an initial model of ErbB2 was constructed from the structure of EGFR. This model consisted of 472 of 510 residues, including 91 side chains truncated from the EGFR equivalent. Structure refinement was performed with CNS (Brunger et al., 1998, Acta Crystallogr. D Biol Crystallogr. 54, 905-921). Initially, rigid body refinement with four groups (residues 1-194, 197-310, 318-510) gave R=0.473, R_{free}=0.482 (5% of the data). Nine rounds of manual refitting were alternated with energy minimisation, B factor refinement and, sometimes, simulated annealing. The resolution was extended in a stepwise manner with a bulk solvent correction applied from round 3 and an overall anisotropic thermal parameter from round 6. The final model contains 506 amino acids, 4 carbohydrate residues and 134 solvent molecules, giving R=0.226, R_{free}=0.264 (data 25-2.5 Å). For residues 1-2, 100-102 and 107-113 294-318 the electron density is weak and there is no density for residues 103-106 or beyond residue 510.

Database Preparation

Databases were generated using information provided by the Supplier, or the NIH developmental therapeutics program. The NCl database was built from the October 2000 release, and the Tripos Leadquest database using the October 2001 release. SDF records were converted into 3-dimensional Sybyl mol2 files using the dbtranslate utility from UNITY environment in sybyl6.7, coordinates were generated using Concord 4.0.2 and the atom typing of resulting mol2 files corrected using our in house tool Mol-prepare. The resulting mol2 files were then protonated, assigned Gasteiger-Huckel charges and minimized (conjugate gradient for a maximum of 500 iterations) using Sybyl 6.7. Databases were then indexed for our database server program.

Assay for Determining ErbB2 Kinase Activity

BaF/3 cells co-expressing K721R-ErbB1 and wtErbB2 are routinely grown in RPMI/10% FCS containing IL-3. Before assay, cells are washed three times to remove residual IL-3 and resuspended in RPMI 1640+10% FCS. Cells are seeded into 96 well plates using a Biomek 2000 (Beckman) at 2×10⁴ cells per 200 μl and incubated for 4 hours at 37° C. in 10% CO₂. Candidate ErbB2 inhibitors are added to the first titration point and titrated in two-fold dilutions across the 96 well plate in duplicate with or without a constant amount of mEGF (1 nM) or IL-3 (1 μl). 3^{H}-Thymidine (0.5 μCi/well) is added and the plates incubated for 20 hours at 37° C. in 5% CO₂. At the end of the incubation the cells are lysed in 0.5M NaOH at room temperature for 30 minutes then harvested onto nitrocellulose filter mats using an automatic harvester (Tomtec, Conn., USA). The mats are dried, placed in a plastic counting bag and scintillant (10 ml) added. Incorporated 3H-Thymidine is determined using a beta counter (1205 Betaplate, Wallac, Finland).

### Example 1

### Description of Structure

The ErbB2 fragment described here comprises the L1, CR1 and L2 domains plus the first module (residues 489-509) from the second cys-rich region CR2. The crystals contained only one molecule of the truncated ErbB2 ectodomain in the asymmetric unit and showed no evidence of dimers. ErbB2 (residues 1-509) adopts a compact bilobed structure reminiscent of the closed conformation of the EGFR ectodomain in its 2:2 complexes with TGFα (Garrett et al., 2002) or EGF (Ogiso et al., 2002, Cell 110, 775-787), but very different from the open conformations seen in the unliganded, full length ErbB3 ectodomain (Cho and Leahy, 2002) or the truncated L1/CR/L2 fragment of the closely related type 1 insulin-like growth factor receptor (Garrett et al., 1998, Nature 394, 395-399).

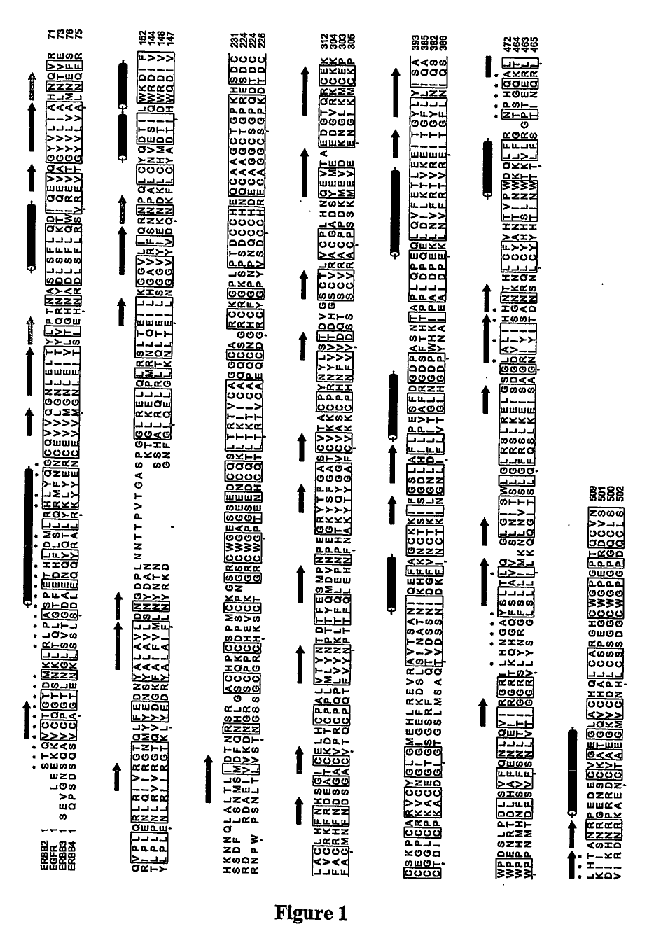

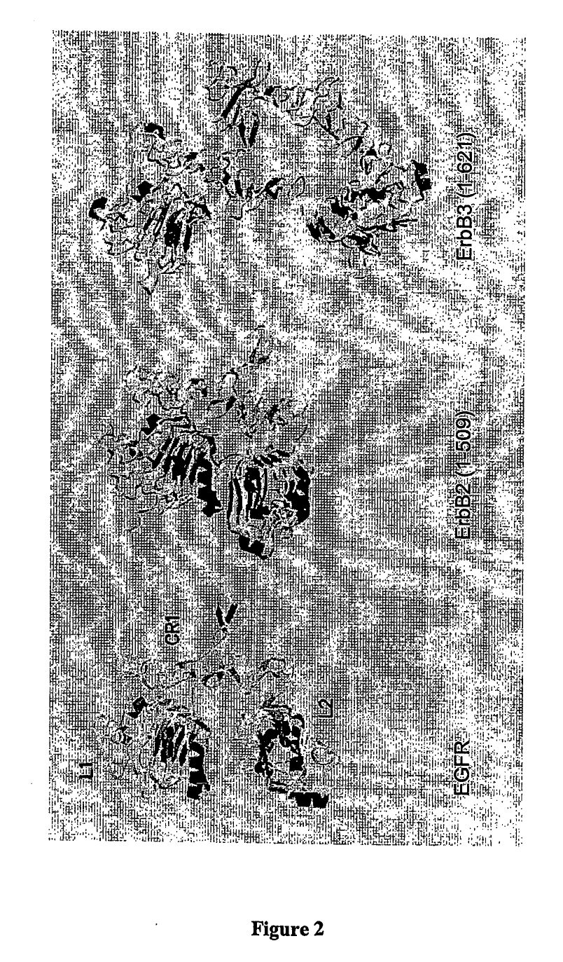

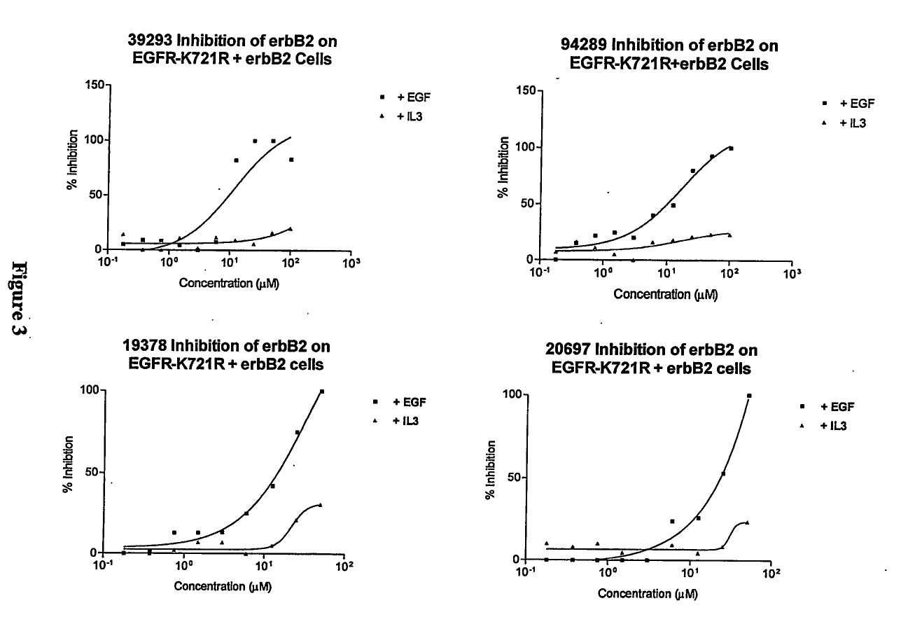

The main chain conformation of each L domain is similar to the corresponding domains of EGFR with the rmsd for the Cα atoms of L1 being 1.14-1.21 (for >91% of the Cα atoms) and for the Cα atoms of L2 being 0.97-1.05 Å (96%). In the ErbB2 L1 domain, the V-shaped region (residues 9-17), which forms a substantial part of the ligand-binding surface in EGFR, is maintained. However there is a small shift in the position of the N-terminal helix (residues 17-30) in ErbB2 and minor differences in residues equivalent to those in EGFR that make a main chain contact with TGFα (Garrett et al., 2002). The position of the large insertion (residues 101-109) specific to ErbB2 (FIG. 1) is in the loop of EGFR (residues 101-106) in the fourth repeat at the corner of the second and third β-sheets of the L1 domain and is predominantly disordered in ErbB2. In the ErbB2 L2 main chain, small movements are seen in the two loops (residues 324-334 and 360-374) equivalent to those that bind ligand in EGFR (Garrett et al., 2002) and in the relative position of the single cys rich module (residues 489-509) that follows the L2 domain.

While the folds of the two L domains are similar in ErbB2 and the EGFR/TGFα complex (Garrett et al., 2002) the relative orientation of these two domains are quite different (FIGS. 3 and 4). This is due to differences in the CR1 domain and the CR1-L2 hinge of ErbB2 which direct the two L domains towards each other, where they make substantial contact (the total accessible surface area buried is 1264 Å² and shape complimentarily, S_{c}=0.63). The overall movement of the ErbB2 L2 domain, with respect to L1, corresponds to a rotation of about 35° (A 37.4°, B 31.8°) around an axis parallel to strands of the L2 large β-sheet and a translation of 7 Å towards CR1 so that in ErbB2 the bottom of the large sheet on L2 sits against the N-terminal end (residues 1-33) of L1. In this conformation an EGF-like ligand cannot bind to sites on either the L1 or L2 domains of ErbB2 (as seen for EGFR) since each site is occluded by the opposing L domain.

Finding ErbB2 in this “closed” form raises the question of whether this could occur in the unligated form of EGFR (or ErbB3/ErbB4). Structural superposition is not straightforward as there are main chain rearrangements in this region of ErbB2, namely a shift in the N-terminal helix of L1 by about 1.8 Å, possibly due to Leu22 of ErbB2 being replaced by an aromatic side chain in ErbB1 (F24), ErbB3 (Y27) and ErbB4 (Y24) (FIG. 1). However, even when superimposing residues 9-17 and the helix separately then comparing with superpositions of L2, complementarity in these regions for other members of the family was not observed. In EGFR, Gln411 is equivalent to Ala419 of ErbB2 and the bulky Gln side chain could not be easily accommodated in the ErbB2 structure as it would sterically clash with Ser26 and Met30 (Met24 and Leu28 in ErbB2). This closed conformation would not pose a problem for ErbB3/ErbB4 where the residues corresponding to ErbB2 Ala419 are Gly residues (FIG. 1). Asn12 in EGPR is a key residue for ligand binding and is strictly conserved in all the EGFR family except ErbB2. If the unliganded form of EGFR were to have the same conformation as ErbB2 then Asn12 (equivalent to Met10 in ErbB2) would sterically clash with His409 (Asn417 in ErbB2) and the side chains of Lys463 and Lys465 (equivalent to the hydrophobic residues Ala471 and Leu473 of ErbB2) on the last strand of the major β-sheet of L2, would overlap with Arg29 and Asp22, respectively. In addition to the steric clash, electrostatic repulsion may also be important as residues 29 and 463 are basic (Lys/Arg) in EGFR/ErbB3/Erb4 but are His and Ala in ErbB2, respectively. Thus it appears that the “closed” conformation seen for domains 1-3 of ErbB2 is unlikely to be a general feature of this receptor family but unique to the ErbB2 molecule.

### Example 2

### Analysis of the Ligand Binding Region

A comparison of the ErbB2 and EGFR structures shows why ErbB2 does not bind ligands such as EGF, TGFα or the neuregulins. The L domains of EGFR, together with the ligand, TGFα, were superimposed on the corresponding L domains of ErbB2 using the strands of the large β-sheet. Residues of ErbB2 L1 which would interfere with ligand binding are Arg13 (replacing Thr, Ser and Ser in ErbB1, ErbB3 and ErbB4) and Pro15 (replacing Leu, Thr and Leu in ErbB1, ErbB3 and ErbB4) on the short N-terminal strand of the L1 domain (FIG. 1). In EGFR, residues 13-15 form a β-sheet with the ligand. The presence of Arg13 alone is likely to prevent ligand binding as this residue lies at the heart of the interface. Unless the receptor side chains are small there is no room for ligand side chains. Another crucial residue in EGFR is Asn12, the side chain of which makes two hydrogen bonds to the ligand's main chain. Asn is present in EGFR, ErbB3 and ErbB4 but in ErbB2 the equivalent residue (Met10) is buried between Val8 and Pro15 and unavailable for ligand interactions. Another residue in the L1 domain which would interfere with EGF-related ligand binding by ErbB2 is Asp98, which is Ser or Leu in the other ErbB family members (FIG. 1) and would clash with Glu27 of TGFα

Observations by Kohda et al., 1993 (J. Biol. Chem. 268, 1976-1981) indicate that ligands can bind to L2 alone albeit with low affinity. For the L2 domain the differences between ErbB2 and the other ErbB receptors are more subtle. Asp355 of EGFR, which makes a salt bridge with the highly conserved Arg42 of TGFα (Garrett et al., 2002), is conserved for all EGFR homologues including ErbB2. However, in ErbB2 movement of residues 324-334 in a neighbouring loop appears to disturb the position of this residue (Asp363). Other residues in the L2 binding site of EGFR such as His346 and Gln384 are smaller in ErbB2 (Ala354 and Ser392), so binding to ErbB2 would be expected to be of lower affinity.

The ligand-binding surfaces of the EGFR homologues are by no means well conserved and each ErbB receptor has its own ligand binding characteristics. ErbB3 and ErbB4 predominantly bind the neuregulin group. Again, ErbB2 fails to interact with this subfamily of ligands and the residues of ErbB2 at positions equivalent to the EGFR ligand binding surface clearly disrupt the L1 and L2 binding surface (FIG. 1).

### Example 3

### Differences in CR1

In the TGFα:EGFR complex, the dominant feature of CR1 is a large loop (residues 242-259) which extends out from the rod-shaped CR1 and plays a key role in homo-dimerisation and signaling for that receptor. This loop contains only limited sequence homology with the other EGFR homologues (33-44%) and it was not clear whether dimerisation of the receptor influenced the conformation of this loop. In the crystal, ErbB2 is present as a monomer and the CR1 loop projects out into solvent, lying against an adjacent molecule in a crystal contact. Superposition of this loop from ErbB2 and EGFR shows that the main chain of the ErbB2 loop adopts a very similar structure to that found in the EGFR dimer (rmsd 0.61 A for 15 Cα's) with small differences seen only at the tip. Thus it seems that this loop has a well defined conformation even in the undimerised state. Residues important in maintaining this structure are prolines found at various positions in different homologues, particularly Pro257 (EGFR), and two completely conserved asparagines (residues 247 and 256 in EGFR) which make hydrogen-bonded contacts with main chain atoms. In ErbB2 this loop is bent slightly (12-13°) relative to the corresponding fifth cys rich module in EGFR.

Overall comparison of the CR1 domain shows that, relative to EGFR, it does not bend smoothly, rather it bends locally at three places. For individual modules rms deviations in Cα positions are less than 1 Å while for the whole domain the rms deviation is 1.7/1.8 Å (106/98 of 117 residues). Cys rich modules 2-4 lie similarly against L1 in both ErbB2 and EGFR and the bends occur at the interfaces of the fourth and seventh modules. The most obvious bend is in the middle of CR1 between the fourth and fifth modules (21/25°) but other differences (between the fifth and sixth modules 11°, between the sixth and seventh modules 15/27°), together with a bend of 37/32° between the seventh module and the L2 domain constitute the set of changes which reorientate L2 with respect to L1.

### Example 4

### Implications for Dimerisation

The rearrangements in CR1 have three effects on the dimer interface as seen in EGFR and the capacity of ErbB2, in this conformation, to form heterodimers with a 1:1 complex of EGFR with ligand. Superposing the fifth cys-rich module from CR1 of ErbB2 on one half of the EGFR dimer, the bend at the fourth and fifth modules of ErbB2 causes the N-terminal tip of ErbB2 to move away from the corresponding region on the other molecule, removing that region from the back-to-back contact. The bend at cys-rich modules 6 and 7 of ErbB2 would bring module 8 in contact with module 7 of EGFR. More significant, however, is that the bend at the fourth and fifth modules of ErbB2 brings the ErbB2 L1domain closer to the tip of the partner's CR1 loop, causing Thr249 of EGFR) to overlap with Thr84 and Gln60 of ErbB2. Therefore it seems unlikely that ErbB2 could interact with EGFR in the closed form. With some minor structural rearrangement the tip of ErbB2's CR1 loop could be accommodated in EGFR.

### Example 5

### In Silico Screening for Compounds That Modulate ErbB2 Activity

Molecular docking of large compound data bases to target proteins of known or modeled 3-dimensional structure is now a common approach in the identification of new lead compounds. This “virtual screening” approach relies on fast and accurate estimation of the ligand binding mode and an estimate of ligand affinity. Typically a large database of compounds, either real or virtual is docked to a target structure and a list of the best potential ligands is produced. This ranking should be highly enriched for active compounds which may then be subject to further experimental validation.

The calculation of the ligand binding mode may carried out by molecular docking programs which are able to dock the ligands in a flexible manner to a static protein structure. The estimation of ligand affinity is typically carried out by the use of a separate scoring function. These scoring functions include empirical functions [DOCK potential energy, Chemscore, Score], or knowledge based potentials of mean force [PMF, SMoG]. Consensus scoring involves re-scoring each ligand with multiple scoring functions and then using a combination of these rankings to generate a hit list.

We used the program DOCK (vers. 4.0.1) for the generation of favorable conformations of ligand binding. Protons and Kollman all-atom charges were added to the protein using the Biopolymer module of Sybyl6.7 and proton positions minimized with all other atoms held fixed. Scoring grids were calculated using the GRID program with a grid resolution of 0.25 Angstrom. All conformations were minimized using the DOCK energy function. Docking of ligand databases were directed towards the sites identified previously. Nine scoring functions were used, including Score, the Score-Quality estimate, DOCK energy function, PMF, PMF-RB (the PMF function with penalties for rotatable bonds), the SMoG function, SMoG/H (the SMOG function scaled by the number of ligand heavy atoms), Chemscore, and the Autodock Scoring function. Ligand conformations were chosen using a rank-by-rank consensus of the nine different scoring methods of the best 25 solutions obtained from the DOCK program using the DOCK potential energy. A ranked list of compounds was generated using a consensus of the individual scores for each ligand (in their best consensus-ranked conformation).

Four of the top-ranked compounds (compounds 39293, 94289, 19378 and 20697) were obtained and tested for their ability to modulate ErbB2 kinase activity according to the method described above. The results of these inhibition assays are shown in FIG. 3. These results show that all four compounds tested inhibited ErbB2 kinase activity at concentrations of between 10⁻¹ and 10² μM.

### CONCLUSION

The availability of 3D structures for the ErbB2(1-509) monomer, the 2:2 dimer complexes of TGFα:sEGFR501 (Garrett et al., 2002) and EGF:EGFR621 (Ogiso et al., 2002), the unliganded ErbB3 ectodomain monomer (Cho and Leahy, 2002) and the related L1/cys-rich/L2 fragment of the IGF-1R (Garrett et al., 1998) provides the framework to explore some of the outstanding issues related to ErbB receptor function. A striking feature in these comparisons is the flexibility that exists at the CR1/L2 junction resulting in major differences in the positioning of the L2 domain relative to the L1/CR1 region.

The structure of the unliganded ErbB3 full length ectodomain is even more open than that of the IGF-1R fragment, with the L2 domain rotated further away from the L1 domain (FIG. 3). This open conformation is very different from the closed arrangement of the L1 and L2 domains seen in the two EGFR/ligand dimer structures and in the ErbB2(1-509) structure reported here. The open conformation is stabilised by a single main chain/main chain hydrogen bond and side chain interactions between residues Tyr246, Phe251 and Gln252 in the CR1 loop (residues 242-259) and Asp562, Gly563, His565 (module 5) and Lys583 (module 6) of CR2. These contact residues are conserved in EGFR and ErbB4 but not in ErbB2 (Cho and Leahy, 2002). This open structure of ErbB3 provides an explanation for the predominantly low affinity ligand binding by soluble full length EGFR ectodomain compared to the high affinity binding shown by sEGFR501, which cannot make these contacts since it lacks CR2 modules 2 to 7. The same CR1 loop is critically involved in formation of the ligand-induced EGFR dimers suggesting that it becomes available for such dimer interactions following ligand binding.

The “closed” structure of the unliganded ErbB2(1-509) fragment seen here, where the bottom of the L2 domain sits against the top of the L1 domain resembles a “pseudo-active” arrangement of domains, similar to that seen in the EGFR/ligand complexes (Garrett et al., 2002; Ogiso et al., 2002). It may represent the conformation of the full length ectodomain, since the residues involved in the ErbB3 CR1 loop/CR2 interactions are not conserved in ErbB2 (Cho and Leahy, 2002) and such a constraining CR1/CR2 interaction may not be tolerated in a receptor that does not bind ligand TGFα:sEGFR501.

The 3D structure of ErbB2 also allows the epitopes for monoclonal antibodies to be mapped and their mode of action inferred, since some inhibit, some stimulate and others have no effect on cell growth. The epitopes for mAbs L87, N28 and N12 have been located to the regions Cys199-Cys214, Thr195-Cys214 and Cys510-Ala565 (mature receptor numbering) respectively (Yip Y L, Smith G, Koch J, Dubel S, Ward R L. Identification of epitope regions recognized by tumor inhibitory and stimulatory anti-ErbB-2 monoclonal antibodies: implications for vaccine design. J Immunol. 166(8):5271-8, (2001)). The epitopes for mAbs L87 and N28 (reported to have no effect or to stimulate growth of a subset of breast cancer cell lines respectively) are located in the second cys rich module of CR1, while the epitope for mAb N12, an inhibitory antibody, is located within a large region comprising cys rich modules 2 to 4 of CR2 (FIG. 2). Similarly the epitope for the potential therapeutic anti-ErbB2 monoclonal antibody MGr6 (Orlandi R, Formantici C, Menard S, Boyer C M, Wiener J R, Colnaghi M. A linear region of a monoclonal antibody conformational epitope mapped on p185HER2 oncoprotein. J. Biol. Chem. 378(11):1387-92, (1997)) has been shown to include residues 207-215 (mature receptor numbers) in the third module of CR1.

The CR2 region has also been implicated as the site of action for a set of inhibitory peptides originally designed to mimic the CDR3 loop of herceptin and shown to compete with herceptin for binding to ErbB2. A subsequent set of inhibitory peptides have been designed which mimic sequences in modules 4 to 6 of CR2, a region shown to contribute to ErbB2 heterodimer formation. Other inhibitors of ErbB2 function include the ErbB2 splice variant herstatin and the small, leucine-rich repeat proteoglycan decorin. The inhibition of ErbB2 function in breast cancer cells by decorin has been shown to be indirect and involves inactivation of ErbB4, presumably by direct binding.

The availability of the 3D structures of these receptors will facilitate the determination of the precise mechanism of action of these inhibitory agents and the design of new approaches to interfering with ErbB receptor function.

The disclosure of all publications referred to in this application are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

APPENDIX I
REMARK Coordinates for ErbB2 1-509 construct 25-2.5 A R = 0.2258

| | | | | | | | | | |
|---|---|---|---|---|---|---|---|---|---|
| ATOM | 1 | CB | SER | 1 | 46.698 | 22.851 | 38.977 | 1.00 | 99.78 |
| ATOM | 2 | OG | SER | 1 | 47.614 | 22.349 | 39.935 | 1.00 | 100.53 |
| ATOM | 3 | C | SER | 1 | 48.261 | 24.420 | 37.763 | 1.00 | 98.75 |
| ATOM | 4 | O | SER | 1 | 48.172 | 24.394 | 36.528 | 1.00 | 98.79 |
| ATOM | 5 | N | SER | 1 | 45.832 | 24.925 | 37.936 | 1.00 | 98.88 |
| ATOM | 6 | CA | SER | 1 | 47.002 | 24.314 | 38.639 | 1.00 | 99.43 |
| ATOM | 7 | N | THR | 2 | 49.431 | 24.529 | 38.395 | 1.00 | 97.11 |
| ATOM | 8 | CA | THR | 2 | 50.669 | 24.691 | 37.637 | 1.00 | 94.13 |
| ATOM | 9 | CB | THR | 2 | 51.169 | 26.163 | 37.747 | 1.00 | 94.56 |
| ATOM | 10 | OG1 | THR | 2 | 52.093 | 26.440 | 36.686 | 1.00 | 94.31 |
| ATOM | 11 | CG2 | THR | 2 | 51.855 | 26.405 | 39.098 | 1.00 | 94.08 |
| ATOM | 12 | C | THR | 2 | 51.853 | 23.755 | 37.936 | 1.00 | 91.66 |
| ATOM | 13 | O | THR | 2 | 52.049 | 23.270 | 39.059 | 1.00 | 91.43 |
| ATOM | 14 | N | GLN | 3 | 52.633 | 23.530 | 36.882 | 1.00 | 88.14 |
| ATOM | 15 | CA | GLN | 3 | 53.835 | 22.705 | 36.892 | 1.00 | 83.26 |
| ATOM | 16 | CB | GLN | 3 | 53.744 | 21.659 | 35.791 | 1.00 | 84.63 |
| ATOM | 17 | CG | GLN | 3 | 53.371 | 22.279 | 34.450 | 1.00 | 84.29 |
| ATOM | 18 | CD | GLN | 3 | 53.596 | 21.346 | 33.287 | 1.00 | 84.81 |
| ATOM | 19 | OE1 | GLN | 3 | 54.731 | 20.987 | 32.980 | 1.00 | 85.45 |
| ATOM | 20 | NE2 | GLN | 3 | 52.514 | 20.944 | 32.631 | 1.00 | 84.85 |
| ATOM | 21 | C | GLN | 3 | 54.972 | 23.670 | 36.561 | 1.00 | 79.21 |
| ATOM | 22 | O | GLN | 3 | 56.142 | 23.289 | 36.494 | 1.00 | 79.22 |
| ATOM | 23 | N | VAL | 4 | 54.597 | 24.925 | 36.338 | 1.00 | 73.41 |
| ATOM | 24 | CA | VAL | 4 | 55.542 | 25.981 | 36.003 | 1.00 | 68.56 |
| ATOM | 25 | CB | VAL | 4 | 55.074 | 26.763 | 34.752 | 1.00 | 68.25 |
| ATOM | 26 | CG1 | VAL | 4 | 55.931 | 28.002 | 34.552 | 1.00 | 66.99 |
| ATOM | 27 | CG2 | VAL | 4 | 55.139 | 25.870 | 33.530 | 1.00 | 68.86 |
| ATOM | 28 | C | VAL | 4 | 55.701 | 26.972 | 37.149 | 1.00 | 64.66 |
| ATOM | 29 | O | VAL | 4 | 54.733 | 27.308 | 37.832 | 1.00 | 65.50 |
| ATOM | 30 | N | CYS | 5 | 56.927 | 27.431 | 37.355 | 1.00 | 58.78 |
| ATOM | 31 | CA | CYS | 5 | 57.212 | 28.406 | 38.392 | 1.00 | 55.26 |
| ATOM | 32 | C | CYS | 5 | 58.186 | 29.414 | 37.822 | 1.00 | 53.65 |
| ATOM | 33 | O | CYS | 5 | 58.623 | 29.297 | 36.680 | 1.00 | 54.51 |
| ATOM | 34 | CB | CYS | 5 | 57.844 | 27.748 | 39.614 | 1.00 | 55.51 |
| ATOM | 35 | SG | CYS | 5 | 59.486 | 27.015 | 39.305 | 1.00 | 53.11 |
| ATOM | 36 | N | THR | 6 | 58.539 | 30.401 | 38.626 | 1.00 | 51.73 |
| ATOM | 37 | CA | THR | 6 | 59.463 | 31.412 | 38.176 | 1.00 | 52.19 |
| ATOM | 38 | CB | THR | 6 | 58.830 | 32.782 | 38.249 | 1.00 | 53.27 |
| ATOM | 39 | OG1 | THR | 6 | 57.717 | 32.817 | 37.348 | 1.00 | 57.20 |
| ATOM | 40 | CG2 | THR | 6 | 59.849 | 33.861 | 37.862 | 1.00 | 56.08 |
| ATOM | 41 | C | THR | 6 | 60.746 | 31.409 | 38.979 | 1.00 | 51.03 |
| ATOM | 42 | O | THR | 6 | 60.743 | 31.182 | 40.189 | 1.00 | 50.21 |
| ATOM | 43 | N | GLY | 7 | 61.847 | 31.670 | 38.290 | 1.00 | 48.21 |
| ATOM | 44 | CA | GLY | 7 | 63.125 | 31.675 | 38.960 | 1.00 | 47.10 |
| ATOM | 45 | C | GLY | 7 | 63.314 | 32.992 | 39.640 | 1.00 | 44.03 |
| ATOM | 46 | O | GLY | 7 | 62.359 | 33.713 | 39.875 | 1.00 | 45.91 |
| ATOM | 47 | N | THR | 8 | 64.556 | 33.312 | 39.946 | 1.00 | 43.65 |
| ATOM | 48 | CA | THR | 8 | 64.863 | 34.561 | 40.610 | 1.00 | 42.53 |
| ATOM | 49 | CB | THR | 8 | 65.520 | 34.293 | 41.963 | 1.00 | 44.28 |
| ATOM | 50 | OG1 | THR | 8 | 66.581 | 33.340 | 41.802 | 1.00 | 46.88 |
| ATOM | 51 | CG2 | THR | 8 | 64.488 | 33.739 | 42.943 | 1.00 | 47.24 |
| ATOM | 52 | C | THR | 8 | 65.781 | 35.417 | 39.754 | 1.00 | 40.10 |
| ATOM | 53 | O | THR | 8 | 66.004 | 35.123 | 38.585 | 1.00 | 37.84 |
| ATOM | 54 | N | ASP | 9 | 66.296 | 36.485 | 40.346 | 1.00 | 40.12 |
| ATOM | 55 | CA | ASP | 9 | 67.195 | 37.394 | 39.655 | 1.00 | 40.60 |
| ATOM | 56 | CB | ASP | 9 | 66.407 | 38.434 | 38.856 | 1.00 | 43.09 |
| ATOM | 57 | CG | ASP | 9 | 67.241 | 39.086 | 37.762 | 1.00 | 44.32 |
| ATOM | 58 | OD1 | ASP | 9 | 68.488 | 39.067 | 37.867 | 1.00 | 43.99 |
| ATOM | 59 | OD2 | ASP | 9 | 66.646 | 39.627 | 36.804 | 1.00 | 45.18 |
| ATOM | 60 | C | ASP | 9 | 68.023 | 38.097 | 40.707 | 1.00 | 39.21 |
| ATOM | 61 | O | ASP | 9 | 68.044 | 39.318 | 40.769 | 1.00 | 40.86 |
| ATOM | 62 | N | MET | 10 | 68.704 | 37.322 | 41.539 | 1.00 | 39.49 |
| ATOM | 63 | CA | MET | 10 | 69.520 | 37.896 | 42.600 | 1.00 | 40.57 |
| ATOM | 64 | CB | MET | 10 | 69.250 | 37.166 | 43.915 | 1.00 | 40.06 |
| ATOM | 65 | CG | MET | 10 | 69.796 | 35.757 | 43.936 | 1.00 | 41.52 |
| ATOM | 66 | SD | MET | 10 | 68.945 | 34.741 | 45.121 | 1.00 | 43.89 |
| ATOM | 67 | CE | MET | 10 | 69.080 | 35.809 | 46.633 | 1.00 | 45.64 |
| ATOM | 68 | C | MET | 10 | 71.013 | 37.836 | 42.300 | 1.00 | 40.42 |
| ATOM | 69 | O | MET | 10 | 71.811 | 38.483 | 42.985 | 1.00 | 38.87 |
| ATOM | 70 | N | LYS | 11 | 71.391 | 37.059 | 41.289 | 1.00 | 39.91 |
| ATOM | 71 | CA | LYS | 11 | 72.798 | 36.926 | 40.940 | 1.00 | 42.86 |
| ATOM | 72 | CB | LYS | 11 | 73.352 | 38.252 | 40.398 | 1.00 | 42.49 |
| ATOM | 73 | CG | LYS | 11 | 72.644 | 38.787 | 39.160 | 1.00 | 46.76 |
| ATOM | 74 | CD | LYS | 11 | 72.753 | 37.820 | 37.995 | 1.00 | 51.31 |
| ATOM | 75 | CE | LYS | 11 | 72.095 | 38.371 | 36.714 | 1.00 | 55.51 |
| ATOM | 76 | NZ | LYS | 11 | 70.623 | 38.092 | 36.600 | 1.00 | 57.96 |
| ATOM | 77 | C | LYS | 11 | 73.570 | 36.517 | 42.200 | 1.00 | 44.00 |
| ATOM | 78 | O | LYS | 11 | 73.198 | 35.550 | 42.877 | 1.00 | 43.65 |
| ATOM | 79 | N | LEU | 12 | 74.624 | 37.261 | 42.523 | 1.00 | 43.59 |
| ATOM | 80 | CA | LEU | 12 | 75.438 | 36.952 | 43.691 | 1.00 | 45.11 |
| ATOM | 81 | CB | LEU | 12 | 76.919 | 37.107 | 43.349 | 1.00 | 45.41 |
| ATOM | 82 | CG | LEU | 12 | 77.420 | 36.244 | 42.186 | 1.00 | 47.19 |
| ATOM | 83 | CD1 | LEU | 12 | 78.899 | 36.526 | 41.919 | 1.00 | 44.25 |
| ATOM | 84 | CD2 | LEU | 12 | 77.188 | 34.778 | 42.516 | 1.00 | 43.16 |
| ATOM | 85 | C | LEU | 12 | 75.108 | 37.808 | 44.912 | 1.00 | 46.99 |
| ATOM | 86 | O | LEU | 12 | 75.836 | 37.779 | 45.908 | 1.00 | 46.09 |
| ATOM | 87 | N | ARG | 13 | 74.016 | 38.571 | 44.830 | 1.00 | 48.04 |
| ATOM | 88 | CA | ARG | 13 | 73.591 | 39.427 | 45.935 | 1.00 | 47.76 |
| ATOM | 89 | CB | ARG | 13 | 72.374 | 40.279 | 45.521 | 1.00 | 47.43 |
| ATOM | 90 | CG | ARG | 13 | 72.683 | 41.496 | 44.633 | 1.00 | 44.43 |
| ATOM | 91 | CD | ARG | 13 | 71.409 | 42.038 | 43.968 | 1.00 | 44.08 |
| ATOM | 92 | NE | ARG | 13 | 71.525 | 42.039 | 42.509 | 1.00 | 44.91 |
| ATOM | 93 | CZ | ARG | 13 | 70.526 | 41.778 | 41.666 | 1.00 | 47.24 |
| ATOM | 94 | NH1 | ARG | 13 | 69.313 | 41.497 | 42.130 | 1.00 | 43.48 |
| ATOM | 95 | NH2 | ARG | 13 | 70.745 | 41.763 | 40.351 | 1.00 | 47.70 |
| ATOM | 96 | C | ARG | 13 | 73.251 | 38.586 | 47.177 | 1.00 | 49.10 |
| ATOM | 97 | O | ARG | 13 | 72.547 | 37.583 | 47.100 | 1.00 | 48.39 |
| ATOM | 98 | N | LEU | 14 | 73.772 | 39.019 | 48.318 | 1.00 | 51.74 |
| ATOM | 99 | CA | LEU | 14 | 73.567 | 38.353 | 49.595 | 1.00 | 52.86 |
| ATOM | 100 | CB | LEU | 14 | 74.407 | 39.058 | 50.662 | 1.00 | 54.52 |
| ATOM | 101 | CG | LEU | 14 | 74.504 | 38.400 | 52.043 | 1.00 | 58.51 |
| ATOM | 102 | CD1 | LEU | 14 | 75.513 | 37.251 | 51.983 | 1.00 | 57.78 |
| ATOM | 103 | CD2 | LEU | 14 | 74.939 | 39.431 | 53.088 | 1.00 | 58.95 |
| ATOM | 104 | C | LEU | 14 | 72.101 | 38.380 | 50.018 | 1.00 | 52.92 |
| ATOM | 105 | O | LEU | 14 | 71.457 | 39.420 | 49.960 | 1.00 | 54.88 |
| ATOM | 106 | N | PRO | 15 | 71.552 | 37.235 | 50.447 | 1.00 | 52.63 |
| ATOM | 107 | CD | PRO | 15 | 72.115 | 35.878 | 50.477 | 1.00 | 53.32 |
| ATOM | 108 | CA | PRO | 15 | 70.150 | 37.218 | 50.872 | 1.00 | 52.89 |
| ATOM | 109 | CB | PRO | 15 | 69.872 | 35.732 | 51.119 | 1.00 | 53.67 |
| ATOM | 110 | CG | PRO | 15 | 70.878 | 35.034 | 50.267 | 1.00 | 54.06 |
| ATOM | 111 | C | PRO | 15 | 70.063 | 38.025 | 52.161 | 1.00 | 53.54 |
| ATOM | 112 | O | PRO | 15 | 70.788 | 37.738 | 53.123 | 1.00 | 52.81 |
| ATOM | 113 | N | ALA | 16 | 69.182 | 39.023 | 52.181 | 1.00 | 54.22 |
| ATOM | 114 | CA | ALA | 16 | 69.018 | 39.885 | 53.355 | 1.00 | 54.19 |
| ATOM | 115 | CB | ALA | 16 | 68.069 | 41.038 | 53.032 | 1.00 | 54.82 |
| ATOM | 116 | C | ALA | 16 | 68.521 | 39.132 | 54.585 | 1.00 | 53.70 |
| ATOM | 117 | O | ALA | 16 | 68.705 | 39.587 | 55.709 | 1.00 | 53.21 |
| ATOM | 118 | N | SER | 17 | 67.902 | 37.976 | 54.371 | 1.00 | 53.95 |
| ATOM | 119 | CA | SER | 17 | 67.384 | 37.181 | 55.478 | 1.00 | 52.73 |
| ATOM | 120 | CB | SER | 17 | 65.859 | 37.301 | 55.521 | 1.00 | 51.32 |
| ATOM | 121 | OG | SER | 17 | 65.320 | 36.576 | 56.610 | 1.00 | 51.26 |
| ATOM | 122 | C | SER | 17 | 67.809 | 35.711 | 55.351 | 1.00 | 52.82 |
| ATOM | 123 | O | SER | 17 | 67.037 | 34.853 | 54.905 | 1.00 | 53.32 |
| ATOM | 124 | N | PRO | 18 | 69.049 | 35.404 | 55.755 | 1.00 | 52.39 |
| ATOM | 125 | CD | PRO | 18 | 70.012 | 36.343 | 56.358 | 1.00 | 52.34 |
| ATOM | 126 | CA | PRO | 18 | 69.603 | 34.047 | 55.697 | 1.00 | 51.18 |
| ATOM | 127 | CB | PRO | 18 | 70.923 | 34.181 | 56.442 | 1.00 | 52.04 |
| ATOM | 128 | CG | PRO | 18 | 71.314 | 35.609 | 56.181 | 1.00 | 53.40 |
| ATOM | 129 | C | PRO | 18 | 68.704 | 33.002 | 56.343 | 1.00 | 52.47 |
| ATOM | 130 | O | PRO | 18 | 68.706 | 31.832 | 55.939 | 1.00 | 52.40 |
| ATOM | 131 | N | GLU | 19 | 67.934 | 33.417 | 57.346 | 1.00 | 52.12 |
| ATOM | 132 | CA | GLU | 19 | 67.052 | 32.486 | 58.045 | 1.00 | 52.59 |
| ATOM | 133 | CB | GLU | 19 | 66.532 | 33.094 | 59.354 | 1.00 | 56.71 |
| ATOM | 134 | CG | GLU | 19 | 67.594 | 33.635 | 60.310 | 1.00 | 63.85 |
| ATOM | 135 | CD | GLU | 19 | 68.074 | 35.031 | 59.930 | 1.00 | 68.23 |
| ATOM | 136 | OE1 | GLU | 19 | 67.220 | 35.871 | 59.559 | 1.00 | 70.68 |
| ATOM | 137 | OE2 | GLU | 19 | 69.298 | 35.293 | 60.018 | 1.00 | 70.34 |
| ATOM | 138 | C | GLU | 19 | 65.856 | 32.036 | 57.215 | 1.00 | 49.61 |
| ATOM | 139 | O | GLU | 19 | 65.287 | 30.977 | 57.478 | 1.00 | 48.77 |
| ATOM | 140 | N | THR | 20 | 65.460 | 32.835 | 56.227 | 1.00 | 46.30 |
| ATOM | 141 | CA | THR | 20 | 64.306 | 32.467 | 55.411 | 1.00 | 44.66 |
| ATOM | 142 | CB | THR | 20 | 63.179 | 33.540 | 55.493 | 1.00 | 44.01 |
| ATOM | 143 | OG1 | THR | 20 | 63.700 | 34.828 | 55.137 | 1.00 | 41.22 |
| ATOM | 144 | CG2 | THR | 20 | 62.603 | 33.592 | 56.905 | 1.00 | 40.61 |
| ATOM | 145 | C | THR | 20 | 64.633 | 32.188 | 53.952 | 1.00 | 42.99 |
| ATOM | 146 | O | THR | 20 | 63.789 | 31.708 | 53.205 | 1.00 | 41.80 |
| ATOM | 147 | N | HIS | 21 | 65.868 | 32.470 | 53.560 | 1.00 | 43.03 |
| ATOM | 148 | CA | HIS | 21 | 66.322 | 32.239 | 52.191 | 1.00 | 43.70 |
| ATOM | 149 | CB | HIS | 21 | 67.838 | 32.414 | 52.104 | 1.00 | 43.12 |
| ATOM | 150 | CG | HIS | 21 | 68.369 | 32.324 | 50.708 | 1.00 | 42.78 |
| ATOM | 151 | CD2 | HIS | 21 | 69.323 | 31.530 | 50.167 | 1.00 | 43.08 |
| ATOM | 152 | ND1 | HIS | 21 | 67.918 | 33.133 | 49.690 | 1.00 | 41.09 |
| ATOM | 153 | CE1 | HIS | 21 | 68.572 | 32.845 | 48.580 | 1.00 | 42.02 |
| ATOM | 154 | NE2 | HIS | 21 | 69.431 | 31.874 | 48.842 | 1.00 | 42.45 |
| ATOM | 155 | C | HIS | 21 | 65.958 | 30.869 | 51.619 | 1.00 | 43.14 |
| ATOM | 156 | O | HIS | 21 | 65.452 | 30.780 | 50.500 | 1.00 | 41.68 |
| ATOM | 157 | N | LEU | 22 | 66.234 | 29.811 | 52.380 | 1.00 | 43.54 |
| ATOM | 158 | CA | LEU | 22 | 65.945 | 28.442 | 51.951 | 1.00 | 44.32 |
| ATOM | 159 | CB | LEU | 22 | 66.583 | 27.434 | 52.909 | 1.00 | 45.68 |
| ATOM | 160 | CG | LEU | 22 | 66.262 | 25.962 | 52.610 | 1.00 | 47.25 |
| ATOM | 161 | CD1 | LEU | 22 | 66.790 | 25.569 | 51.234 | 1.00 | 46.02 |
| ATOM | 162 | CD2 | LEU | 22 | 66.878 | 25.089 | 53.676 | 1.00 | 46.91 |
| ATOM | 163 | C | LEU | 22 | 64.457 | 28.158 | 51.865 | 1.00 | 43.69 |
| ATOM | 164 | O | LEU | 22 | 63.988 | 27.439 | 50.981 | 1.00 | 44.10 |
| ATOM | 165 | N | ASP | 23 | 63.718 | 28.720 | 52.804 | 1.00 | 45.13 |
| ATOM | 166 | CA | ASP | 23 | 62.278 | 28.543 | 52.861 | 1.00 | 45.72 |
| ATOM | 167 | CB | ASP | 23 | 61.762 | 29.165 | 54.159 | 1.00 | 48.31 |
| ATOM | 168 | CG | ASP | 23 | 60.347 | 28.763 | 54.483 | 1.00 | 52.03 |
| ATOM | 169 | OD1 | ASP | 23 | 59.718 | 28.037 | 53.679 | 1.00 | 53.43 |
| ATOM | 170 | OD2 | ASP | 23 | 59.861 | 29.185 | 55.556 | 1.00 | 55.74 |
| ATOM | 171 | C | ASP | 23 | 61.663 | 29.215 | 51.625 | 1.00 | 45.83 |
| ATOM | 172 | O | ASP | 23 | 60.607 | 28.805 | 51.124 | 1.00 | 46.01 |
| ATOM | 173 | N | MET | 24 | 62.344 | 30.241 | 51.121 | 1.00 | 44.06 |
| ATOM | 174 | CA | MET | 24 | 61.875 | 30.945 | 49.937 | 1.00 | 41.04 |
| ATOM | 175 | CB | MET | 24 | 62.681 | 32.229 | 49.717 | 1.00 | 41.33 |
| ATOM | 176 | CG | MET | 24 | 62.282 | 33.010 | 48.459 | 1.00 | 41.54 |
| ATOM | 177 | SD | MET | 24 | 63.189 | 32.480 | 46.990 | 1.00 | 41.03 |
| ATOM | 178 | CE | MET | 24 | 64.724 | 33.373 | 47.263 | 1.00 | 38.15 |
| ATOM | 179 | C | MET | 24 | 62.029 | 30.028 | 48.746 | 1.00 | 39.66 |
| ATOM | 180 | O | MET | 24 | 61.076 | 29.809 | 47.996 | 1.00 | 38.86 |
| ATOM | 181 | N | LEU | 25 | 63.237 | 29.487 | 48.585 | 1.00 | 39.89 |
| ATOM | 182 | CA | LEU | 25 | 63.548 | 28.582 | 47.479 | 1.00 | 39.32 |
| ATOM | 183 | CB | LEU | 25 | 64.979 | 28.071 | 47.609 | 1.00 | 38.16 |
| ATOM | 184 | CG | LEU | 25 | 66.063 | 29.137 | 47.425 | 1.00 | 38.16 |
| ATOM | 185 | CD1 | LEU | 25 | 67.423 | 28.570 | 47.822 | 1.00 | 36.13 |
| ATOM | 186 | CD2 | LEU | 25 | 66.061 | 29.619 | 45.977 | 1.00 | 35.36 |
| ATOM | 187 | C | LEU | 25 | 62.584 | 27.412 | 47.471 | 1.00 | 40.05 |
| ATOM | 188 | O | LEU | 25 | 62.011 | 27.072 | 46.437 | 1.00 | 41.35 |
| ATOM | 189 | N | ARG | 26 | 62.409 | 26.802 | 48.636 | 1.00 | 41.18 |
| ATOM | 190 | CA | ARG | 26 | 61.502 | 25.679 | 48.780 | 1.00 | 43.57 |
| ATOM | 191 | CB | ARG | 26 | 61.430 | 25.236 | 50.249 | 1.00 | 47.19 |
| ATOM | 192 | CG | ARG | 26 | 60.308 | 24.228 | 50.530 | 1.00 | 53.01 |
| ATOM | 193 | CD | ARG | 26 | 60.193 | 23.867 | 52.015 | 1.00 | 57.96 |
| ATOM | 194 | NE | ARG | 26 | 61.417 | 23.249 | 52.523 | 1.00 | 63.73 |
| ATOM | 195 | CZ | ARG | 26 | 62.397 | 23.911 | 53.135 | 1.00 | 67.15 |
| ATOM | 196 | NH1 | ARG | 26 | 62.293 | 25.222 | 53.329 | 1.00 | 69.09 |
| ATOM | 197 | NH2 | ARG | 26 | 63.492 | 23.269 | 53.539 | 1.00 | 67.64 |
| ATOM | 198 | C | ARG | 26 | 60.107 | 26.045 | 48.276 | 1.00 | 42.82 |
| ATOM | 199 | O | ARG | 26 | 59.517 | 25.299 | 47.497 | 1.00 | 42.76 |
| ATOM | 200 | N | HIS | 27 | 59.580 | 27.185 | 48.725 | 1.00 | 41.65 |
| ATOM | 201 | CA | HIS | 27 | 58.249 | 27.639 | 48.306 | 1.00 | 41.19 |
| ATOM | 202 | CB | HIS | 27 | 57.892 | 28.971 | 48.982 | 1.00 | 41.27 |
| ATOM | 203 | CG | HIS | 27 | 57.358 | 28.831 | 50.374 | 1.00 | 40.54 |
| ATOM | 204 | CD2 | HIS | 27 | 57.915 | 29.116 | 51.575 | 1.00 | 39.10 |
| ATOM | 205 | ND1 | HIS | 27 | 56.087 | 28.363 | 50.643 | 1.00 | 40.15 |
| ATOM | 206 | CE1 | HIS | 27 | 55.887 | 28.368 | 51.949 | 1.00 | 40.37 |
| ATOM | 207 | NE2 | HIS | 27 | 56.981 | 28.819 | 52.537 | 1.00 | 37.81 |
| ATOM | 208 | C | HIS | 27 | 58.244 | 27.846 | 46.801 | 1.00 | 41.58 |
| ATOM | 209 | O | HIS | 27 | 57.318 | 27.455 | 46.089 | 1.00 | 42.62 |
| ATOM | 210 | N | LEU | 28 | 59.307 | 28.470 | 46.328 | 1.00 | 42.34 |
| ATOM | 211 | CA | LEU | 28 | 59.476 | 28.770 | 44.920 | 1.00 | 44.22 |
| ATOM | 212 | CB | LEU | 28 | 60.763 | 29.576 | 44.750 | 1.00 | 46.18 |
| ATOM | 213 | CG | LEU | 28 | 60.849 | 30.528 | 43.572 | 1.00 | 46.79 |
| ATOM | 214 | CD1 | LEU | 28 | 59.751 | 31.570 | 43.697 | 1.00 | 46.45 |
| ATOM | 215 | CD2 | LEU | 28 | 62.221 | 31.194 | 43.558 | 1.00 | 48.92 |
| ATOM | 216 | C | LEU | 28 | 59.516 | 27.554 | 43.989 | 1.00 | 44.27 |
| ATOM | 217 | O | LEU | 28 | 58.762 | 27.494 | 43.016 | 1.00 | 45.44 |
| ATOM | 218 | N | TYR | 29 | 60.377 | 26.581 | 44.292 | 1.00 | 43.09 |
| ATOM | 219 | CA | TYR | 29 | 60.534 | 25.415 | 43.417 | 1.00 | 42.13 |
| ATOM | 220 | CB | TYR | 29 | 62.022 | 25.135 | 43.236 | 1.00 | 39.72 |
| ATOM | 221 | CG | TYR | 29 | 62.757 | 26.302 | 42.636 | 1.00 | 38.19 |
| ATOM | 222 | CD1 | TYR | 29 | 63.746 | 26.977 | 43.353 | 1.00 | 34.60 |
| ATOM | 223 | CE1 | TYR | 29 | 64.383 | 28.101 | 42.816 | 1.00 | 35.85 |
| ATOM | 224 | CD2 | TYR | 29 | 62.420 | 26.770 | 41.369 | 1.00 | 36.03 |
| ATOM | 225 | CE2 | TYR | 29 | 63.042 | 27.888 | 40.825 | 1.00 | 38.16 |
| ATOM | 226 | CZ | TYR | 29 | 64.021 | 28.554 | 41.550 | 1.00 | 36.60 |
| ATOM | 227 | OH | TYR | 29 | 64.599 | 29.683 | 41.011 | 1.00 | 34.62 |
| ATOM | 228 | C | TYR | 29 | 59.832 | 24.094 | 43.717 | 1.00 | 43.10 |
| ATOM | 229 | O | TYR | 29 | 59.749 | 23.235 | 42.845 | 1.00 | 42.11 |
| ATOM | 230 | N | GLN | 30 | 59.322 | 23.914 | 44.928 | 1.00 | 44.62 |
| ATOM | 231 | CA | GLN | 30 | 58.675 | 22.651 | 45.258 | 1.00 | 45.73 |
| ATOM | 232 | CB | GLN | 30 | 58.057 | 22.722 | 46.651 | 1.00 | 49.77 |
| ATOM | 233 | CG | GLN | 30 | 57.902 | 21.367 | 47.316 | 1.00 | 54.17 |
| ATOM | 234 | CD | GLN | 30 | 57.827 | 21.494 | 48.820 | 1.00 | 59.15 |
| ATOM | 235 | OE1 | GLN | 30 | 56.949 | 22.186 | 49.353 | 1.00 | 61.69 |
| ATOM | 236 | NE2 | GLN | 30 | 58.757 | 20.837 | 49.523 | 1.00 | 60.08 |
| ATOM | 237 | C | GLN | 30 | 57.616 | 22.241 | 44.246 | 1.00 | 43.52 |
| ATOM | 238 | O | GLN | 30 | 56.666 | 22.971 | 43.988 | 1.00 | 43.46 |
| ATOM | 239 | N | GLY | 31 | 57.791 | 21.062 | 43.670 | 1.00 | 42.70 |
| ATOM | 240 | CA | GLY | 31 | 56.833 | 20.579 | 42.698 | 1.00 | 44.21 |
| ATOM | 241 | C | GLY | 31 | 57.004 | 21.189 | 41.324 | 1.00 | 45.65 |
| ATOM | 242 | O | GLY | 31 | 56.423 | 20.704 | 40.349 | 1.00 | 46.11 |
| ATOM | 243 | N | CYS | 32 | 57.798 | 22.250 | 41.232 | 1.00 | 45.88 |
| ATOM | 244 | CA | CYS | 32 | 58.026 | 22.905 | 39.946 | 1.00 | 47.34 |
| ATOM | 245 | C | CYS | 32 | 58.777 | 22.007 | 38.947 | 1.00 | 47.39 |
| ATOM | 246 | O | CYS | 32 | 59.699 | 21.272 | 39.319 | 1.00 | 45.44 |
| ATOM | 247 | CB | CYS | 32 | 58.819 | 24.193 | 40.147 | 1.00 | 48.51 |
| ATOM | 248 | SG | CYS | 32 | 58.990 | 25.178 | 38.624 | 1.00 | 50.51 |
| ATOM | 249 | N | GLN | 33 | 58.378 | 22.078 | 37.679 | 1.00 | 47.35 |
| ATOM | 250 | CA | GLN | 33 | 59.012 | 21.292 | 36.620 | 1.00 | 48.75 |
| ATOM | 251 | CB | GLN | 33 | 57.989 | 20.400 | 35.932 | 1.00 | 49.20 |
| ATOM | 252 | CG | GLN | 33 | 57.576 | 19.204 | 36.755 | 1.00 | 54.87 |
| ATOM | 253 | CD | GLN | 33 | 56.562 | 18.341 | 36.036 | 1.00 | 57.52 |
| ATOM | 254 | OE1 | GLN | 33 | 56.554 | 17.117 | 36.185 | 1.00 | 57.65 |
| ATOM | 255 | NE2 | GLN | 33 | 55.690 | 18.978 | 35.255 | 1.00 | 58.17 |
| ATOM | 256 | C | GLN | 33 | 59.679 | 22.183 | 35.572 | 1.00 | 49.12 |
| ATOM | 257 | O | GLN | 33 | 60.759 | 21.859 | 35.066 | 1.00 | 48.02 |
| ATOM | 258 | N | VAL | 34 | 59.024 | 23.296 | 35.240 | 1.00 | 47.34 |
| ATOM | 259 | CA | VAL | 34 | 59.559 | 24.236 | 34.260 | 1.00 | 44.43 |
| ATOM | 260 | CB | VAL | 34 | 58.583 | 24.448 | 33.091 | 1.00 | 42.79 |
| ATOM | 261 | CG1 | VAL | 34 | 59.141 | 25.488 | 32.129 | 1.00 | 42.57 |
| ATOM | 262 | CG2 | VAL | 34 | 58.355 | 23.141 | 32.369 | 1.00 | 39.31 |
| ATOM | 263 | C | VAL | 34 | 59.817 | 25.577 | 34.930 | 1.00 | 44.27 |
| ATOM | 264 | O | VAL | 34 | 58.895 | 26.216 | 35.424 | 1.00 | 46.02 |
| ATOM | 265 | N | VAL | 35 | 61.073 | 25.998 | 34.946 | 1.00 | 42.27 |
| ATOM | 266 | CA | VAL | 35 | 61.434 | 27.255 | 35.568 | 1.00 | 42.31 |
| ATOM | 267 | CB | VAL | 35 | 62.742 | 27.117 | 36.360 | 1.00 | 41.24 |
| ATOM | 268 | CG1 | VAL | 35 | 63.170 | 28.473 | 36.909 | 1.00 | 40.12 |
| ATOM | 269 | CG2 | VAL | 35 | 62.555 | 26.125 | 37.480 | 1.00 | 39.17 |
| ATOM | 270 | C | VAL | 35 | 61.601 | 28.409 | 34.583 | 1.00 | 43.64 |
| ATOM | 271 | O | VAL | 35 | 62.617 | 28.494 | 33.890 | 1.00 | 42.15 |
| ATOM | 272 | N | GLN | 36 | 60.591 | 29.276 | 34.511 | 1.00 | 45.54 |
| ATOM | 273 | CA | GLN | 36 | 60.666 | 30.453 | 33.653 | 1.00 | 48.03 |
| ATOM | 274 | CB | GLN | 36 | 59.339 | 31.226 | 33.650 | 1.00 | 52.27 |
| ATOM | 275 | CG | GLN | 36 | 58.117 | 30.387 | 33.323 | 1.00 | 58.81 |
| ATOM | 276 | CD | GLN | 36 | 58.043 | 29.982 | 31.861 | 1.00 | 62.93 |
| ATOM | 277 | OE1 | GLN | 36 | 59.060 | 29.949 | 31.155 | 1.00 | 66.35 |
| ATOM | 278 | NE2 | GLN | 36 | 56.837 | 29.651 | 31.402 | 1.00 | 63.08 |
| ATOM | 279 | C | GLN | 36 | 61.714 | 31.268 | 34.398 | 1.00 | 46.27 |
| ATOM | 280 | O | GLN | 36 | 61.757 | 31.237 | 35.630 | 1.00 | 49.00 |
| ATOM | 281 | N | GLY | 37 | 62.567 | 31.989 | 33.694 | 1.00 | 41.30 |
| ATOM | 282 | CA | GLY | 37 | 63.556 | 32.739 | 34.437 | 1.00 | 40.34 |
| ATOM | 283 | C | GLY | 37 | 64.762 | 31.895 | 34.812 | 1.00 | 39.23 |
| ATOM | 284 | O | GLY | 37 | 65.041 | 30.870 | 34.187 | 1.00 | 39.87 |
| ATOM | 285 | N | ASN | 38 | 65.466 | 32.305 | 35.853 | 1.00 | 37.71 |
| ATOM | 286 | CA | ASN | 38 | 66.681 | 31.617 | 36.253 | 1.00 | 37.65 |
| ATOM | 287 | CB | ASN | 38 | 67.796 | 32.647 | 36.355 | 1.00 | 38.08 |
| ATOM | 288 | CG | ASN | 38 | 67.723 | 33.667 | 35.259 | 1.00 | 40.43 |
| ATOM | 289 | OD1 | ASN | 38 | 67.923 | 33.356 | 34.087 | 1.00 | 40.88 |
| ATOM | 290 | ND2 | ASN | 38 | 67.412 | 34.897 | 35.628 | 1.00 | 42.58 |
| ATOM | 291 | C | ASN | 38 | 66.645 | 30.809 | 37.542 | 1.00 | 37.32 |
| ATOM | 292 | O | ASN | 38 | 65.976 | 31.175 | 38.509 | 1.00 | 37.72 |
| ATOM | 293 | N | LEU | 39 | 67.385 | 29.706 | 37.543 | 1.00 | 35.57 |
| ATOM | 294 | CA | LEU | 39 | 67.488 | 28.861 | 38.719 | 1.00 | 36.14 |
| ATOM | 295 | CB | LEU | 39 | 67.563 | 27.386 | 38.334 | 1.00 | 35.37 |
| ATOM | 296 | CG | LEU | 39 | 67.785 | 26.494 | 39.559 | 1.00 | 36.47 |
| ATOM | 297 | CD1 | LEU | 39 | 66.662 | 26.738 | 40.561 | 1.00 | 36.90 |
| ATOM | 298 | CD2 | LEU | 39 | 67.840 | 25.027 | 39.153 | 1.00 | 33.34 |
| ATOM | 299 | C | LEU | 39 | 68.776 | 29.274 | 39.411 | 1.00 | 37.41 |
| ATOM | 300 | O | LEU | 39 | 69.873 | 28.952 | 38.933 | 1.00 | 36.96 |
| ATOM | 301 | N | GLU | 40 | 68.644 | 29.998 | 40.524 | 1.00 | 37.19 |
| ATOM | 302 | CA | GLU | 40 | 69.808 | 30.471 | 41.266 | 1.00 | 38.08 |
| ATOM | 303 | CB | GLU | 40 | 69.806 | 32.003 | 41.350 | 1.00 | 38.80 |
| ATOM | 304 | CG | GLU | 40 | 69.494 | 32.703 | 40.013 | 1.00 | 42.78 |
| ATOM | 305 | CD | GLU | 40 | 69.452 | 34.223 | 40.117 | 1.00 | 41.78 |
| ATOM | 306 | OE1 | GLU | 40 | 70.459 | 34.885 | 39.779 | 1.00 | 43.35 |
| ATOM | 307 | OE2 | GLU | 40 | 68.411 | 34.755 | 40.546 | 1.00 | 42.97 |
| ATOM | 308 | C | GLU | 40 | 69.846 | 29.875 | 42.666 | 1.00 | 39.52 |
| ATOM | 309 | O | GLU | 40 | 69.009 | 30.185 | 43.509 | 1.00 | 38.50 |
| ATOM | 310 | N | LEU | 41 | 70.826 | 29.005 | 42.893 | 1.00 | 40.86 |
| ATOM | 311 | CA | LEU | 41 | 71.017 | 28.346 | 44.173 | 1.00 | 41.39 |
| ATOM | 312 | CB | LEU | 41 | 71.104 | 26.846 | 43.942 | 1.00 | 41.49 |
| ATOM | 313 | CG | LEU | 41 | 69.800 | 26.337 | 43.304 | 1.00 | 42.78 |
| ATOM | 314 | CD1 | LEU | 41 | 69.983 | 24.930 | 42.767 | 1.00 | 39.41 |
| ATOM | 315 | CD2 | LEU | 41 | 68.667 | 26.390 | 44.342 | 1.00 | 40.37 |
| ATOM | 316 | C | LEU | 41 | 72.302 | 28.895 | 44.784 | 1.00 | 43.48 |
| ATOM | 317 | O | LEU | 41 | 73.414 | 28.483 | 44.423 | 1.00 | 43.60 |
| ATOM | 318 | N | THR | 42 | 72.137 | 29.837 | 45.711 | 1.00 | 42.93 |
| ATOM | 319 | CA | THR | 42 | 73.271 | 30.494 | 46.337 | 1.00 | 42.71 |
| ATOM | 320 | CB | THR | 42 | 73.405 | 31.940 | 45.809 | 1.00 | 42.41 |
| ATOM | 321 | OG1 | THR | 42 | 72.214 | 32.679 | 46.124 | 1.00 | 44.65 |
| ATOM | 322 | CG2 | THR | 42 | 73.597 | 31.940 | 44.299 | 1.00 | 40.36 |
| ATOM | 323 | C | THR | 42 | 73.228 | 30.547 | 47.859 | 1.00 | 44.56 |
| ATOM | 324 | O | THR | 42 | 72.170 | 30.371 | 48.474 | 1.00 | 44.84 |
| ATOM | 325 | N | TYR | 43 | 74.402 | 30.793 | 48.444 | 1.00 | 45.06 |
| ATOM | 326 | CA | TYR | 43 | 74.599 | 30.912 | 49.889 | 1.00 | 45.25 |
| ATOM | 327 | CB | TYR | 43 | 74.222 | 32.326 | 50.355 | 1.00 | 43.21 |
| ATOM | 328 | CG | TYR | 43 | 75.011 | 33.417 | 49.663 | 1.00 | 45.31 |
| ATOM | 329 | CD1 | TYR | 43 | 74.534 | 34.021 | 48.495 | 1.00 | 44.65 |
| ATOM | 330 | CE1 | TYR | 43 | 75.295 | 34.970 | 47.809 | 1.00 | 44.72 |
| ATOM | 331 | CD2 | TYR | 43 | 76.271 | 33.795 | 50.133 | 1.00 | 46.31 |
| ATOM | 332 | CE2 | TYR | 43 | 77.047 | 34.742 | 49.452 | 1.00 | 45.38 |
| ATOM | 333 | CZ | TYR | 43 | 76.557 | 35.323 | 48.291 | 1.00 | 46.22 |
| ATOM | 334 | OH | TYR | 43 | 77.342 | 36.227 | 47.598 | 1.00 | 42.92 |
| ATOM | 335 | C | TYR | 43 | 73.883 | 29.886 | 50.768 | 1.00 | 46.52 |
| ATOM | 336 | O | TYR | 43 | 73.432 | 30.216 | 51.864 | 1.00 | 46.70 |
| ATOM | 337 | N | LEU | 44 | 73.795 | 28.645 | 50.310 | 1.00 | 47.21 |
| ATOM | 338 | CA | LEU | 44 | 73.126 | 27.622 | 51.098 | 1.00 | 50.64 |
| ATOM | 339 | CB | LEU | 44 | 72.463 | 26.592 | 50.181 | 1.00 | 49.29 |
| ATOM | 340 | CG | LEU | 44 | 71.380 | 27.177 | 49.271 | 1.00 | 47.48 |
| ATOM | 341 | CD1 | LEU | 44 | 70.697 | 26.077 | 48.489 | 1.00 | 47.96 |
| ATOM | 342 | CD2 | LEU | 44 | 70.371 | 27.914 | 50.117 | 1.00 | 46.43 |
| ATOM | 343 | C | LEU | 44 | 74.098 | 26.931 | 52.044 | 1.00 | 53.36 |
| ATOM | 344 | O | LEU | 44 | 75.175 | 26.517 | 51.634 | 1.00 | 53.59 |
| ATOM | 345 | N | PRO | 45 | 73.725 | 26.807 | 53.334 | 1.00 | 56.73 |
| ATOM | 346 | CD | PRO | 45 | 72.430 | 27.232 | 53.900 | 1.00 | 58.00 |
| ATOM | 347 | CA | PRO | 45 | 74.548 | 26.168 | 54.368 | 1.00 | 57.37 |
| ATOM | 348 | CB | PRO | 45 | 73.802 | 26.506 | 55.652 | 1.00 | 57.48 |
| ATOM | 349 | CG | PRO | 45 | 72.373 | 26.447 | 55.205 | 1.00 | 59.17 |
| ATOM | 350 | C | PRO | 45 | 74.677 | 24.659 | 54.141 | 1.00 | 58.12 |
| ATOM | 351 | O | PRO | 45 | 73.874 | 24.053 | 53.425 | 1.00 | 57.47 |
| ATOM | 352 | N | THR | 46 | 75.680 | 24.060 | 54.772 | 1.00 | 57.76 |
| ATOM | 353 | CA | THR | 46 | 75.945 | 22.641 | 54.610 | 1.00 | 57.98 |
| ATOM | 354 | CB | THR | 46 | 77.255 | 22.257 | 55.363 | 1.00 | 56.25 |
| ATOM | 355 | OG1 | THR | 46 | 77.894 | 21.172 | 54.683 | 1.00 | 57.72 |
| ATOM | 356 | CG2 | THR | 46 | 76.972 | 21.841 | 56.791 | 1.00 | 55.98 |
| ATOM | 357 | C | THR | 46 | 74.799 | 21.696 | 55.003 | 1.00 | 59.34 |
| ATOM | 358 | O | THR | 46 | 74.720 | 20.572 | 54.508 | 1.00 | 59.26 |
| ATOM | 359 | N | ASN | 47 | 73.895 | 22.147 | 55.864 | 1.00 | 62.38 |
| ATOM | 360 | CA | ASN | 47 | 72.791 | 21.291 | 56.300 | 1.00 | 66.35 |
| ATOM | 361 | CB | ASN | 47 | 72.480 | 21.549 | 57.781 | 1.00 | 72.57 |
| ATOM | 362 | CG | ASN | 47 | 72.136 | 23.006 | 58.064 | 1.00 | 78.54 |
| ATOM | 363 | OD1 | ASN | 47 | 71.287 | 23.596 | 57.391 | 1.00 | 79.41 |
| ATOM | 364 | ND2 | ASN | 47 | 72.792 | 23.586 | 59.066 | 1.00 | 83.60 |
| ATOM | 365 | C | ASN | 47 | 71.509 | 21.462 | 55.488 | 1.00 | 65.90 |
| ATOM | 366 | O | ASN | 47 | 70.466 | 20.906 | 55.838 | 1.00 | 66.68 |
| ATOM | 367 | N | ALA | 48 | 71.593 | 22.218 | 54.398 | 1.00 | 64.94 |
| ATOM | 368 | CA | ALA | 48 | 70.435 | 22.491 | 53.549 | 1.00 | 61.77 |
| ATOM | 369 | CB | ALA | 48 | 70.783 | 23.582 | 52.548 | 1.00 | 61.89 |
| ATOM | 370 | C | ALA | 48 | 69.845 | 21.294 | 52.813 | 1.00 | 60.59 |
| ATOM | 371 | O | ALA | 48 | 70.524 | 20.630 | 52.030 | 1.00 | 60.88 |
| ATOM | 372 | N | SER | 49 | 68.572 | 21.022 | 53.075 | 1.00 | 58.17 |
| ATOM | 373 | CA | SER | 49 | 67.875 | 19.937 | 52.405 | 1.00 | 57.25 |
| ATOM | 374 | CB | SER | 49 | 66.781 | 19.367 | 53.304 | 1.00 | 59.13 |
| ATOM | 375 | OG | SER | 49 | 65.951 | 18.483 | 52.566 | 1.00 | 62.13 |
| ATOM | 376 | C | SER | 49 | 67.245 | 20.536 | 51.150 | 1.00 | 55.84 |
| ATOM | 377 | O | SER | 49 | 66.339 | 21.365 | 51.247 | 1.00 | 56.85 |
| ATOM | 378 | N | LEU | 50 | 67.710 | 20.099 | 49.983 | 1.00 | 53.85 |
| ATOM | 379 | CA | LEU | 50 | 67.236 | 20.609 | 48.702 | 1.00 | 50.83 |
| ATOM | 380 | CB | LEU | 50 | 68.432 | 21.048 | 47.872 | 1.00 | 48.70 |
| ATOM | 381 | CG | LEU | 50 | 69.386 | 22.023 | 48.554 | 1.00 | 50.27 |
| ATOM | 382 | CD1 | LEU | 50 | 70.539 | 22.339 | 47.601 | 1.00 | 48.46 |
| ATOM | 383 | CD2 | LEU | 50 | 68.635 | 23.298 | 48.948 | 1.00 | 50.37 |
| ATOM | 384 | C | LEU | 50 | 66.396 | 19.651 | 47.863 | 1.00 | 50.80 |
| ATOM | 385 | O | LEU | 50 | 66.304 | 19.812 | 46.652 | 1.00 | 51.99 |
| ATOM | 386 | N | SER | 51 | 65.777 | 18.666 | 48.492 | 1.00 | 50.31 |
| ATOM | 387 | CA | SER | 51 | 64.972 | 17.696 | 47.758 | 1.00 | 49.12 |
| ATOM | 388 | CB | SER | 51 | 64.342 | 16.710 | 48.737 | 1.00 | 49.94 |
| ATOM | 389 | OG | SER | 51 | 63.326 | 17.355 | 49.486 | 1.00 | 54.77 |
| ATOM | 390 | C | SER | 51 | 63.861 | 18.311 | 46.897 | 1.00 | 48.09 |
| ATOM | 391 | O | SER | 51 | 63.420 | 17.710 | 45.920 | 1.00 | 49.53 |
| ATOM | 392 | N | PHE | 52 | 63.398 | 19.500 | 47.252 | 1.00 | 45.59 |
| ATOM | 393 | CA | PHE | 52 | 62.330 | 20.121 | 46.487 | 1.00 | 44.82 |
| ATOM | 394 | CB | PHE | 52 | 61.796 | 21.335 | 47.245 | 1.00 | 45.04 |
| ATOM | 395 | CG | PHE | 52 | 62.850 | 22.338 | 47.608 | 1.00 | 44.14 |
| ATOM | 396 | CD1 | PHE | 52 | 63.365 | 23.208 | 46.652 | 1.00 | 41.89 |
| ATOM | 397 | CD2 | PHE | 52 | 63.322 | 22.424 | 48.919 | 1.00 | 43.61 |
| ATOM | 398 | CE1 | PHE | 52 | 64.342 | 24.158 | 47.000 | 1.00 | 40.61 |
| ATOM | 399 | CE2 | PHE | 52 | 64.300 | 23.368 | 49.275 | 1.00 | 41.30 |
| ATOM | 400 | CZ | PHE | 52 | 64.806 | 24.235 | 48.313 | 1.00 | 39.55 |
| ATOM | 401 | C | PHE | 52 | 62.712 | 20.520 | 45.059 | 1.00 | 44.76 |
| ATOM | 402 | O | PHE | 52 | 61.859 | 20.959 | 44.289 | 1.00 | 44.08 |
| ATOM | 403 | N | LEU | 53 | 63.986 | 20.360 | 44.710 | 1.00 | 43.96 |
| ATOM | 404 | CA | LEU | 53 | 64.485 | 20.701 | 43.382 | 1.00 | 43.72 |
| ATOM | 405 | CB | LEU | 53 | 65.931 | 21.183 | 43.481 | 1.00 | 40.39 |
| ATOM | 406 | CG | LEU | 53 | 66.283 | 22.422 | 44.307 | 1.00 | 40.76 |
| ATOM | 407 | CD1 | LEU | 53 | 67.788 | 22.494 | 44.453 | 1.00 | 39.34 |
| ATOM | 408 | CD2 | LEU | 53 | 65.757 | 23.691 | 43.631 | 1.00 | 40.38 |
| ATOM | 409 | C | LEU | 53 | 64.449 | 19.506 | 42.419 | 1.00 | 46.76 |
| ATOM | 410 | O | LEU | 53 | 64.773 | 19.643 | 41.233 | 1.00 | 47.47 |
| ATOM | 411 | N | GLN | 54 | 64.048 | 18.343 | 42.927 | 1.00 | 48.46 |
| ATOM | 412 | CA | GLN | 54 | 64.029 | 17.108 | 42.140 | 1.00 | 49.89 |
| ATOM | 413 | CB | GLN | 54 | 63.710 | 15.913 | 43.050 | 1.00 | 52.52 |
| ATOM | 414 | CG | GLN | 54 | 62.363 | 16.008 | 43.756 | 1.00 | 57.71 |
| ATOM | 415 | CD | GLN | 54 | 61.976 | 14.718 | 44.471 | 1.00 | 60.56 |
| ATOM | 416 | OE1 | GLN | 54 | 62.778 | 14.139 | 45.213 | 1.00 | 59.21 |
| ATOM | 417 | NE2 | GLN | 54 | 60.735 | 14.269 | 44.259 | 1.00 | 61.81 |
| ATOM | 418 | C | GLN | 54 | 63.138 | 17.026 | 40.904 | 1.00 | 48.57 |
| ATOM | 419 | O | GLN | 54 | 63.359 | 16.159 | 40.050 | 1.00 | 47.17 |
| ATOM | 420 | N | ASP | 55 | 62.147 | 17.907 | 40.786 | 1.00 | 47.48 |
| ATOM | 421 | CA | ASP | 55 | 61.248 | 17.853 | 39.630 | 1.00 | 47.88 |
| ATOM | 422 | CB | ASP | 55 | 59.814 | 18.076 | 40.082 | 1.00 | 51.15 |
| ATOM | 423 | CG | ASP | 55 | 59.371 | 17.062 | 41.089 | 1.00 | 55.30 |
| ATOM | 424 | OD1 | ASP | 55 | 59.391 | 15.852 | 40.759 | 1.00 | 55.97 |
| ATOM | 425 | OD2 | ASP | 55 | 59.012 | 17.480 | 42.213 | 1.00 | 58.38 |
| ATOM | 426 | C | ASP | 55 | 61.557 | 18.801 | 38.475 | 1.00 | 46.37 |
| ATOM | 427 | O | ASP | 55 | 60.917 | 18.725 | 37.423 | 1.00 | 47.17 |
| ATOM | 428 | N | ILE | 56 | 62.523 | 19.693 | 38.672 | 1.00 | 44.29 |
| ATOM | 429 | CA | ILE | 56 | 62.928 | 20.648 | 37.643 | 1.00 | 43.21 |
| ATOM | 430 | CB | ILE | 56 | 64.033 | 21.585 | 38.197 | 1.00 | 40.52 |
| ATOM | 431 | CG2 | ILE | 56 | 64.534 | 22.528 | 37.117 | 1.00 | 37.41 |
| ATOM | 432 | CG1 | ILE | 56 | 63.486 | 22.350 | 39.401 | 1.00 | 37.70 |
| ATOM | 433 | CD1 | ILE | 56 | 64.501 | 23.185 | 40.099 | 1.00 | 37.33 |
| ATOM | 434 | C | ILE | 56 | 63.453 | 19.887 | 36.409 | 1.00 | 44.92 |
| ATOM | 435 | O | ILE | 56 | 64.379 | 19.077 | 36.516 | 1.00 | 45.32 |
| ATOM | 436 | N | GLN | 57 | 62.842 | 20.139 | 35.254 | 1.00 | 44.74 |
| ATOM | 437 | CA | GLN | 57 | 63.225 | 19.496 | 33.998 | 1.00 | 46.02 |
| ATOM | 438 | CB | GLN | 57 | 62.017 | 18.887 | 33.307 | 1.00 | 48.48 |
| ATOM | 439 | CG | GLN | 57 | 61.555 | 17.577 | 33.863 | 1.00 | 54.00 |
| ATOM | 440 | CD | GLN | 57 | 60.559 | 16.928 | 32.937 | 1.00 | 56.38 |
| ATOM | 441 | OE1 | GLN | 57 | 60.682 | 17.034 | 31.713 | 1.00 | 58.36 |
| ATOM | 442 | NE2 | GLN | 57 | 59.572 | 16.245 | 33.505 | 1.00 | 57.50 |
| ATOM | 443 | C | GLN | 57 | 63.862 | 20.473 | 33.027 | 1.00 | 45.57 |
| ATOM | 444 | O | GLN | 57 | 64.740 | 20.100 | 32.249 | 1.00 | 44.05 |
| ATOM | 445 | N | GLU | 58 | 63.377 | 21.711 | 33.033 | 1.00 | 45.73 |
| ATOM | 446 | CA | GLU | 58 | 63.934 | 22.738 | 32.169 | 1.00 | 45.78 |
| ATOM | 447 | CB | GLU | 58 | 63.166 | 22.825 | 30.844 | 1.00 | 48.71 |
| ATOM | 448 | CG | GLU | 58 | 61.654 | 22.860 | 30.928 | 1.00 | 53.11 |
| ATOM | 449 | CD | GLU | 58 | 60.996 | 22.732 | 29.546 | 1.00 | 56.02 |
| ATOM | 450 | OE1 | GLU | 58 | 61.070 | 21.635 | 28.932 | 1.00 | 53.20 |
| ATOM | 451 | OE2 | GLU | 58 | 60.410 | 23.737 | 29.072 | 1.00 | 58.53 |
| ATOM | 452 | C | GLU | 58 | 63.998 | 24.099 | 32.844 | 1.00 | 43.99 |
| ATOM | 453 | O | GLU | 58 | 63.206 | 24.406 | 33.729 | 1.00 | 43.84 |
| ATOM | 454 | N | VAL | 59 | 64.985 | 24.888 | 32.445 | 1.00 | 42.10 |
| ATOM | 455 | CA | VAL | 59 | 65.180 | 26.224 | 32.982 | 1.00 | 41.08 |
| ATOM | 456 | CB | VAL | 59 | 66.464 | 26.297 | 33.820 | 1.00 | 38.49 |
| ATOM | 457 | CG1 | VAL | 59 | 66.931 | 27.751 | 33.959 | 1.00 | 36.98 |
| ATOM | 458 | CG2 | VAL | 59 | 66.212 | 25.680 | 35.189 | 1.00 | 36.83 |
| ATOM | 459 | C | VAL | 59 | 65.291 | 27.164 | 31.786 | 1.00 | 43.13 |
| ATOM | 460 | O | VAL | 59 | 66.204 | 27.025 | 30.968 | 1.00 | 43.59 |
| ATOM | 461 | N | GLN | 60 | 64.355 | 28.107 | 31.685 | 1.00 | 43.10 |
| ATOM | 462 | CA | GLN | 60 | 64.335 | 29.048 | 30.577 | 1.00 | 44.01 |
| ATOM | 463 | CB | GLN | 60 | 63.006 | 29.797 | 30.573 | 1.00 | 48.84 |
| ATOM | 464 | CG | GLN | 60 | 62.831 | 30.739 | 29.394 | 1.00 | 52.91 |
| ATOM | 465 | CD | GLN | 60 | 62.559 | 32.139 | 29.866 | 1.00 | 57.79 |
| ATOM | 466 | OE1 | GLN | 60 | 61.503 | 32.414 | 30.436 | 1.00 | 60.40 |
| ATOM | 467 | NE2 | GLN | 60 | 63.523 | 33.037 | 29.659 | 1.00 | 60.28 |
| ATOM | 468 | C | GLN | 60 | 65.510 | 30.033 | 30.592 | 1.00 | 43.30 |
| ATOM | 469 | O | GLN | 60 | 66.031 | 30.416 | 29.537 | 1.00 | 41.41 |
| ATOM | 470 | N | GLY | 61 | 65.928 | 30.441 | 31.786 | 1.00 | 42.03 |
| ATOM | 471 | CA | GLY | 61 | 67.053 | 31.352 | 31.892 | 1.00 | 40.78 |
| ATOM | 472 | C | GLY | 61 | 68.354 | 30.577 | 32.012 | 1.00 | 40.97 |
| ATOM | 473 | O | GLY | 61 | 68.605 | 29.643 | 31.246 | 1.00 | 40.44 |
| ATOM | 474 | N | TYR | 62 | 69.180 | 30.970 | 32.978 | 1.00 | 40.18 |
| ATOM | 475 | CA | TYR | 62 | 70.459 | 30.319 | 33.237 | 1.00 | 40.06 |
| ATOM | 476 | CB | TYR | 62 | 71.589 | 31.350 | 33.229 | 1.00 | 39.70 |
| ATOM | 477 | CG | TYR | 62 | 71.461 | 32.427 | 34.287 | 1.00 | 39.27 |
| ATOM | 478 | CD1 | TYR | 62 | 71.835 | 32.188 | 35.610 | 1.00 | 38.87 |
| ATOM | 479 | CE1 | TYR | 62 | 71.724 | 33.190 | 36.589 | 1.00 | 39.73 |
| ATOM | 480 | CD2 | TYR | 62 | 70.965 | 33.698 | 33.960 | 1.00 | 40.72 |
| ATOM | 481 | CE2 | TYR | 62 | 70.847 | 34.710 | 34.925 | 1.00 | 40.09 |
| ATOM | 482 | CZ | TYR | 62 | 71.227 | 34.448 | 36.239 | 1.00 | 41.39 |
| ATOM | 483 | OH | TYR | 62 | 71.094 | 35.431 | 37.197 | 1.00 | 39.21 |
| ATOM | 484 | C | TYR | 62 | 70.410 | 29.634 | 34.598 | 1.00 | 40.89 |
| ATOM | 485 | O | TYR | 62 | 69.461 | 29.823 | 35.361 | 1.00 | 41.00 |
| ATOM | 486 | N | VAL | 63 | 71.439 | 28.841 | 34.896 | 1.00 | 41.43 |
| ATOM | 487 | CA | VAL | 63 | 71.530 | 28.122 | 36.167 | 1.00 | 39.60 |
| ATOM | 488 | CB | VAL | 63 | 71.634 | 26.596 | 35.959 | 1.00 | 37.74 |
| ATOM | 489 | CG1 | VAL | 63 | 71.827 | 25.916 | 37.298 | 1.00 | 36.52 |
| ATOM | 490 | CG2 | VAL | 63 | 70.376 | 26.063 | 35.276 | 1.00 | 37.06 |
| ATOM | 491 | C | VAL | 63 | 72.759 | 28.568 | 36.944 | 1.00 | 40.62 |
| ATOM | 492 | O | VAL | 63 | 73.886 | 28.332 | 36.526 | 1.00 | 40.79 |
| ATOM | 493 | N | LEU | 64 | 72.532 | 29.210 | 38.083 | 1.00 | 42.23 |
| ATOM | 494 | CA | LEU | 64 | 73.613 | 29.689 | 38.928 | 1.00 | 41.00 |
| ATOM | 495 | CB | LEU | 64 | 73.435 | 31.182 | 39.213 | 1.00 | 41.07 |
| ATOM | 496 | CG | LEU | 64 | 74.493 | 31.862 | 40.092 | 1.00 | 42.23 |
| ATOM | 497 | CD1 | LEU | 64 | 75.867 | 31.792 | 39.419 | 1.00 | 37.35 |
| ATOM | 498 | CD2 | LEU | 64 | 74.070 | 33.317 | 40.343 | 1.00 | 37.32 |
| ATOM | 499 | C | LEU | 64 | 73.605 | 28.906 | 40.232 | 1.00 | 42.23 |
| ATOM | 500 | O | LEU | 64 | 72.571 | 28.781 | 40.894 | 1.00 | 41.03 |
| ATOM | 501 | N | ILE | 65 | 74.766 | 28.361 | 40.574 | 1.00 | 42.40 |
| ATOM | 502 | CA | ILE | 65 | 74.960 | 27.587 | 41.791 | 1.00 | 41.32 |
| ATOM | 503 | CB | ILE | 65 | 75.166 | 26.101 | 41.494 | 1.00 | 39.96 |
| ATOM | 504 | CG2 | ILE | 65 | 75.532 | 25.368 | 42.783 | 1.00 | 41.29 |
| ATOM | 505 | CG1 | ILE | 65 | 73.906 | 25.517 | 40.855 | 1.00 | 40.19 |
| ATOM | 506 | CD1 | ILE | 65 | 74.037 | 24.057 | 40.414 | 1.00 | 36.26 |
| ATOM | 507 | C | ILE | 65 | 76.255 | 28.140 | 42.336 | 1.00 | 42.20 |
| ATOM | 508 | O | ILE | 65 | 77.323 | 27.816 | 41.827 | 1.00 | 42.24 |
| ATOM | 509 | N | ALA | 66 | 76.173 | 28.976 | 43.363 | 1.00 | 43.21 |
| ATOM | 510 | CA | ALA | 66 | 77.383 | 29.579 | 43.892 | 1.00 | 44.14 |
| ATOM | 511 | CB | ALA | 66 | 77.696 | 30.853 | 43.103 | 1.00 | 39.19 |
| ATOM | 512 | C | ALA | 66 | 77.348 | 29.890 | 45.379 | 1.00 | 46.15 |
| ATOM | 513 | O | ALA | 66 | 76.285 | 30.089 | 45.963 | 1.00 | 46.35 |
| ATOM | 514 | N | HIS | 67 | 78.537 | 29.936 | 45.976 | 1.00 | 48.07 |
| ATOM | 515 | CA | HIS | 67 | 78.697 | 30.243 | 47.386 | 1.00 | 49.29 |
| ATOM | 516 | CB | HIS | 67 | 78.336 | 31.705 | 47.636 | 1.00 | 51.64 |
| ATOM | 517 | CG | HIS | 67 | 79.390 | 32.669 | 47.190 | 1.00 | 54.81 |
| ATOM | 518 | CD2 | HIS | 67 | 79.392 | 33.596 | 46.203 | 1.00 | 56.63 |
| ATOM | 519 | ND1 | HIS | 67 | 80.625 | 32.753 | 47.797 | 1.00 | 57.63 |
| ATOM | 520 | CE1 | HIS | 67 | 81.343 | 33.692 | 47.205 | 1.00 | 57.69 |
| ATOM | 521 | NE2 | HIS | 67 | 80.617 | 34.219 | 46.234 | 1.00 | 57.89 |
| ATOM | 522 | C | HIS | 67 | 77.892 | 29.338 | 48.305 | 1.00 | 49.42 |
| ATOM | 523 | O | HIS | 67 | 77.306 | 29.786 | 49.289 | 1.00 | 50.64 |
| ATOM | 524 | N | ASN | 68 | 77.871 | 28.055 | 47.987 | 1.00 | 49.48 |
| ATOM | 525 | CA | ASN | 68 | 77.147 | 27.111 | 48.810 | 1.00 | 49.88 |
| ATOM | 526 | CB | ASN | 68 | 76.222 | 26.266 | 47.942 | 1.00 | 48.94 |
| ATOM | 527 | CG | ASN | 68 | 75.243 | 27.113 | 47.157 | 1.00 | 50.57 |
| ATOM | 528 | OD1 | ASN | 68 | 74.477 | 27.890 | 47.737 | 1.00 | 48.22 |
| ATOM | 529 | ND2 | ASN | 68 | 75.266 | 26.977 | 45.830 | 1.00 | 48.81 |
| ATOM | 530 | C | ASN | 68 | 78.135 | 26.222 | 49.543 | 1.00 | 51.48 |
| ATOM | 531 | O | ASN | 68 | 79.266 | 26.020 | 49.093 | 1.00 | 51.22 |
| ATOM | 532 | N | GLN | 69 | 77.704 | 25.713 | 50.689 | 1.00 | 52.43 |
| ATOM | 533 | CA | GLN | 69 | 78.525 | 24.825 | 51.488 | 1.00 | 53.43 |
| ATOM | 534 | CB | GLN | 69 | 78.554 | 25.292 | 52.944 | 1.00 | 55.53 |
| ATOM | 535 | CG | GLN | 69 | 79.220 | 26.638 | 53.133 | 1.00 | 60.31 |
| ATOM | 536 | CD | GLN | 69 | 80.547 | 26.714 | 52.402 | 1.00 | 64.35 |
| ATOM | 537 | OE1 | GLN | 69 | 81.401 | 25.840 | 52.559 | 1.00 | 66.60 |
| ATOM | 538 | NE2 | GLN | 69 | 80.728 | 27.759 | 51.595 | 1.00 | 65.26 |
| ATOM | 539 | C | GLN | 69 | 77.910 | 23.439 | 51.390 | 1.00 | 52.50 |
| ATOM | 540 | O | GLN | 69 | 78.518 | 22.450 | 51.788 | 1.00 | 55.45 |
| ATOM | 541 | N | VAL | 70 | 76.698 | 23.375 | 50.848 | 1.00 | 49.65 |
| ATOM | 542 | CA | VAL | 70 | 75.990 | 22.112 | 50.689 | 1.00 | 47.14 |
| ATOM | 543 | CB | VAL | 70 | 74.576 | 22.365 | 50.098 | 1.00 | 45.05 |
| ATOM | 544 | CG1 | VAL | 70 | 74.673 | 22.681 | 48.614 | 1.00 | 43.90 |
| ATOM | 545 | CG2 | VAL | 70 | 73.676 | 21.176 | 50.366 | 1.00 | 45.15 |
| ATOM | 546 | C | VAL | 70 | 76.814 | 21.173 | 49.787 | 1.00 | 47.49 |
| ATOM | 547 | O | VAL | 70 | 77.539 | 21.622 | 48.905 | 1.00 | 44.75 |
| ATOM | 548 | N | ARG | 71 | 76.702 | 19.869 | 50.002 | 1.00 | 50.39 |
| ATOM | 549 | CA | ARG | 71 | 77.497 | 18.931 | 49.219 | 1.00 | 55.11 |
| ATOM | 550 | CB | ARG | 71 | 77.877 | 17.720 | 50.078 | 1.00 | 56.84 |
| ATOM | 551 | CG | ARG | 71 | 78.651 | 18.115 | 51.328 | 1.00 | 60.61 |
| ATOM | 552 | CD | ARG | 71 | 79.305 | 16.937 | 52.011 | 1.00 | 63.62 |
| ATOM | 553 | NE | ARG | 71 | 79.895 | 17.351 | 53.279 | 1.00 | 67.41 |
| ATOM | 554 | CZ | ARG | 71 | 79.191 | 17.586 | 54.383 | 1.00 | 70.48 |
| ATOM | 555 | NH1 | ARG | 71 | 77.869 | 17.439 | 54.377 | 1.00 | 71.09 |
| ATOM | 556 | NH2 | ARG | 71 | 79.806 | 17.984 | 55.490 | 1.00 | 70.91 |
| ATOM | 557 | C | ARG | 71 | 76.874 | 18.470 | 47.916 | 1.00 | 56.48 |
| ATOM | 558 | O | ARG | 71 | 77.592 | 18.082 | 46.984 | 1.00 | 55.56 |
| ATOM | 559 | N | GLN | 72 | 75.546 | 18.511 | 47.846 | 1.00 | 57.00 |
| ATOM | 560 | CA | GLN | 72 | 74.855 | 18.110 | 46.632 | 1.00 | 56.93 |
| ATOM | 561 | CB | GLN | 72 | 74.520 | 16.616 | 46.662 | 1.00 | 57.34 |
| ATOM | 562 | CG | GLN | 72 | 73.595 | 16.185 | 45.505 | 1.00 | 61.22 |
| ATOM | 563 | CD | GLN | 72 | 74.186 | 16.421 | 44.097 | 1.00 | 62.35 |
| ATOM | 564 | OE1 | GLN | 72 | 74.791 | 17.467 | 43.810 | 1.00 | 59.65 |
| ATOM | 565 | NE2 | GLN | 72 | 73.988 | 15.444 | 43.210 | 1.00 | 63.04 |
| ATOM | 566 | C | GLN | 72 | 73.582 | 18.902 | 46.365 | 1.00 | 55.55 |
| ATOM | 567 | O | GLN | 72 | 72.872 | 19.300 | 47.284 | 1.00 | 55.69 |
| ATOM | 568 | N | VAL | 73 | 73.319 | 19.124 | 45.082 | 1.00 | 54.03 |
| ATOM | 569 | CA | VAL | 73 | 72.147 | 19.842 | 44.608 | 1.00 | 52.00 |
| ATOM | 570 | CB | VAL | 73 | 72.571 | 21.137 | 43.888 | 1.00 | 52.61 |
| ATOM | 571 | CG1 | VAL | 73 | 73.274 | 22.065 | 44.869 | 1.00 | 51.82 |
| ATOM | 572 | CG2 | VAL | 73 | 73.537 | 20.806 | 42.753 | 1.00 | 57.12 |
| ATOM | 573 | C | VAL | 73 | 71.460 | 18.881 | 43.637 | 1.00 | 50.04 |
| ATOM | 574 | O | VAL | 73 | 71.853 | 18.758 | 42.489 | 1.00 | 49.44 |
| ATOM | 575 | N | PRO | 74 | 70.425 | 18.173 | 44.103 | 1.00 | 50.40 |
| ATOM | 576 | CD | PRO | 74 | 69.847 | 18.290 | 45.453 | 1.00 | 49.59 |
| ATOM | 577 | CA | PRO | 74 | 69.670 | 17.202 | 43.304 | 1.00 | 50.32 |
| ATOM | 578 | CB | PRO | 74 | 68.715 | 16.589 | 44.330 | 1.00 | 50.82 |
| ATOM | 579 | CG | PRO | 74 | 68.455 | 17.735 | 45.253 | 1.00 | 50.68 |
| ATOM | 580 | C | PRO | 74 | 68.936 | 17.697 | 42.056 | 1.00 | 49.40 |
| ATOM | 581 | O | PRO | 74 | 67.708 | 17.846 | 42.051 | 1.00 | 50.49 |
| ATOM | 582 | N | LEU | 75 | 69.694 | 17.925 | 40.991 | 1.00 | 47.89 |
| ATOM | 583 | CA | LEU | 75 | 69.124 | 18.366 | 39.724 | 1.00 | 46.86 |
| ATOM | 584 | CB | LEU | 75 | 69.824 | 19.644 | 39.254 | 1.00 | 43.48 |
| ATOM | 585 | CG | LEU | 75 | 69.457 | 20.850 | 40.125 | 1.00 | 43.50 |
| ATOM | 586 | CD1 | LEU | 75 | 70.382 | 22.007 | 39.846 | 1.00 | 40.95 |
| ATOM | 587 | CD2 | LEU | 75 | 68.004 | 21.235 | 39.862 | 1.00 | 42.32 |
| ATOM | 588 | C | LEU | 75 | 69.253 | 17.264 | 38.675 | 1.00 | 46.45 |
| ATOM | 589 | O | LEU | 75 | 69.535 | 17.541 | 37.510 | 1.00 | 46.20 |
| ATOM | 590 | N | GLN | 76 | 69.034 | 16.016 | 39.094 | 1.00 | 47.00 |
| ATOM | 591 | CA | GLN | 76 | 69.138 | 14.871 | 38.191 | 1.00 | 46.56 |
| ATOM | 592 | CB | GLN | 76 | 69.037 | 13.545 | 38.949 | 1.00 | 48.03 |
| ATOM | 593 | CG | GLN | 76 | 70.267 | 13.160 | 39.761 | 1.00 | 52.56 |
| ATOM | 594 | CD | GLN | 76 | 70.356 | 13.903 | 41.088 | 1.00 | 54.82 |
| ATOM | 595 | OE1 | GLN | 76 | 69.411 | 14.601 | 41.485 | 1.00 | 56.30 |
| ATOM | 596 | NE2 | GLN | 76 | 71.486 | 13.746 | 41.789 | 1.00 | 51.88 |
| ATOM | 597 | C | GLN | 76 | 68.128 | 14.842 | 37.054 | 1.00 | 45.92 |
| ATOM | 598 | O | GLN | 76 | 68.341 | 14.118 | 36.087 | 1.00 | 46.57 |
| ATOM | 599 | N | ARG | 77 | 67.037 | 15.605 | 37.145 | 1.00 | 44.53 |
| ATOM | 600 | CA | ARG | 77 | 66.058 | 15.592 | 36.053 | 1.00 | 42.84 |
| ATOM | 601 | CB | ARG | 77 | 64.645 | 15.385 | 36.586 | 1.00 | 45.72 |
| ATOM | 602 | CG | ARG | 77 | 64.453 | 14.065 | 37.281 | 1.00 | 50.95 |
| ATOM | 603 | CD | ARG | 77 | 62.984 | 13.699 | 37.368 | 1.00 | 55.58 |
| ATOM | 604 | NE | ARG | 77 | 62.768 | 12.654 | 38.363 | 1.00 | 61.00 |
| ATOM | 605 | CZ | ARG | 77 | 62.080 | 12.832 | 39.487 | 1.00 | 64.13 |
| ATOM | 606 | NH1 | ARG | 77 | 61.538 | 14.017 | 39.746 | 1.00 | 65.36 |
| ATOM | 607 | NH2 | ARG | 77 | 61.940 | 11.835 | 40.357 | 1.00 | 64.68 |
| ATOM | 608 | C | ARG | 77 | 66.083 | 16.825 | 35.155 | 1.00 | 40.56 |
| ATOM | 609 | O | ARG | 77 | 65.224 | 16.997 | 34.288 | 1.00 | 38.61 |
| ATOM | 610 | N | LEU | 78 | 67.068 | 17.687 | 35.361 | 1.00 | 38.63 |
| ATOM | 611 | CA | LEU | 78 | 67.191 | 18.877 | 34.539 | 1.00 | 37.84 |
| ATOM | 612 | CB | LEU | 78 | 68.222 | 19.828 | 35.133 | 1.00 | 35.63 |
| ATOM | 613 | CG | LEU | 78 | 68.500 | 21.074 | 34.304 | 1.00 | 34.86 |
| ATOM | 614 | CD1 | LEU | 78 | 67.195 | 21.841 | 34.072 | 1.00 | 33.94 |
| ATOM | 615 | CD2 | LEU | 78 | 69.524 | 21.923 | 35.024 | 1.00 | 32.14 |
| ATOM | 616 | C | LEU | 78 | 67.631 | 18.424 | 33.157 | 1.00 | 39.74 |
| ATOM | 617 | O | LEU | 78 | 68.751 | 17.947 | 32.968 | 1.00 | 38.58 |
| ATOM | 618 | N | ARG | 79 | 66.731 | 18.563 | 32.194 | 1.00 | 42.07 |
| ATOM | 619 | CA | ARG | 79 | 67.001 | 18.154 | 30.824 | 1.00 | 43.44 |
| ATOM | 620 | CB | ARG | 79 | 65.719 | 17.640 | 30.186 | 1.00 | 44.33 |
| ATOM | 621 | CG | ARG | 79 | 65.967 | 16.780 | 29.007 | 1.00 | 49.41 |
| ATOM | 622 | CD | ARG | 79 | 66.291 | 15.400 | 29.506 | 1.00 | 57.39 |
| ATOM | 623 | NE | ARG | 79 | 67.309 | 14.738 | 28.707 | 1.00 | 58.07 |
| ATOM | 624 | CZ | ARG | 79 | 67.735 | 13.513 | 28.956 | 1.00 | 60.12 |
| ATOM | 625 | NH1 | ARG | 79 | 67.215 | 12.848 | 29.977 | 1.00 | 61.40 |
| ATOM | 626 | NH2 | ARG | 79 | 68.677 | 12.965 | 28.198 | 1.00 | 62.59 |
| ATOM | 627 | C | ARG | 79 | 67.554 | 19.259 | 29.925 | 1.00 | 44.07 |
| ATOM | 628 | O | ARG | 79 | 68.445 | 19.024 | 29.110 | 1.00 | 46.42 |
| ATOM | 629 | N | ILE | 80 | 67.034 | 20.469 | 30.074 | 1.00 | 44.22 |
| ATOM | 630 | CA | ILE | 80 | 67.449 | 21.547 | 29.197 | 1.00 | 44.40 |
| ATOM | 631 | CB | ILE | 80 | 66.516 | 21.562 | 27.968 | 1.00 | 45.97 |
| ATOM | 632 | CG2 | ILE | 80 | 65.072 | 21.389 | 28.421 | 1.00 | 45.82 |
| ATOM | 633 | CG1 | ILE | 80 | 66.676 | 22.857 | 27.179 | 1.00 | 47.04 |
| ATOM | 634 | CD1 | ILE | 80 | 65.668 | 22.989 | 26.046 | 1.00 | 49.16 |
| ATOM | 635 | C | ILE | 80 | 67.473 | 22.937 | 29.813 | 1.00 | 43.53 |
| ATOM | 636 | O | ILE | 80 | 66.607 | 23.297 | 30.606 | 1.00 | 46.12 |
| ATOM | 637 | N | VAL | 81 | 68.492 | 23.707 | 29.453 | 1.00 | 41.12 |
| ATOM | 638 | CA | VAL | 81 | 68.617 | 25.078 | 29.909 | 1.00 | 39.98 |
| ATOM | 639 | CB | VAL | 81 | 69.940 | 25.317 | 30.637 | 1.00 | 37.75 |
| ATOM | 640 | CG1 | VAL | 81 | 70.063 | 26.790 | 31.019 | 1.00 | 33.86 |
| ATOM | 641 | CG2 | VAL | 81 | 70.005 | 24.441 | 31.880 | 1.00 | 37.92 |
| ATOM | 642 | C | VAL | 81 | 68.591 | 25.895 | 28.622 | 1.00 | 41.11 |
| ATOM | 643 | O | VAL | 81 | 69.516 | 25.805 | 27.817 | 1.00 | 41.60 |
| ATOM | 644 | N | ARG | 82 | 67.525 | 26.669 | 28.422 | 1.00 | 40.63 |
| ATOM | 645 | CA | ARG | 82 | 67.366 | 27.474 | 27.213 | 1.00 | 41.36 |
| ATOM | 646 | CB | ARG | 82 | 65.906 | 27.914 | 27.057 | 1.00 | 41.22 |
| ATOM | 647 | CG | ARG | 82 | 64.970 | 26.741 | 26.805 | 1.00 | 41.15 |
| ATOM | 648 | CD | ARG | 82 | 63.507 | 27.152 | 26.721 | 1.00 | 39.21 |
| ATOM | 649 | NE | ARG | 82 | 62.642 | 25.974 | 26.746 | 1.00 | 39.74 |
| ATOM | 650 | CZ | ARG | 82 | 62.532 | 25.094 | 25.755 | 1.00 | 37.15 |
| ATOM | 651 | NH1 | ARG | 82 | 63.223 | 25.249 | 24.641 | 1.00 | 36.57 |
| ATOM | 652 | NH2 | ARG | 82 | 61.735 | 24.047 | 25.888 | 1.00 | 39.83 |
| ATOM | 653 | C | ARG | 82 | 68.279 | 28.682 | 27.126 | 1.00 | 40.96 |
| ATOM | 654 | O | ARG | 82 | 68.615 | 29.123 | 26.035 | 1.00 | 43.30 |
| ATOM | 655 | N | GLY | 83 | 68.677 | 29.222 | 28.267 | 1.00 | 41.75 |
| ATOM | 656 | CA | GLY | 83 | 69.573 | 30.363 | 28.252 | 1.00 | 42.37 |
| ATOM | 657 | C | GLY | 83 | 69.023 | 31.619 | 27.601 | 1.00 | 43.59 |
| ATOM | 658 | O | GLY | 83 | 69.744 | 32.317 | 26.878 | 1.00 | 41.39 |
| ATOM | 659 | N | THR | 84 | 67.747 | 31.906 | 27.854 | 1.00 | 43.83 |
| ATOM | 660 | CA | THR | 84 | 67.111 | 33.099 | 27.311 | 1.00 | 43.71 |
| ATOM | 661 | CB | THR | 84 | 65.603 | 33.090 | 27.582 | 1.00 | 43.70 |
| ATOM | 662 | OG1 | THR | 84 | 64.976 | 32.119 | 26.732 | 1.00 | 44.53 |
| ATOM | 663 | CG2 | THR | 84 | 64.997 | 34.446 | 27.312 | 1.00 | 46.08 |
| ATOM | 664 | C | THR | 84 | 67.748 | 34.291 | 27.999 | 1.00 | 45.07 |
| ATOM | 665 | O | THR | 84 | 67.577 | 35.433 | 27.590 | 1.00 | 45.57 |
| ATOM | 666 | N | GLN | 85 | 68.499 | 33.999 | 29.053 | 1.00 | 46.19 |
| ATOM | 667 | CA | GLN | 85 | 69.202 | 35.009 | 29.827 | 1.00 | 47.00 |
| ATOM | 668 | CB | GLN | 85 | 68.398 | 35.387 | 31.064 | 1.00 | 48.71 |
| ATOM | 669 | CG | GLN | 85 | 67.018 | 35.903 | 30.776 | 1.00 | 54.49 |
| ATOM | 670 | CD | GLN | 85 | 66.134 | 35.842 | 32.007 | 1.00 | 59.66 |
| ATOM | 671 | OE1 | GLN | 85 | 66.463 | 36.415 | 33.053 | 1.00 | 59.91 |
| ATOM | 672 | NE2 | GLN | 85 | 65.005 | 35.138 | 31.893 | 1.00 | 60.96 |
| ATOM | 673 | C | GLN | 85 | 70.497 | 34.343 | 30.249 | 1.00 | 46.30 |
| ATOM | 674 | O | GLN | 85 | 70.515 | 33.135 | 30.496 | 1.00 | 45.05 |
| ATOM | 675 | N | LEU | 86 | 71.575 | 35.113 | 30.350 | 1.00 | 44.87 |
| ATOM | 676 | CA | LEU | 86 | 72.850 | 34.522 | 30.724 | 1.00 | 44.82 |
| ATOM | 677 | CB | LEU | 86 | 73.803 | 34.532 | 29.535 | 1.00 | 42.60 |
| ATOM | 678 | CG | LEU | 86 | 73.321 | 33.774 | 28.304 | 1.00 | 44.41 |
| ATOM | 679 | CD1 | LEU | 86 | 74.329 | 33.988 | 27.186 | 1.00 | 47.40 |
| ATOM | 680 | CD2 | LEU | 86 | 73.161 | 32.287 | 28.620 | 1.00 | 42.93 |
| ATOM | 681 | C | LEU | 86 | 73.523 | 35.216 | 31.879 | 1.00 | 45.57 |
| ATOM | 682 | O | LEU | 86 | 73.408 | 36.429 | 32.045 | 1.00 | 48.26 |
| ATOM | 683 | N | PHE | 87 | 74.225 | 34.434 | 32.683 | 1.00 | 43.87 |
| ATOM | 684 | CA | PHE | 87 | 74.959 | 34.987 | 33.800 | 1.00 | 43.63 |
| ATOM | 685 | CB | PHE | 87 | 75.444 | 33.853 | 34.689 | 1.00 | 40.78 |
| ATOM | 686 | CG | PHE | 87 | 76.231 | 34.313 | 35.857 | 1.00 | 40.85 |
| ATOM | 687 | CD1 | PHE | 87 | 75.626 | 35.038 | 36.869 | 1.00 | 40.47 |
| ATOM | 688 | CD2 | PHE | 87 | 77.586 | 34.037 | 35.946 | 1.00 | 41.63 |
| ATOM | 689 | CE1 | PHE | 87 | 76.362 | 35.481 | 37.955 | 1.00 | 41.04 |
| ATOM | 690 | CE2 | PHE | 87 | 78.332 | 34.476 | 37.031 | 1.00 | 40.73 |
| ATOM | 691 | CZ | PHE | 87 | 77.719 | 35.200 | 38.037 | 1.00 | 40.21 |
| ATOM | 692 | C | PHE | 87 | 76.143 | 35.729 | 33.154 | 1.00 | 45.18 |
| ATOM | 693 | O | PHE | 87 | 76.785 | 35.199 | 32.244 | 1.00 | 44.05 |
| ATOM | 694 | N | GLU | 88 | 76.420 | 36.951 | 33.610 | 1.00 | 46.88 |
| ATOM | 695 | CA | GLU | 88 | 77.496 | 37.769 | 33.046 | 1.00 | 50.12 |
| ATOM | 696 | CB | GLU | 88 | 78.869 | 37.225 | 33.436 | 1.00 | 50.94 |
| ATOM | 697 | CG | GLU | 88 | 79.183 | 37.333 | 34.915 | 1.00 | 54.47 |
| ATOM | 698 | CD | GLU | 88 | 80.582 | 36.842 | 35.252 | 1.00 | 56.68 |
| ATOM | 699 | OE1 | GLU | 88 | 80.903 | 36.719 | 36.461 | 1.00 | 58.66 |
| ATOM | 700 | OE2 | GLU | 88 | 81.363 | 36.584 | 34.308 | 1.00 | 56.70 |
| ATOM | 701 | C | GLU | 88 | 77.398 | 37.837 | 31.524 | 1.00 | 52.26 |
| ATOM | 702 | O | GLU | 88 | 78.383 | 38.105 | 30.838 | 1.00 | 53.70 |
| ATOM | 703 | N | ASP | 89 | 76.205 | 37.575 | 31.004 | 1.00 | 53.97 |
| ATOM | 704 | CA | ASP | 89 | 75.954 | 37.622 | 29.566 | 1.00 | 55.67 |
| ATOM | 705 | CB | ASP | 89 | 76.369 | 38.988 | 29.012 | 1.00 | 56.99 |
| ATOM | 706 | CG | ASP | 89 | 75.403 | 40.082 | 29.404 | 1.00 | 59.00 |
| ATOM | 707 | OD1 | ASP | 89 | 74.189 | 39.894 | 29.157 | 1.00 | 60.54 |
| ATOM | 708 | OD2 | ASP | 89 | 75.850 | 41.115 | 29.956 | 1.00 | 56.93 |
| ATOM | 709 | C | ASP | 89 | 76.579 | 36.527 | 28.706 | 1.00 | 54.74 |
| ATOM | 710 | O | ASP | 89 | 76.530 | 36.604 | 27.478 | 1.00 | 55.07 |
| ATOM | 711 | N | ASN | 90 | 77.143 | 35.497 | 29.325 | 1.00 | 54.15 |
| ATOM | 712 | CA | ASN | 90 | 77.764 | 34.438 | 28.538 | 1.00 | 52.57 |
| ATOM | 713 | CB | ASN | 90 | 79.277 | 34.595 | 28.584 | 1.00 | 52.47 |
| ATOM | 714 | CG | ASN | 90 | 79.737 | 35.890 | 27.980 | 1.00 | 53.25 |
| ATOM | 715 | OD1 | ASN | 90 | 79.531 | 36.135 | 26.794 | 1.00 | 54.48 |
| ATOM | 716 | ND2 | ASN | 90 | 80.359 | 36.736 | 28.791 | 1.00 | 52.67 |
| ATOM | 717 | C | ASN | 90 | 77.426 | 33.015 | 28.949 | 1.00 | 51.81 |
| ATOM | 718 | O | ASN | 90 | 77.363 | 32.115 | 28.114 | 1.00 | 51.39 |
| ATOM | 719 | N | TYR | 91 | 77.187 | 32.803 | 30.231 | 1.00 | 50.25 |
| ATOM | 720 | CA | TYR | 91 | 76.949 | 31.453 | 30.677 | 1.00 | 49.51 |
| ATOM | 721 | CB | TYR | 91 | 77.887 | 31.174 | 31.845 | 1.00 | 51.13 |
| ATOM | 722 | CG | TYR | 91 | 79.291 | 31.674 | 31.567 | 1.00 | 52.63 |
| ATOM | 723 | CD1 | TYR | 91 | 79.699 | 32.943 | 31.984 | 1.00 | 53.04 |
| ATOM | 724 | CE1 | TYR | 91 | 80.979 | 33.427 | 31.696 | 1.00 | 53.62 |
| ATOM | 725 | CD2 | TYR | 91 | 80.202 | 30.893 | 30.851 | 1.00 | 54.91 |
| ATOM | 726 | CE2 | TYR | 91 | 81.489 | 31.368 | 30.554 | 1.00 | 55.55 |
| ATOM | 727 | CZ | TYR | 91 | 81.869 | 32.634 | 30.981 | 1.00 | 55.51 |
| ATOM | 728 | OH | TYR | 91 | 83.136 | 33.101 | 30.703 | 1.00 | 54.62 |
| ATOM | 729 | C | TYR | 91 | 75.535 | 31.020 | 31.013 | 1.00 | 47.39 |
| ATOM | 730 | O | TYR | 91 | 74.803 | 31.713 | 31.719 | 1.00 | 47.32 |
| ATOM | 731 | N | ALA | 92 | 75.161 | 29.864 | 30.471 | 1.00 | 44.48 |
| ATOM | 732 | CA | ALA | 92 | 73.863 | 29.281 | 30.730 | 1.00 | 43.50 |
| ATOM | 733 | CB | ALA | 92 | 73.474 | 28.356 | 29.613 | 1.00 | 40.46 |
| ATOM | 734 | C | ALA | 92 | 74.012 | 28.499 | 32.030 | 1.00 | 44.42 |
| ATOM | 735 | O | ALA | 92 | 73.025 | 28.223 | 32.716 | 1.00 | 46.43 |
| ATOM | 736 | N | LEU | 93 | 75.252 | 28.136 | 32.359 | 1.00 | 42.65 |
| ATOM | 737 | CA | LEU | 93 | 75.539 | 27.395 | 33.583 | 1.00 | 41.17 |
| ATOM | 738 | CB | LEU | 93 | 75.734 | 25.917 | 33.284 | 1.00 | 38.62 |
| ATOM | 739 | CG | LEU | 93 | 76.143 | 25.090 | 34.505 | 1.00 | 37.50 |
| ATOM | 740 | CD1 | LEU | 93 | 75.067 | 25.170 | 35.582 | 1.00 | 38.29 |
| ATOM | 741 | CD2 | LEU | 93 | 76.360 | 23.652 | 34.087 | 1.00 | 36.61 |
| ATOM | 742 | C | LEU | 93 | 76.776 | 27.934 | 34.298 | 1.00 | 41.94 |
| ATOM | 743 | O | LEU | 93 | 77.865 | 27.986 | 33.730 | 1.00 | 42.96 |
| ATOM | 744 | N | ALA | 94 | 76.599 | 28.325 | 35.554 | 1.00 | 40.94 |
| ATOM | 745 | CA | ALA | 94 | 77.686 | 28.878 | 36.335 | 1.00 | 41.22 |
| ATOM | 746 | CB | ALA | 94 | 77.506 | 30.378 | 36.461 | 1.00 | 39.61 |
| ATOM | 747 | C | ALA | 94 | 77.766 | 28.241 | 37.720 | 1.00 | 43.35 |
| ATOM | 748 | O | ALA | 94 | 76.814 | 28.299 | 38.499 | 1.00 | 42.96 |
| ATOM | 749 | N | VAL | 95 | 78.911 | 27.632 | 38.017 | 1.00 | 44.35 |
| ATOM | 750 | CA | VAL | 95 | 79.143 | 26.996 | 39.307 | 1.00 | 45.11 |
| ATOM | 751 | CB | VAL | 95 | 79.388 | 25.500 | 39.125 | 1.00 | 43.05 |
| ATOM | 752 | CG1 | VAL | 95 | 79.533 | 24.819 | 40.479 | 1.00 | 39.27 |
| ATOM | 753 | CG2 | VAL | 95 | 78.240 | 24.899 | 38.312 | 1.00 | 40.86 |
| ATOM | 754 | C | VAL | 95 | 80.369 | 27.669 | 39.925 | 1.00 | 48.52 |
| ATOM | 755 | O | VAL | 95 | 81.508 | 27.415 | 39.517 | 1.00 | 48.34 |
| ATOM | 756 | N | LEU | 96 | 80.128 | 28.524 | 40.916 | 1.00 | 50.32 |
| ATOM | 757 | CA | LEU | 96 | 81.207 | 29.272 | 41.542 | 1.00 | 51.50 |
| ATOM | 758 | CB | LEU | 96 | 81.074 | 30.739 | 41.150 | 1.00 | 51.70 |
| ATOM | 759 | CG | LEU | 96 | 80.684 | 30.959 | 39.690 | 1.00 | 52.86 |
| ATOM | 760 | CD1 | LEU | 96 | 80.346 | 32.418 | 39.457 | 1.00 | 52.63 |
| ATOM | 761 | CD2 | LEU | 96 | 81.821 | 30.512 | 38.793 | 1.00 | 53.82 |
| ATOM | 762 | C | LEU | 96 | 81.323 | 29.181 | 43.059 | 1.00 | 53.24 |
| ATOM | 763 | O | LEU | 96 | 80.344 | 28.970 | 43.774 | 1.00 | 53.15 |
| ATOM | 764 | N | ASP | 97 | 82.555 | 29.359 | 43.524 | 1.00 | 54.82 |
| ATOM | 765 | CA | ASP | 97 | 82.906 | 29.347 | 44.938 | 1.00 | 55.39 |
| ATOM | 766 | CB | ASP | 97 | 82.927 | 30.780 | 45.456 | 1.00 | 53.38 |
| ATOM | 767 | CG | ASP | 97 | 83.658 | 31.711 | 44.521 | 1.00 | 53.93 |
| ATOM | 768 | OD1 | ASP | 97 | 83.020 | 32.242 | 43.586 | 1.00 | 52.94 |
| ATOM | 769 | OD2 | ASP | 97 | 84.879 | 31.892 | 44.706 | 1.00 | 53.79 |
| ATOM | 770 | C | ASP | 97 | 82.055 | 28.476 | 45.850 | 1.00 | 57.18 |
| ATOM | 771 | O | ASP | 97 | 81.505 | 28.948 | 46.849 | 1.00 | 55.28 |
| ATOM | 772 | N | ASN | 98 | 81.962 | 27.197 | 45.510 | 1.00 | 59.71 |
| ATOM | 773 | CA | ASN | 98 | 81.208 | 26.253 | 46.318 | 1.00 | 63.26 |
| ATOM | 774 | CB | ASN | 98 | 80.327 | 25.394 | 45.421 | 1.00 | 61.80 |
| ATOM | 775 | CG | ASN | 98 | 79.265 | 26.203 | 44.735 | 1.00 | 60.81 |
| ATOM | 776 | OD1 | ASN | 98 | 78.351 | 26.712 | 45.383 | 1.00 | 62.45 |
| ATOM | 777 | ND2 | ASN | 98 | 79.382 | 26.350 | 43.419 | 1.00 | 59.92 |
| ATOM | 778 | C | ASN | 98 | 82.186 | 25.391 | 47.108 | 1.00 | 65.67 |
| ATOM | 779 | O | ASN | 98 | 82.574 | 24.313 | 46.673 | 1.00 | 65.42 |
| ATOM | 780 | N | GLY | 99 | 82.579 | 25.894 | 48.272 | 1.00 | 70.08 |
| ATOM | 781 | CA | GLY | 99 | 83.520 | 25.197 | 49.125 | 1.00 | 76.22 |
| ATOM | 782 | C | GLY | 99 | 84.105 | 26.187 | 50.116 | 1.00 | 81.42 |
| ATOM | 783 | O | GLY | 99 | 84.487 | 27.299 | 49.740 | 1.00 | 81.92 |
| ATOM | 784 | N | ASP | 100 | 84.176 | 25.774 | 51.374 | 1.00 | 86.13 |
| ATOM | 785 | CA | ASP | 100 | 84.686 | 26.590 | 52.475 | 1.00 | 90.98 |
| ATOM | 786 | CB | ASP | 100 | 85.046 | 25.680 | 53.656 | 1.00 | 92.47 |
| ATOM | 787 | CG | ASP | 100 | 83.883 | 24.805 | 54.092 | 1.00 | 94.56 |
| ATOM | 788 | OD1 | ASP | 100 | 82.875 | 25.358 | 54.582 | 1.00 | 96.09 |
| ATOM | 789 | OD2 | ASP | 100 | 83.973 | 23.566 | 53.941 | 1.00 | 94.94 |
| ATOM | 790 | C | ASP | 100 | 85.861 | 27.531 | 52.179 | 1.00 | 93.79 |
| ATOM | 791 | O | ASP | 100 | 85.739 | 28.743 | 52.365 | 1.00 | 93.83 |
| ATOM | 792 | N | PRO | 101 | 87.015 | 26.998 | 51.735 | 1.00 | 96.96 |
| ATOM | 793 | CD | PRO | 101 | 87.480 | 25.609 | 51.851 | 1.00 | 98.24 |
| ATOM | 794 | CA | PRO | 101 | 88.159 | 27.878 | 51.444 | 1.00 | 99.43 |
| ATOM | 795 | CB | PRO | 101 | 89.366 | 26.938 | 51.564 | 1.00 | 98.97 |
| ATOM | 796 | CG | PRO | 101 | 88.860 | 25.813 | 52.424 | 1.00 | 99.32 |
| ATOM | 797 | C | PRO | 101 | 88.097 | 28.543 | 50.071 | 1.00 | 101.35 |
| ATOM | 798 | O | PRO | 101 | 87.014 | 28.846 | 49.567 | 1.00 | 102.76 |
| ATOM | 799 | N | LEU | 102 | 89.270 | 28.769 | 49.477 | 1.00 | 102.83 |
| ATOM | 800 | CA | LEU | 102 | 89.374 | 29.398 | 48.156 | 1.00 | 104.27 |
| ATOM | 801 | CB | LEU | 102 | 89.462 | 30.925 | 48.291 | 1.00 | 104.27 |
| ATOM | 802 | CG | LEU | 102 | 89.534 | 31.548 | 47.017 | 1.00 | 103.47 |
| ATOM | 803 | C | LEU | 102 | 90.587 | 28.889 | 47.369 | 1.00 | 104.76 |
| ATOM | 804 | O | LEU | 102 | 91.737 | 29.157 | 47.724 | 1.00 | 104.83 |
| ATOM | 805 | N | PRO | 107 | 90.748 | 18.959 | 64.154 | 1.00 | 126.18 |
| ATOM | 806 | CA | PRO | 107 | 89.727 | 17.932 | 63.974 | 1.00 | 126.66 |
| ATOM | 807 | CB | PRO | 107 | 88.542 | 18.209 | 64.898 | 1.00 | 126.29 |
| ATOM | 808 | C | PRO | 107 | 89.260 | 17.878 | 62.517 | 1.00 | 126.87 |
| ATOM | 809 | O | PRO | 107 | 88.076 | 18.060 | 62.223 | 1.00 | 127.11 |
| ATOM | 810 | N | VAL | 108 | 90.202 | 17.618 | 61.613 | 1.00 | 126.89 |
| ATOM | 811 | CA | VAL | 108 | 89.916 | 17.548 | 60.181 | 1.00 | 126.48 |
| ATOM | 812 | CB | VAL | 108 | 91.231 | 17.551 | 59.391 | 1.00 | 126.25 |
| ATOM | 813 | C | VAL | 108 | 89.074 | 16.331 | 59.790 | 1.00 | 125.86 |
| ATOM | 814 | O | VAL | 108 | 87.889 | 16.464 | 59.470 | 1.00 | 125.34 |
| ATOM | 815 | N | THR | 109 | 89.698 | 15.154 | 59.819 | 1.00 | 125.17 |
| ATOM | 816 | CA | THR | 109 | 89.049 | 13.890 | 59.461 | 1.00 | 123.95 |
| ATOM | 817 | CB | THR | 109 | 89.792 | 12.719 | 60.113 | 1.00 | 124.08 |
| ATOM | 818 | C | THR | 109 | 87.566 | 13.828 | 59.824 | 1.00 | 122.63 |
| ATOM | 819 | O | THR | 109 | 86.754 | 13.313 | 59.050 | 1.00 | 122.80 |
| ATOM | 820 | N | GLY | 110 | 87.224 | 14.351 | 60.999 | 1.00 | 120.61 |
| ATOM | 821 | CA | GLY | 110 | 85.844 | 14.343 | 61.449 | 1.00 | 117.57 |
| ATOM | 822 | C | GLY | 110 | 84.853 | 14.918 | 60.453 | 1.00 | 115.50 |
| ATOM | 823 | O | GLY | 110 | 84.260 | 14.183 | 59.659 | 1.00 | 116.01 |
| ATOM | 824 | N | ALA | 111 | 84.673 | 16.234 | 60.490 | 1.00 | 112.70 |
| ATOM | 825 | CA | ALA | 111 | 83.734 | 16.913 | 59.600 | 1.00 | 109.22 |
| ATOM | 826 | CB | ALA | 111 | 83.549 | 18.363 | 60.053 | 1.00 | 109.74 |
| ATOM | 827 | C | ALA | 111 | 84.146 | 16.876 | 58.127 | 1.00 | 106.68 |
| ATOM | 828 | O | ALA | 111 | 85.218 | 17.366 | 57.754 | 1.00 | 106.45 |
| ATOM | 829 | N | SER | 112 | 83.285 | 16.294 | 57.293 | 1.00 | 103.09 |
| ATOM | 830 | CA | SER | 112 | 83.538 | 16.211 | 55.858 | 1.00 | 99.27 |
| ATOM | 831 | CB | SER | 112 | 82.483 | 15.337 | 55.175 | 1.00 | 99.51 |
| ATOM | 832 | OG | SER | 112 | 82.457 | 14.037 | 55.735 | 1.00 | 101.30 |
| ATOM | 833 | C | SER | 112 | 83.476 | 17.622 | 55.285 | 1.00 | 96.09 |
| ATOM | 834 | O | SER | 112 | 82.460 | 18.310 | 55.408 | 1.00 | 96.48 |
| ATOM | 835 | N | PRO | 113 | 84.564 | 18.071 | 54.644 | 1.00 | 92.15 |
| ATOM | 836 | CD | PRO | 113 | 85.785 | 17.320 | 54.307 | 1.00 | 91.00 |
| ATOM | 837 | CA | PRO | 113 | 84.601 | 19.415 | 54.061 | 1.00 | 88.77 |
| ATOM | 838 | CB | PRO | 113 | 85.804 | 19.344 | 53.123 | 1.00 | 89.27 |
| ATOM | 839 | CG | PRO | 113 | 86.720 | 18.417 | 53.848 | 1.00 | 90.34 |
| ATOM | 840 | C | PRO | 113 | 83.309 | 19.782 | 53.333 | 1.00 | 84.95 |
| ATOM | 841 | O | PRO | 113 | 82.662 | 18.929 | 52.720 | 1.00 | 84.81 |
| ATOM | 842 | N | GLY | 114 | 82.930 | 21.052 | 53.422 | 1.00 | 80.54 |
| ATOM | 843 | CA | GLY | 114 | 81.729 | 21.506 | 52.754 | 1.00 | 75.55 |
| ATOM | 844 | C | GLY | 114 | 82.044 | 21.832 | 51.310 | 1.00 | 72.23 |
| ATOM | 845 | O | GLY | 114 | 83.199 | 22.076 | 50.961 | 1.00 | 71.35 |
| ATOM | 846 | N | GLY | 115 | 81.021 | 21.834 | 50.465 | 1.00 | 69.91 |
| ATOM | 847 | CA | GLY | 115 | 81.232 | 22.137 | 49.061 | 1.00 | 66.25 |
| ATOM | 848 | C | GLY | 115 | 80.635 | 21.080 | 48.157 | 1.00 | 63.68 |
| ATOM | 849 | O | GLY | 115 | 80.481 | 19.926 | 48.557 | 1.00 | 62.37 |
| ATOM | 850 | N | LEU | 116 | 80.292 | 21.484 | 46.937 | 1.00 | 62.02 |
| ATOM | 851 | CA | LEU | 116 | 79.709 | 20.582 | 45.948 | 1.00 | 59.67 |
| ATOM | 852 | CB | LEU | 116 | 79.271 | 21.387 | 44.723 | 1.00 | 57.68 |
| ATOM | 853 | CG | LEU | 116 | 78.388 | 20.692 | 43.691 | 1.00 | 56.19 |
| ATOM | 854 | CD1 | LEU | 116 | 77.022 | 20.411 | 44.296 | 1.00 | 54.53 |
| ATOM | 855 | CD2 | LEU | 116 | 78.261 | 21.582 | 42.456 | 1.00 | 56.83 |
| ATOM | 856 | C | LEU | 116 | 80.764 | 19.544 | 45.551 | 1.00 | 59.55 |
| ATOM | 857 | O | LEU | 116 | 81.900 | 19.901 | 45.220 | 1.00 | 58.10 |
| ATOM | 858 | N | ARG | 117 | 80.390 | 18.267 | 45.587 | 1.00 | 59.24 |
| ATOM | 859 | CA | ARG | 117 | 81.327 | 17.195 | 45.253 | 1.00 | 59.70 |
| ATOM | 860 | CB | ARG | 117 | 81.109 | 15.992 | 46.174 | 1.00 | 60.56 |
| ATOM | 861 | CG | ARG | 117 | 81.021 | 16.374 | 47.632 | 1.00 | 62.94 |
| ATOM | 862 | CD | ARG | 117 | 81.319 | 15.216 | 48.560 | 1.00 | 67.09 |
| ATOM | 863 | NE | ARG | 117 | 82.602 | 15.403 | 49.230 | 1.00 | 72.49 |
| ATOM | 864 | CZ | ARG | 117 | 82.917 | 16.462 | 49.975 | 1.00 | 75.62 |
| ATOM | 865 | NH1 | ARG | 117 | 82.042 | 17.447 | 50.157 | 1.00 | 76.20 |
| ATOM | 866 | NH2 | ARG | 117 | 84.118 | 16.545 | 50.530 | 1.00 | 76.65 |
| ATOM | 867 | C | ARG | 117 | 81.209 | 16.765 | 43.798 | 1.00 | 58.65 |
| ATOM | 868 | O | ARG | 117 | 82.219 | 16.549 | 43.121 | 1.00 | 58.41 |
| ATOM | 869 | N | GLU | 118 | 79.972 | 16.644 | 43.326 | 1.00 | 57.45 |
| ATOM | 870 | CA | GLU | 118 | 79.703 | 16.264 | 41.946 | 1.00 | 56.49 |
| ATOM | 871 | CB | GLU | 118 | 79.584 | 14.737 | 41.840 | 1.00 | 56.46 |
| ATOM | 872 | CG | GLU | 118 | 78.782 | 14.074 | 42.940 | 1.00 | 57.68 |
| ATOM | 873 | CD | GLU | 118 | 78.794 | 12.550 | 42.845 | 1.00 | 58.93 |
| ATOM | 874 | OE1 | GLU | 118 | 79.886 | 11.944 | 42.929 | 1.00 | 59.44 |
| ATOM | 875 | OE2 | GLU | 118 | 77.709 | 11.951 | 42.689 | 1.00 | 59.58 |
| ATOM | 876 | C | GLU | 118 | 78.442 | 16.959 | 41.423 | 1.00 | 55.19 |
| ATOM | 877 | O | GLU | 118 | 77.498 | 17.192 | 42.176 | 1.00 | 57.56 |
| ATOM | 878 | N | LEU | 119 | 78.432 | 17.304 | 40.139 | 1.00 | 52.98 |
| ATOM | 879 | CA | LEU | 119 | 77.283 | 17.983 | 39.536 | 1.00 | 50.77 |
| ATOM | 880 | CB | LEU | 119 | 77.689 | 18.563 | 38.184 | 1.00 | 47.76 |
| ATOM | 881 | CG | LEU | 119 | 78.554 | 19.824 | 38.302 | 1.00 | 45.09 |
| ATOM | 882 | CD1 | LEU | 119 | 79.187 | 20.143 | 36.971 | 1.00 | 43.63 |
| ATOM | 883 | CD2 | LEU | 119 | 77.704 | 20.989 | 38.787 | 1.00 | 41.01 |
| ATOM | 884 | C | LEU | 119 | 76.038 | 17.103 | 39.391 | 1.00 | 51.28 |
| ATOM | 885 | O | LEU | 119 | 74.925 | 17.532 | 39.676 | 1.00 | 50.10 |
| ATOM | 886 | N | GLN | 120 | 76.220 | 15.872 | 38.941 | 1.00 | 53.25 |
| ATOM | 887 | CA | GLN | 120 | 75.100 | 14.944 | 38.815 | 1.00 | 56.61 |
| ATOM | 888 | CB | GLN | 120 | 74.588 | 14.560 | 40.210 | 1.00 | 60.68 |
| ATOM | 889 | CG | GLN | 120 | 75.660 | 13.965 | 41.121 | 1.00 | 64.56 |
| ATOM | 890 | CD | GLN | 120 | 75.098 | 12.947 | 42.109 | 1.00 | 68.65 |
| ATOM | 891 | OE1 | GLN | 120 | 74.466 | 11.961 | 41.711 | 1.00 | 70.06 |
| ATOM | 892 | NE2 | GLN | 120 | 75.337 | 13.174 | 43.402 | 1.00 | 69.09 |
| ATOM | 893 | C | GLN | 120 | 73.921 | 15.404 | 37.951 | 1.00 | 56.07 |
| ATOM | 894 | O | GLN | 120 | 72.769 | 15.038 | 38.207 | 1.00 | 56.45 |
| ATOM | 895 | N | LEU | 121 | 74.220 | 16.197 | 36.927 | 1.00 | 55.01 |
| ATOM | 896 | CA | LEU | 121 | 73.213 | 16.687 | 35.987 | 1.00 | 53.16 |
| ATOM | 897 | CB | LEU | 121 | 73.667 | 18.017 | 35.383 | 1.00 | 52.15 |
| ATOM | 898 | CG | LEU | 121 | 73.852 | 19.173 | 36.360 | 1.00 | 51.71 |
| ATOM | 899 | CD1 | LEU | 121 | 74.598 | 20.300 | 35.673 | 1.00 | 49.86 |
| ATOM | 900 | CD2 | LEU | 121 | 72.485 | 19.632 | 36.868 | 1.00 | 50.80 |
| ATOM | 901 | C | LEU | 121 | 73.154 | 15.636 | 34.890 | 1.00 | 52.58 |
| ATOM | 902 | O | LEU | 121 | 73.450 | 15.919 | 33.729 | 1.00 | 53.15 |
| ATOM | 903 | N | ARG | 122 | 72.765 | 14.422 | 35.253 | 1.00 | 52.13 |
| ATOM | 904 | CA | ARG | 122 | 72.741 | 13.336 | 34.288 | 1.00 | 51.57 |
| ATOM | 905 | CB | ARG | 122 | 72.730 | 12.005 | 35.029 | 1.00 | 53.46 |
| ATOM | 906 | CG | ARG | 122 | 71.586 | 11.827 | 35.995 | 1.00 | 55.67 |
| ATOM | 907 | CD | ARG | 122 | 72.026 | 10.906 | 37.111 | 1.00 | 58.36 |
| ATOM | 908 | NE | ARG | 122 | 70.896 | 10.320 | 37.815 | 1.00 | 62.96 |
| ATOM | 909 | CZ | ARG | 122 | 70.976 | 9.795 | 39.032 | 1.00 | 65.49 |
| ATOM | 910 | NH1 | ARG | 122 | 72.140 | 9.790 | 39.680 | 1.00 | 64.20 |
| ATOM | 911 | NH2 | ARG | 122 | 69.891 | 9.273 | 39.596 | 1.00 | 67.00 |
| ATOM | 912 | C | ARG | 122 | 71.656 | 13.358 | 33.222 | 1.00 | 49.90 |
| ATOM | 913 | O | ARG | 122 | 71.640 | 12.504 | 32.333 | 1.00 | 50.83 |
| ATOM | 914 | N | SER | 123 | 70.756 | 14.330 | 33.292 | 1.00 | 46.61 |
| ATOM | 915 | CA | SER | 123 | 69.701 | 14.434 | 32.293 | 1.00 | 42.82 |
| ATOM | 916 | CB | SER | 123 | 68.335 | 14.530 | 32.967 | 1.00 | 42.47 |
| ATOM | 917 | OG | SER | 123 | 68.038 | 13.341 | 33.670 | 1.00 | 38.70 |
| ATOM | 918 | C | SER | 123 | 69.934 | 15.663 | 31.430 | 1.00 | 41.29 |
| ATOM | 919 | O | SER | 123 | 69.282 | 15.850 | 30.407 | 1.00 | 40.43 |
| ATOM | 920 | N | LEU | 124 | 70.879 | 16.496 | 31.849 | 1.00 | 40.71 |
| ATOM | 921 | CA | LEU | 124 | 71.187 | 17.715 | 31.119 | 1.00 | 40.84 |
| ATOM | 922 | CB | LEU | 124 | 72.055 | 18.649 | 31.956 | 1.00 | 37.35 |
| ATOM | 923 | CG | LEU | 124 | 72.435 | 19.929 | 31.211 | 1.00 | 37.32 |
| ATOM | 924 | CD1 | LEU | 124 | 71.166 | 20.651 | 30.771 | 1.00 | 35.84 |
| ATOM | 925 | CD2 | LEU | 124 | 73.291 | 20.821 | 32.089 | 1.00 | 35.21 |
| ATOM | 926 | C | LEU | 124 | 71.888 | 17.428 | 29.809 | 1.00 | 43.38 |
| ATOM | 927 | O | LEU | 124 | 73.109 | 17.252 | 29.769 | 1.00 | 43.33 |
| ATOM | 928 | N | THR | 125 | 71.104 | 17.407 | 28.733 | 1.00 | 45.11 |
| ATOM | 929 | CA | THR | 125 | 71.621 | 17.138 | 27.397 | 1.00 | 43.33 |
| ATOM | 930 | CB | THR | 125 | 70.913 | 15.918 | 26.783 | 1.00 | 43.30 |
| ATOM | 931 | OG1 | THR | 125 | 69.496 | 16.104 | 26.878 | 1.00 | 42.09 |
| ATOM | 932 | CG2 | THR | 125 | 71.305 | 14.639 | 27.510 | 1.00 | 39.71 |
| ATOM | 933 | C | THR | 125 | 71.409 | 18.319 | 26.456 | 1.00 | 43.96 |
| ATOM | 934 | O | THR | 125 | 71.624 | 18.198 | 25.253 | 1.00 | 44.38 |
| ATOM | 935 | N | GLU | 126 | 70.994 | 19.465 | 26.986 | 1.00 | 43.81 |
| ATOM | 936 | CA | GLU | 126 | 70.745 | 20.598 | 26.106 | 1.00 | 42.67 |
| ATOM | 937 | CB | GLU | 126 | 69.350 | 20.470 | 25.500 | 1.00 | 41.42 |
| ATOM | 938 | CG | GLU | 126 | 69.020 | 21.521 | 24.453 | 1.00 | 41.40 |
| ATOM | 939 | CD | GLU | 126 | 69.512 | 21.153 | 23.061 | 1.00 | 43.48 |
| ATOM | 940 | OE1 | GLU | 126 | 68.830 | 20.358 | 22.370 | 1.00 | 41.80 |
| ATOM | 941 | OE2 | GLU | 126 | 70.587 | 21.658 | 22.661 | 1.00 | 42.13 |
| ATOM | 942 | C | GLU | 126 | 70.879 | 21.990 | 26.709 | 1.00 | 43.68 |
| ATOM | 943 | O | GLU | 126 | 70.234 | 22.317 | 27.704 | 1.00 | 45.80 |
| ATOM | 944 | N | ILE | 127 | 71.736 | 22.798 | 26.094 | 1.00 | 42.47 |
| ATOM | 945 | CA | ILE | 127 | 71.933 | 24.192 | 26.479 | 1.00 | 41.14 |
| ATOM | 946 | CB | ILE | 127 | 73.318 | 24.426 | 27.085 | 1.00 | 38.54 |
| ATOM | 947 | CG2 | ILE | 127 | 73.574 | 25.911 | 27.245 | 1.00 | 33.94 |
| ATOM | 948 | CG1 | ILE | 127 | 73.406 | 23.722 | 28.437 | 1.00 | 36.30 |
| ATOM | 949 | CD1 | ILE | 127 | 74.784 | 23.818 | 29.093 | 1.00 | 36.27 |
| ATOM | 950 | C | ILE | 127 | 71.790 | 24.944 | 25.149 | 1.00 | 43.31 |
| ATOM | 951 | O | ILE | 127 | 72.715 | 24.974 | 24.325 | 1.00 | 42.28 |
| ATOM | 952 | N | LEU | 128 | 70.609 | 25.523 | 24.935 | 1.00 | 44.48 |
| ATOM | 953 | CA | LEU | 128 | 70.312 | 26.222 | 23.690 | 1.00 | 44.79 |
| ATOM | 954 | CB | LEU | 128 | 68.851 | 26.661 | 23.678 | 1.00 | 44.23 |
| ATOM | 955 | CG | LEU | 128 | 67.875 | 25.475 | 23.692 | 1.00 | 45.11 |
| ATOM | 956 | CD1 | LEU | 128 | 66.427 | 25.981 | 23.643 | 1.00 | 41.10 |
| ATOM | 957 | CD2 | LEU | 128 | 68.183 | 24.555 | 22.499 | 1.00 | 41.18 |
| ATOM | 958 | C | LEU | 128 | 71.203 | 27.400 | 23.376 | 1.00 | 45.64 |
| ATOM | 959 | O | LEU | 128 | 71.668 | 27.543 | 22.247 | 1.00 | 47.14 |
| ATOM | 960 | N | LYS | 129 | 71.454 | 28.232 | 24.378 | 1.00 | 45.50 |
| ATOM | 961 | CA | LYS | 129 | 72.283 | 29.415 | 24.201 | 1.00 | 45.15 |
| ATOM | 962 | CB | LYS | 129 | 71.368 | 30.629 | 23.983 | 1.00 | 47.19 |
| ATOM | 963 | CG | LYS | 129 | 72.057 | 31.989 | 23.869 | 1.00 | 51.77 |
| ATOM | 964 | CD | LYS | 129 | 71.021 | 33.124 | 23.825 | 1.00 | 53.80 |
| ATOM | 965 | CE | LYS | 129 | 71.666 | 34.507 | 23.978 | 1.00 | 56.48 |
| ATOM | 966 | NZ | LYS | 129 | 70.669 | 35.618 | 24.130 | 1.00 | 55.90 |
| ATOM | 967 | C | LYS | 129 | 73.143 | 29.607 | 25.447 | 1.00 | 44.99 |
| ATOM | 968 | O | LYS | 129 | 72.694 | 29.343 | 26.559 | 1.00 | 44.46 |
| ATOM | 969 | N | GLY | 130 | 74.384 | 30.049 | 25.267 | 1.00 | 45.43 |
| ATOM | 970 | CA | GLY | 130 | 75.240 | 30.275 | 26.422 | 1.00 | 45.56 |
| ATOM | 971 | C | GLY | 130 | 76.270 | 29.201 | 26.713 | 1.00 | 45.03 |
| ATOM | 972 | O | GLY | 130 | 76.214 | 28.104 | 26.161 | 1.00 | 44.57 |
| ATOM | 973 | N | GLY | 131 | 77.205 | 29.519 | 27.605 | 1.00 | 44.82 |
| ATOM | 974 | CA | GLY | 131 | 78.261 | 28.581 | 27.934 | 1.00 | 46.22 |
| ATOM | 975 | C | GLY | 131 | 78.315 | 28.070 | 29.361 | 1.00 | 45.93 |
| ATOM | 976 | O | GLY | 131 | 77.367 | 28.197 | 30.126 | 1.00 | 47.97 |
| ATOM | 977 | N | VAL | 132 | 79.449 | 27.493 | 29.724 | 1.00 | 45.40 |
| ATOM | 978 | CA | VAL | 132 | 79.615 | 26.936 | 31.050 | 1.00 | 45.70 |
| ATOM | 979 | CB | VAL | 132 | 79.794 | 25.405 | 30.980 | 1.00 | 44.20 |
| ATOM | 980 | CG1 | VAL | 132 | 80.046 | 24.846 | 32.360 | 1.00 | 43.09 |
| ATOM | 981 | CG2 | VAL | 132 | 78.549 | 24.768 | 30.366 | 1.00 | 45.15 |
| ATOM | 982 | C | VAL | 132 | 80.804 | 27.534 | 31.777 | 1.00 | 47.30 |
| ATOM | 983 | O | VAL | 132 | 81.938 | 27.507 | 31.282 | 1.00 | 47.68 |
| ATOM | 984 | N | LEU | 133 | 80.524 | 28.071 | 32.961 | 1.00 | 47.58 |
| ATOM | 985 | CA | LEU | 133 | 81.535 | 28.675 | 33.807 | 1.00 | 47.58 |
| ATOM | 986 | CB | LEU | 133 | 81.189 | 30.136 | 34.095 | 1.00 | 47.73 |
| ATOM | 987 | CG | LEU | 133 | 82.080 | 30.807 | 35.148 | 1.00 | 49.20 |
| ATOM | 988 | CD1 | LEU | 133 | 83.501 | 30.936 | 34.599 | 1.00 | 47.21 |
| ATOM | 989 | CD2 | LEU | 133 | 81.509 | 32.171 | 35.525 | 1.00 | 46.27 |
| ATOM | 990 | C | LEU | 133 | 81.590 | 27.902 | 35.120 | 1.00 | 48.57 |
| ATOM | 991 | O | LEU | 133 | 80.625 | 27.889 | 35.888 | 1.00 | 48.62 |
| ATOM | 992 | N | ILE | 134 | 82.715 | 27.246 | 35.369 | 1.00 | 47.15 |
| ATOM | 993 | CA | ILE | 134 | 82.876 | 26.496 | 36.599 | 1.00 | 47.05 |
| ATOM | 994 | CB | ILE | 134 | 82.847 | 24.974 | 36.328 | 1.00 | 43.42 |
| ATOM | 995 | CG2 | ILE | 134 | 82.963 | 24.210 | 37.633 | 1.00 | 41.94 |
| ATOM | 996 | CG1 | ILE | 134 | 81.537 | 24.603 | 35.622 | 1.00 | 42.18 |
| ATOM | 997 | CD1 | ILE | 134 | 81.321 | 23.115 | 35.409 | 1.00 | 36.15 |
| ATOM | 998 | C | ILE | 134 | 84.205 | 26.916 | 37.221 | 1.00 | 48.77 |
| ATOM | 999 | O | ILE | 134 | 85.273 | 26.640 | 36.673 | 1.00 | 49.10 |
| ATOM | 1000 | N | GLN | 135 | 84.130 | 27.594 | 38.363 | 1.00 | 49.58 |
| ATOM | 1001 | CA | GLN | 135 | 85.326 | 28.080 | 39.040 | 1.00 | 50.96 |
| ATOM | 1002 | CB | GLN | 135 | 85.527 | 29.572 | 38.738 | 1.00 | 51.43 |
| ATOM | 1003 | CG | GLN | 135 | 85.562 | 29.950 | 37.267 | 1.00 | 54.23 |
| ATOM | 1004 | CD | GLN | 135 | 86.913 | 29.700 | 36.610 | 1.00 | 56.24 |
| ATOM | 1005 | OE1 | GLN | 135 | 87.910 | 30.347 | 36.939 | 1.00 | 58.47 |
| ATOM | 1006 | NE2 | GLN | 135 | 86.948 | 28.761 | 35.670 | 1.00 | 57.30 |
| ATOM | 1007 | C | GLN | 135 | 85.286 | 27.904 | 40.557 | 1.00 | 51.50 |
| ATOM | 1008 | O | GLN | 135 | 84.223 | 27.833 | 41.172 | 1.00 | 53.24 |
| ATOM | 1009 | N | ARG | 136 | 86.472 | 27.840 | 41.146 | 1.00 | 51.51 |
| ATOM | 1010 | CA | ARG | 136 | 86.641 | 27.734 | 42.588 | 1.00 | 49.89 |
| ATOM | 1011 | CB | ARG | 136 | 86.613 | 29.149 | 43.175 | 1.00 | 48.50 |
| ATOM | 1012 | CG | ARG | 136 | 87.537 | 30.115 | 42.420 | 1.00 | 47.11 |
| ATOM | 1013 | CD | ARG | 136 | 87.473 | 31.547 | 42.951 | 1.00 | 47.61 |
| ATOM | 1014 | NE | ARG | 136 | 86.249 | 32.246 | 42.566 | 1.00 | 48.00 |
| ATOM | 1015 | CZ | ARG | 136 | 85.970 | 32.657 | 41.331 | 1.00 | 49.03 |
| ATOM | 1016 | NH1 | ARG | 136 | 86.826 | 32.447 | 40.338 | 1.00 | 48.98 |
| ATOM | 1017 | NH2 | ARG | 136 | 84.825 | 33.280 | 41.087 | 1.00 | 48.99 |
| ATOM | 1018 | C | ARG | 136 | 85.680 | 26.814 | 43.344 | 1.00 | 49.80 |
| ATOM | 1019 | O | ARG | 136 | 84.969 | 27.239 | 44.255 | 1.00 | 49.62 |
| ATOM | 1020 | N | ASN | 137 | 85.681 | 25.542 | 42.965 | 1.00 | 50.11 |
| ATOM | 1021 | CA | ASN | 137 | 84.852 | 24.531 | 43.614 | 1.00 | 51.52 |
| ATOM | 1022 | CB | ASN | 137 | 83.836 | 23.984 | 42.619 | 1.00 | 49.76 |
| ATOM | 1023 | CG | ASN | 137 | 82.946 | 25.068 | 42.071 | 1.00 | 47.43 |
| ATOM | 1024 | OD1 | ASN | 137 | 82.109 | 25.615 | 42.792 | 1.00 | 47.56 |
| ATOM | 1025 | ND2 | ASN | 137 | 83.138 | 25.411 | 40.800 | 1.00 | 44.58 |
| ATOM | 1026 | C | ASN | 137 | 85.791 | 23.425 | 44.098 | 1.00 | 53.46 |
| ATOM | 1027 | O | ASN | 137 | 86.001 | 22.417 | 43.416 | 1.00 | 54.02 |
| ATOM | 1028 | N | PRO | 138 | 86.378 | 23.620 | 45.291 | 1.00 | 54.39 |
| ATOM | 1029 | CD | PRO | 138 | 86.090 | 24.835 | 46.069 | 1.00 | 54.92 |
| ATOM | 1030 | CA | PRO | 138 | 87.324 | 22.770 | 46.023 | 1.00 | 55.10 |
| ATOM | 1031 | CB | PRO | 138 | 87.391 | 23.445 | 47.388 | 1.00 | 56.13 |
| ATOM | 1032 | CG | PRO | 138 | 87.246 | 24.875 | 47.030 | 1.00 | 55.77 |
| ATOM | 1033 | C | PRO | 138 | 87.057 | 21.267 | 46.141 | 1.00 | 54.62 |
| ATOM | 1034 | O | PRO | 138 | 87.997 | 20.474 | 46.095 | 1.00 | 55.62 |
| ATOM | 1035 | N | GLN | 139 | 85.798 | 20.873 | 46.309 | 1.00 | 52.71 |
| ATOM | 1036 | CA | GLN | 139 | 85.470 | 19.457 | 46.445 | 1.00 | 50.41 |
| ATOM | 1037 | CB | GLN | 139 | 84.503 | 19.245 | 47.600 | 1.00 | 54.28 |
| ATOM | 1038 | CG | GLN | 139 | 84.965 | 19.780 | 48.927 | 1.00 | 57.44 |
| ATOM | 1039 | CD | GLN | 139 | 86.251 | 19.143 | 49.366 | 1.00 | 60.16 |
| ATOM | 1040 | OE1 | GLN | 139 | 86.410 | 17.920 | 49.286 | 1.00 | 59.39 |
| ATOM | 1041 | NE2 | GLN | 139 | 87.183 | 19.964 | 49.843 | 1.00 | 61.11 |
| ATOM | 1042 | C | GLN | 139 | 84.812 | 18.881 | 45.212 | 1.00 | 48.99 |
| ATOM | 1043 | O | GLN | 139 | 84.227 | 17.806 | 45.291 | 1.00 | 49.44 |
| ATOM | 1044 | N | LEU | 140 | 84.910 | 19.573 | 44.080 | 1.00 | 46.61 |
| ATOM | 1045 | CA | LEU | 140 | 84.249 | 19.127 | 42.851 | 1.00 | 45.80 |
| ATOM | 1046 | CB | LEU | 140 | 83.748 | 20.353 | 42.079 | 1.00 | 42.36 |
| ATOM | 1047 | CG | LEU | 140 | 82.885 | 20.080 | 40.845 | 1.00 | 38.55 |
| ATOM | 1048 | CD1 | LEU | 140 | 81.566 | 19.439 | 41.262 | 1.00 | 36.83 |
| ATOM | 1049 | CD2 | LEU | 140 | 82.638 | 21.376 | 40.108 | 1.00 | 36.26 |
| ATOM | 1050 | C | LEU | 140 | 85.024 | 18.220 | 41.887 | 1.00 | 48.03 |
| ATOM | 1051 | O | LEU | 140 | 86.151 | 18.521 | 41.491 | 1.00 | 48.11 |
| ATOM | 1052 | N | CYS | 141 | 84.387 | 17.120 | 41.487 | 1.00 | 49.94 |
| ATOM | 1053 | CA | CYS | 141 | 84.983 | 16.165 | 40.553 | 1.00 | 51.42 |
| ATOM | 1054 | C | CYS | 141 | 84.155 | 15.955 | 39.281 | 1.00 | 52.28 |
| ATOM | 1055 | O | CYS | 141 | 82.986 | 16.342 | 39.201 | 1.00 | 52.04 |
| ATOM | 1056 | CB | CYS | 141 | 85.161 | 14.804 | 41.227 | 1.00 | 53.37 |
| ATOM | 1057 | SG | CYS | 141 | 86.472 | 14.727 | 42.484 | 1.00 | 55.47 |
| ATOM | 1058 | N | TYR | 142 | 84.790 | 15.342 | 38.288 | 1.00 | 52.23 |
| ATOM | 1059 | CA | TYR | 142 | 84.153 | 14.988 | 37.024 | 1.00 | 53.02 |
| ATOM | 1060 | CB | TYR | 142 | 82.922 | 14.114 | 37.307 | 1.00 | 55.55 |
| ATOM | 1061 | CG | TYR | 142 | 83.220 | 13.021 | 38.306 | 1.00 | 57.71 |
| ATOM | 1062 | CD1 | TYR | 142 | 82.807 | 13.131 | 39.633 | 1.00 | 58.55 |
| ATOM | 1063 | CE1 | TYR | 142 | 83.154 | 12.162 | 40.580 | 1.00 | 60.12 |
| ATOM | 1064 | CD2 | TYR | 142 | 83.986 | 11.911 | 37.944 | 1.00 | 59.09 |
| ATOM | 1065 | CE2 | TYR | 142 | 84.340 | 10.937 | 38.882 | 1.00 | 59.55 |
| ATOM | 1066 | CZ | TYR | 142 | 83.922 | 11.069 | 40.197 | 1.00 | 60.59 |
| ATOM | 1067 | OH | TYR | 142 | 84.281 | 10.119 | 41.130 | 1.00 | 61.97 |
| ATOM | 1068 | C | TYR | 142 | 83.777 | 16.077 | 36.038 | 1.00 | 52.13 |
| ATOM | 1069 | O | TYR | 142 | 83.566 | 15.787 | 34.862 | 1.00 | 52.66 |
| ATOM | 1070 | N | GLN | 143 | 83.696 | 17.323 | 36.484 | 1.00 | 51.73 |
| ATOM | 1071 | CA | GLN | 143 | 83.326 | 18.392 | 35.563 | 1.00 | 51.08 |
| ATOM | 1072 | CB | GLN | 143 | 83.391 | 19.754 | 36.259 | 1.00 | 48.67 |
| ATOM | 1073 | CG | GLN | 143 | 84.799 | 20.275 | 36.459 | 1.00 | 47.89 |
| ATOM | 1074 | CD | GLN | 143 | 85.361 | 19.948 | 37.822 | 1.00 | 47.25 |
| ATOM | 1075 | OE1 | GLN | 143 | 85.186 | 18.839 | 38.337 | 1.00 | 46.97 |
| ATOM | 1076 | NE2 | GLN | 143 | 86.056 | 20.913 | 38.415 | 1.00 | 45.50 |
| ATOM | 1077 | C | GLN | 143 | 84.213 | 18.426 | 34.310 | 1.00 | 51.56 |
| ATOM | 1078 | O | GLN | 143 | 83.800 | 18.931 | 33.267 | 1.00 | 50.46 |
| ATOM | 1079 | N | ASP | 144 | 85.427 | 17.889 | 34.414 | 1.00 | 53.39 |
| ATOM | 1080 | CA | ASP | 144 | 86.362 | 17.888 | 33.284 | 1.00 | 54.95 |
| ATOM | 1081 | CB | ASP | 144 | 87.806 | 18.001 | 33.795 | 1.00 | 57.46 |
| ATOM | 1082 | CG | ASP | 144 | 88.158 | 16.928 | 34.823 | 1.00 | 60.87 |
| ATOM | 1083 | OD1 | ASP | 144 | 87.240 | 16.420 | 35.508 | 1.00 | 61.77 |
| ATOM | 1084 | OD2 | ASP | 144 | 89.361 | 16.605 | 34.956 | 1.00 | 61.23 |
| ATOM | 1085 | C | ASP | 144 | 86.227 | 16.698 | 32.336 | 1.00 | 54.14 |
| ATOM | 1086 | O | ASP | 144 | 86.718 | 16.746 | 31.211 | 1.00 | 54.76 |
| ATOM | 1087 | N | THR | 145 | 85.548 | 15.644 | 32.779 | 1.00 | 52.72 |
| ATOM | 1088 | CA | THR | 145 | 85.344 | 14.456 | 31.954 | 1.00 | 51.50 |
| ATOM | 1089 | CB | THR | 145 | 85.184 | 13.194 | 32.806 | 1.00 | 52.77 |
| ATOM | 1090 | OG1 | THR | 145 | 83.810 | 13.078 | 33.220 | 1.00 | 53.52 |
| ATOM | 1091 | CG2 | THR | 145 | 86.095 | 13.250 | 34.025 | 1.00 | 50.79 |
| ATOM | 1092 | C | THR | 145 | 84.072 | 14.547 | 31.114 | 1.00 | 51.38 |
| ATOM | 1093 | O | THR | 145 | 83.705 | 13.593 | 30.428 | 1.00 | 51.92 |
| ATOM | 1094 | N | ILE | 146 | 83.386 | 15.682 | 31.180 | 1.00 | 51.88 |
| ATOM | 1095 | CA | ILE | 146 | 82.136 | 15.856 | 30.444 | 1.00 | 49.69 |
| ATOM | 1096 | CB | ILE | 146 | 81.122 | 16.710 | 31.258 | 1.00 | 49.09 |
| ATOM | 1097 | CG2 | ILE | 146 | 79.967 | 17.145 | 30.367 | 1.00 | 48.38 |
| ATOM | 1098 | CG1 | ILE | 146 | 80.609 | 15.914 | 32.467 | 1.00 | 48.35 |
| ATOM | 1099 | CD1 | ILE | 146 | 79.764 | 14.689 | 32.112 | 1.00 | 47.73 |
| ATOM | 1100 | C | ILE | 146 | 82.349 | 16.511 | 29.093 | 1.00 | 49.10 |
| ATOM | 1101 | O | ILE | 146 | 83.057 | 17.515 | 28.982 | 1.00 | 49.69 |
| ATOM | 1102 | N | LEU | 147 | 81.739 | 15.942 | 28.061 | 1.00 | 47.61 |
| ATOM | 1103 | CA | LEU | 147 | 81.864 | 16.512 | 26.735 | 1.00 | 46.57 |
| ATOM | 1104 | CB | LEU | 147 | 81.662 | 15.435 | 25.669 | 1.00 | 46.78 |
| ATOM | 1105 | CG | LEU | 147 | 81.972 | 15.868 | 24.228 | 1.00 | 48.10 |
| ATOM | 1106 | CD1 | LEU | 147 | 83.424 | 16.331 | 24.128 | 1.00 | 44.45 |
| ATOM | 1107 | CD2 | LEU | 147 | 81.706 | 14.709 | 23.274 | 1.00 | 46.61 |
| ATOM | 1108 | C | LEU | 147 | 80.793 | 17.589 | 26.617 | 1.00 | 47.11 |
| ATOM | 1109 | O | LEU | 147 | 79.653 | 17.317 | 26.230 | 1.00 | 47.80 |
| ATOM | 1110 | N | TRP | 148 | 81.163 | 18.815 | 26.966 | 1.00 | 47.15 |
| ATOM | 1111 | CA | TRP | 148 | 80.231 | 19.940 | 26.920 | 1.00 | 48.80 |
| ATOM | 1112 | CB | TRP | 148 | 80.876 | 21.170 | 27.571 | 1.00 | 49.02 |
| ATOM | 1113 | CG | TRP | 148 | 81.187 | 20.945 | 29.021 | 1.00 | 48.42 |
| ATOM | 1114 | CD2 | TRP | 148 | 80.251 | 20.955 | 30.107 | 1.00 | 49.28 |
| ATOM | 1115 | CE2 | TRP | 148 | 80.966 | 20.608 | 31.276 | 1.00 | 48.49 |
| ATOM | 1116 | CE3 | TRP | 148 | 78.877 | 21.222 | 30.204 | 1.00 | 48.27 |
| ATOM | 1117 | CD1 | TRP | 148 | 82.391 | 20.609 | 29.557 | 1.00 | 48.34 |
| ATOM | 1118 | NE1 | TRP | 148 | 82.269 | 20.403 | 30.911 | 1.00 | 48.74 |
| ATOM | 1119 | CZ2 | TRP | 148 | 80.358 | 20.519 | 32.530 | 1.00 | 47.35 |
| ATOM | 1120 | CZ3 | TRP | 148 | 78.271 | 21.136 | 31.449 | 1.00 | 47.60 |
| ATOM | 1121 | CH2 | TRP | 148 | 79.014 | 20.787 | 32.597 | 1.00 | 49.76 |
| ATOM | 1122 | C | TRP | 148 | 79.721 | 20.293 | 25.523 | 1.00 | 48.75 |
| ATOM | 1123 | O | TRP | 148 | 78.595 | 20.765 | 25.367 | 1.00 | 47.54 |
| ATOM | 1124 | N | LYS | 149 | 80.553 | 20.059 | 24.512 | 1.00 | 49.96 |
| ATOM | 1125 | CA | LYS | 149 | 80.179 | 20.348 | 23.137 | 1.00 | 48.40 |
| ATOM | 1126 | CB | LYS | 149 | 81.324 | 19.993 | 22.203 | 1.00 | 52.26 |
| ATOM | 1127 | CG | LYS | 149 | 82.665 | 20.533 | 22.659 | 1.00 | 60.02 |
| ATOM | 1128 | CD | LYS | 149 | 82.621 | 22.046 | 22.881 | 1.00 | 62.98 |
| ATOM | 1129 | CE | LYS | 149 | 84.004 | 22.644 | 22.710 | 1.00 | 64.21 |
| ATOM | 1130 | NZ | LYS | 149 | 84.536 | 22.371 | 21.334 | 1.00 | 63.79 |
| ATOM | 1131 | C | LYS | 149 | 78.947 | 19.551 | 22.755 | 1.00 | 47.38 |
| ATOM | 1132 | O | LYS | 149 | 78.135 | 19.997 | 21.955 | 1.00 | 47.43 |
| ATOM | 1133 | N | ASP | 150 | 78.801 | 18.360 | 23.319 | 1.00 | 46.10 |
| ATOM | 1134 | CA | ASP | 150 | 77.637 | 17.556 | 22.994 | 1.00 | 45.08 |
| ATOM | 1135 | CB | ASP | 150 | 77.750 | 16.163 | 23.596 | 1.00 | 45.12 |
| ATOM | 1136 | CG | ASP | 150 | 76.718 | 15.205 | 23.024 | 1.00 | 45.94 |
| ATOM | 1137 | OD1 | ASP | 150 | 76.966 | 14.701 | 21.912 | 1.00 | 45.25 |
| ATOM | 1138 | OD2 | ASP | 150 | 75.664 | 14.968 | 23.664 | 1.00 | 42.14 |
| ATOM | 1139 | C | ASP | 150 | 76.397 | 18.229 | 23.565 | 1.00 | 45.07 |
| ATOM | 1140 | O | ASP | 150 | 75.352 | 18.301 | 22.916 | 1.00 | 45.29 |
| ATOM | 1141 | N | ILE | 151 | 76.523 | 18.721 | 24.791 | 1.00 | 43.99 |
| ATOM | 1142 | CA | ILE | 151 | 75.411 | 19.371 | 25.453 | 1.00 | 42.56 |
| ATOM | 1143 | CB | ILE | 151 | 75.757 | 19.677 | 26.921 | 1.00 | 40.70 |
| ATOM | 1144 | CG2 | ILE | 151 | 74.555 | 20.304 | 27.615 | 1.00 | 41.16 |
| ATOM | 1145 | CG1 | ILE | 151 | 76.154 | 18.373 | 27.635 | 1.00 | 40.42 |
| ATOM | 1146 | CD1 | ILE | 151 | 76.586 | 18.529 | 29.095 | 1.00 | 35.25 |
| ATOM | 1147 | C | ILE | 151 | 75.018 | 20.645 | 24.717 | 1.00 | 43.30 |
| ATOM | 1148 | O | ILE | 151 | 73.838 | 20.860 | 24.450 | 1.00 | 42.65 |
| ATOM | 1149 | N | PHE | 152 | 76.003 | 21.481 | 24.386 | 1.00 | 43.93 |
| ATOM | 1150 | CA | PHE | 152 | 75.747 | 22.724 | 23.658 | 1.00 | 44.46 |
| ATOM | 1151 | CB | PHE | 152 | 77.064 | 23.426 | 23.292 | 1.00 | 43.79 |
| ATOM | 1152 | CG | PHE | 152 | 77.805 | 24.014 | 24.464 | 1.00 | 46.32 |
| ATOM | 1153 | CD1 | PHE | 152 | 79.197 | 24.083 | 24.452 | 1.00 | 47.49 |
| ATOM | 1154 | CD2 | PHE | 152 | 77.125 | 24.517 | 25.570 | 1.00 | 47.25 |
| ATOM | 1155 | CE1 | PHE | 152 | 79.903 | 24.640 | 25.521 | 1.00 | 46.51 |
| ATOM | 1156 | CE2 | PHE | 152 | 77.824 | 25.079 | 26.646 | 1.00 | 46.64 |
| ATOM | 1157 | CZ | PHE | 152 | 79.216 | 25.138 | 26.618 | 1.00 | 46.97 |
| ATOM | 1158 | C | PHE | 152 | 75.010 | 22.376 | 22.370 | 1.00 | 44.97 |
| ATOM | 1159 | O | PHE | 152 | 75.380 | 21.436 | 21.672 | 1.00 | 45.07 |
| ATOM | 1160 | N | HIS | 153 | 73.971 | 23.135 | 22.054 | 1.00 | 47.34 |
| ATOM | 1161 | CA | HIS | 153 | 73.193 | 22.913 | 20.840 | 1.00 | 49.91 |
| ATOM | 1162 | CB | HIS | 153 | 71.905 | 23.729 | 20.906 | 1.00 | 51.90 |
| ATOM | 1163 | CG | HIS | 153 | 70.927 | 23.409 | 19.821 | 1.00 | 52.97 |
| ATOM | 1164 | CD2 | HIS | 153 | 70.035 | 22.396 | 19.697 | 1.00 | 53.16 |
| ATOM | 1165 | ND1 | HIS | 153 | 70.776 | 24.194 | 18.697 | 1.00 | 53.71 |
| ATOM | 1166 | CE1 | HIS | 153 | 69.831 | 23.680 | 17.931 | 1.00 | 55.24 |
| ATOM | 1167 | NE2 | HIS | 153 | 69.365 | 22.589 | 18.515 | 1.00 | 55.29 |
| ATOM | 1168 | C | HIS | 153 | 74.004 | 23.334 | 19.622 | 1.00 | 51.87 |
| ATOM | 1169 | O | HIS | 153 | 74.923 | 24.146 | 19.735 | 1.00 | 51.83 |
| ATOM | 1170 | N | LYS | 154 | 73.673 | 22.790 | 18.456 | 1.00 | 55.48 |
| ATOM | 1171 | CA | LYS | 154 | 74.407 | 23.144 | 17.247 | 1.00 | 60.47 |
| ATOM | 1172 | CB | LYS | 154 | 73.829 | 22.445 | 16.015 | 1.00 | 63.01 |
| ATOM | 1173 | CG | LYS | 154 | 74.206 | 20.976 | 15.895 | 1.00 | 68.55 |
| ATOM | 1174 | CD | LYS | 154 | 74.099 | 20.487 | 14.449 | 1.00 | 73.23 |
| ATOM | 1175 | CE | LYS | 154 | 72.700 | 20.712 | 13.862 | 1.00 | 76.00 |
| ATOM | 1176 | NZ | LYS | 154 | 72.575 | 20.188 | 12.467 | 1.00 | 75.37 |
| ATOM | 1177 | C | LYS | 154 | 74.381 | 24.642 | 17.024 | 1.00 | 62.14 |
| ATOM | 1178 | O | LYS | 154 | 75.423 | 25.262 | 16.808 | 1.00 | 63.02 |
| ATOM | 1179 | N | ASN | 155 | 73.189 | 25.225 | 17.095 | 1.00 | 63.91 |
| ATOM | 1180 | CA | ASN | 155 | 73.031 | 26.659 | 16.883 | 1.00 | 66.44 |
| ATOM | 1181 | CB | ASN | 155 | 71.566 | 26.981 | 16.573 | 1.00 | 68.56 |
| ATOM | 1182 | CG | ASN | 155 | 71.112 | 26.419 | 15.230 | 1.00 | 71.59 |
| ATOM | 1183 | OD1 | ASN | 155 | 71.122 | 25.209 | 15.014 | 1.00 | 73.47 |
| ATOM | 1184 | ND2 | ASN | 155 | 70.714 | 27.304 | 14.319 | 1.00 | 73.15 |
| ATOM | 1185 | C | ASN | 155 | 73.516 | 27.532 | 18.042 | 1.00 | 66.84 |
| ATOM | 1186 | O | ASN | 155 | 73.488 | 28.762 | 17.946 | 1.00 | 66.67 |
| ATOM | 1187 | N | ASN | 156 | 73.965 | 26.912 | 19.131 | 1.00 | 67.57 |
| ATOM | 1188 | CA | ASN | 156 | 74.440 | 27.684 | 20.273 | 1.00 | 68.50 |
| ATOM | 1189 | CB | ASN | 156 | 74.905 | 26.773 | 21.412 | 1.00 | 66.33 |
| ATOM | 1190 | CG | ASN | 156 | 75.184 | 27.542 | 22.694 | 1.00 | 63.56 |
| ATOM | 1191 | OD1 | ASN | 156 | 75.866 | 28.557 | 22.678 | 1.00 | 62.87 |
| ATOM | 1192 | ND2 | ASN | 156 | 74.662 | 27.054 | 23.811 | 1.00 | 63.14 |
| ATOM | 1193 | C | ASN | 156 | 75.595 | 28.557 | 19.807 | 1.00 | 70.55 |
| ATOM | 1194 | O | ASN | 156 | 76.671 | 28.057 | 19.457 | 1.00 | 70.12 |
| ATOM | 1195 | N | GLN | 157 | 75.350 | 29.865 | 19.802 | 1.00 | 72.48 |
| ATOM | 1196 | CA | GLN | 157 | 76.330 | 30.855 | 19.369 | 1.00 | 73.78 |
| ATOM | 1197 | CB | GLN | 157 | 75.600 | 32.098 | 18.863 | 1.00 | 74.49 |
| ATOM | 1198 | CG | GLN | 157 | 74.721 | 31.834 | 17.660 | 1.00 | 76.92 |
| ATOM | 1199 | CD | GLN | 157 | 75.500 | 31.859 | 16.363 | 1.00 | 79.62 |
| ATOM | 1200 | OE1 | GLN | 157 | 75.897 | 32.929 | 15.893 | 1.00 | 82.63 |
| ATOM | 1201 | NE2 | GLN | 157 | 75.732 | 30.683 | 15.777 | 1.00 | 79.15 |
| ATOM | 1202 | C | GLN | 157 | 77.295 | 31.251 | 20.479 | 1.00 | 74.47 |
| ATOM | 1203 | O | GLN | 157 | 78.237 | 32.013 | 20.248 | 1.00 | 75.49 |
| ATOM | 1204 | N | LEU | 158 | 77.063 | 30.728 | 21.680 | 1.00 | 73.60 |
| ATOM | 1205 | CA | LEU | 158 | 77.904 | 31.040 | 22.829 | 1.00 | 72.29 |
| ATOM | 1206 | CB | LEU | 158 | 77.104 | 31.885 | 23.820 | 1.00 | 71.73 |
| ATOM | 1207 | CG | LEU | 158 | 76.730 | 33.257 | 23.254 | 1.00 | 70.67 |
| ATOM | 1208 | CD1 | LEU | 158 | 75.489 | 33.791 | 23.939 | 1.00 | 71.16 |
| ATOM | 1209 | CD2 | LEU | 158 | 77.910 | 34.201 | 23.418 | 1.00 | 70.11 |
| ATOM | 1210 | C | LEU | 158 | 78.444 | 29.785 | 23.509 | 1.00 | 72.29 |
| ATOM | 1211 | O | LEU | 158 | 78.572 | 29.732 | 24.738 | 1.00 | 72.03 |
| ATOM | 1212 | N | ALA | 159 | 78.761 | 28.780 | 22.696 | 1.00 | 71.81 |
| ATOM | 1213 | CA | ALA | 159 | 79.296 | 27.512 | 23.188 | 1.00 | 70.92 |
| ATOM | 1214 | CB | ALA | 159 | 79.338 | 26.489 | 22.055 | 1.00 | 71.11 |
| ATOM | 1215 | C | ALA | 159 | 80.698 | 27.749 | 23.740 | 1.00 | 70.01 |
| ATOM | 1216 | O | ALA | 159 | 81.677 | 27.176 | 23.269 | 1.00 | 69.90 |
| ATOM | 1217 | N | LEU | 160 | 80.778 | 28.609 | 24.745 | 1.00 | 68.85 |
| ATOM | 1218 | CA | LEU | 160 | 82.040 | 28.953 | 25.368 | 1.00 | 68.85 |
| ATOM | 1219 | CB | LEU | 160 | 82.051 | 30.459 | 25.652 | 1.00 | 69.61 |
| ATOM | 1220 | CG | LEU | 160 | 83.289 | 31.167 | 26.209 | 1.00 | 71.41 |
| ATOM | 1221 | CD1 | LEU | 160 | 83.155 | 32.672 | 25.979 | 1.00 | 70.97 |
| ATOM | 1222 | CD2 | LEU | 160 | 83.448 | 30.864 | 27.695 | 1.00 | 71.17 |
| ATOM | 1223 | C | LEU | 160 | 82.201 | 28.142 | 26.653 | 1.00 | 68.43 |
| ATOM | 1224 | O | LEU | 160 | 81.221 | 27.638 | 27.202 | 1.00 | 67.78 |
| ATOM | 1225 | N | THR | 161 | 83.437 | 28.007 | 27.122 | 1.00 | 67.93 |
| ATOM | 1226 | CA | THR | 161 | 83.715 | 27.252 | 28.340 | 1.00 | 67.32 |
| ATOM | 1227 | CB | THR | 161 | 84.015 | 25.769 | 28.011 | 1.00 | 67.61 |
| ATOM | 1228 | OG1 | THR | 161 | 82.822 | 25.127 | 27.545 | 1.00 | 68.81 |
| ATOM | 1229 | CG2 | THR | 161 | 84.513 | 25.039 | 29.236 | 1.00 | 69.29 |
| ATOM | 1230 | C | THR | 161 | 84.883 | 27.815 | 29.156 | 1.00 | 66.25 |
| ATOM | 1231 | O | THR | 161 | 85.851 | 28.342 | 28.606 | 1.00 | 66.57 |
| ATOM | 1232 | N | LEU | 162 | 84.765 | 27.709 | 30.477 | 1.00 | 64.73 |
| ATOM | 1233 | CA | LEU | 162 | 85.797 | 28.154 | 31.409 | 1.00 | 63.16 |
| ATOM | 1234 | CB | LEU | 162 | 85.587 | 29.599 | 31.844 | 1.00 | 63.88 |
| ATOM | 1235 | CG | LEU | 162 | 86.150 | 30.685 | 30.936 | 1.00 | 65.76 |
| ATOM | 1236 | CD1 | LEU | 162 | 86.247 | 31.982 | 31.736 | 1.00 | 64.20 |
| ATOM | 1237 | CD2 | LEU | 162 | 87.533 | 30.275 | 30.428 | 1.00 | 66.77 |
| ATOM | 1238 | C | LEU | 162 | 85.699 | 27.260 | 32.621 | 1.00 | 62.84 |
| ATOM | 1239 | O | LEU | 162 | 85.062 | 27.610 | 33.617 | 1.00 | 63.15 |
| ATOM | 1240 | N | ILE | 163 | 86.339 | 26.102 | 32.538 | 1.00 | 61.64 |
| ATOM | 1241 | CA | ILE | 163 | 86.276 | 25.148 | 33.622 | 1.00 | 61.03 |
| ATOM | 1242 | CB | ILE | 163 | 85.839 | 23.779 | 33.091 | 1.00 | 58.91 |
| ATOM | 1243 | CG2 | ILE | 163 | 85.660 | 22.808 | 34.245 | 1.00 | 57.25 |
| ATOM | 1244 | CG1 | ILE | 163 | 84.527 | 23.947 | 32.317 | 1.00 | 58.52 |
| ATOM | 1245 | CD1 | ILE | 163 | 84.036 | 22.705 | 31.620 | 1.00 | 59.29 |
| ATOM | 1246 | C | ILE | 163 | 87.556 | 25.004 | 34.414 | 1.00 | 61.98 |
| ATOM | 1247 | O | ILE | 163 | 88.598 | 24.637 | 33.877 | 1.00 | 62.41 |
| ATOM | 1248 | N | ASP | 164 | 87.453 | 25.303 | 35.706 | 1.00 | 63.32 |
| ATOM | 1249 | CA | ASP | 164 | 88.567 | 25.210 | 36.641 | 1.00 | 63.14 |
| ATOM | 1250 | CB | ASP | 164 | 88.375 | 26.237 | 37.768 | 1.00 | 61.88 |
| ATOM | 1251 | CG | ASP | 164 | 89.374 | 26.069 | 38.899 | 1.00 | 62.81 |
| ATOM | 1252 | OD1 | ASP | 164 | 90.576 | 25.893 | 38.605 | 1.00 | 62.88 |
| ATOM | 1253 | OD2 | ASP | 164 | 88.961 | 26.126 | 40.084 | 1.00 | 60.84 |
| ATOM | 1254 | C | ASP | 164 | 88.584 | 23.786 | 37.196 | 1.00 | 63.55 |
| ATOM | 1255 | O | ASP | 164 | 87.533 | 23.198 | 37.440 | 1.00 | 63.01 |
| ATOM | 1256 | N | THR | 165 | 89.773 | 23.224 | 37.383 | 1.00 | 64.53 |
| ATOM | 1257 | CA | THR | 165 | 89.871 | 21.868 | 37.904 | 1.00 | 64.48 |
| ATOM | 1258 | CB | THR | 165 | 90.503 | 20.924 | 36.875 | 1.00 | 64.86 |
| ATOM | 1259 | OG1 | THR | 165 | 91.734 | 21.484 | 36.410 | 1.00 | 65.61 |
| ATOM | 1260 | CG2 | THR | 165 | 89.557 | 20.717 | 35.695 | 1.00 | 64.19 |
| ATOM | 1261 | C | THR | 165 | 90.638 | 21.766 | 39.211 | 1.00 | 64.43 |
| ATOM | 1262 | O | THR | 165 | 90.703 | 20.691 | 39.803 | 1.00 | 63.97 |
| ATOM | 1263 | N | ASN | 166 | 91.210 | 22.878 | 39.667 | 1.00 | 65.17 |
| ATOM | 1264 | CA | ASN | 166 | 91.945 | 22.878 | 40.929 | 1.00 | 65.86 |
| ATOM | 1265 | CB | ASN | 166 | 92.328 | 24.302 | 41.359 | 1.00 | 68.67 |
| ATOM | 1266 | CG | ASN | 166 | 93.218 | 25.006 | 40.351 | 1.00 | 73.52 |
| ATOM | 1267 | OD1 | ASN | 166 | 94.005 | 24.367 | 39.651 | 1.00 | 71.97 |
| ATOM | 1268 | ND2 | ASN | 166 | 93.109 | 26.332 | 40.294 | 1.00 | 78.68 |
| ATOM | 1269 | C | ASN | 166 | 91.058 | 22.262 | 42.005 | 1.00 | 65.19 |
| ATOM | 1270 | O | ASN | 166 | 90.106 | 22.888 | 42.470 | 1.00 | 65.75 |
| ATOM | 1271 | N | ARG | 167 | 91.368 | 21.030 | 42.389 | 1.00 | 64.79 |
| ATOM | 1272 | CA | ARG | 167 | 90.598 | 20.330 | 43.408 | 1.00 | 64.71 |
| ATOM | 1273 | CB | ARG | 167 | 90.313 | 18.897 | 42.971 | 1.00 | 64.49 |
| ATOM | 1274 | CG | ARG | 167 | 89.594 | 18.791 | 41.663 | 1.00 | 65.00 |
| ATOM | 1275 | CD | ARG | 167 | 88.994 | 17.420 | 41.511 | 1.00 | 65.82 |
| ATOM | 1276 | NE | ARG | 167 | 88.143 | 17.344 | 40.332 | 1.00 | 67.22 |
| ATOM | 1277 | CZ | ARG | 167 | 88.591 | 17.452 | 39.089 | 1.00 | 68.51 |
| ATOM | 1278 | NH1 | ARG | 167 | 89.883 | 17.640 | 38.867 | 1.00 | 70.72 |
| ATOM | 1279 | NH2 | ARG | 167 | 87.749 | 17.365 | 38.067 | 1.00 | 70.00 |
| ATOM | 1280 | C | ARG | 167 | 91.349 | 20.299 | 44.727 | 1.00 | 64.78 |
| ATOM | 1281 | O | ARG | 167 | 92.543 | 20.583 | 44.776 | 1.00 | 65.42 |
| ATOM | 1282 | N | SER | 168 | 90.645 | 19.934 | 45.792 | 1.00 | 64.29 |
| ATOM | 1283 | CA | SER | 168 | 91.247 | 19.875 | 47.112 | 1.00 | 63.70 |
| ATOM | 1284 | CB | SER | 168 | 90.600 | 20.931 | 48.010 | 1.00 | 64.85 |
| ATOM | 1285 | OG | SER | 168 | 91.251 | 21.029 | 49.262 | 1.00 | 63.91 |
| ATOM | 1286 | C | SER | 168 | 91.078 | 18.478 | 47.705 | 1.00 | 64.32 |
| ATOM | 1287 | O | SER | 168 | 91.202 | 18.281 | 48.914 | 1.00 | 65.24 |
| ATOM | 1288 | N | ARG | 169 | 90.789 | 17.511 | 46.838 | 1.00 | 63.72 |
| ATOM | 1289 | CA | ARG | 169 | 90.618 | 16.115 | 47.233 | 1.00 | 62.92 |
| ATOM | 1290 | CB | ARG | 169 | 89.186 | 15.838 | 47.700 | 1.00 | 61.69 |
| ATOM | 1291 | CG | ARG | 169 | 88.159 | 15.928 | 46.585 | 1.00 | 60.81 |
| ATOM | 1292 | CD | ARG | 169 | 86.850 | 15.227 | 46.941 | 1.00 | 60.29 |
| ATOM | 1293 | NE | ARG | 169 | 85.978 | 15.115 | 45.771 | 1.00 | 60.63 |
| ATOM | 1294 | CZ | ARG | 169 | 84.979 | 14.246 | 45.652 | 1.00 | 58.93 |
| ATOM | 1295 | NH1 | ARG | 169 | 84.712 | 13.403 | 46.634 | 1.00 | 61.04 |
| ATOM | 1296 | NH2 | ARG | 169 | 84.251 | 14.216 | 44.544 | 1.00 | 58.88 |
| ATOM | 1297 | C | ARG | 169 | 90.910 | 15.271 | 45.999 | 1.00 | 63.61 |
| ATOM | 1298 | O | ARG | 169 | 91.042 | 15.802 | 44.896 | 1.00 | 64.62 |
| ATOM | 1299 | N | ALA | 170 | 91.013 | 13.961 | 46.182 | 1.00 | 63.09 |
| ATOM | 1300 | CA | ALA | 170 | 91.284 | 13.061 | 45.068 | 1.00 | 62.15 |
| ATOM | 1301 | CB | ALA | 170 | 92.188 | 11.929 | 45.533 | 1.00 | 61.97 |
| ATOM | 1302 | C | ALA | 170 | 89.964 | 12.503 | 44.546 | 1.00 | 61.36 |
| ATOM | 1303 | O | ALA | 170 | 89.064 | 12.213 | 45.333 | 1.00 | 62.33 |
| ATOM | 1304 | N | CYS | 171 | 89.843 | 12.347 | 43.229 | 1.00 | 60.67 |
| ATOM | 1305 | CA | CYS | 171 | 88.603 | 11.825 | 42.650 | 1.00 | 60.64 |
| ATOM | 1306 | C | CYS | 171 | 88.645 | 10.338 | 42.335 | 1.00 | 60.94 |
| ATOM | 1307 | O | CYS | 171 | 89.622 | 9.848 | 41.773 | 1.00 | 60.26 |
| ATOM | 1308 | CB | CYS | 171 | 88.249 | 12.547 | 41.338 | 1.00 | 59.51 |
| ATOM | 1309 | SG | CYS | 171 | 88.141 | 14.366 | 41.386 | 1.00 | 59.95 |
| ATOM | 1310 | N | HIS | 172 | 87.589 | 9.617 | 42.695 | 1.00 | 61.81 |
| ATOM | 1311 | CA | HIS | 172 | 87.520 | 8.203 | 42.347 | 1.00 | 62.68 |
| ATOM | 1312 | CB | HIS | 172 | 86.412 | 7.488 | 43.124 | 1.00 | 64.14 |
| ATOM | 1313 | CG | HIS | 172 | 86.736 | 7.251 | 44.568 | 1.00 | 67.75 |
| ATOM | 1314 | CD2 | HIS | 172 | 87.818 | 7.591 | 45.309 | 1.00 | 68.32 |
| ATOM | 1315 | ND1 | HIS | 172 | 85.879 | 6.588 | 45.422 | 1.00 | 68.70 |
| ATOM | 1316 | CE1 | HIS | 172 | 86.418 | 6.531 | 46.627 | 1.00 | 69.06 |
| ATOM | 1317 | NE2 | HIS | 172 | 87.594 | 7.132 | 46.585 | 1.00 | 70.47 |
| ATOM | 1318 | C | HIS | 172 | 87.161 | 8.243 | 40.861 | 1.00 | 63.27 |
| ATOM | 1319 | O | HIS | 172 | 86.545 | 9.200 | 40.392 | 1.00 | 63.84 |
| ATOM | 1320 | N | PRO | 173 | 87.541 | 7.213 | 40.096 | 1.00 | 63.36 |
| ATOM | 1321 | CD | PRO | 173 | 88.332 | 6.022 | 40.453 | 1.00 | 63.35 |
| ATOM | 1322 | CA | PRO | 173 | 87.219 | 7.218 | 38.666 | 1.00 | 62.52 |
| ATOM | 1323 | CB | PRO | 173 | 88.147 | 6.139 | 38.119 | 1.00 | 63.08 |
| ATOM | 1324 | CG | PRO | 173 | 88.152 | 5.142 | 39.235 | 1.00 | 62.82 |
| ATOM | 1325 | C | PRO | 173 | 85.745 | 6.941 | 38.345 | 1.00 | 61.30 |
| ATOM | 1326 | O | PRO | 173 | 85.018 | 6.394 | 39.174 | 1.00 | 60.21 |
| ATOM | 1327 | N | CYS | 174 | 85.319 | 7.321 | 37.138 | 1.00 | 60.56 |
| ATOM | 1328 | CA | CYS | 174 | 83.944 | 7.091 | 36.694 | 1.00 | 60.85 |
| ATOM | 1329 | C | CYS | 174 | 83.643 | 5.605 | 36.800 | 1.00 | 62.57 |
| ATOM | 1330 | O | CYS | 174 | 84.559 | 4.787 | 36.806 | 1.00 | 64.23 |
| ATOM | 1331 | CB | CYS | 174 | 83.750 | 7.506 | 35.227 | 1.00 | 58.70 |
| ATOM | 1332 | SG | CYS | 174 | 83.709 | 9.288 | 34.837 | 1.00 | 56.01 |
| ATOM | 1333 | N | SER | 175 | 82.362 | 5.254 | 36.877 | 1.00 | 64.18 |
| ATOM | 1334 | CA | SER | 175 | 81.961 | 3.852 | 36.956 | 1.00 | 65.15 |
| ATOM | 1335 | CB | SER | 175 | 80.433 | 3.738 | 37.084 | 1.00 | 65.03 |
| ATOM | 1336 | OG | SER | 175 | 79.996 | 2.385 | 37.055 | 1.00 | 62.93 |
| ATOM | 1337 | C | SER | 175 | 82.437 | 3.126 | 35.689 | 1.00 | 66.82 |
| ATOM | 1338 | O | SER | 175 | 82.599 | 3.742 | 34.627 | 1.00 | 65.55 |
| ATOM | 1339 | N | PRO | 176 | 82.677 | 1.805 | 35.793 | 1.00 | 67.86 |
| ATOM | 1340 | CD | PRO | 176 | 82.600 | 0.994 | 37.021 | 1.00 | 67.63 |
| ATOM | 1341 | CA | PRO | 176 | 83.136 | 0.991 | 34.661 | 1.00 | 68.04 |
| ATOM | 1342 | CB | PRO | 176 | 83.251 | −0.408 | 35.266 | 1.00 | 68.70 |
| ATOM | 1343 | CG | PRO | 176 | 83.552 | −0.132 | 36.711 | 1.00 | 69.00 |
| ATOM | 1344 | C | PRO | 176 | 82.126 | 1.039 | 33.530 | 1.00 | 68.50 |
| ATOM | 1345 | O | PRO | 176 | 82.476 | 0.908 | 32.358 | 1.00 | 68.63 |
| ATOM | 1346 | N | MET | 177 | 80.867 | 1.233 | 33.908 | 1.00 | 69.98 |
| ATOM | 1347 | CA | MET | 177 | 79.753 | 1.307 | 32.966 | 1.00 | 71.03 |
| ATOM | 1348 | CB | MET | 177 | 78.442 | 1.427 | 33.740 | 1.00 | 74.07 |
| ATOM | 1349 | CG | MET | 177 | 78.160 | 0.279 | 34.697 | 1.00 | 76.88 |
| ATOM | 1350 | SD | MET | 177 | 77.587 | −1.175 | 33.823 | 1.00 | 79.28 |
| ATOM | 1351 | CE | MET | 177 | 76.129 | −0.467 | 32.980 | 1.00 | 76.83 |
| ATOM | 1352 | C | MET | 177 | 79.860 | 2.489 | 32.002 | 1.00 | 69.93 |
| ATOM | 1353 | O | MET | 177 | 79.295 | 2.452 | 30.905 | 1.00 | 69.07 |
| ATOM | 1354 | N | CYS | 178 | 80.574 | 3.535 | 32.413 | 1.00 | 68.38 |
| ATOM | 1355 | CA | CYS | 178 | 80.721 | 4.721 | 31.581 | 1.00 | 67.86 |
| ATOM | 1356 | C | CYS | 178 | 81.723 | 4.536 | 30.454 | 1.00 | 70.14 |
| ATOM | 1357 | O | CYS | 178 | 82.929 | 4.410 | 30.679 | 1.00 | 70.44 |
| ATOM | 1358 | CB | CYS | 178 | 81.131 | 5.927 | 32.419 | 1.00 | 64.77 |
| ATOM | 1359 | SG | CYS | 178 | 79.973 | 6.377 | 33.746 | 1.00 | 60.59 |
| ATOM | 1360 | N | LYS | 179 | 81.193 | 4.552 | 29.237 | 1.00 | 71.44 |
| ATOM | 1361 | CA | LYS | 179 | 81.961 | 4.392 | 28.009 | 1.00 | 72.58 |
| ATOM | 1362 | CB | LYS | 179 | 81.111 | 4.872 | 26.823 | 1.00 | 74.72 |
| ATOM | 1363 | CG | LYS | 179 | 79.759 | 4.447 | 26.948 | 1.00 | 76.95 |
| ATOM | 1364 | C | LYS | 179 | 83.320 | 5.107 | 27.981 | 1.00 | 71.24 |
| ATOM | 1365 | O | LYS | 179 | 84.348 | 4.529 | 28.346 | 1.00 | 71.78 |
| ATOM | 1366 | N | GLY | 180 | 83.319 | 6.362 | 27.539 | 1.00 | 68.84 |
| ATOM | 1367 | CA | GLY | 180 | 84.557 | 7.113 | 27.436 | 1.00 | 66.76 |
| ATOM | 1368 | C | GLY | 180 | 85.009 | 7.880 | 28.665 | 1.00 | 65.59 |
| ATOM | 1369 | O | GLY | 180 | 85.504 | 9.001 | 28.546 | 1.00 | 65.62 |
| ATOM | 1370 | N | SER | 181 | 84.855 | 7.284 | 29.843 | 1.00 | 63.98 |
| ATOM | 1371 | CA | SER | 181 | 85.268 | 7.942 | 31.076 | 1.00 | 62.30 |
| ATOM | 1372 | CB | SER | 181 | 86.785 | 8.155 | 31.085 | 1.00 | 63.70 |
| ATOM | 1373 | OG | SER | 181 | 87.479 | 6.920 | 31.015 | 1.00 | 67.09 |
| ATOM | 1374 | C | SER | 181 | 84.577 | 9.289 | 31.233 | 1.00 | 60.44 |
| ATOM | 1375 | O | SER | 181 | 85.179 | 10.250 | 31.714 | 1.00 | 60.86 |
| ATOM | 1376 | N | ARG | 182 | 83.316 | 9.358 | 30.816 | 1.00 | 57.20 |
| ATOM | 1377 | CA | ARG | 182 | 82.541 | 10.586 | 30.922 | 1.00 | 53.54 |
| ATOM | 1378 | CB | ARG | 182 | 81.994 | 10.977 | 29.547 | 1.00 | 52.60 |
| ATOM | 1379 | CG | ARG | 182 | 83.079 | 11.307 | 28.526 | 1.00 | 51.05 |
| ATOM | 1380 | CD | ARG | 182 | 82.480 | 11.761 | 27.208 | 1.00 | 49.89 |
| ATOM | 1381 | NE | ARG | 182 | 83.487 | 12.061 | 26.190 | 1.00 | 50.63 |
| ATOM | 1382 | CZ | ARG | 182 | 84.426 | 12.997 | 26.313 | 1.00 | 53.22 |
| ATOM | 1383 | NH1 | ARG | 182 | 84.501 | 13.733 | 27.417 | 1.00 | 53.61 |
| ATOM | 1384 | NH2 | ARG | 182 | 85.285 | 13.213 | 25.323 | 1.00 | 52.35 |
| ATOM | 1385 | C | ARG | 182 | 81.392 | 10.399 | 31.914 | 1.00 | 52.13 |
| ATOM | 1386 | O | ARG | 182 | 80.448 | 9.652 | 31.647 | 1.00 | 49.98 |
| ATOM | 1387 | N | CYS | 183 | 81.477 | 11.067 | 33.063 | 1.00 | 50.35 |
| ATOM | 1388 | CA | CYS | 183 | 80.424 | 10.954 | 34.067 | 1.00 | 50.36 |
| ATOM | 1389 | C | CYS | 183 | 80.235 | 12.234 | 34.876 | 1.00 | 50.14 |
| ATOM | 1390 | O | CYS | 183 | 81.110 | 13.113 | 34.878 | 1.00 | 49.09 |
| ATOM | 1391 | CB | CYS | 183 | 80.721 | 9.791 | 35.017 | 1.00 | 51.52 |
| ATOM | 1392 | SG | CYS | 183 | 82.222 | 10.011 | 36.025 | 1.00 | 53.52 |
| ATOM | 1393 | N | TRP | 184 | 79.091 | 12.326 | 35.561 | 1.00 | 49.39 |
| ATOM | 1394 | CA | TRP | 184 | 78.754 | 13.485 | 36.391 | 1.00 | 50.35 |
| ATOM | 1395 | CB | TRP | 184 | 77.272 | 13.844 | 36.280 | 1.00 | 48.39 |
| ATOM | 1396 | CG | TRP | 184 | 76.840 | 14.320 | 34.949 | 1.00 | 46.10 |
| ATOM | 1397 | CD2 | TRP | 184 | 77.000 | 15.639 | 34.425 | 1.00 | 46.71 |
| ATOM | 1398 | CE2 | TRP | 184 | 76.450 | 15.640 | 33.128 | 1.00 | 46.63 |
| ATOM | 1399 | CE3 | TRP | 184 | 77.556 | 16.825 | 34.927 | 1.00 | 46.81 |
| ATOM | 1400 | CD1 | TRP | 184 | 76.220 | 13.593 | 33.981 | 1.00 | 44.32 |
| ATOM | 1401 | NE1 | TRP | 184 | 75.982 | 14.376 | 32.882 | 1.00 | 45.69 |
| ATOM | 1402 | CZ2 | TRP | 184 | 76.436 | 16.784 | 32.320 | 1.00 | 46.95 |
| ATOM | 1403 | CZ3 | TRP | 184 | 77.545 | 17.962 | 34.125 | 1.00 | 45.69 |
| ATOM | 1404 | CH2 | TRP | 184 | 76.987 | 17.932 | 32.834 | 1.00 | 46.50 |
| ATOM | 1405 | C | TRP | 184 | 79.034 | 13.182 | 37.845 | 1.00 | 52.63 |
| ATOM | 1406 | O | TRP | 184 | 78.997 | 14.072 | 38.693 | 1.00 | 52.34 |
| ATOM | 1407 | N | GLY | 185 | 79.283 | 11.912 | 38.130 | 1.00 | 55.68 |
| ATOM | 1408 | CA | GLY | 185 | 79.559 | 11.497 | 39.490 | 1.00 | 58.79 |
| ATOM | 1409 | C | GLY | 185 | 80.110 | 10.091 | 39.502 | 1.00 | 61.63 |
| ATOM | 1410 | O | GLY | 185 | 80.183 | 9.435 | 38.461 | 1.00 | 63.55 |
| ATOM | 1411 | N | GLU | 186 | 80.498 | 9.621 | 40.680 | 1.00 | 63.97 |
| ATOM | 1412 | CA | GLU | 186 | 81.057 | 8.282 | 40.821 | 1.00 | 65.52 |
| ATOM | 1413 | CB | GLU | 186 | 81.624 | 8.113 | 42.238 | 1.00 | 68.21 |
| ATOM | 1414 | CG | GLU | 186 | 81.996 | 6.687 | 42.614 | 1.00 | 73.34 |
| ATOM | 1415 | CD | GLU | 186 | 82.845 | 6.608 | 43.877 | 1.00 | 76.15 |
| ATOM | 1416 | OE1 | GLU | 186 | 82.521 | 7.314 | 44.864 | 1.00 | 75.86 |
| ATOM | 1417 | OE2 | GLU | 186 | 83.830 | 5.829 | 43.877 | 1.00 | 76.23 |
| ATOM | 1418 | C | GLU | 186 | 80.052 | 7.172 | 40.510 | 1.00 | 64.74 |
| ATOM | 1419 | O | GLU | 186 | 80.430 | 6.108 | 40.023 | 1.00 | 64.79 |
| ATOM | 1420 | N | SER | 187 | 78.773 | 7.421 | 40.768 | 1.00 | 64.00 |
| ATOM | 1421 | CA | SER | 187 | 77.748 | 6.412 | 40.524 | 1.00 | 64.83 |
| ATOM | 1422 | CB | SER | 187 | 76.360 | 6.992 | 40.784 | 1.00 | 64.09 |
| ATOM | 1423 | OG | SER | 187 | 75.364 | 6.029 | 40.506 | 1.00 | 65.85 |
| ATOM | 1424 | C | SER | 187 | 77.781 | 5.788 | 39.131 | 1.00 | 65.54 |
| ATOM | 1425 | O | SER | 187 | 78.370 | 6.334 | 38.193 | 1.00 | 67.53 |
| ATOM | 1426 | N | SER | 188 | 77.145 | 4.627 | 39.011 | 1.00 | 65.72 |
| ATOM | 1427 | CA | SER | 188 | 77.081 | 3.903 | 37.747 | 1.00 | 65.37 |
| ATOM | 1428 | CB | SER | 188 | 76.828 | 2.414 | 37.997 | 1.00 | 65.58 |
| ATOM | 1429 | OG | SER | 188 | 75.640 | 2.213 | 38.745 | 1.00 | 66.29 |
| ATOM | 1430 | C | SER | 188 | 75.941 | 4.485 | 36.942 | 1.00 | 65.07 |
| ATOM | 1431 | O | SER | 188 | 75.706 | 4.102 | 35.798 | 1.00 | 63.32 |
| ATOM | 1432 | N | GLU | 189 | 75.232 | 5.419 | 37.563 | 1.00 | 65.54 |
| ATOM | 1433 | CA | GLU | 189 | 74.108 | 6.074 | 36.922 | 1.00 | 65.45 |
| ATOM | 1434 | CB | GLU | 189 | 72.909 | 6.075 | 37.863 | 1.00 | 68.22 |
| ATOM | 1435 | CG | GLU | 189 | 72.379 | 4.704 | 38.223 | 1.00 | 71.35 |
| ATOM | 1436 | CD | GLU | 189 | 71.195 | 4.799 | 39.166 | 1.00 | 74.87 |
| ATOM | 1437 | OE1 | GLU | 189 | 70.549 | 3.760 | 39.433 | 1.00 | 76.57 |
| ATOM | 1438 | OE2 | GLU | 189 | 70.913 | 5.923 | 39.645 | 1.00 | 74.44 |
| ATOM | 1439 | C | GLU | 189 | 74.428 | 7.509 | 36.522 | 1.00 | 63.65 |
| ATOM | 1440 | O | GLU | 189 | 73.521 | 8.274 | 36.198 | 1.00 | 64.33 |
| ATOM | 1441 | N | ASP | 190 | 75.705 | 7.879 | 36.543 | 1.00 | 61.27 |
| ATOM | 1442 | CA | ASP | 190 | 76.087 | 9.237 | 36.185 | 1.00 | 59.70 |
| ATOM | 1443 | CB | ASP | 190 | 76.898 | 9.868 | 37.309 | 1.00 | 60.26 |
| ATOM | 1444 | CG | ASP | 190 | 76.138 | 9.915 | 38.607 | 1.00 | 62.99 |
| ATOM | 1445 | OD1 | ASP | 190 | 74.995 | 10.429 | 38.622 | 1.00 | 62.79 |
| ATOM | 1446 | OD2 | ASP | 190 | 76.691 | 9.440 | 39.619 | 1.00 | 65.30 |
| ATOM | 1447 | C | ASP | 190 | 76.867 | 9.339 | 34.884 | 1.00 | 58.34 |
| ATOM | 1448 | O | ASP | 190 | 77.533 | 10.344 | 34.629 | 1.00 | 58.18 |
| ATOM | 1449 | N | CYS | 191 | 76.785 | 8.305 | 34.059 | 1.00 | 56.49 |
| ATOM | 1450 | CA | CYS | 191 | 77.491 | 8.324 | 32.787 | 1.00 | 55.82 |
| ATOM | 1451 | C | CYS | 191 | 76.855 | 9.351 | 31.850 | 1.00 | 55.00 |
| ATOM | 1452 | O | CYS | 191 | 75.640 | 9.316 | 31.625 | 1.00 | 54.59 |
| ATOM | 1453 | CB | CYS | 191 | 77.450 | 6.937 | 32.142 | 1.00 | 56.66 |
| ATOM | 1454 | SG | CYS | 191 | 78.189 | 5.611 | 33.156 | 1.00 | 56.79 |
| ATOM | 1455 | N | GLN | 192 | 77.663 | 10.269 | 31.319 | 1.00 | 52.00 |
| ATOM | 1456 | CA | GLN | 192 | 77.144 | 11.272 | 30.398 | 1.00 | 50.36 |
| ATOM | 1457 | CB | GLN | 192 | 78.229 | 12.283 | 29.975 | 1.00 | 48.42 |
| ATOM | 1458 | CG | GLN | 192 | 77.836 | 13.130 | 28.750 | 1.00 | 44.65 |
| ATOM | 1459 | CD | GLN | 192 | 78.811 | 14.268 | 28.423 | 1.00 | 45.67 |
| ATOM | 1460 | OE1 | GLN | 192 | 79.992 | 14.214 | 28.761 | 1.00 | 45.08 |
| ATOM | 1461 | NE2 | GLN | 192 | 78.312 | 15.294 | 27.735 | 1.00 | 45.93 |
| ATOM | 1462 | C | GLN | 192 | 76.606 | 10.573 | 29.166 | 1.00 | 50.85 |
| ATOM | 1463 | O | GLN | 192 | 77.304 | 9.789 | 28.531 | 1.00 | 52.31 |
| ATOM | 1464 | N | SER | 193 | 75.347 | 10.838 | 28.848 | 1.00 | 50.48 |
| ATOM | 1465 | CA | SER | 193 | 74.721 | 10.255 | 27.674 | 1.00 | 50.36 |
| ATOM | 1466 | CB | SER | 193 | 73.231 | 10.007 | 27.941 | 1.00 | 51.05 |
| ATOM | 1467 | OG | SER | 193 | 72.622 | 9.330 | 26.856 | 1.00 | 54.42 |
| ATOM | 1468 | C | SER | 193 | 74.896 | 11.284 | 26.558 | 1.00 | 49.91 |
| ATOM | 1469 | O | SER | 193 | 74.477 | 12.437 | 26.703 | 1.00 | 51.97 |
| ATOM | 1470 | N | LEU | 194 | 75.525 | 10.885 | 25.456 | 1.00 | 47.52 |
| ATOM | 1471 | CA | LEU | 194 | 75.743 | 11.802 | 24.340 | 1.00 | 45.90 |
| ATOM | 1472 | CB | LEU | 194 | 77.042 | 11.425 | 23.618 | 1.00 | 45.34 |
| ATOM | 1473 | CG | LEU | 194 | 78.254 | 11.307 | 24.561 | 1.00 | 45.76 |
| ATOM | 1474 | CD1 | LED | 194 | 79.524 | 10.936 | 23.792 | 1.00 | 46.27 |
| ATOM | 1475 | CD2 | LEU | 194 | 78.453 | 12.621 | 25.281 | 1.00 | 43.23 |
| ATOM | 1476 | C | LED | 194 | 74.541 | 11.767 | 23.388 | 1.00 | 45.22 |
| ATOM | 1477 | O | LED | 194 | 73.954 | 10.706 | 23.174 | 1.00 | 44.15 |
| ATOM | 1478 | N | THR | 195 | 74.158 | 12.922 | 22.836 | 1.00 | 45.06 |
| ATOM | 1479 | CA | THR | 195 | 73.004 | 12.977 | 21.932 | 1.00 | 45.39 |
| ATOM | 1480 | CB | THR | 195 | 71.721 | 13.462 | 22.677 | 1.00 | 42.78 |
| ATOM | 1481 | OG1 | THR | 195 | 71.877 | 14.824 | 23.092 | 1.00 | 42.26 |
| ATOM | 1482 | CG2 | THR | 195 | 71.454 | 12.610 | 23.888 | 1.00 | 40.24 |
| ATOM | 1483 | C | THR | 195 | 73.188 | 13.848 | 20.685 | 1.00 | 48.13 |
| ATOM | 1484 | O | THR | 195 | 72.240 | 14.046 | 19.906 | 1.00 | 47.32 |
| ATOM | 1485 | N | ARG | 196 | 74.398 | 14.366 | 20.496 | 1.00 | 50.47 |
| ATOM | 1486 | CA | ARG | 196 | 74.700 | 15.206 | 19.339 | 1.00 | 54.64 |
| ATOM | 1487 | CB | ARG | 196 | 74.945 | 16.661 | 19.774 | 1.00 | 55.44 |
| ATOM | 1488 | CG | ARG | 196 | 75.186 | 17.639 | 18.615 | 1.00 | 57.51 |
| ATOM | 1489 | CD | ARG | 196 | 75.521 | 19.042 | 19.111 | 1.00 | 56.78 |
| ATOM | 1490 | NE | ARG | 196 | 76.599 | 19.642 | 18.328 | 1.00 | 60.42 |
| ATOM | 1491 | CZ | ARG | 196 | 77.246 | 20.759 | 18.661 | 1.00 | 61.82 |
| ATOM | 1492 | NH1 | ARG | 196 | 76.924 | 21.413 | 19.769 | 1.00 | 61.40 |
| ATOM | 1493 | NH2 | ARG | 196 | 78.235 | 21.214 | 17.897 | 1.00 | 61.58 |
| ATOM | 1494 | C | ARG | 196 | 75.934 | 14.673 | 18.611 | 1.00 | 56.36 |
| ATOM | 1495 | O | ARG | 196 | 75.830 | 14.136 | 17.514 | 1.00 | 56.46 |
| ATOM | 1496 | N | THR | 197 | 77.097 | 14.817 | 19.244 | 1.00 | 59.50 |
| ATOM | 1497 | CA | THR | 197 | 78.374 | 14.370 | 18.687 | 1.00 | 61.37 |
| ATOM | 1498 | CB | THR | 197 | 79.531 | 14.549 | 19.719 | 1.00 | 61.39 |
| ATOM | 1499 | OG1 | THR | 197 | 79.214 | 13.846 | 20.927 | 1.00 | 63.55 |
| ATOM | 1500 | CG2 | THR | 197 | 79.752 | 16.020 | 20.039 | 1.00 | 60.20 |
| ATOM | 1501 | C | THR | 197 | 78.428 | 12.922 | 18.167 | 1.00 | 62.07 |
| ATOM | 1502 | O | THR | 197 | 79.360 | 12.556 | 17.447 | 1.00 | 63.66 |
| ATOM | 1503 | N | VAL | 198 | 77.450 | 12.094 | 18.515 | 1.00 | 62.36 |
| ATOM | 1504 | CA | VAL | 198 | 77.464 | 10.705 | 18.051 | 1.00 | 63.59 |
| ATOM | 1505 | CB | VAL | 198 | 77.466 | 9.733 | 19.239 | 1.00 | 63.41 |
| ATOM | 1506 | CG1 | VAL | 198 | 78.619 | 10.061 | 20.169 | 1.00 | 65.40 |
| ATOM | 1507 | CG2 | VAL | 198 | 76.141 | 9.817 | 19.981 | 1.00 | 63.36 |
| ATOM | 1508 | C | VAL | 198 | 76.269 | 10.365 | 17.168 | 1.00 | 64.53 |
| ATOM | 1509 | O | VAL | 198 | 75.768 | 9.237 | 17.200 | 1.00 | 65.15 |
| ATOM | 1510 | N | CYS | 199 | 75.820 | 11.332 | 16.372 | 1.00 | 66.05 |
| ATOM | 1511 | CA | CYS | 199 | 74.659 | 11.128 | 15.511 | 1.00 | 67.00 |
| ATOM | 1512 | C | CYS | 199 | 74.962 | 10.869 | 14.040 | 1.00 | 69.27 |
| ATOM | 1513 | O | CYS | 199 | 75.999 | 11.288 | 13.510 | 1.00 | 67.96 |
| ATOM | 1514 | CB | CYS | 199 | 73.705 | 12.325 | 15.613 | 1.00 | 64.26 |
| ATOM | 1515 | SG | CYS | 199 | 73.095 | 12.674 | 17.292 | 1.00 | 58.90 |
| ATOM | 1516 | N | ALA | 200 | 74.024 | 10.176 | 13.397 | 1.00 | 71.92 |
| ATOM | 1517 | CA | ALA | 200 | 74.109 | 9.831 | 11.984 | 1.00 | 74.79 |
| ATOM | 1518 | CB | ALA | 200 | 73.163 | 8.670 | 11.668 | 1.00 | 75.33 |
| ATOM | 1519 | C | ALA | 200 | 73.732 | 11.052 | 11.157 | 1.00 | 76.31 |
| ATOM | 1520 | O | ALA | 200 | 73.453 | 12.119 | 11.711 | 1.00 | 76.44 |
| ATOM | 1521 | N | GLY | 201 | 73.713 | 10.887 | 9.834 | 1.00 | 78.51 |
| ATOM | 1522 | CA | GLY | 201 | 73.382 | 11.992 | 8.952 | 1.00 | 78.51 |
| ATOM | 1523 | C | GLY | 201 | 74.024 | 13.240 | 9.517 | 1.00 | 79.24 |
| ATOM | 1524 | O | GLY | 201 | 75.241 | 13.284 | 9.716 | 1.00 | 79.77 |
| ATOM | 1525 | N | GLY | 202 | 73.207 | 14.249 | 9.801 | 1.00 | 78.75 |
| ATOM | 1526 | CA | GLY | 202 | 73.729 | 15.475 | 10.372 | 1.00 | 77.26 |
| ATOM | 1527 | C | GLY | 202 | 72.721 | 16.040 | 11.350 | 1.00 | 75.70 |
| ATOM | 1528 | O | GLY | 202 | 72.779 | 17.222 | 11.699 | 1.00 | 75.31 |
| ATOM | 1529 | N | CYS | 203 | 71.801 | 15.192 | 11.807 | 1.00 | 73.40 |
| ATOM | 1530 | CA | CYS | 203 | 70.758 | 15.645 | 12.718 | 1.00 | 71.81 |
| ATOM | 1531 | C | CYS | 203 | 71.308 | 16.143 | 14.045 | 1.00 | 68.86 |
| ATOM | 1532 | O | CYS | 203 | 72.292 | 15.613 | 14.558 | 1.00 | 68.82 |
| ATOM | 1533 | CB | CYS | 203 | 69.734 | 14.540 | 12.948 | 1.00 | 72.59 |
| ATOM | 1534 | SG | CYS | 203 | 70.319 | 13.145 | 13.941 | 1.00 | 75.84 |
| ATOM | 1535 | N | ALA | 204 | 70.652 | 17.169 | 14.590 | 1.00 | 65.90 |
| ATOM | 1536 | CA | ALA | 204 | 71.050 | 17.813 | 15.845 | 1.00 | 62.55 |
| ATOM | 1537 | CB | ALA | 204 | 70.169 | 19.037 | 16.091 | 1.00 | 60.92 |
| ATOM | 1538 | C | ALA | 204 | 71.054 | 16.921 | 17.087 | 1.00 | 60.13 |
| ATOM | 1539 | O | ALA | 204 | 71.996 | 16.970 | 17.880 | 1.00 | 60.20 |
| ATOM | 1540 | N | ARG | 205 | 69.999 | 16.129 | 17.261 | 1.00 | 57.62 |
| ATOM | 1541 | CA | ARG | 205 | 69.882 | 15.233 | 18.409 | 1.00 | 56.05 |
| ATOM | 1542 | CB | ARG | 205 | 68.831 | 15.759 | 19.393 | 1.00 | 53.61 |
| ATOM | 1543 | CG | ARG | 205 | 69.196 | 17.073 | 20.070 | 1.00 | 52.57 |
| ATOM | 1544 | CD | ARG | 205 | 70.444 | 16.932 | 20.939 | 1.00 | 49.89 |
| ATOM | 1545 | NE | ARG | 205 | 70.879 | 18.209 | 21.501 | 1.00 | 47.37 |
| ATOM | 1546 | CZ | ARG | 205 | 72.046 | 18.391 | 22.109 | 1.00 | 49.35 |
| ATOM | 1547 | NH1 | ARG | 205 | 72.892 | 17.371 | 22.236 | 1.00 | 51.06 |
| ATOM | 1548 | NH2 | ARG | 205 | 72.378 | 19.584 | 22.584 | 1.00 | 47.62 |
| ATOM | 1549 | C | ARG | 205 | 69.499 | 13.830 | 17.960 | 1.00 | 56.33 |
| ATOM | 1550 | O | ARG | 205 | 68.872 | 13.654 | 16.917 | 1.00 | 55.93 |
| ATOM | 1551 | N | CYS | 206 | 69.866 | 12.828 | 18.750 | 1.00 | 56.86 |
| ATOM | 1552 | CA | CYS | 206 | 69.545 | 11.459 | 18.383 | 1.00 | 59.23 |
| ATOM | 1553 | C | CYS | 206 | 69.528 | 10.509 | 19.578 | 1.00 | 62.16 |
| ATOM | 1554 | O | CYS | 206 | 70.103 | 10.799 | 20.625 | 1.00 | 62.41 |
| ATOM | 1555 | CB | CYS | 206 | 70.535 | 10.970 | 17.326 | 1.00 | 56.45 |
| ATOM | 1556 | SG | CYS | 206 | 72.269 | 10.911 | 17.882 | 1.00 | 56.15 |
| ATOM | 1557 | N | LYS | 207 | 68.848 | 9.378 | 19.408 | 1.00 | 65.56 |
| ATOM | 1558 | CA | LYS | 207 | 68.723 | 8.356 | 20.445 | 1.00 | 68.79 |
| ATOM | 1559 | CB | LYS | 207 | 67.347 | 7.683 | 20.343 | 1.00 | 67.04 |
| ATOM | 1560 | CG | LYS | 207 | 67.173 | 6.436 | 21.195 | 0.01 | 67.73 |
| ATOM | 1561 | CD | LYS | 207 | 65.772 | 5.855 | 21.043 | 0.01 | 67.54 |
| ATOM | 1562 | CE | LYS | 207 | 65.436 | 5.561 | 19.586 | 0.01 | 67.59 |
| ATOM | 1563 | NZ | LYS | 207 | 66.392 | 4.601 | 18.974 | 0.01 | 67.49 |
| ATOM | 1564 | C | LYS | 207 | 69.830 | 7.312 | 20.293 | 1.00 | 71.75 |
| ATOM | 1565 | O | LYS | 207 | 70.112 | 6.550 | 21.218 | 1.00 | 72.00 |
| ATOM | 1566 | N | GLY | 208 | 70.460 | 7.287 | 19.123 | 1.00 | 74.88 |
| ATOM | 1567 | CA | GLY | 208 | 71.523 | 6.330 | 18.881 | 1.00 | 78.08 |
| ATOM | 1568 | C | GLY | 208 | 72.444 | 6.705 | 17.733 | 1.00 | 80.40 |
| ATOM | 1569 | O | GLY | 208 | 72.566 | 7.880 | 17.386 | 1.00 | 80.35 |
| ATOM | 1570 | N | PRO | 209 | 73.116 | 5.718 | 17.122 | 1.00 | 82.07 |
| ATOM | 1571 | CD | PRO | 209 | 73.269 | 4.347 | 17.650 | 1.00 | 81.75 |
| ATOM | 1572 | CA | PRO | 209 | 74.037 | 5.952 | 16.004 | 1.00 | 82.82 |
| ATOM | 1573 | CB | PRO | 209 | 75.095 | 4.884 | 16.225 | 1.00 | 83.11 |
| ATOM | 1574 | CG | PRO | 209 | 74.248 | 3.718 | 16.670 | 1.00 | 83.12 |
| ATOM | 1575 | C | PRO | 209 | 73.388 | 5.830 | 14.623 | 1.00 | 83.61 |
| ATOM | 1576 | O | PRO | 209 | 73.866 | 6.418 | 13.647 | 1.00 | 82.88 |
| ATOM | 1577 | N | LEU | 210 | 72.302 | 5.063 | 14.554 | 1.00 | 84.52 |
| ATOM | 1578 | CA | LEU | 210 | 71.586 | 4.825 | 13.301 | 1.00 | 86.20 |
| ATOM | 1579 | CB | LEU | 210 | 70.631 | 3.640 | 13.468 | 1.00 | 86.91 |
| ATOM | 1580 | CG | LEU | 210 | 71.320 | 2.497 | 13.943 | 1.00 | 88.07 |
| ATOM | 1581 | C | LEU | 210 | 70.799 | 6.033 | 12.796 | 1.00 | 86.53 |
| ATOM | 1582 | O | LEU | 210 | 70.273 | 6.822 | 13.584 | 1.00 | 86.64 |
| ATOM | 1583 | N | PRO | 211 | 70.705 | 6.189 | 11.464 | 1.00 | 86.67 |
| ATOM | 1584 | CD | PRO | 211 | 71.348 | 5.377 | 10.417 | 1.00 | 86.41 |
| ATOM | 1585 | CA | PRO | 211 | 69.969 | 7.313 | 10.873 | 1.00 | 86.21 |
| ATOM | 1586 | CB | PRO | 211 | 70.054 | 7.029 | 9.375 | 1.00 | 86.12 |
| ATOM | 1587 | CG | PRO | 211 | 71.381 | 6.339 | 9.249 | 1.00 | 86.71 |
| ATOM | 1588 | C | PRO | 211 | 68.531 | 7.335 | 11.378 | 1.00 | 84.87 |
| ATOM | 1589 | O | PRO | 211 | 67.890 | 8.383 | 11.416 | 1.00 | 84.36 |
| ATOM | 1590 | N | THR | 212 | 68.038 | 6.166 | 11.770 | 1.00 | 83.57 |
| ATOM | 1591 | CA | THR | 212 | 66.680 | 6.039 | 12.276 | 1.00 | 82.59 |
| ATOM | 1592 | CB | THR | 212 | 66.202 | 4.575 | 12.229 | 1.00 | 82.81 |
| ATOM | 1593 | OG1 | THR | 212 | 64.904 | 4.479 | 12.833 | 1.00 | 83.74 |
| ATOM | 1594 | CG2 | THR | 212 | 67.180 | 3.671 | 12.974 | 1.00 | 81.77 |
| ATOM | 1595 | C | THR | 212 | 66.584 | 6.526 | 13.713 | 1.00 | 81.46 |
| ATOM | 1596 | O | THR | 212 | 65.499 | 6.847 | 14.193 | 1.00 | 81.45 |
| ATOM | 1597 | N | ASP | 213 | 67.720 | 6.568 | 14.401 | 1.00 | 80.37 |
| ATOM | 1598 | CA | ASP | 213 | 67.740 | 7.020 | 15.788 | 1.00 | 79.08 |
| ATOM | 1599 | CB | ASP | 213 | 69.002 | 6.524 | 16.505 | 1.00 | 79.78 |
| ATOM | 1600 | CG | ASP | 213 | 69.053 | 5.010 | 16.621 | 1.00 | 80.99 |
| ATOM | 1601 | OD1 | ASP | 213 | 68.014 | 4.400 | 16.968 | 1.00 | 81.86 |
| ATOM | 1602 | OD2 | ASP | 213 | 70.134 | 4.433 | 16.374 | 1.00 | 80.16 |
| ATOM | 1603 | C | ASP | 213 | 67.665 | 8.542 | 15.874 | 1.00 | 77.22 |
| ATOM | 1604 | O | ASP | 213 | 67.365 | 9.092 | 16.935 | 1.00 | 77.31 |
| ATOM | 1605 | N | CYS | 214 | 67.940 | 9.216 | 14.759 | 1.00 | 74.07 |
| ATOM | 1606 | CA | CYS | 214 | 67.883 | 10.672 | 14.719 | 1.00 | 70.05 |
| ATOM | 1607 | C | CYS | 214 | 66.541 | 11.159 | 15.230 | 1.00 | 66.41 |
| ATOM | 1608 | O | CYS | 214 | 65.513 | 10.513 | 15.021 | 1.00 | 65.64 |
| ATOM | 1609 | CB | CYS | 214 | 68.084 | 11.193 | 13.299 | 1.00 | 70.85 |
| ATOM | 1610 | SG | CYS | 214 | 69.811 | 11.527 | 12.823 | 1.00 | 76.21 |
| ATOM | 1611 | N | CYS | 215 | 66.566 | 12.305 | 15.902 | 1.00 | 62.55 |
| ATOM | 1612 | CA | CYS | 215 | 65.363 | 12.913 | 16.455 | 1.00 | 59.05 |
| ATOM | 1613 | C | CYS | 215 | 64.740 | 13.900 | 15.460 | 1.00 | 56.72 |
| ATOM | 1614 | O | CYS | 215 | 65.420 | 14.435 | 14.581 | 1.00 | 55.51 |
| ATOM | 1615 | CB | CYS | 215 | 65.697 | 13.668 | 17.751 | 1.00 | 57.47 |
| ATOM | 1616 | SG | CYS | 215 | 66.369 | 12.690 | 19.136 | 1.00 | 53.64 |
| ATOM | 1617 | N | HIS | 216 | 63.444 | 14.141 | 15.607 | 1.00 | 54.42 |
| ATOM | 1618 | CA | HIS | 216 | 62.757 | 15.096 | 14.748 | 1.00 | 54.48 |
| ATOM | 1619 | CB | HIS | 216 | 61.269 | 15.142 | 15.115 | 1.00 | 53.10 |
| ATOM | 1620 | CG | HIS | 216 | 60.438 | 15.983 | 14.195 | 1.00 | 55.87 |
| ATOM | 1621 | CD2 | HIS | 216 | 59.589 | 15.638 | 13.196 | 1.00 | 55.67 |
| ATOM | 1622 | ND1 | HIS | 216 | 60.406 | 17.360 | 14.266 | 1.00 | 56.51 |
| ATOM | 1623 | CE1 | HIS | 216 | 59.570 | 17.827 | 13.354 | 1.00 | 56.11 |
| ATOM | 1624 | NE2 | HIS | 216 | 59.061 | 16.803 | 12.692 | 1.00 | 56.64 |
| ATOM | 1625 | C | HIS | 216 | 63.410 | 16.478 | 14.934 | 1.00 | 54.60 |
| ATOM | 1626 | O | HIS | 216 | 63.856 | 16.837 | 16.034 | 1.00 | 53.79 |
| ATOM | 1627 | N | GLU | 217 | 63.470 | 17.236 | 13.847 | 1.00 | 53.81 |
| ATOM | 1628 | CA | GLU | 217 | 64.067 | 18.566 | 13.833 | 1.00 | 54.35 |
| ATOM | 1629 | CB | GLU | 217 | 63.849 | 19.193 | 12.444 | 1.00 | 58.36 |
| ATOM | 1630 | CG | GLU | 217 | 64.190 | 20.676 | 12.323 | 1.00 | 63.65 |
| ATOM | 1631 | CD | GLU | 217 | 64.738 | 21.027 | 10.948 | 1.00 | 67.93 |
| ATOM | 1632 | OE1 | GLU | 217 | 64.097 | 20.634 | 9.945 | 1.00 | 71.26 |
| ATOM | 1633 | OE2 | GLU | 217 | 65.805 | 21.692 | 10.868 | 1.00 | 67.02 |
| ATOM | 1634 | C | GLU | 217 | 63.564 | 19.513 | 14.928 | 1.00 | 52.90 |
| ATOM | 1635 | O | GLU | 217 | 64.338 | 20.313 | 15.464 | 1.00 | 52.25 |
| ATOM | 1636 | N | GLN | 218 | 62.275 | 19.421 | 15.251 | 1.00 | 51.31 |
| ATOM | 1637 | CA | GLN | 218 | 61.661 | 20.271 | 16.269 | 1.00 | 51.12 |
| ATOM | 1638 | CB | GLN | 218 | 60.139 | 20.318 | 16.080 | 1.00 | 50.40 |
| ATOM | 1639 | CG | GLN | 218 | 59.688 | 21.323 | 15.034 | 1.00 | 50.78 |
| ATOM | 1640 | CD | GLN | 218 | 60.338 | 22.687 | 15.232 | 1.00 | 52.99 |
| ATOM | 1641 | OE1 | GLN | 218 | 60.327 | 23.238 | 16.335 | 1.00 | 53.76 |
| ATOM | 1642 | NE2 | GLN | 218 | 60.905 | 23.238 | 14.164 | 1.00 | 52.00 |
| ATOM | 1643 | C | GLN | 218 | 61.983 | 19.866 | 17.705 | 1.00 | 51.26 |
| ATOM | 1644 | O | GLN | 218 | 61.619 | 20.557 | 18.654 | 1.00 | 51.69 |
| ATOM | 1645 | N | CYS | 219 | 62.665 | 18.742 | 17.863 | 1.00 | 51.32 |
| ATOM | 1646 | CA | CYS | 219 | 63.039 | 18.271 | 19.185 | 1.00 | 50.59 |
| ATOM | 1647 | C | CYS | 219 | 64.281 | 18.988 | 19.678 | 1.00 | 49.99 |
| ATOM | 1648 | O | CYS | 219 | 65.053 | 19.540 | 18.893 | 1.00 | 48.92 |
| ATOM | 1649 | CB | CYS | 219 | 63.369 | 16.787 | 19.161 | 1.00 | 51.60 |
| ATOM | 1650 | SG | CYS | 219 | 62.010 | 15.615 | 18.926 | 1.00 | 53.95 |
| ATOM | 1651 | N | ALA | 220 | 64.471 | 18.944 | 20.991 | 1.00 | 49.92 |
| ATOM | 1652 | CA | ALA | 220 | 65.628 | 19.530 | 21.644 | 1.00 | 48.74 |
| ATOM | 1653 | CB | ALA | 220 | 65.293 | 20.916 | 22.174 | 1.00 | 50.16 |
| ATOM | 1654 | C | ALA | 220 | 65.995 | 18.590 | 22.796 | 1.00 | 48.82 |
| ATOM | 1655 | O | ALA | 220 | 65.121 | 17.925 | 23.363 | 1.00 | 45.90 |
| ATOM | 1656 | N | ALA | 221 | 67.286 | 18.523 | 23.112 | 1.00 | 48.62 |
| ATOM | 1657 | CA | ALA | 221 | 67.795 | 17.684 | 24.195 | 1.00 | 49.39 |
| ATOM | 1658 | CB | ALA | 221 | 67.019 | 17.963 | 25.486 | 1.00 | 48.33 |
| ATOM | 1659 | C | ALA | 221 | 67.785 | 16.183 | 23.879 | 1.00 | 50.36 |
| ATOM | 1660 | O | ALA | 221 | 68.672 | 15.444 | 24.314 | 1.00 | 51.30 |
| ATOM | 1661 | N | GLY | 222 | 66.796 | 15.720 | 23.129 | 1.00 | 49.99 |
| ATOM | 1662 | CA | GLY | 222 | 66.770 | 14.308 | 22.800 | 1.00 | 51.23 |
| ATOM | 1663 | C | GLY | 222 | 65.397 | 13.768 | 22.472 | 1.00 | 51.50 |
| ATOM | 1664 | O | GLY | 222 | 64.432 | 14.528 | 22.375 | 1.00 | 50.44 |
| ATOM | 1665 | N | CYS | 223 | 65.306 | 12.448 | 22.313 | 1.00 | 52.91 |
| ATOM | 1666 | CA | CYS | 223 | 64.037 | 11.804 | 21.979 | 1.00 | 54.68 |
| ATOM | 1667 | C | CYS | 223 | 64.028 | 10.307 | 22.259 | 1.00 | 55.32 |
| ATOM | 1668 | O | CYS | 223 | 65.075 | 9.692 | 22.470 | 1.00 | 55.55 |
| ATOM | 1669 | CB | CYS | 223 | 63.729 | 12.016 | 20.503 | 1.00 | 53.19 |
| ATOM | 1670 | SG | CYS | 223 | 64.960 | 11.250 | 19.401 | 1.00 | 54.63 |
| ATOM | 1671 | N | THR | 224 | 62.831 | 9.729 | 22.256 | 1.00 | 56.47 |
| ATOM | 1672 | CA | THR | 224 | 62.664 | 8.296 | 22.473 | 1.00 | 58.28 |
| ATOM | 1673 | CB | THR | 224 | 61.517 | 8.008 | 23.440 | 1.00 | 54.80 |
| ATOM | 1674 | OG1 | THR | 224 | 60.305 | 8.560 | 22.917 | 1.00 | 50.55 |
| ATOM | 1675 | CG2 | THR | 224 | 61.812 | 8.620 | 24.796 | 1.00 | 53.35 |
| ATOM | 1676 | C | THR | 224 | 62.335 | 7.677 | 21.117 | 1.00 | 61.82 |
| ATOM | 1677 | O | THR | 224 | 62.844 | 6.613 | 20.758 | 1.00 | 63.89 |
| ATOM | 1678 | N | GLY | 225 | 61.480 | 8.364 | 20.367 | 1.00 | 64.77 |
| ATOM | 1679 | CA | GLY | 225 | 61.100 | 7.899 | 19.044 | 1.00 | 66.41 |
| ATOM | 1680 | C | GLY | 225 | 61.548 | 8.883 | 17.973 | 1.00 | 66.85 |
| ATOM | 1681 | O | GLY | 225 | 62.223 | 9.873 | 18.275 | 1.00 | 66.37 |
| ATOM | 1682 | N | PRO | 226 | 61.195 | 8.633 | 16.705 | 1.00 | 66.86 |
| ATOM | 1683 | CD | PRO | 226 | 60.659 | 7.353 | 16.199 | 1.00 | 66.51 |
| ATOM | 1684 | CA | PRO | 226 | 61.575 | 9.521 | 15.599 | 1.00 | 66.01 |
| ATOM | 1685 | CB | PRO | 226 | 61.713 | 8.556 | 14.431 | 1.00 | 67.21 |
| ATOM | 1686 | CG | PRO | 226 | 60.571 | 7.596 | 14.694 | 1.00 | 67.17 |
| ATOM | 1687 | C | PRO | 226 | 60.537 | 10.615 | 15.319 | 1.00 | 64.69 |
| ATOM | 1688 | O | PRO | 226 | 60.805 | 11.560 | 14.577 | 1.00 | 63.09 |
| ATOM | 1689 | N | LYS | 227 | 59.356 | 10.478 | 15.917 | 1.00 | 63.65 |
| ATOM | 1690 | CA | LYS | 227 | 58.276 | 11.438 | 15.716 | 1.00 | 63.98 |
| ATOM | 1691 | CB | LYS | 227 | 56.930 | 10.785 | 16.082 | 1.00 | 64.77 |
| ATOM | 1692 | CG | LYS | 227 | 56.580 | 9.579 | 15.200 | 1.00 | 66.25 |
| ATOM | 1693 | CD | LYS | 227 | 55.152 | 9.061 | 15.416 | 1.00 | 69.05 |
| ATOM | 1694 | CE | LYS | 227 | 54.965 | 8.378 | 16.775 | 1.00 | 70.66 |
| ATOM | 1695 | NZ | LYS | 227 | 55.844 | 7.181 | 16.951 | 1.00 | 70.84 |
| ATOM | 1696 | C | LYS | 227 | 58.467 | 12.748 | 16.487 | 1.00 | 63.25 |
| ATOM | 1697 | O | LYS | 227 | 59.191 | 12.792 | 17.485 | 1.00 | 62.89 |
| ATOM | 1698 | N | HIS | 228 | 57.823 | 13.815 | 16.014 | 1.00 | 61.92 |
| ATOM | 1699 | CA | HIS | 228 | 57.927 | 15.117 | 16.665 | 1.00 | 61.52 |
| ATOM | 1700 | CB | HIS | 228 | 57.443 | 16.236 | 15.721 | 1.00 | 61.00 |
| ATOM | 1701 | CG | HIS | 228 | 55.996 | 16.149 | 15.342 | 1.00 | 62.26 |
| ATOM | 1702 | CD2 | HIS | 228 | 55.015 | 15.296 | 15.728 | 1.00 | 62.88 |
| ATOM | 1703 | ND1 | HIS | 228 | 55.404 | 17.043 | 14.477 | 1.00 | 62.93 |
| ATOM | 1704 | CE1 | HIS | 228 | 54.123 | 16.748 | 14.347 | 1.00 | 62.35 |
| ATOM | 1705 | NE2 | HIS | 228 | 53.861 | 15.692 | 15.097 | 1.00 | 62.26 |
| ATOM | 1706 | C | HIS | 228 | 57.133 | 15.114 | 17.972 | 1.00 | 61.48 |
| ATOM | 1707 | O | HIS | 228 | 56.898 | 16.156 | 18.589 | 1.00 | 60.05 |
| ATOM | 1708 | N | SER | 229 | 56.733 | 13.915 | 18.384 | 1.00 | 61.60 |
| ATOM | 1709 | CA | SER | 229 | 55.969 | 13.713 | 19.609 | 1.00 | 61.01 |
| ATOM | 1710 | CB | SER | 229 | 54.644 | 13.010 | 19.286 | 1.00 | 61.25 |
| ATOM | 1711 | OG | SER | 229 | 54.855 | 11.816 | 18.539 | 1.00 | 61.44 |
| ATOM | 1712 | C | SER | 229 | 56.783 | 12.871 | 20.591 | 1.00 | 59.99 |
| ATOM | 1713 | O | SER | 229 | 56.332 | 12.581 | 21.698 | 1.00 | 59.55 |
| ATOM | 1714 | N | ASP | 230 | 57.984 | 12.482 | 20.174 | 1.00 | 59.28 |
| ATOM | 1715 | CA | ASP | 230 | 58.866 | 11.674 | 21.011 | 1.00 | 59.27 |
| ATOM | 1716 | CB | ASP | 230 | 59.423 | 10.492 | 20.212 | 1.00 | 60.87 |
| ATOM | 1717 | CG | ASP | 230 | 58.346 | 9.719 | 19.479 | 1.00 | 62.55 |
| ATOM | 1718 | OD1 | ASP | 230 | 57.331 | 9.352 | 20.118 | 1.00 | 62.34 |
| ATOM | 1719 | OD2 | ASP | 230 | 58.525 | 9.473 | 18.263 | 1.00 | 62.79 |
| ATOM | 1720 | C | ASP | 230 | 60.031 | 12.511 | 21.535 | 1.00 | 58.31 |
| ATOM | 1721 | O | ASP | 230 | 61.065 | 11.980 | 21.945 | 1.00 | 59.10 |
| ATOM | 1722 | N | CYS | 231 | 59.863 | 13.826 | 21.501 | 1.00 | 56.84 |
| ATOM | 1723 | CA | CYS | 231 | 60.889 | 14.741 | 21.969 | 1.00 | 53.72 |
| ATOM | 1724 | C | CYS | 231 | 60.990 | 14.742 | 23.497 | 1.00 | 52.21 |
| ATOM | 1725 | O | CYS | 231 | 59.996 | 14.525 | 24.200 | 1.00 | 49.54 |
| ATOM | 1726 | CB | CYS | 231 | 60.562 | 16.166 | 21.524 | 1.00 | 54.60 |
| ATOM | 1727 | SG | CYS | 231 | 60.392 | 16.502 | 19.744 | 1.00 | 53.23 |
| ATOM | 1728 | N | LEU | 232 | 62.191 | 14.992 | 24.009 | 1.00 | 50.70 |
| ATOM | 1729 | CA | LEU | 232 | 62.382 | 15.078 | 25.452 | 1.00 | 50.35 |
| ATOM | 1730 | CB | LEU | 232 | 63.822 | 14.746 | 25.836 | 1.00 | 49.26 |
| ATOM | 1731 | CG | LEU | 232 | 64.221 | 13.274 | 25.726 | 1.00 | 51.66 |
| ATOM | 1732 | CD1 | LEU | 232 | 65.665 | 13.105 | 26.204 | 1.00 | 50.91 |
| ATOM | 1733 | CD2 | LEU | 232 | 63.279 | 12.416 | 26.561 | 1.00 | 49.83 |
| ATOM | 1734 | C | LEU | 232 | 62.064 | 16.514 | 25.856 | 1.00 | 49.63 |
| ATOM | 1735 | O | LEU | 232 | 61.730 | 16.798 | 27.003 | 1.00 | 49.78 |
| ATOM | 1736 | N | ALA | 233 | 62.178 | 17.414 | 24.887 | 1.00 | 50.22 |
| ATOM | 1737 | CA | ALA | 233 | 61.910 | 18.834 | 25.084 | 1.00 | 49.90 |
| ATOM | 1738 | CB | ALA | 233 | 63.099 | 19.507 | 25.759 | 1.00 | 49.52 |
| ATOM | 1739 | C | ALA | 233 | 61.692 | 19.441 | 23.711 | 1.00 | 49.74 |
| ATOM | 1740 | O | ALA | 233 | 62.251 | 18.962 | 22.718 | 1.00 | 50.22 |
| ATOM | 1741 | N | CYS | 234 | 60.883 | 20.493 | 23.654 | 1.00 | 48.80 |
| ATOM | 1742 | CA | CYS | 234 | 60.605 | 21.168 | 22.391 | 1.00 | 46.07 |
| ATOM | 1743 | C | CYS | 234 | 61.573 | 22.320 | 22.159 | 1.00 | 45.21 |
| ATOM | 1744 | O | CYS | 234 | 61.745 | 23.189 | 23.019 | 1.00 | 44.39 |
| ATOM | 1745 | CB | CYS | 234 | 59.182 | 21.704 | 22.383 | 1.00 | 44.96 |
| ATOM | 1746 | SG | CYS | 234 | 57.878 | 20.445 | 22.262 | 1.00 | 49.23 |
| ATOM | 1747 | N | LEU | 235 | 62.206 | 22.321 | 20.991 | 1.00 | 44.46 |
| ATOM | 1748 | CA | LEU | 235 | 63.154 | 23.373 | 20.635 | 1.00 | 42.64 |
| ATOM | 1749 | CB | LEU | 235 | 63.753 | 23.087 | 19.259 | 1.00 | 38.15 |
| ATOM | 1750 | CG | LEU | 235 | 64.722 | 24.119 | 18.670 | 1.00 | 40.61 |
| ATOM | 1751 | CD1 | LEU | 235 | 65.957 | 24.301 | 19.542 | 1.00 | 38.57 |
| ATOM | 1752 | CD2 | LEU | 235 | 65.127 | 23.644 | 17.284 | 1.00 | 39.44 |
| ATOM | 1753 | C | LEU | 235 | 62.486 | 24.753 | 20.647 | 1.00 | 42.98 |
| ATOM | 1754 | O | LEU | 235 | 63.126 | 25.748 | 20.976 | 1.00 | 44.50 |
| ATOM | 1755 | N | HIS | 236 | 61.200 | 24.805 | 20.300 | 1.00 | 43.28 |
| ATOM | 1756 | CA | HIS | 236 | 60.448 | 26.064 | 20.273 | 1.00 | 43.13 |
| ATOM | 1757 | CB | HIS | 236 | 60.140 | 26.472 | 18.828 | 1.00 | 41.66 |
| ATOM | 1758 | CG | HIS | 236 | 61.362 | 26.675 | 17.981 | 1.00 | 41.09 |
| ATOM | 1759 | CD2 | HIS | 236 | 61.972 | 25.857 | 17.093 | 1.00 | 37.73 |
| ATOM | 1760 | ND1 | HIS | 236 | 62.124 | 27.825 | 18.032 | 1.00 | 39.96 |
| ATOM | 1761 | CE1 | HIS | 236 | 63.151 | 27.704 | 17.212 | 1.00 | 38.38 |
| ATOM | 1762 | NE2 | HIS | 236 | 63.082 | 26.519 | 16.631 | 1.00 | 38.79 |
| ATOM | 1763 | C | HIS | 236 | 59.136 | 25.958 | 21.048 | 1.00 | 43.69 |
| ATOM | 1764 | O | HIS | 236 | 59.016 | 26.492 | 22.140 | 1.00 | 43.60 |
| ATOM | 1765 | N | PHE | 237 | 58.148 | 25.259 | 20.501 | 1.00 | 45.04 |
| ATOM | 1766 | CA | PHE | 237 | 56.881 | 25.168 | 21.208 | 1.00 | 47.08 |
| ATOM | 1767 | CB | PHE | 237 | 55.866 | 26.126 | 20.576 | 1.00 | 46.02 |
| ATOM | 1768 | CG | PHE | 237 | 56.309 | 27.549 | 20.593 | 1.00 | 44.88 |
| ATOM | 1769 | CD1 | PHE | 237 | 56.765 | 28.160 | 19.435 | 1.00 | 44.85 |
| ATOM | 1770 | CD2 | PHE | 237 | 56.337 | 28.262 | 21.786 | 1.00 | 43.81 |
| ATOM | 1771 | CE1 | PHE | 237 | 57.248 | 29.467 | 19.465 | 1.00 | 45.44 |
| ATOM | 1772 | CE2 | PHE | 237 | 56.820 | 29.569 | 21.830 | 1.00 | 43.85 |
| ATOM | 1773 | CZ | PHE | 237 | 57.276 | 30.174 | 20.670 | 1.00 | 44.65 |
| ATOM | 1774 | C | PHE | 237 | 56.244 | 23.804 | 21.335 | 1.00 | 48.24 |
| ATOM | 1775 | O | PHE | 237 | 56.268 | 23.002 | 20.405 | 1.00 | 50.43 |
| ATOM | 1776 | N | ASN | 238 | 55.681 | 23.543 | 22.508 | 1.00 | 49.47 |
| ATOM | 1777 | CA | ASN | 238 | 54.973 | 22.296 | 22.741 | 1.00 | 51.45 |
| ATOM | 1778 | CB | ASN | 238 | 55.082 | 21.856 | 24.202 | 1.00 | 54.33 |
| ATOM | 1779 | CG | ASN | 238 | 54.388 | 20.529 | 24.458 | 1.00 | 59.95 |
| ATOM | 1780 | OD1 | ASN | 238 | 53.556 | 20.093 | 23.652 | 1.00 | 61.47 |
| ATOM | 1781 | ND2 | ASN | 238 | 54.713 | 19.885 | 25.579 | 1.00 | 63.18 |
| ATOM | 1782 | C | ASN | 238 | 53.526 | 22.658 | 22.418 | 1.00 | 50.77 |
| ATOM | 1783 | O | ASN | 238 | 52.926 | 23.500 | 23.089 | 1.00 | 49.69 |
| ATOM | 1784 | N | HIS | 239 | 52.983 | 22.049 | 21.372 | 1.00 | 50.70 |
| ATOM | 1785 | CA | HIS | 239 | 51.616 | 22.319 | 20.953 | 1.00 | 51.93 |
| ATOM | 1786 | CB | HIS | 239 | 51.581 | 22.562 | 19.443 | 1.00 | 51.88 |
| ATOM | 1787 | CG | HIS | 239 | 50.227 | 22.916 | 18.912 | 1.00 | 52.05 |
| ATOM | 1788 | CD2 | HIS | 239 | 49.115 | 23.373 | 19.533 | 1.00 | 50.98 |
| ATOM | 1789 | ND1 | HIS | 239 | 49.913 | 22.838 | 17.571 | 1.00 | 53.11 |
| ATOM | 1790 | CE1 | HIS | 239 | 48.664 | 23.234 | 17.392 | 1.00 | 52.58 |
| ATOM | 1791 | NE2 | HIS | 239 | 48.159 | 23.564 | 18.566 | 1.00 | 50.88 |
| ATOM | 1792 | C | HIS | 239 | 50.728 | 21.134 | 21.313 | 1.00 | 54.08 |
| ATOM | 1793 | O | HIS | 239 | 50.366 | 20.324 | 20.455 | 1.00 | 55.11 |
| ATOM | 1794 | N | SER | 240 | 50.390 | 21.025 | 22.590 | 1.00 | 55.26 |
| ATOM | 1795 | CA | SER | 240 | 49.547 | 19.932 | 23.046 | 1.00 | 57.11 |
| ATOM | 1796 | CB | SER | 240 | 48.118 | 20.101 | 22.510 | 1.00 | 57.75 |
| ATOM | 1797 | OG | SER | 240 | 47.417 | 21.127 | 23.197 | 1.00 | 60.07 |
| ATOM | 1798 | C | SER | 240 | 50.086 | 18.567 | 22.619 | 1.00 | 57.00 |
| ATOM | 1799 | O | SER | 240 | 49.352 | 17.756 | 22.058 | 1.00 | 57.19 |
| ATOM | 1800 | N | GLY | 241 | 51.366 | 18.314 | 22.870 | 1.00 | 56.21 |
| ATOM | 1801 | CA | GLY | 241 | 51.921 | 17.022 | 22.514 | 1.00 | 55.65 |
| ATOM | 1802 | C | GLY | 241 | 52.931 | 17.001 | 21.384 | 1.00 | 56.21 |
| ATOM | 1803 | O | GLY | 241 | 53.783 | 16.108 | 21.332 | 1.00 | 56.34 |
| ATOM | 1804 | N | ILE | 242 | 52.850 | 17.959 | 20.467 | 1.00 | 55.70 |
| ATOM | 1805 | CA | ILE | 242 | 53.804 | 17.982 | 19.368 | 1.00 | 56.07 |
| ATOM | 1806 | CB | ILE | 242 | 53.100 | 17.806 | 17.992 | 1.00 | 57.14 |
| ATOM | 1807 | CG2 | ILE | 242 | 52.197 | 16.579 | 18.027 | 1.00 | 58.47 |
| ATOM | 1808 | CG1 | ILE | 242 | 52.266 | 19.035 | 17.653 | 1.00 | 57.33 |
| ATOM | 1809 | CD1 | ILE | 242 | 51.636 | 18.967 | 16.284 | 1.00 | 59.21 |
| ATOM | 1810 | C | ILE | 242 | 54.632 | 19.264 | 19.355 | 1.00 | 54.41 |
| ATOM | 1811 | O | ILE | 242 | 54.119 | 20.352 | 19.594 | 1.00 | 54.64 |
| ATOM | 1812 | N | CYS | 243 | 55.925 | 19.118 | 19.098 | 1.00 | 52.85 |
| ATOM | 1813 | CA | CYS | 243 | 56.824 | 20.258 | 19.045 | 1.00 | 51.87 |
| ATOM | 1814 | C | CYS | 243 | 56.746 | 20.886 | 17.658 | 1.00 | 52.41 |
| ATOM | 1815 | O | CYS | 243 | 56.821 | 20.184 | 16.649 | 1.00 | 52.32 |
| ATOM | 1816 | CB | CYS | 243 | 58.268 | 19.818 | 19.310 | 1.00 | 51.08 |
| ATOM | 1817 | SG | CYS | 243 | 58.637 | 19.090 | 20.946 | 1.00 | 46.96 |
| ATOM | 1818 | N | GLU | 244 | 56.598 | 22.207 | 17.612 | 1.00 | 52.51 |
| ATOM | 1819 | CA | GLU | 244 | 56.521 | 22.935 | 16.349 | 1.00 | 52.16 |
| ATOM | 1820 | CB | GLU | 244 | 55.057 | 23.264 | 16.028 | 1.00 | 55.07 |
| ATOM | 1821 | CG | GLU | 244 | 54.287 | 22.078 | 15.419 | 1.00 | 57.94 |
| ATOM | 1822 | CD | GLU | 244 | 52.773 | 22.235 | 15.473 | 1.00 | 59.16 |
| ATOM | 1823 | OE1 | GLU | 244 | 52.070 | 21.444 | 14.813 | 1.00 | 61.89 |
| ATOM | 1824 | OE2 | GLU | 244 | 52.280 | 23.133 | 16.180 | 1.00 | 60.11 |
| ATOM | 1825 | C | GLU | 244 | 57.362 | 24.210 | 16.398 | 1.00 | 50.67 |
| ATOM | 1826 | O | GLU | 244 | 57.778 | 24.650 | 17.477 | 1.00 | 50.55 |
| ATOM | 1827 | N | LEU | 245 | 57.626 | 24.792 | 15.229 | 1.00 | 47.79 |
| ATOM | 1828 | CA | LEU | 245 | 58.418 | 26.019 | 15.143 | 1.00 | 45.05 |
| ATOM | 1829 | CB | LEU | 245 | 58.850 | 26.268 | 13.697 | 1.00 | 44.16 |
| ATOM | 1830 | CG | LEU | 245 | 59.673 | 27.522 | 13.385 | 1.00 | 46.91 |
| ATOM | 1831 | CD1 | LEU | 245 | 61.006 | 27.525 | 14.158 | 1.00 | 46.09 |
| ATOM | 1832 | CD2 | LEU | 245 | 59.921 | 27.569 | 11.887 | 1.00 | 44.84 |
| ATOM | 1833 | C | LEU | 245 | 57.641 | 27.226 | 15.684 | 1.00 | 43.01 |
| ATOM | 1834 | O | LEU | 245 | 58.227 | 28.138 | 16.254 | 1.00 | 43.29 |
| ATOM | 1835 | N | HIS | 246 | 56.328 | 27.245 | 15.498 | 1.00 | 42.54 |
| ATOM | 1836 | CA | HIS | 246 | 55.516 | 28.336 | 16.033 | 1.00 | 43.79 |
| ATOM | 1837 | CB | HIS | 246 | 55.446 | 29.533 | 15.066 | 1.00 | 43.90 |
| ATOM | 1838 | CG | HIS | 246 | 54.822 | 29.218 | 13.744 | 1.00 | 46.48 |
| ATOM | 1839 | CD2 | HIS | 246 | 55.345 | 29.205 | 12.493 | 1.00 | 47.31 |
| ATOM | 1840 | ND1 | HIS | 246 | 53.504 | 28.832 | 13.613 | 1.00 | 46.81 |
| ATOM | 1841 | CE1 | HIS | 246 | 53.244 | 28.591 | 12.339 | 1.00 | 48.43 |
| ATOM | 1842 | NE2 | HIS | 246 | 54.343 | 28.809 | 11.639 | 1.00 | 47.81 |
| ATOM | 1843 | C | HIS | 246 | 54.122 | 27.822 | 16.345 | 1.00 | 43.37 |
| ATOM | 1844 | O | HIS | 246 | 53.756 | 26.727 | 15.936 | 1.00 | 42.87 |
| ATOM | 1845 | N | CYS | 247 | 53.352 | 28.607 | 17.086 | 1.00 | 44.91 |
| ATOM | 1846 | CA | CYS | 247 | 52.001 | 28.198 | 17.441 | 1.00 | 46.69 |
| ATOM | 1847 | C | CYS | 247 | 51.029 | 28.620 | 16.363 | 1.00 | 46.85 |
| ATOM | 1848 | O | CYS | 247 | 51.390 | 29.345 | 15.440 | 1.00 | 47.26 |
| ATOM | 1849 | CB | CYS | 247 | 51.574 | 28.828 | 18.766 | 1.00 | 45.32 |
| ATOM | 1850 | SG | CYS | 247 | 52.606 | 28.367 | 20.196 | 1.00 | 48.87 |
| ATOM | 1851 | N | PRO | 248 | 49.777 | 28.155 | 16.458 | 1.00 | 47.67 |
| ATOM | 1852 | CD | PRO | 248 | 49.277 | 27.029 | 17.261 | 1.00 | 48.14 |
| ATOM | 1853 | CA | PRO | 248 | 48.792 | 28.539 | 15.447 | 1.00 | 48.14 |
| ATOM | 1854 | CB | PRO | 248 | 47.665 | 27.525 | 15.650 | 1.00 | 47.79 |
| ATOM | 1855 | CG | PRO | 248 | 48.355 | 26.353 | 16.284 | 1.00 | 48.90 |
| ATOM | 1856 | C | PRO | 248 | 48.339 | 29.954 | 15.770 | 1.00 | 48.84 |
| ATOM | 1857 | O | PRO | 248 | 47.903 | 30.219 | 16.888 | 1.00 | 49.62 |
| ATOM | 1858 | N | ALA | 249 | 48.448 | 30.857 | 14.806 | 1.00 | 49.08 |
| ATOM | 1859 | CA | ALA | 249 | 48.029 | 32.239 | 15.012 | 1.00 | 51.41 |
| ATOM | 1860 | CB | ALA | 249 | 48.198 | 33.025 | 13.711 | 1.00 | 51.96 |
| ATOM | 1861 | C | ALA | 249 | 46.576 | 32.337 | 15.507 | 1.00 | 51.87 |
| ATOM | 1862 | O | ALA | 249 | 45.776 | 31.417 | 15.326 | 1.00 | 51.47 |
| ATOM | 1863 | N | LEU | 250 | 46.245 | 33.458 | 16.139 | 1.00 | 53.36 |
| ATOM | 1864 | CA | LEU | 250 | 44.898 | 33.677 | 16.656 | 1.00 | 55.64 |
| ATOM | 1865 | CB | LEU | 250 | 44.905 | 34.778 | 17.720 | 1.00 | 54.87 |
| ATOM | 1866 | CG | LEU | 250 | 45.630 | 34.462 | 19.021 | 1.00 | 54.91 |
| ATOM | 1867 | CD1 | LEU | 250 | 45.485 | 35.628 | 19.978 | 1.00 | 56.42 |
| ATOM | 1868 | CD2 | LEU | 250 | 45.049 | 33.202 | 19.631 | 1.00 | 56.57 |
| ATOM | 1869 | C | LEU | 250 | 43.902 | 34.060 | 15.566 | 1.00 | 56.65 |
| ATOM | 1870 | O | LEU | 250 | 42.691 | 33.966 | 15.760 | 1.00 | 55.99 |
| ATOM | 1871 | N | VAL | 251 | 44.405 | 34.499 | 14.422 | 1.00 | 58.57 |
| ATOM | 1872 | CA | VAL | 251 | 43.512 | 34.896 | 13.347 | 1.00 | 61.37 |
| ATOM | 1873 | CB | VAL | 251 | 43.509 | 36.425 | 13.164 | 1.00 | 61.36 |
| ATOM | 1874 | CG1 | VAL | 251 | 43.124 | 37.110 | 14.470 | 1.00 | 59.22 |
| ATOM | 1875 | CG2 | VAL | 251 | 44.883 | 36.889 | 12.694 | 1.00 | 61.93 |
| ATOM | 1876 | C | VAL | 251 | 43.886 | 34.276 | 12.018 | 1.00 | 63.15 |
| ATOM | 1877 | O | VAL | 251 | 45.061 | 34.033 | 11.746 | 1.00 | 64.19 |
| ATOM | 1878 | N | THR | 252 | 42.873 | 34.013 | 11.199 | 1.00 | 65.01 |
| ATOM | 1879 | CA | THR | 252 | 43.076 | 33.461 | 9.865 | 1.00 | 66.08 |
| ATOM | 1880 | CB | THR | 252 | 42.151 | 32.259 | 9.584 | 1.00 | 67.82 |
| ATOM | 1881 | OG1 | THR | 252 | 42.265 | 31.301 | 10.640 | 1.00 | 70.86 |
| ATOM | 1882 | CG2 | THR | 252 | 42.532 | 31.590 | 8.272 | 1.00 | 68.46 |
| ATOM | 1883 | C | THR | 252 | 42.660 | 34.601 | 8.946 | 1.00 | 65.57 |
| ATOM | 1884 | O | THR | 252 | 41.695 | 35.311 | 9.248 | 1.00 | 66.04 |
| ATOM | 1885 | N | TYR | 253 | 43.379 | 34.796 | 7.845 | 1.00 | 63.88 |
| ATOM | 1886 | CA | TYR | 253 | 43.024 | 35.864 | 6.922 | 1.00 | 62.24 |
| ATOM | 1887 | CB | TYR | 253 | 44.272 | 36.620 | 6.463 | 1.00 | 61.85 |
| ATOM | 1888 | CG | TYR | 253 | 44.977 | 37.329 | 7.592 | 1.00 | 62.35 |
| ATOM | 1889 | CD1 | TYR | 253 | 46.223 | 36.891 | 8.046 | 1.00 | 61.84 |
| ATOM | 1890 | CE1 | TYR | 253 | 46.859 | 37.517 | 9.113 | 1.00 | 61.95 |
| ATOM | 1891 | CD2 | TYR | 253 | 44.382 | 38.416 | 8.237 | 1.00 | 61.08 |
| ATOM | 1892 | CE2 | TYR | 253 | 45.007 | 39.048 | 9.307 | 1.00 | 61.11 |
| ATOM | 1893 | CZ | TYR | 253 | 46.245 | 38.594 | 9.740 | 1.00 | 62.39 |
| ATOM | 1894 | OH | TYR | 253 | 46.873 | 39.213 | 10.797 | 1.00 | 63.23 |
| ATOM | 1895 | C | TYR | 253 | 42.250 | 35.362 | 5.713 | 1.00 | 60.86 |
| ATOM | 1896 | O | TYR | 253 | 42.409 | 34.215 | 5.283 | 1.00 | 58.01 |
| ATOM | 1897 | N | ASN | 254 | 41.403 | 36.242 | 5.183 | 1.00 | 60.70 |
| ATOM | 1898 | CA | ASN | 254 | 40.587 | 35.942 | 4.020 | 1.00 | 61.15 |
| ATOM | 1899 | CB | ASN | 254 | 39.425 | 36.939 | 3.922 | 1.00 | 62.18 |
| ATOM | 1900 | CG | ASN | 254 | 38.575 | 36.728 | 2.675 | 1.00 | 63.32 |
| ATOM | 1901 | OD1 | ASN | 254 | 38.771 | 37.386 | 1.648 | 1.00 | 60.67 |
| ATOM | 1902 | ND2 | ASN | 254 | 37.636 | 35.792 | 2.757 | 1.00 | 63.52 |
| ATOM | 1903 | C | ASN | 254 | 41.450 | 36.013 | 2.764 | 1.00 | 61.98 |
| ATOM | 1904 | O | ASN | 254 | 42.152 | 37.002 | 2.529 | 1.00 | 60.65 |
| ATOM | 1905 | N | THR | 255 | 41.385 | 34.954 | 1.965 | 1.00 | 62.41 |
| ATOM | 1906 | CA | THR | 255 | 42.149 | 34.841 | 0.726 | 1.00 | 63.35 |
| ATOM | 1907 | CB | THR | 255 | 41.705 | 33.572 | −0.030 | 1.00 | 63.45 |
| ATOM | 1908 | OG1 | THR | 255 | 42.419 | 32.447 | 0.496 | 1.00 | 63.38 |
| ATOM | 1909 | CG2 | THR | 255 | 41.947 | 33.700 | −1.535 | 1.00 | 63.89 |
| ATOM | 1910 | C | THR | 255 | 42.098 | 36.045 | −0.220 | 1.00 | 64.24 |
| ATOM | 1911 | O | THR | 255 | 43.107 | 36.404 | −0.826 | 1.00 | 63.50 |
| ATOM | 1912 | N | ASP | 256 | 40.929 | 36.669 | −0.337 | 1.00 | 66.28 |
| ATOM | 1913 | CA | ASP | 256 | 40.753 | 37.806 | −1.239 | 1.00 | 68.16 |
| ATOM | 1914 | CB | ASP | 256 | 39.442 | 37.660 | −2.018 | 1.00 | 69.86 |
| ATOM | 1915 | CG | ASP | 256 | 39.327 | 36.325 | −2.722 | 1.00 | 72.07 |
| ATOM | 1916 | OD1 | ASP | 256 | 40.068 | 36.111 | −3.712 | 1.00 | 73.36 |
| ATOM | 1917 | OD2 | ASP | 256 | 38.501 | 35.490 | −2.277 | 1.00 | 71.56 |
| ATOM | 1918 | C | ASP | 256 | 40.725 | 39.139 | −0.527 | 1.00 | 69.08 |
| ATOM | 1919 | O | ASP | 256 | 41.317 | 40.115 | −0.984 | 1.00 | 69.16 |
| ATOM | 1920 | N | THR | 257 | 40.020 | 39.176 | 0.593 | 1.00 | 70.47 |
| ATOM | 1921 | CA | THR | 257 | 39.874 | 40.400 | 1.357 | 1.00 | 72.75 |
| ATOM | 1922 | CB | THR | 257 | 38.563 | 40.381 | 2.149 | 1.00 | 73.56 |
| ATOM | 1923 | OG1 | THR | 257 | 37.564 | 39.674 | 1.403 | 1.00 | 74.14 |
| ATOM | 1924 | CG2 | THR | 257 | 38.086 | 41.791 | 2.403 | 1.00 | 73.14 |
| ATOM | 1925 | C | THR | 257 | 41.004 | 40.626 | 2.344 | 1.00 | 73.48 |
| ATOM | 1926 | O | THR | 257 | 41.428 | 41.760 | 2.568 | 1.00 | 74.03 |
| ATOM | 1927 | N | PHE | 258 | 41.492 | 39.537 | 2.927 | 1.00 | 74.08 |
| ATOM | 1928 | CA | PHE | 258 | 42.540 | 39.608 | 3.933 | 1.00 | 74.96 |
| ATOM | 1929 | CB | PHE | 258 | 43.801 | 40.300 | 3.396 | 1.00 | 73.47 |
| ATOM | 1930 | CG | PHE | 258 | 44.676 | 39.383 | 2.594 | 1.00 | 73.25 |
| ATOM | 1931 | CD1 | PHE | 258 | 44.419 | 39.154 | 1.248 | 1.00 | 72.60 |
| ATOM | 1932 | CD2 | PHE | 258 | 45.701 | 38.670 | 3.208 | 1.00 | 72.78 |
| ATOM | 1933 | CE1 | PHE | 258 | 45.163 | 38.223 | 0.526 | 1.00 | 73.28 |
| ATOM | 1934 | CE2 | PHE | 258 | 46.450 | 37.738 | 2.496 | 1.00 | 71.82 |
| ATOM | 1935 | CZ | PHE | 258 | 46.179 | 37.511 | 1.153 | 1.00 | 72.81 |
| ATOM | 1936 | C | PHE | 258 | 41.991 | 40.339 | 5.140 | 1.00 | 76.08 |
| ATOM | 1937 | O | PHE | 258 | 42.578 | 41.298 | 5.639 | 1.00 | 75.95 |
| ATOM | 1938 | N | GLU | 259 | 40.829 | 39.878 | 5.579 | 1.00 | 78.07 |
| ATOM | 1939 | CA | GLU | 259 | 40.171 | 40.435 | 6.740 | 1.00 | 80.33 |
| ATOM | 1940 | CB | GLU | 259 | 38.744 | 40.869 | 6.403 | 1.00 | 82.99 |
| ATOM | 1941 | CG | GLU | 259 | 38.635 | 42.348 | 6.032 | 1.00 | 88.00 |
| ATOM | 1942 | CD | GLU | 259 | 37.196 | 42.806 | 5.809 | 1.00 | 90.89 |
| ATOM | 1943 | OE1 | GLU | 259 | 36.577 | 42.381 | 4.807 | 1.00 | 91.78 |
| ATOM | 1944 | OE2 | GLU | 259 | 36.684 | 43.590 | 6.641 | 1.00 | 92.51 |
| ATOM | 1945 | C | GLU | 259 | 40.166 | 39.351 | 7.802 | 1.00 | 80.14 |
| ATOM | 1946 | O | GLU | 259 | 39.849 | 38.192 | 7.519 | 1.00 | 80.17 |
| ATOM | 1947 | N | SER | 260 | 40.535 | 39.741 | 9.018 | 1.00 | 79.91 |
| ATOM | 1948 | CA | SER | 260 | 40.613 | 38.830 | 10.152 | 1.00 | 79.51 |
| ATOM | 1949 | CB | SER | 260 | 40.948 | 39.603 | 11.435 | 1.00 | 79.83 |
| ATOM | 1950 | OG | SER | 260 | 42.295 | 40.041 | 11.451 | 1.00 | 82.07 |
| ATOM | 1951 | C | SER | 260 | 39.383 | 37.982 | 10.417 | 1.00 | 79.07 |
| ATOM | 1952 | O | SER | 260 | 38.247 | 38.383 | 10.162 | 1.00 | 78.31 |
| ATOM | 1953 | N | MET | 261 | 39.647 | 36.795 | 10.947 | 1.00 | 79.30 |
| ATOM | 1954 | CA | MET | 261 | 38.629 | 35.836 | 11.332 | 1.00 | 79.39 |
| ATOM | 1955 | CB | MET | 261 | 38.224 | 34.946 | 10.149 | 1.00 | 82.44 |
| ATOM | 1956 | CG | MET | 261 | 37.133 | 35.551 | 9.255 | 1.00 | 86.60 |
| ATOM | 1957 | SD | MET | 261 | 35.583 | 35.989 | 10.146 | 1.00 | 89.52 |
| ATOM | 1958 | CE | MET | 261 | 34.711 | 34.386 | 10.152 | 1.00 | 88.94 |
| ATOM | 1959 | C | MET | 261 | 39.249 | 35.004 | 12.446 | 1.00 | 77.40 |
| ATOM | 1960 | O | MET | 261 | 40.337 | 34.443 | 12.285 | 1.00 | 76.93 |
| ATOM | 1961 | N | PRO | 262 | 38.574 | 34.931 | 13.601 | 1.00 | 75.08 |
| ATOM | 1962 | CD | PRO | 262 | 37.184 | 35.340 | 13.847 | 1.00 | 74.38 |
| ATOM | 1963 | CA | PRO | 262 | 39.089 | 34.158 | 14.732 | 1.00 | 73.19 |
| ATOM | 1964 | CB | PRO | 262 | 37.940 | 34.212 | 15.734 | 1.00 | 73.46 |
| ATOM | 1965 | CG | PRO | 262 | 36.740 | 34.314 | 14.858 | 1.00 | 74.32 |
| ATOM | 1966 | C | PRO | 262 | 39.463 | 32.737 | 14.333 | 1.00 | 71.04 |
| ATOM | 1967 | O | PRO | 262 | 38.839 | 32.140 | 13.454 | 1.00 | 70.71 |
| ATOM | 1968 | N | ASN | 263 | 40.496 | 32.210 | 14.978 | 1.00 | 68.58 |
| ATOM | 1969 | CA | ASN | 263 | 40.962 | 30.864 | 14.689 | 1.00 | 67.27 |
| ATOM | 1970 | CB | ASN | 263 | 42.470 | 30.865 | 14.436 | 1.00 | 66.62 |
| ATOM | 1971 | CG | ASN | 263 | 42.986 | 29.507 | 14.004 | 1.00 | 65.52 |
| ATOM | 1972 | OD1 | ASN | 263 | 42.506 | 28.474 | 14.462 | 1.00 | 65.69 |
| ATOM | 1973 | ND2 | ASN | 263 | 43.980 | 29.502 | 13.129 | 1.00 | 65.08 |
| ATOM | 1974 | C | ASN | 263 | 40.653 | 29.941 | 15.857 | 1.00 | 66.33 |
| ATOM | 1975 | O | ASN | 263 | 41.212 | 30.099 | 16.941 | 1.00 | 65.65 |
| ATOM | 1976 | N | PRO | 264 | 39.761 | 28.959 | 15.647 | 1.00 | 66.51 |
| ATOM | 1977 | CD | PRO | 264 | 39.157 | 28.575 | 14.357 | 1.00 | 65.55 |
| ATOM | 1978 | CA | PRO | 264 | 39.390 | 28.010 | 16.702 | 1.00 | 65.99 |
| ATOM | 1979 | CB | PRO | 264 | 38.407 | 27.078 | 15.991 | 1.00 | 66.25 |
| ATOM | 1980 | CG | PRO | 264 | 38.883 | 27.105 | 14.561 | 1.00 | 65.20 |
| ATOM | 1981 | C | PRO | 264 | 40.612 | 27.267 | 17.251 | 1.00 | 66.56 |
| ATOM | 1982 | O | PRO | 264 | 40.627 | 26.838 | 18.407 | 1.00 | 65.94 |
| ATOM | 1983 | N | GLU | 265 | 41.637 | 27.131 | 16.411 | 1.00 | 66.53 |
| ATOM | 1984 | CA | GLU | 265 | 42.872 | 26.450 | 16.781 | 1.00 | 65.73 |
| ATOM | 1985 | CB | GLU | 265 | 43.401 | 25.655 | 15.585 | 1.00 | 68.14 |
| ATOM | 1986 | CG | GLU | 265 | 42.471 | 24.553 | 15.090 | 1.00 | 72.53 |
| ATOM | 1987 | CD | GLU | 265 | 42.368 | 23.379 | 16.059 | 1.00 | 76.42 |
| ATOM | 1988 | OE1 | GLU | 265 | 43.414 | 22.773 | 16.391 | 1.00 | 78.03 |
| ATOM | 1989 | OE2 | GLU | 265 | 41.238 | 23.057 | 16.487 | 1.00 | 78.72 |
| ATOM | 1990 | C | GLU | 265 | 43.940 | 27.440 | 17.250 | 1.00 | 63.96 |
| ATOM | 1991 | O | GLU | 265 | 45.083 | 27.057 | 17.509 | 1.00 | 63.86 |
| ATOM | 1992 | N | GLY | 266 | 43.566 | 28.710 | 17.356 | 1.00 | 61.36 |
| ATOM | 1993 | CA | GLY | 266 | 44.514 | 29.722 | 17.789 | 1.00 | 58.94 |
| ATOM | 1994 | C | GLY | 266 | 44.947 | 29.550 | 19.231 | 1.00 | 57.21 |
| ATOM | 1995 | O | GLY | 266 | 44.139 | 29.165 | 20.071 | 1.00 | 57.33 |
| ATOM | 1996 | N | ARG | 267 | 46.222 | 29.830 | 19.511 | 1.00 | 56.18 |
| ATOM | 1997 | CA | ARG | 267 | 46.793 | 29.724 | 20.861 | 1.00 | 53.52 |
| ATOM | 1998 | CB | ARG | 267 | 47.494 | 28.376 | 21.049 | 1.00 | 55.28 |
| ATOM | 1999 | CG | ARG | 267 | 46.634 | 27.156 | 20.756 | 1.00 | 59.39 |
| ATOM | 2000 | CD | ARG | 267 | 45.362 | 27.124 | 21.604 | 1.00 | 61.46 |
| ATOM | 2001 | NE | ARG | 267 | 44.597 | 25.914 | 21.346 | 1.00 | 61.95 |
| ATOM | 2002 | CZ | ARG | 267 | 45.085 | 24.696 | 21.543 | 1.00 | 66.21 |
| ATOM | 2003 | NH1 | ARG | 267 | 46.326 | 24.547 | 21.999 | 1.00 | 66.55 |
| ATOM | 2004 | NH2 | ARG | 267 | 44.347 | 23.626 | 21.280 | 1.00 | 66.39 |
| ATOM | 2005 | C | ARG | 267 | 47.827 | 30.824 | 21.096 | 1.00 | 52.01 |
| ATOM | 2006 | O | ARG | 267 | 48.474 | 31.287 | 20.153 | 1.00 | 50.48 |
| ATOM | 2007 | N | TYR | 268 | 47.988 | 31.247 | 22.347 | 1.00 | 49.34 |
| ATOM | 2008 | CA | TYR | 268 | 48.990 | 32.267 | 22.645 | 1.00 | 48.57 |
| ATOM | 2009 | CB | TYR | 268 | 48.583 | 33.135 | 23.838 | 1.00 | 49.99 |
| ATOM | 2010 | CG | TYR | 268 | 47.271 | 33.870 | 23.686 | 1.00 | 50.84 |
| ATOM | 2011 | CD1 | TYR | 268 | 46.055 | 33.181 | 23.695 | 1.00 | 50.27 |
| ATOM | 2012 | CE1 | TYR | 268 | 44.847 | 33.853 | 23.549 | 1.00 | 50.81 |
| ATOM | 2013 | CD2 | TYR | 268 | 47.245 | 35.253 | 23.533 | 1.00 | 49.75 |
| ATOM | 2014 | CE2 | TYR | 268 | 46.044 | 35.937 | 23.387 | 1.00 | 51.75 |
| ATOM | 2015 | CZ | TYR | 268 | 44.849 | 35.231 | 23.393 | 1.00 | 52.26 |
| ATOM | 2016 | OH | TYR | 268 | 43.661 | 35.903 | 23.213 | 1.00 | 54.20 |
| ATOM | 2017 | C | TYR | 268 | 50.324 | 31.594 | 22.975 | 1.00 | 48.52 |
| ATOM | 2018 | O | TYR | 268 | 50.384 | 30.390 | 23.261 | 1.00 | 46.36 |
| ATOM | 2019 | N | THR | 269 | 51.397 | 32.376 | 22.920 | 1.00 | 48.17 |
| ATOM | 2020 | CA | THR | 269 | 52.707 | 31.853 | 23.252 | 1.00 | 46.34 |
| ATOM | 2021 | CB | THR | 269 | 53.817 | 32.473 | 22.379 | 1.00 | 44.79 |
| ATOM | 2022 | OG1 | THR | 269 | 53.783 | 33.898 | 22.488 | 1.00 | 45.68 |
| ATOM | 2023 | CG2 | THR | 269 | 53.632 | 32.083 | 20.938 | 1.00 | 45.34 |
| ATOM | 2024 | C | THR | 269 | 52.960 | 32.186 | 24.721 | 1.00 | 47.04 |
| ATOM | 2025 | O | THR | 269 | 52.713 | 33.309 | 25.170 | 1.00 | 47.56 |
| ATOM | 2026 | N | PHE | 270 | 53.411 | 31.186 | 25.469 | 1.00 | 45.80 |
| ATOM | 2027 | CA | PHE | 270 | 53.719 | 31.342 | 26.881 | 1.00 | 46.48 |
| ATOM | 2028 | CB | PHE | 270 | 52.532 | 30.926 | 27.754 | 1.00 | 47.64 |
| ATOM | 2029 | CG | PHE | 270 | 52.853 | 30.875 | 29.221 | 1.00 | 47.84 |
| ATOM | 2030 | CD1 | PHE | 270 | 53.395 | 31.978 | 29.865 | 1.00 | 49.86 |
| ATOM | 2031 | CD2 | PHE | 270 | 52.604 | 29.727 | 29.962 | 1.00 | 50.22 |
| ATOM | 2032 | CE1 | PHE | 270 | 53.682 | 31.941 | 31.234 | 1.00 | 51.58 |
| ATOM | 2033 | CE2 | PHE | 270 | 52.888 | 29.679 | 31.326 | 1.00 | 50.74 |
| ATOM | 2034 | CZ | PHE | 270 | 53.427 | 30.791 | 31.963 | 1.00 | 50.30 |
| ATOM | 2035 | C | PHE | 270 | 54.902 | 30.433 | 27.152 | 1.00 | 45.41 |
| ATOM | 2036 | O | PHE | 270 | 54.750 | 29.220 | 27.261 | 1.00 | 45.07 |
| ATOM | 2037 | N | GLY | 271 | 56.083 | 31.024 | 27.261 | 1.00 | 45.52 |
| ATOM | 2038 | CA | GLY | 271 | 57.263 | 30.217 | 27.475 | 1.00 | 46.26 |
| ATOM | 2039 | C | GLY | 271 | 57.451 | 29.372 | 26.227 | 1.00 | 46.08 |
| ATOM | 2040 | O | GLY | 271 | 57.336 | 29.881 | 25.113 | 1.00 | 47.07 |
| ATOM | 2041 | N | ALA | 272 | 57.711 | 28.081 | 26.401 | 1.00 | 45.01 |
| ATOM | 2042 | CA | ALA | 272 | 57.910 | 27.207 | 25.262 | 1.00 | 44.35 |
| ATOM | 2043 | CB | ALA | 272 | 59.112 | 26.288 | 25.503 | 1.00 | 42.29 |
| ATOM | 2044 | C | ALA | 272 | 56.671 | 26.382 | 24.970 | 1.00 | 45.01 |
| ATOM | 2045 | O | ALA | 272 | 56.778 | 25.213 | 24.595 | 1.00 | 46.73 |
| ATOM | 2046 | N | SER | 273 | 55.493 | 26.973 | 25.134 | 1.00 | 44.94 |
| ATOM | 2047 | CA | SER | 273 | 54.278 | 26.216 | 24.855 | 1.00 | 47.72 |
| ATOM | 2048 | CB | SER | 273 | 53.795 | 25.497 | 26.120 | 1.00 | 48.16 |
| ATOM | 2049 | OG | SER | 273 | 53.297 | 26.428 | 27.055 | 1.00 | 53.37 |
| ATOM | 2050 | C | SER | 273 | 53.141 | 27.053 | 24.283 | 1.00 | 46.71 |
| ATOM | 2051 | O | SER | 273 | 53.121 | 28.271 | 24.435 | 1.00 | 44.26 |
| ATOM | 2052 | N | CYS | 274 | 52.210 | 26.378 | 23.608 | 1.00 | 45.77 |
| ATOM | 2053 | CA | CYS | 274 | 51.050 | 27.039 | 23.025 | 1.00 | 47.39 |
| ATOM | 2054 | C | CYS | 274 | 49.870 | 26.777 | 23.947 | 1.00 | 47.37 |
| ATOM | 2055 | O | CYS | 274 | 49.494 | 25.631 | 24.163 | 1.00 | 49.49 |
| ATOM | 2056 | CB | CYS | 274 | 50.735 | 26.476 | 21.639 | 1.00 | 47.43 |
| ATOM | 2057 | SG | CYS | 274 | 52.155 | 26.421 | 20.504 | 1.00 | 46.90 |
| ATOM | 2058 | N | VAL | 275 | 49.287 | 27.838 | 24.486 | 1.00 | 46.52 |
| ATOM | 2059 | CA | VAL | 275 | 48.168 | 27.690 | 25.403 | 1.00 | 46.86 |
| ATOM | 2060 | CB | VAL | 275 | 48.516 | 28.276 | 26.771 | 1.00 | 44.33 |
| ATOM | 2061 | CG1 | VAL | 275 | 49.818 | 27.687 | 27.253 | 1.00 | 43.46 |
| ATOM | 2062 | CG2 | VAL | 275 | 48.630 | 29.783 | 26.669 | 1.00 | 44.17 |
| ATOM | 2063 | C | VAL | 275 | 46.937 | 28.401 | 24.879 | 1.00 | 47.21 |
| ATOM | 2064 | O | VAL | 275 | 47.047 | 29.367 | 24.130 | 1.00 | 48.64 |
| ATOM | 2065 | N | THR | 276 | 45.768 | 27.925 | 25.291 | 1.00 | 47.78 |
| ATOM | 2066 | CA | THR | 276 | 44.497 | 28.509 | 24.864 | 1.00 | 47.96 |
| ATOM | 2067 | CB | THR | 276 | 43.337 | 27.562 | 25.180 | 1.00 | 48.33 |
| ATOM | 2068 | OG1 | THR | 276 | 43.221 | 27.419 | 26.603 | 1.00 | 47.75 |
| ATOM | 2069 | CG2 | THR | 276 | 43.592 | 26.188 | 24.555 | 1.00 | 47.06 |
| ATOM | 2070 | C | THR | 276 | 44.227 | 29.841 | 25.554 | 1.00 | 47.72 |
| ATOM | 2071 | O | THR | 276 | 43.563 | 30.710 | 25.005 | 1.00 | 47.53 |
| ATOM | 2072 | N | ALA | 277 | 44.743 | 29.990 | 26.768 | 1.00 | 48.65 |
| ATOM | 2073 | CA | ALA | 277 | 44.558 | 31.217 | 27.529 | 1.00 | 48.95 |
| ATOM | 2074 | CB | ALA | 277 | 43.339 | 31.091 | 28.434 | 1.00 | 49.82 |
| ATOM | 2075 | C | ALA | 277 | 45.788 | 31.497 | 28.371 | 1.00 | 48.96 |
| ATOM | 2076 | O | ALA | 277 | 46.396 | 30.572 | 28.921 | 1.00 | 48.47 |
| ATOM | 2077 | N | CYS | 278 | 46.157 | 32.771 | 28.472 | 1.00 | 49.32 |
| ATOM | 2078 | CA | CYS | 278 | 47.314 | 33.146 | 29.281 | 1.00 | 50.33 |
| ATOM | 2079 | C | CYS | 278 | 47.017 | 32.867 | 30.753 | 1.00 | 51.58 |
| ATOM | 2080 | O | CYS | 278 | 45.878 | 33.010 | 31.205 | 1.00 | 51.96 |
| ATOM | 2081 | CB | CYS | 278 | 47.650 | 34.626 | 29.095 | 1.00 | 46.73 |
| ATOM | 2082 | SG | CYS | 278 | 48.295 | 35.016 | 27.442 | 1.00 | 45.43 |
| ATOM | 2083 | N | PRO | 279 | 48.033 | 32.440 | 31.517 | 1.00 | 52.20 |
| ATOM | 2084 | CD | PRO | 279 | 49.389 | 32.030 | 31.107 | 1.00 | 53.86 |
| ATOM | 2085 | CA | PRO | 279 | 47.807 | 32.159 | 32.936 | 1.00 | 51.15 |
| ATOM | 2086 | CB | PRO | 279 | 49.062 | 31.390 | 33.343 | 1.00 | 51.40 |
| ATOM | 2087 | CG | PRO | 279 | 50.119 | 31.958 | 32.442 | 1.00 | 53.87 |
| ATOM | 2088 | C | PRO | 279 | 47.590 | 33.422 | 33.770 | 1.00 | 50.59 |
| ATOM | 2089 | O | PRO | 279 | 47.843 | 34.543 | 33.320 | 1.00 | 48.69 |
| ATOM | 2090 | N | TYR | 280 | 47.118 | 33.222 | 34.995 | 1.00 | 50.86 |
| ATOM | 2091 | CA | TYR | 280 | 46.841 | 34.318 | 35.905 | 1.00 | 49.82 |
| ATOM | 2092 | CB | TYR | 280 | 46.464 | 33.784 | 37.284 | 1.00 | 48.50 |
| ATOM | 2093 | CG | TYR | 280 | 45.914 | 34.859 | 38.180 | 1.00 | 48.40 |
| ATOM | 2094 | CD1 | TYR | 280 | 44.638 | 35.374 | 37.970 | 1.00 | 49.04 |
| ATOM | 2095 | CE1 | TYR | 280 | 44.144 | 36.418 | 38.747 | 1.00 | 49.87 |
| ATOM | 2096 | CD2 | TYR | 280 | 46.689 | 35.413 | 39.194 | 1.00 | 48.78 |
| ATOM | 2097 | CE2 | TYR | 280 | 46.206 | 36.459 | 39.979 | 1.00 | 50.12 |
| ATOM | 2098 | CZ | TYR | 280 | 44.933 | 36.957 | 39.747 | 1.00 | 50.73 |
| ATOM | 2099 | OH | TYR | 280 | 44.448 | 37.995 | 40.512 | 1.00 | 53.36 |
| ATOM | 2100 | C | TYR | 280 | 47.991 | 35.297 | 36.066 | 1.00 | 50.72 |
| ATOM | 2101 | O | TYR | 280 | 49.149 | 34.899 | 36.210 | 1.00 | 50.74 |
| ATOM | 2102 | N | ASN | 281 | 47.637 | 36.579 | 36.043 | 1.00 | 50.37 |
| ATOM | 2103 | CA | ASN | 281 | 48.562 | 37.690 | 36.219 | 1.00 | 51.25 |
| ATOM | 2104 | CB | ASN | 281 | 49.495 | 37.430 | 37.413 | 1.00 | 51.06 |
| ATOM | 2105 | CG | ASN | 281 | 49.975 | 38.723 | 38.068 | 1.00 | 51.36 |
| ATOM | 2106 | OD1 | ASN | 281 | 49.221 | 39.689 | 38.171 | 1.00 | 51.17 |
| ATOM | 2107 | ND2 | ASN | 281 | 51.220 | 38.739 | 38.526 | 1.00 | 51.60 |
| ATOM | 2108 | C | ASN | 281 | 49.379 | 38.071 | 34.989 | 1.00 | 51.58 |
| ATOM | 2109 | O | ASN | 281 | 50.151 | 39.036 | 35.027 | 1.00 | 50.94 |
| ATOM | 2110 | N | TYR | 282 | 49.211 | 37.326 | 33.902 | 1.00 | 51.57 |
| ATOM | 2111 | CA | TYR | 282 | 49.927 | 37.644 | 32.671 | 1.00 | 52.72 |
| ATOM | 2112 | CB | TYR | 282 | 50.364 | 36.368 | 31.948 | 1.00 | 52.57 |
| ATOM | 2113 | CG | TYR | 282 | 51.685 | 35.802 | 32.430 | 1.00 | 53.88 |
| ATOM | 2114 | CD1 | TYR | 282 | 51.791 | 35.168 | 33.670 | 1.00 | 54.63 |
| ATOM | 2115 | CE1 | TYR | 282 | 53.020 | 34.658 | 34.121 | 1.00 | 53.76 |
| ATOM | 2116 | CD2 | TYR | 282 | 52.838 | 35.915 | 31.647 | 1.00 | 53.83 |
| ATOM | 2117 | CE2 | TYR | 282 | 54.064 | 35.413 | 32.085 | 1.00 | 53.98 |
| ATOM | 2118 | CZ | TYR | 282 | 54.151 | 34.786 | 33.321 | 1.00 | 54.93 |
| ATOM | 2119 | OH | TYR | 282 | 55.363 | 34.284 | 33.751 | 1.00 | 54.27 |
| ATOM | 2120 | C | TYR | 282 | 49.061 | 38.495 | 31.742 | 1.00 | 53.12 |
| ATOM | 2121 | O | TYR | 282 | 47.845 | 38.578 | 31.904 | 1.00 | 51.51 |
| ATOM | 2122 | N | LEU | 283 | 49.699 | 39.132 | 30.770 | 1.00 | 54.89 |
| ATOM | 2123 | CA | LEU | 283 | 48.993 | 39.976 | 29.820 | 1.00 | 56.63 |
| ATOM | 2124 | CB | LEU | 283 | 49.729 | 41.311 | 29.671 | 1.00 | 58.42 |
| ATOM | 2125 | CG | LEU | 283 | 49.807 | 42.232 | 30.900 | 1.00 | 59.48 |
| ATOM | 2126 | CD1 | LEU | 283 | 50.883 | 43.288 | 30.699 | 1.00 | 59.69 |
| ATOM | 2127 | CD2 | LEU | 283 | 48.461 | 42.892 | 31.133 | 1.00 | 60.34 |
| ATOM | 2128 | C | LEU | 283 | 48.875 | 39.299 | 28.459 | 1.00 | 57.32 |
| ATOM | 2129 | O | LEU | 283 | 49.858 | 38.793 | 27.921 | 1.00 | 58.54 |
| ATOM | 2130 | N | SER | 284 | 47.663 | 39.273 | 27.913 | 1.00 | 58.71 |
| ATOM | 2131 | CA | SER | 284 | 47.422 | 38.681 | 26.600 | 1.00 | 59.30 |
| ATOM | 2132 | CB | SER | 284 | 45.977 | 38.197 | 26.481 | 1.00 | 58.31 |
| ATOM | 2133 | OG | SER | 284 | 45.749 | 37.090 | 27.340 | 1.00 | 58.17 |
| ATOM | 2134 | C | SER | 284 | 47.688 | 39.795 | 25.608 | 1.00 | 60.28 |
| ATOM | 2135 | O | SER | 284 | 47.313 | 40.939 | 25.855 | 1.00 | 60.43 |
| ATOM | 2136 | N | THR | 285 | 48.332 | 39.476 | 24.490 | 1.00 | 61.69 |
| ATOM | 2137 | CA | THR | 285 | 48.660 | 40.506 | 23.514 | 1.00 | 62.74 |
| ATOM | 2138 | CB | THR | 285 | 50.177 | 40.574 | 23.288 | 1.00 | 61.20 |
| ATOM | 2139 | OG1 | THR | 285 | 50.617 | 39.392 | 22.605 | 1.00 | 60.25 |
| ATOM | 2140 | CG2 | THR | 285 | 50.893 | 40.683 | 24.616 | 1.00 | 59.34 |
| ATOM | 2141 | C | THR | 285 | 47.997 | 40.382 | 22.155 | 1.00 | 65.35 |
| ATOM | 2142 | O | THR | 285 | 47.310 | 39.405 | 21.863 | 1.00 | 66.92 |
| ATOM | 2143 | N | ASP | 286 | 48.217 | 41.401 | 21.328 | 1.00 | 68.96 |
| ATOM | 2144 | CA | ASP | 286 | 47.680 | 41.456 | 19.974 | 1.00 | 70.35 |
| ATOM | 2145 | CB | ASP | 286 | 48.064 | 42.779 | 19.292 | 1.00 | 73.32 |
| ATOM | 2146 | CG | ASP | 286 | 47.489 | 44.000 | 19.991 | 1.00 | 75.72 |
| ATOM | 2147 | OD1 | ASP | 286 | 46.253 | 44.191 | 19.922 | 1.00 | 75.54 |
| ATOM | 2148 | OD2 | ASP | 286 | 48.276 | 44.763 | 20.603 | 1.00 | 76.45 |
| ATOM | 2149 | C | ASP | 286 | 48.318 | 40.325 | 19.191 | 1.00 | 70.06 |
| ATOM | 2150 | O | ASP | 286 | 47.648 | 39.581 | 18.467 | 1.00 | 70.44 |
| ATOM | 2151 | N | VAL | 287 | 49.631 | 40.210 | 19.356 | 1.00 | 68.31 |
| ATOM | 2152 | CA | VAL | 287 | 50.420 | 39.212 | 18.655 | 1.00 | 66.57 |
| ATOM | 2153 | CB | VAL | 287 | 51.900 | 39.626 | 18.622 | 1.00 | 68.51 |
| ATOM | 2154 | CG1 | VAL | 287 | 52.583 | 38.997 | 17.402 | 1.00 | 70.27 |
| ATOM | 2155 | CG2 | VAL | 287 | 52.016 | 41.147 | 18.609 | 1.00 | 67.05 |
| ATOM | 2156 | C | VAL | 287 | 50.330 | 37.807 | 19.237 | 1.00 | 64.30 |
| ATOM | 2157 | O | VAL | 287 | 51.238 | 36.999 | 19.048 | 1.00 | 64.78 |
| ATOM | 2158 | N | GLY | 288 | 49.247 | 37.522 | 19.953 | 1.00 | 62.01 |
| ATOM | 2159 | CA | GLY | 288 | 49.056 | 36.201 | 20.533 | 1.00 | 59.45 |
| ATOM | 2160 | C | GLY | 288 | 50.131 | 35.683 | 21.476 | 1.00 | 57.09 |
| ATOM | 2161 | O | GLY | 288 | 50.586 | 34.542 | 21.348 | 1.00 | 55.74 |
| ATOM | 2162 | N | SER | 289 | 50.546 | 36.509 | 22.426 | 1.00 | 55.12 |
| ATOM | 2163 | CA | SER | 289 | 51.557 | 36.082 | 23.381 | 1.00 | 55.42 |
| ATOM | 2164 | CB | SER | 289 | 52.903 | 36.772 | 23.091 | 1.00 | 55.58 |
| ATOM | 2165 | OG | SER | 289 | 52.827 | 38.181 | 23.246 | 1.00 | 54.26 |
| ATOM | 2166 | C | SER | 289 | 51.119 | 36.373 | 24.815 | 1.00 | 54.82 |
| ATOM | 2167 | O | SER | 289 | 50.156 | 37.109 | 25.051 | 1.00 | 52.55 |
| ATOM | 2168 | N | CYS | 290 | 51.822 | 35.771 | 25.768 | 1.00 | 54.19 |
| ATOM | 2169 | CA | CYS | 290 | 51.535 | 35.989 | 27.176 | 1.00 | 54.40 |
| ATOM | 2170 | C | CYS | 290 | 52.756 | 36.694 | 27.740 | 1.00 | 56.69 |
| ATOM | 2171 | O | CYS | 290 | 53.846 | 36.119 | 27.849 | 1.00 | 56.87 |
| ATOM | 2172 | CB | CYS | 290 | 51.286 | 34.661 | 27.884 | 1.00 | 51.20 |
| ATOM | 2173 | SG | CYS | 290 | 49.861 | 33.745 | 27.217 | 1.00 | 48.94 |
| ATOM | 2174 | N | THR | 291 | 52.576 | 37.957 | 28.088 | 1.00 | 58.96 |
| ATOM | 2175 | CA | THR | 291 | 53.692 | 38.732 | 28.586 | 1.00 | 62.71 |
| ATOM | 2176 | CB | THR | 291 | 54.214 | 39.664 | 27.492 | 1.00 | 62.87 |
| ATOM | 2177 | OG1 | THR | 291 | 55.427 | 40.284 | 27.929 | 1.00 | 67.19 |
| ATOM | 2178 | CG2 | THR | 291 | 53.181 | 40.738 | 27.185 | 1.00 | 62.81 |
| ATOM | 2179 | C | THR | 291 | 53.350 | 39.570 | 29.800 | 1.00 | 64.32 |
| ATOM | 2180 | O | THR | 291 | 52.178 | 39.807 | 30.105 | 1.00 | 64.63 |
| ATOM | 2181 | N | LEU | 292 | 54.396 | 40.024 | 30.482 | 1.00 | 65.88 |
| ATOM | 2182 | CA | LEU | 292 | 54.249 | 40.857 | 31.665 | 1.00 | 67.54 |
| ATOM | 2183 | CB | LEU | 292 | 55.361 | 40.537 | 32.663 | 1.00 | 64.73 |
| ATOM | 2184 | CG | LEU | 292 | 55.548 | 39.042 | 32.941 | 1.00 | 65.08 |
| ATOM | 2185 | CD1 | LEU | 292 | 56.703 | 38.835 | 33.898 | 1.00 | 64.62 |
| ATOM | 2186 | CD2 | LEU | 292 | 54.270 | 38.454 | 33.512 | 1.00 | 64.78 |
| ATOM | 2187 | C | LEU | 292 | 54.300 | 42.338 | 31.280 | 1.00 | 69.22 |
| ATOM | 2188 | O | LEU | 292 | 53.964 | 43.204 | 32.085 | 1.00 | 70.92 |
| ATOM | 2189 | N | VAL | 293 | 54.707 | 42.625 | 30.047 | 1.00 | 71.80 |
| ATOM | 2190 | CA | VAL | 293 | 54.803 | 44.003 | 29.577 | 1.00 | 75.12 |
| ATOM | 2191 | CB | VAL | 293 | 56.269 | 44.492 | 29.586 | 1.00 | 75.39 |
| ATOM | 2192 | CG1 | VAL | 293 | 56.303 | 46.012 | 29.559 | 1.00 | 75.24 |
| ATOM | 2193 | CG2 | VAL | 293 | 57.009 | 43.945 | 30.809 | 1.00 | 74.43 |
| ATOM | 2194 | C | VAL | 293 | 54.264 | 44.109 | 28.155 | 1.00 | 77.87 |
| ATOM | 2195 | O | VAL | 293 | 54.600 | 43.294 | 27.303 | 1.00 | 78.41 |
| ATOM | 2196 | N | CYS | 294 | 53.441 | 45.120 | 27.899 | 1.00 | 81.86 |
| ATOM | 2197 | CA | CYS | 294 | 52.840 | 45.315 | 26.581 | 1.00 | 87.12 |
| ATOM | 2198 | C | CYS | 294 | 53.783 | 45.780 | 25.455 | 1.00 | 91.22 |
| ATOM | 2199 | O | CYS | 294 | 54.965 | 46.052 | 25.685 | 1.00 | 91.41 |
| ATOM | 2200 | CB | CYS | 294 | 51.676 | 46.299 | 26.685 | 1.00 | 86.96 |
| ATOM | 2201 | SG | CYS | 294 | 50.194 | 45.704 | 27.562 | 1.00 | 88.97 |
| ATOM | 2202 | N | PRO | 295 | 53.258 | 45.854 | 24.208 | 1.00 | 95.10 |
| ATOM | 2203 | CD | PRO | 295 | 52.018 | 45.149 | 23.830 | 1.00 | 96.29 |
| ATOM | 2204 | CA | PRO | 295 | 53.979 | 46.276 | 22.996 | 1.00 | 97.21 |
| ATOM | 2205 | CB | PRO | 295 | 53.069 | 45.788 | 21.863 | 1.00 | 96.88 |
| ATOM | 2206 | CG | PRO | 295 | 52.360 | 44.617 | 22.463 | 1.00 | 96.55 |
| ATOM | 2207 | C | PRO | 295 | 54.246 | 47.781 | 22.887 | 1.00 | 98.82 |
| ATOM | 2208 | O | PRO | 295 | 53.619 | 48.585 | 23.581 | 1.00 | 98.72 |
| ATOM | 2209 | N | LEU | 296 | 55.172 | 48.137 | 21.992 | 1.00 | 100.53 |
| ATOM | 2210 | CA | LEU | 296 | 55.571 | 49.524 | 21.731 | 1.00 | 102.46 |
| ATOM | 2211 | CB | LEU | 296 | 55.670 | 49.784 | 20.223 | 1.00 | 103.54 |
| ATOM | 2212 | CG | LEU | 296 | 56.912 | 49.335 | 19.452 | 1.00 | 104.08 |
| ATOM | 2213 | CD1 | LEU | 296 | 56.713 | 49.602 | 17.963 | 1.00 | 103.26 |
| ATOM | 2214 | CD2 | LEU | 296 | 58.138 | 50.078 | 19.974 | 1.00 | 103.86 |
| ATOM | 2215 | C | LEU | 296 | 54.635 | 50.560 | 22.321 | 1.00 | 103.21 |
| ATOM | 2216 | O | LEU | 296 | 54.820 | 51.017 | 23.449 | 1.00 | 103.65 |
| ATOM | 2217 | N | HIS | 297 | 53.629 | 50.933 | 21.539 | 1.00 | 104.02 |
| ATOM | 2218 | CA | HIS | 297 | 52.665 | 51.925 | 21.973 | 1.00 | 104.87 |
| ATOM | 2219 | CB | HIS | 297 | 52.446 | 52.953 | 20.871 | 1.00 | 105.33 |
| ATOM | 2220 | C | HIS | 297 | 51.338 | 51.301 | 22.387 | 1.00 | 105.28 |
| ATOM | 2221 | O | HIS | 297 | 50.342 | 52.005 | 22.515 | 1.00 | 105.58 |
| ATOM | 2222 | N | ASP | 298 | 51.323 | 49.984 | 22.585 | 1.00 | 105.05 |
| ATOM | 2223 | CA | ASP | 298 | 50.108 | 49.290 | 23.019 | 1.00 | 104.04 |
| ATOM | 2224 | CB | ASP | 298 | 50.140 | 47.815 | 22.602 | 1.00 | 103.55 |
| ATOM | 2225 | CG | ASP | 298 | 49.793 | 47.659 | 21.238 | 1.00 | 102.45 |
| ATOM | 2226 | C | ASP | 298 | 50.009 | 49.395 | 24.538 | 1.00 | 103.29 |
| ATOM | 2227 | O | ASP | 298 | 51.029 | 49.358 | 25.229 | 1.00 | 103.87 |
| ATOM | 2228 | N | GLN | 299 | 48.794 | 49.527 | 25.064 | 1.00 | 101.99 |
| ATOM | 2229 | CA | GLN | 299 | 48.634 | 49.649 | 26.506 | 1.00 | 101.04 |
| ATOM | 2230 | CB | GLN | 299 | 48.371 | 51.107 | 26.866 | 1.00 | 101.54 |
| ATOM | 2231 | CG | GLN | 299 | 49.451 | 52.031 | 26.331 | 1.00 | 102.81 |
| ATOM | 2232 | CD | GLN | 299 | 49.618 | 53.289 | 27.159 | 1.00 | 104.15 |
| ATOM | 2233 | OE1 | GLN | 299 | 48.641 | 53.969 | 27.483 | 1.00 | 104.75 |
| ATOM | 2234 | NE2 | GLN | 299 | 50.865 | 53.614 | 27.498 | 1.00 | 103.49 |
| ATOM | 2235 | C | GLN | 299 | 47.576 | 48.747 | 27.142 | 1.00 | 100.08 |
| ATOM | 2236 | O | GLN | 299 | 46.638 | 48.293 | 26.483 | 1.00 | 100.27 |
| ATOM | 2237 | N | GLU | 300 | 47.749 | 48.501 | 28.438 | 1.00 | 98.05 |
| ATOM | 2238 | CA | GLU | 300 | 46.867 | 47.635 | 29.215 | 1.00 | 96.41 |
| ATOM | 2239 | CB | GLU | 300 | 47.407 | 47.512 | 30.641 | 1.00 | 95.18 |
| ATOM | 2240 | CG | GLU | 300 | 48.771 | 46.846 | 30.711 | 1.00 | 93.52 |
| ATOM | 2241 | CD | GLU | 300 | 49.335 | 46.801 | 32.111 | 1.00 | 91.95 |
| ATOM | 2242 | OE1 | GLU | 300 | 48.591 | 46.407 | 33.031 | 1.00 | 92.20 |
| ATOM | 2243 | OE2 | GLU | 300 | 50.523 | 47.148 | 32.291 | 1.00 | 90.84 |
| ATOM | 2244 | C | GLU | 300 | 45.407 | 48.061 | 29.261 | 1.00 | 95.94 |
| ATOM | 2245 | O | GLU | 300 | 45.092 | 49.242 | 29.346 | 1.00 | 95.78 |
| ATOM | 2246 | N | VAL | 301 | 44.519 | 47.074 | 29.209 | 1.00 | 95.93 |
| ATOM | 2247 | CA | VAL | 301 | 43.081 | 47.307 | 29.251 | 1.00 | 96.56 |
| ATOM | 2248 | CB | VAL | 301 | 42.452 | 47.153 | 27.857 | 1.00 | 96.60 |
| ATOM | 2249 | CG1 | VAL | 301 | 40.966 | 47.486 | 27.915 | 1.00 | 96.39 |
| ATOM | 2250 | CG2 | VAL | 301 | 43.174 | 48.049 | 26.866 | 1.00 | 96.93 |
| ATOM | 2251 | C | VAL | 301 | 42.432 | 46.290 | 30.183 | 1.00 | 97.30 |
| ATOM | 2252 | O | VAL | 301 | 42.285 | 45.118 | 29.833 | 1.00 | 96.75 |
| ATOM | 2253 | N | THR | 302 | 42.038 | 46.747 | 31.367 | 1.00 | 98.44 |
| ATOM | 2254 | CA | THR | 302 | 41.426 | 45.875 | 32.360 | 1.00 | 99.64 |
| ATOM | 2255 | CB | THR | 302 | 41.483 | 46.500 | 33.767 | 1.00 | 99.04 |
| ATOM | 2256 | OG1 | THR | 302 | 42.818 | 46.934 | 34.050 | 1.00 | 98.39 |
| ATOM | 2257 | CG2 | THR | 302 | 41.053 | 45.484 | 34.812 | 1.00 | 97.82 |
| ATOM | 2258 | C | THR | 302 | 39.972 | 45.554 | 32.063 | 1.00 | 101.32 |
| ATOM | 2259 | O | THR | 302 | 39.126 | 46.448 | 32.038 | 1.00 | 101.86 |
| ATOM | 2260 | N | ALA | 303 | 39.687 | 44.275 | 31.836 | 1.00 | 103.31 |
| ATOM | 2261 | CA | ALA | 303 | 38.322 | 43.828 | 31.583 | 1.00 | 105.07 |
| ATOM | 2262 | CB | ALA | 303 | 38.320 | 42.572 | 30.714 | 1.00 | 104.35 |
| ATOM | 2263 | C | ALA | 303 | 37.733 | 43.530 | 32.961 | 1.00 | 106.78 |
| ATOM | 2264 | O | ALA | 303 | 38.480 | 43.314 | 33.918 | 1.00 | 106.64 |
| ATOM | 2265 | N | GLU | 304 | 36.406 | 43.529 | 33.065 | 1.00 | 108.54 |
| ATOM | 2266 | CA | GLU | 304 | 35.729 | 43.274 | 34.339 | 1.00 | 109.78 |
| ATOM | 2267 | CB | GLU | 304 | 34.251 | 42.959 | 34.107 | 1.00 | 110.34 |
| ATOM | 2268 | CG | GLU | 304 | 33.458 | 44.049 | 33.413 | 1.00 | 110.81 |
| ATOM | 2269 | CD | GLU | 304 | 31.973 | 43.724 | 33.360 | 1.00 | 111.13 |
| ATOM | 2270 | OE1 | GLU | 304 | 31.614 | 42.628 | 32.869 | 1.00 | 110.92 |
| ATOM | 2271 | OE2 | GLU | 304 | 31.167 | 44.567 | 33.814 | 1.00 | 111.09 |
| ATOM | 2272 | C | GLU | 304 | 36.345 | 42.118 | 35.117 | 1.00 | 110.69 |
| ATOM | 2273 | O | GLU | 304 | 36.803 | 42.290 | 36.251 | 1.00 | 111.35 |
| ATOM | 2274 | N | ASP | 305 | 36.338 | 40.939 | 34.500 | 1.00 | 110.93 |
| ATOM | 2275 | CA | ASP | 305 | 36.874 | 39.725 | 35.109 | 1.00 | 110.67 |
| ATOM | 2276 | CB | ASP | 305 | 36.698 | 38.541 | 34.144 | 1.00 | 110.84 |
| ATOM | 2277 | CG | ASP | 305 | 37.331 | 38.790 | 32.782 | 1.00 | 111.14 |
| ATOM | 2278 | OD1 | ASP | 305 | 37.777 | 39.927 | 32.519 | 1.00 | 111.75 |
| ATOM | 2279 | OD2 | ASP | 305 | 37.373 | 37.843 | 31.968 | 1.00 | 111.02 |
| ATOM | 2280 | C | ASP | 305 | 38.336 | 39.826 | 35.559 | 1.00 | 110.28 |
| ATOM | 2281 | O | ASP | 305 | 38.787 | 39.039 | 36.394 | 1.00 | 110.13 |
| ATOM | 2282 | N | GLY | 306 | 39.074 | 40.789 | 35.013 | 1.00 | 109.66 |
| ATOM | 2283 | CA | GLY | 306 | 40.467 | 40.948 | 35.395 | 1.00 | 108.63 |
| ATOM | 2284 | C | GLY | 306 | 41.439 | 40.719 | 34.257 | 1.00 | 107.62 |
| ATOM | 2285 | O | GLY | 306 | 42.580 | 41.180 | 34.305 | 1.00 | 108.33 |
| ATOM | 2286 | N | THR | 307 | 40.992 | 40.003 | 33.231 | 1.00 | 106.17 |
| ATOM | 2287 | CA | THR | 307 | 41.831 | 39.722 | 32.074 | 1.00 | 104.19 |
| ATOM | 2288 | CB | THR | 307 | 41.062 | 38.924 | 31.004 | 1.00 | 104.68 |
| ATOM | 2289 | OG1 | THR | 307 | 40.574 | 37.703 | 31.574 | 1.00 | 104.14 |
| ATOM | 2290 | CG2 | THR | 307 | 41.971 | 38.606 | 29.823 | 1.00 | 104.37 |
| ATOM | 2291 | C | THR | 307 | 42.300 | 41.026 | 31.442 | 1.00 | 103.03 |
| ATOM | 2292 | O | THR | 307 | 41.551 | 41.672 | 30.708 | 1.00 | 102.75 |
| ATOM | 2293 | N | GLN | 308 | 43.536 | 41.417 | 31.735 | 1.00 | 101.57 |
| ATOM | 2294 | CA | GLN | 308 | 44.087 | 42.643 | 31.175 | 1.00 | 100.15 |
| ATOM | 2295 | CB | GLN | 308 | 45.086 | 43.281 | 32.137 | 1.00 | 99.43 |
| ATOM | 2296 | CG | GLN | 308 | 44.471 | 43.874 | 33.385 | 1.00 | 99.19 |
| ATOM | 2297 | CD | GLN | 308 | 45.501 | 44.589 | 34.240 | 1.00 | 100.05 |
| ATOM | 2298 | OE1 | GLN | 308 | 46.469 | 43.983 | 34.697 | 1.00 | 100.13 |
| ATOM | 2299 | NE2 | GLN | 308 | 45.301 | 45.884 | 34.454 | 1.00 | 100.28 |
| ATOM | 2300 | C | GLN | 308 | 44.784 | 42.338 | 29.860 | 1.00 | 99.59 |
| ATOM | 2301 | O | GLN | 308 | 45.744 | 41.570 | 29.820 | 1.00 | 99.52 |
| ATOM | 2302 | N | ARG | 309 | 44.288 | 42.938 | 28.783 | 1.00 | 98.88 |
| ATOM | 2303 | CA | ARG | 309 | 44.867 | 42.733 | 27.467 | 1.00 | 97.82 |
| ATOM | 2304 | CB | ARG | 309 | 43.767 | 42.612 | 26.407 | 1.00 | 97.73 |
| ATOM | 2305 | CG | ARG | 309 | 42.669 | 41.612 | 26.738 | 1.00 | 98.41 |
| ATOM | 2306 | CD | ARG | 309 | 41.374 | 42.306 | 27.164 | 1.00 | 99.02 |
| ATOM | 2307 | NE | ARG | 309 | 40.752 | 43.094 | 26.010 | 1.00 | 98.96 |
| ATOM | 2308 | CZ | ARG | 309 | 39.449 | 43.716 | 26.380 | 1.00 | 98.63 |
| ATOM | 2309 | C | ARG | 309 | 45.747 | 43.922 | 27.132 | 1.00 | 97.75 |
| ATOM | 2310 | O | ARG | 309 | 45.816 | 44.889 | 27.889 | 1.00 | 97.57 |
| ATOM | 2311 | N | CYS | 310 | 46.433 | 43.832 | 26.000 | 1.00 | 97.78 |
| ATOM | 2312 | CA | CYs | 310 | 47.282 | 44.915 | 25.527 | 1.00 | 97.65 |
| ATOM | 2313 | C | CYS | 310 | 46.661 | 45.373 | 24.214 | 1.00 | 100.22 |
| ATOM | 2314 | O | CYS | 310 | 46.607 | 44.603 | 23.259 | 1.00 | 101.04 |
| ATOM | 2315 | CB | CYS | 310 | 48.704 | 44.428 | 25.254 | 1.00 | 94.17 |
| ATOM | 2316 | SG | CYS | 310 | 49.711 | 43.939 | 26.685 | 1.00 | 88.15 |
| ATOM | 2317 | N | GLU | 311 | 46.185 | 46.613 | 24.162 | 1.00 | 103.10 |
| ATOM | 2318 | CA | GLU | 311 | 45.573 | 47.134 | 22.942 | 1.00 | 105.28 |
| ATOM | 2319 | CB | GLU | 311 | 44.196 | 47.730 | 23.254 | 1.00 | 104.85 |
| ATOM | 2320 | CG | GLU | 311 | 43.048 | 46.757 | 23.046 | 1.00 | 105.54 |
| ATOM | 2321 | CD | GLU | 311 | 41.703 | 47.337 | 23.442 | 1.00 | 106.78 |
| ATOM | 2322 | OE1 | GLU | 311 | 41.383 | 48.469 | 23.014 | 1.00 | 107.43 |
| ATOM | 2323 | OE2 | GLU | 311 | 40.960 | 46.654 | 24.178 | 1.00 | 107.11 |
| ATOM | 2324 | C | GLU | 311 | 46.441 | 48.174 | 22.238 | 1.00 | 106.82 |
| ATOM | 2325 | O | GLU | 311 | 47.365 | 48.729 | 22.832 | 1.00 | 106.91 |
| ATOM | 2326 | N | LYS | 312 | 46.139 | 48.426 | 20.965 | 1.00 | 108.60 |
| ATOM | 2327 | CA | LYS | 312 | 46.883 | 49.401 | 20.174 | 1.00 | 109.53 |
| ATOM | 2328 | CB | LYS | 312 | 46.471 | 49.321 | 18.705 | 1.00 | 109.04 |
| ATOM | 2329 | C | LYS | 312 | 46.625 | 50.799 | 20.719 | 1.00 | 110.29 |
| ATOM | 2330 | O | LYS | 312 | 45.501 | 51.303 | 20.686 | 1.00 | 110.37 |
| ATOM | 2331 | N | CYS | 313 | 47.683 | 51.414 | 21.227 | 1.00 | 111.11 |
| ATOM | 2332 | CA | CYS | 313 | 47.609 | 52.749 | 21.796 | 1.00 | 111.89 |
| ATOM | 2333 | C | CYS | 313 | 48.738 | 53.504 | 21.100 | 1.00 | 112.43 |
| ATOM | 2334 | O | CYS | 313 | 49.352 | 54.408 | 21.664 | 1.00 | 112.94 |
| ATOM | 2335 | CB | CYS | 313 | 47.847 | 52.650 | 23.308 | 1.00 | 111.88 |
| ATOM | 2336 | SG | CYS | 313 | 47.192 | 53.962 | 24.389 | 1.00 | 112.06 |
| ATOM | 2337 | N | SER | 314 | 49.008 | 53.097 | 19.863 | 0.01 | 112.82 |
| ATOM | 2338 | CA | SER | 314 | 50.062 | 53.689 | 19.053 | 0.01 | 113.18 |
| ATOM | 2339 | CB | SER | 314 | 50.033 | 53.096 | 17.650 | 0.01 | 113.17 |
| ATOM | 2340 | C | SER | 314 | 49.953 | 55.202 | 18.979 | 0.01 | 113.47 |
| ATOM | 2341 | O | SER | 314 | 50.797 | 55.926 | 19.509 | 0.01 | 113.48 |
| ATOM | 2342 | N | LYS | 315 | 48.904 | 55.675 | 18.321 | 1.00 | 113.74 |
| ATOM | 2343 | CA | LYS | 315 | 48.694 | 57.105 | 18.165 | 1.00 | 114.23 |
| ATOM | 2344 | CB | LYS | 315 | 47.988 | 57.382 | 16.832 | 1.00 | 114.86 |
| ATOM | 2345 | CG | LYS | 315 | 48.771 | 56.924 | 15.609 | 1.00 | 114.98 |
| ATOM | 2346 | CD | LYS | 315 | 50.098 | 57.663 | 15.500 | 1.00 | 114.63 |
| ATOM | 2347 | CE | LYS | 315 | 50.837 | 57.298 | 14.225 | 1.00 | 114.17 |
| ATOM | 2348 | NZ | LYS | 315 | 52.118 | 58.046 | 14.103 | 1.00 | 113.31 |
| ATOM | 2349 | C | LYS | 315 | 47.936 | 57.753 | 19.328 | 1.00 | 113.77 |
| ATOM | 2350 | O | LYS | 315 | 48.546 | 58.446 | 20.144 | 1.00 | 114.04 |
| ATOM | 2351 | N | PRO | 316 | 46.605 | 57.544 | 19.428 | 1.00 | 113.20 |
| ATOM | 2352 | CD | PRO | 316 | 45.675 | 56.793 | 18.572 | 1.00 | 112.87 |
| ATOM | 2353 | CA | PRO | 316 | 45.874 | 58.165 | 20.541 | 1.00 | 112.47 |
| ATOM | 2354 | CB | PRO | 316 | 44.404 | 57.964 | 20.155 | 1.00 | 112.47 |
| ATOM | 2355 | CG | PRO | 316 | 44.443 | 57.634 | 18.679 | 1.00 | 112.56 |
| ATOM | 2356 | C | PRO | 316 | 46.213 | 57.433 | 21.836 | 1.00 | 111.90 |
| ATOM | 2357 | O | PRO | 316 | 45.640 | 56.374 | 22.116 | 1.00 | 112.00 |
| ATOM | 2358 | N | CYS | 317 | 47.132 | 57.984 | 22.627 | 1.00 | 110.08 |
| ATOM | 2359 | CA | CYS | 317 | 47.515 | 57.312 | 23.862 | 1.00 | 107.76 |
| ATOM | 2360 | C | CYS | 317 | 47.772 | 58.186 | 25.090 | 1.00 | 105.03 |
| ATOM | 2361 | O | CYS | 317 | 48.751 | 58.935 | 25.145 | 1.00 | 104.64 |
| ATOM | 2362 | CB | CYS | 317 | 48.735 | 56.431 | 23.602 | 1.00 | 108.86 |
| ATOM | 2363 | SG | CYS | 317 | 48.819 | 55.102 | 24.827 | 1.00 | 111.58 |
| ATOM | 2364 | N | ALA | 318 | 46.892 | 58.053 | 26.082 | 1.00 | 101.44 |
| ATOM | 2365 | CA | ALA | 318 | 46.982 | 58.809 | 27.331 | 1.00 | 98.34 |
| ATOM | 2366 | CB | ALA | 318 | 45.746 | 58.546 | 28.185 | 1.00 | 97.94 |
| ATOM | 2367 | C | ALA | 318 | 48.241 | 58.464 | 28.122 | 1.00 | 95.80 |
| ATOM | 2368 | O | ALA | 318 | 48.574 | 57.291 | 28.293 | 1.00 | 95.52 |
| ATOM | 2369 | N | ARG | 319 | 48.930 | 59.493 | 28.609 | 1.00 | 92.64 |
| ATOM | 2370 | CA | ARG | 319 | 50.154 | 59.315 | 29.387 | 1.00 | 89.08 |
| ATOM | 2371 | CB | ARG | 319 | 50.795 | 60.680 | 29.692 | 1.00 | 90.56 |
| ATOM | 2372 | CG | ARG | 319 | 49.875 | 61.703 | 30.371 | 1.00 | 91.95 |
| ATOM | 2373 | CD | ARG | 319 | 49.847 | 61.586 | 31.899 | 1.00 | 92.71 |
| ATOM | 2374 | NE | ARG | 319 | 51.142 | 61.897 | 32.506 | 1.00 | 93.30 |
| ATOM | 2375 | CZ | ARG | 319 | 51.349 | 62.018 | 33.815 | 1.00 | 93.09 |
| ATOM | 2376 | NH1 | ARG | 319 | 50.345 | 61.860 | 34.671 | 1.00 | 91.92 |
| ATOM | 2377 | NH2 | ARG | 319 | 52.567 | 62.289 | 34.270 | 1.00 | 92.71 |
| ATOM | 2378 | C | ARG | 319 | 49.899 | 58.559 | 30.688 | 1.00 | 85.21 |
| ATOM | 2379 | O | ARG | 319 | 48.840 | 58.692 | 31.306 | 1.00 | 85.67 |
| ATOM | 2380 | N | VAL | 320 | 50.878 | 57.760 | 31.097 | 1.00 | 79.32 |
| ATOM | 2381 | CA | VAL | 320 | 50.754 | 56.986 | 32.323 | 1.00 | 72.87 |
| ATOM | 2382 | CB | VAL | 320 | 50.689 | 55.473 | 32.024 | 1.00 | 71.59 |
| ATOM | 2383 | CG1 | VAL | 320 | 49.490 | 55.170 | 31.142 | 1.00 | 69.01 |
| ATOM | 2384 | CG2 | VAL | 320 | 51.980 | 55.014 | 31.360 | 1.00 | 68.56 |
| ATOM | 2385 | C | VAL | 320 | 51.936 | 57.248 | 33.242 | 1.00 | 69.92 |
| ATOM | 2386 | O | VAL | 320 | 52.937 | 57.843 | 32.841 | 1.00 | 68.57 |
| ATOM | 2387 | N | CYS | 321 | 51.811 | 56.805 | 34.485 | 1.00 | 65.95 |
| ATOM | 2388 | CA | CYS | 321 | 52.882 | 56.973 | 35.449 | 1.00 | 62.47 |
| ATOM | 2389 | C | CYS | 321 | 53.787 | 55.748 | 35.418 | 1.00 | 60.43 |
| ATOM | 2390 | O | CYS | 321 | 53.364 | 54.654 | 35.785 | 1.00 | 59.95 |
| ATOM | 2391 | CB | CYS | 321 | 52.303 | 57.122 | 36.845 | 1.00 | 62.31 |
| ATOM | 2392 | SG | CYS | 321 | 51.145 | 58.507 | 37.041 | 1.00 | 60.91 |
| ATOM | 2393 | N | TYR | 322 | 55.021 | 55.925 | 34.966 | 1.00 | 58.69 |
| ATOM | 2394 | CA | TYR | 322 | 55.962 | 54.821 | 34.929 | 1.00 | 57.74 |
| ATOM | 2395 | CB | TYR | 322 | 56.921 | 54.951 | 33.747 | 1.00 | 60.17 |
| ATOM | 2396 | CG | TYR | 322 | 56.281 | 54.638 | 32.420 | 1.00 | 62.41 |
| ATOM | 2397 | CD1 | TYR | 322 | 55.945 | 55.656 | 31.528 | 1.00 | 63.63 |
| ATOM | 2398 | CE1 | TYR | 322 | 55.315 | 55.372 | 30.318 | 1.00 | 64.44 |
| ATOM | 2399 | CD2 | TYR | 322 | 55.975 | 53.323 | 32.069 | 1.00 | 61.83 |
| ATOM | 2400 | CE2 | TYR | 322 | 55.343 | 53.029 | 30.865 | 1.00 | 63.82 |
| ATOM | 2401 | CZ | TYR | 322 | 55.016 | 54.057 | 29.995 | 1.00 | 64.36 |
| ATOM | 2402 | OH | TYR | 322 | 54.374 | 53.778 | 28.813 | 1.00 | 64.79 |
| ATOM | 2403 | C | TYR | 322 | 56.749 | 54.830 | 36.224 | 1.00 | 56.52 |
| ATOM | 2404 | O | TYR | 322 | 57.066 | 55.892 | 36.754 | 1.00 | 57.52 |
| ATOM | 2405 | N | GLY | 323 | 57.062 | 53.646 | 36.737 | 1.00 | 54.51 |
| ATOM | 2406 | CA | GLY | 323 | 57.803 | 53.570 | 37.979 | 1.00 | 50.35 |
| ATOM | 2407 | C | GLY | 323 | 59.208 | 53.077 | 37.749 | 1.00 | 48.48 |
| ATOM | 2408 | O | GLY | 323 | 59.654 | 52.971 | 36.605 | 1.00 | 46.95 |
| ATOM | 2409 | N | LEU | 324 | 59.906 | 52.775 | 38.840 | 1.00 | 47.45 |
| ATOM | 2410 | CA | LEU | 324 | 61.271 | 52.287 | 38.755 | 1.00 | 46.55 |
| ATOM | 2411 | CB | LEU | 324 | 61.821 | 52.000 | 40.154 | 1.00 | 47.15 |
| ATOM | 2412 | CG | LEU | 324 | 61.766 | 53.157 | 41.170 | 1.00 | 48.69 |
| ATOM | 2413 | CD1 | LEU | 324 | 62.424 | 52.736 | 42.484 | 1.00 | 48.11 |
| ATOM | 2414 | CD2 | LEU | 324 | 62.477 | 54.386 | 40.606 | 1.00 | 48.58 |
| ATOM | 2415 | C | LEU | 324 | 61.301 | 51.030 | 37.897 | 1.00 | 46.35 |
| ATOM | 2416 | O | LEU | 324 | 60.404 | 50.189 | 37.977 | 1.00 | 45.07 |
| ATOM | 2417 | N | GLY | 325 | 62.329 | 50.932 | 37.058 | 1.00 | 47.27 |
| ATOM | 2418 | CA | GLY | 325 | 62.486 | 49.793 | 36.178 | 1.00 | 47.12 |
| ATOM | 2419 | C | GLY | 325 | 61.836 | 50.005 | 34.824 | 1.00 | 49.50 |
| ATOM | 2420 | O | GLY | 325 | 62.022 | 49.204 | 33.904 | 1.00 | 49.51 |
| ATOM | 2421 | N | MET | 326 | 61.073 | 51.085 | 34.687 | 1.00 | 50.15 |
| ATOM | 2422 | CA | MET | 326 | 60.395 | 51.357 | 33.424 | 1.00 | 51.67 |
| ATOM | 2423 | CB | MET | 326 | 58.881 | 51.323 | 33.634 | 1.00 | 52.43 |
| ATOM | 2424 | CG | MET | 326 | 58.332 | 49.938 | 33.912 | 1.00 | 54.09 |
| ATOM | 2425 | SD | MET | 326 | 58.490 | 48.857 | 32.476 | 1.00 | 58.51 |
| ATOM | 2426 | CE | MET | 326 | 56.995 | 49.301 | 31.579 | 1.00 | 57.02 |
| ATOM | 2427 | C | MET | 326 | 60.790 | 52.676 | 32.764 | 1.00 | 52.23 |
| ATOM | 2428 | O | MET | 326 | 61.170 | 53.637 | 33.438 | 1.00 | 51.49 |
| ATOM | 2429 | N | GLU | 327 | 60.693 | 52.701 | 31.436 | 1.00 | 53.25 |
| ATOM | 2430 | CA | GLU | 327 | 61.017 | 53.875 | 30.624 | 1.00 | 54.18 |
| ATOM | 2431 | CB | GLU | 327 | 59.795 | 54.790 | 30.529 | 1.00 | 55.32 |
| ATOM | 2432 | CG | GLU | 327 | 58.654 | 54.175 | 29.738 | 1.00 | 58.20 |
| ATOM | 2433 | CD | GLU | 327 | 59.108 | 53.717 | 28.368 | 1.00 | 60.14 |
| ATOM | 2434 | OE1 | GLU | 327 | 59.744 | 54.531 | 27.673 | 1.00 | 62.16 |
| ATOM | 2435 | OE2 | GLU | 327 | 58.833 | 52.557 | 27.981 | 1.00 | 60.33 |
| ATOM | 2436 | C | GLU | 327 | 62.232 | 54.669 | 31.099 | 1.00 | 53.77 |
| ATOM | 2437 | O | GLU | 327 | 63.343 | 54.135 | 31.155 | 1.00 | 53.63 |
| ATOM | 2438 | N | HIS | 328 | 62.034 | 55.939 | 31.436 | 1.00 | 53.71 |
| ATOM | 2439 | CA | HIS | 328 | 63.160 | 56.741 | 31.882 | 1.00 | 54.74 |
| ATOM | 2440 | CB | HIS | 328 | 62.815 | 58.240 | 31.910 | 1.00 | 54.63 |
| ATOM | 2441 | CG | HIS | 328 | 61.685 | 58.603 | 32.817 | 1.00 | 53.25 |
| ATOM | 2442 | CD2 | HIS | 328 | 61.656 | 59.345 | 33.950 | 1.00 | 53.62 |
| ATOM | 2443 | ND1 | HIS | 328 | 60.378 | 58.240 | 32.565 | 1.00 | 53.71 |
| ATOM | 2444 | CE1 | HIS | 328 | 59.593 | 58.745 | 33.501 | 1.00 | 53.04 |
| ATOM | 2445 | NE2 | HIS | 328 | 60.343 | 59.420 | 34.353 | 1.00 | 55.48 |
| ATOM | 2446 | C | HIS | 328 | 63.707 | 56.311 | 33.223 | 1.00 | 55.75 |
| ATOM | 2447 | O | HIS | 328 | 64.892 | 56.498 | 33.488 | 1.00 | 57.58 |
| ATOM | 2448 | N | LEU | 329 | 62.862 | 55.709 | 34.058 | 1.00 | 56.08 |
| ATOM | 2449 | CA | LEU | 329 | 63.291 | 55.261 | 35.384 | 1.00 | 55.37 |
| ATOM | 2450 | CB | LEU | 329 | 62.110 | 55.322 | 36.362 | 1.00 | 54.06 |
| ATOM | 2451 | CG | LEU | 329 | 61.512 | 56.725 | 36.547 | 1.00 | 54.58 |
| ATOM | 2452 | CD1 | LEU | 329 | 60.185 | 56.665 | 37.287 | 1.00 | 53.35 |
| ATOM | 2453 | CD2 | LEU | 329 | 62.513 | 57.587 | 37.296 | 1.00 | 53.68 |
| ATOM | 2454 | C | LEU | 329 | 63.884 | 53.855 | 35.374 | 1.00 | 55.48 |
| ATOM | 2455 | O | LEU | 329 | 64.213 | 53.313 | 36.426 | 1.00 | 56.56 |
| ATOM | 2456 | N | ARG | 330 | 64.035 | 53.278 | 34.185 | 1.00 | 54.51 |
| ATOM | 2457 | CA | ARG | 330 | 64.567 | 51.929 | 34.042 | 1.00 | 55.09 |
| ATOM | 2458 | CB | ARG | 330 | 64.811 | 51.610 | 32.558 | 1.00 | 57.19 |
| ATOM | 2459 | CG | ARG | 330 | 65.548 | 50.287 | 32.322 | 1.00 | 61.45 |
| ATOM | 2460 | CD | ARG | 330 | 65.206 | 49.616 | 30.982 | 1.00 | 67.13 |
| ATOM | 2461 | NE | ARG | 330 | 65.373 | 50.493 | 29.817 | 1.00 | 72.58 |
| ATOM | 2462 | CZ | ARG | 330 | 64.371 | 51.106 | 29.183 | 1.00 | 75.31 |
| ATOM | 2463 | NH1 | ARG | 330 | 63.118 | 50.940 | 29.595 | 1.00 | 75.51 |
| ATOM | 2464 | NH2 | ARG | 330 | 64.616 | 51.887 | 28.132 | 1.00 | 76.41 |
| ATOM | 2465 | C | ARG | 330 | 65.825 | 51.598 | 34.841 | 1.00 | 54.88 |
| ATOM | 2466 | O | ARG | 330 | 66.009 | 50.454 | 35.257 | 1.00 | 55.54 |
| ATOM | 2467 | N | GLU | 331 | 66.693 | 52.581 | 35.065 | 1.00 | 55.30 |
| ATOM | 2468 | CA | GLU | 331 | 67.935 | 52.323 | 35.798 | 1.00 | 52.86 |
| ATOM | 2469 | CB | GLU | 331 | 69.144 | 52.838 | 35.001 | 1.00 | 54.33 |
| ATOM | 2470 | CG | GLU | 331 | 69.397 | 52.157 | 33.653 | 1.00 | 56.11 |
| ATOM | 2471 | CD | GLU | 331 | 68.403 | 52.567 | 32.570 | 1.00 | 58.11 |
| ATOM | 2472 | OE1 | GLU | 331 | 67.824 | 53.674 | 32.682 | 1.00 | 58.17 |
| ATOM | 2473 | OE2 | GLU | 331 | 68.220 | 51.793 | 31.596 | 1.00 | 56.57 |
| ATOM | 2474 | C | GLU | 331 | 67.970 | 52.930 | 37.196 | 1.00 | 51.54 |
| ATOM | 2475 | O | GLU | 331 | 68.918 | 52.702 | 37.951 | 1.00 | 50.00 |
| ATOM | 2476 | N | VAL | 332 | 66.950 | 53.717 | 37.530 | 1.00 | 51.04 |
| ATOM | 2477 | CA | VAL | 332 | 66.871 | 54.350 | 38.844 | 1.00 | 51.96 |
| ATOM | 2478 | CB | VAL | 332 | 65.764 | 55.414 | 38.886 | 1.00 | 51.13 |
| ATOM | 2479 | CG1 | VAL | 332 | 65.749 | 56.088 | 40.250 | 1.00 | 49.43 |
| ATOM | 2480 | CG2 | VAL | 332 | 65.984 | 56.435 | 37.780 | 1.00 | 48.05 |
| ATOM | 2481 | C | VAL | 332 | 66.574 | 53.285 | 39.892 | 1.00 | 53.80 |
| ATOM | 2482 | O | VAL | 332 | 65.620 | 52.518 | 39.756 | 1.00 | 54.40 |
| ATOM | 2483 | N | ARG | 333 | 67.384 | 53.236 | 40.942 | 1.00 | 56.37 |
| ATOM | 2484 | CA | ARG | 333 | 67.189 | 52.219 | 41.966 | 1.00 | 59.44 |
| ATOM | 2485 | CB | ARG | 333 | 68.532 | 51.602 | 42.370 | 1.00 | 60.59 |
| ATOM | 2486 | CG | ARG | 333 | 69.412 | 52.502 | 43.215 | 1.00 | 66.44 |
| ATOM | 2487 | CD | ARG | 333 | 69.823 | 53.776 | 42.474 | 1.00 | 71.63 |
| ATOM | 2488 | NE | ARG | 333 | 70.882 | 54.496 | 43.184 | 1.00 | 76.84 |
| ATOM | 2489 | CZ | ARG | 333 | 72.081 | 53.985 | 43.470 | 1.00 | 79.39 |
| ATOM | 2490 | NH1 | ARG | 333 | 72.387 | 52.739 | 43.107 | 1.00 | 79.11 |
| ATOM | 2491 | NH2 | ARG | 333 | 72.982 | 54.720 | 44.116 | 1.00 | 79.77 |
| ATOM | 2492 | C | ARG | 333 | 66.455 | 52.675 | 43.220 | 1.00 | 58.57 |
| ATOM | 2493 | O | ARG | 333 | 66.384 | 51.916 | 44.183 | 1.00 | 59.16 |
| ATOM | 2494 | N | ALA | 334 | 65.902 | 53.888 | 43.212 | 1.00 | 57.03 |
| ATOM | 2495 | CA | ALA | 334 | 65.191 | 54.383 | 44.388 | 1.00 | 55.85 |
| ATOM | 2496 | CB | ALA | 334 | 66.177 | 54.615 | 45.519 | 1.00 | 52.63 |
| ATOM | 2497 | C | ALA | 334 | 64.362 | 55.644 | 44.175 | 1.00 | 56.09 |
| ATOM | 2498 | O | ALA | 334 | 64.617 | 56.436 | 43.272 | 1.00 | 56.32 |
| ATOM | 2499 | N | VAL | 335 | 63.355 | 55.820 | 45.024 | 1.00 | 57.27 |
| ATOM | 2500 | CA | VAL | 335 | 62.498 | 56.997 | 44.965 | 1.00 | 57.41 |
| ATOM | 2501 | CB | VAL | 335 | 61.114 | 56.714 | 45.602 | 1.00 | 57.83 |
| ATOM | 2502 | CG1 | VAL | 335 | 60.357 | 58.006 | 45.828 | 1.00 | 56.01 |
| ATOM | 2503 | CG2 | VAL | 335 | 60.310 | 55.792 | 44.694 | 1.00 | 56.93 |
| ATOM | 2504 | C | VAL | 335 | 63.222 | 58.083 | 45.748 | 1.00 | 58.30 |
| ATOM | 2505 | O | VAL | 335 | 63.682 | 57.852 | 46.871 | 1.00 | 58.06 |
| ATOM | 2506 | N | THR | 336 | 63.342 | 59.263 | 45.149 | 1.00 | 59.37 |
| ATOM | 2507 | CA | THR | 336 | 64.039 | 60.365 | 45.800 | 1.00 | 59.83 |
| ATOM | 2508 | CB | THR | 336 | 65.463 | 60.511 | 45.266 | 1.00 | 58.36 |
| ATOM | 2509 | OG1 | THR | 336 | 65.402 | 61.041 | 43.939 | 1.00 | 59.09 |
| ATOM | 2510 | CG2 | THR | 336 | 66.175 | 59.159 | 45.232 | 1.00 | 57.89 |
| ATOM | 2511 | C | THR | 336 | 63.334 | 61.687 | 45.542 | 1.00 | 61.53 |
| ATOM | 2512 | O | THR | 336 | 62.333 | 61.747 | 44.822 | 1.00 | 62.10 |
| ATOM | 2513 | N | SER | 337 | 63.876 | 62.747 | 46.136 | 1.00 | 62.16 |
| ATOM | 2514 | CA | SER | 337 | 63.337 | 64.091 | 45.977 | 1.00 | 61.32 |
| ATOM | 2515 | CB | SER | 337 | 64.154 | 65.077 | 46.807 | 1.00 | 60.42 |
| ATOM | 2516 | OG | SER | 337 | 64.087 | 64.756 | 48.183 | 1.00 | 62.18 |
| ATOM | 2517 | C | SER | 337 | 63.413 | 64.485 | 44.508 | 1.00 | 61.14 |
| ATOM | 2518 | O | SER | 337 | 62.650 | 65.334 | 44.035 | 1.00 | 59.75 |
| ATOM | 2519 | N | ALA | 338 | 64.336 | 63.846 | 43.796 | 1.00 | 61.33 |
| ATOM | 2520 | CA | ALA | 338 | 64.556 | 64.114 | 42.381 | 1.00 | 62.09 |
| ATOM | 2521 | CB | ALA | 338 | 65.957 | 63.649 | 41.985 | 1.00 | 61.47 |
| ATOM | 2522 | C | ALA | 338 | 63.515 | 63.486 | 41.453 | 1.00 | 62.48 |
| ATOM | 2523 | O | ALA | 338 | 63.526 | 63.747 | 40.247 | 1.00 | 63.76 |
| ATOM | 2524 | N | ASN | 339 | 62.624 | 62.658 | 41.996 | 1.00 | 61.25 |
| ATOM | 2525 | CA | ASN | 339 | 61.597 | 62.017 | 41.173 | 1.00 | 60.09 |
| ATOM | 2526 | CB | ASN | 339 | 62.092 | 60.652 | 40.672 | 1.00 | 58.96 |
| ATOM | 2527 | CG | ASN | 339 | 62.569 | 59.741 | 41.801 | 1.00 | 59.46 |
| ATOM | 2528 | OD1 | ASN | 339 | 61.845 | 59.486 | 42.755 | 1.00 | 60.66 |
| ATOM | 2529 | ND2 | ASN | 339 | 63.791 | 59.239 | 41.682 | 1.00 | 59.53 |
| ATOM | 2530 | C | ASN | 339 | 60.248 | 61.851 | 41.873 | 1.00 | 60.07 |
| ATOM | 2531 | O | ASN | 339 | 59.242 | 61.546 | 41.239 | 1.00 | 60.23 |
| ATOM | 2532 | N | ILE | 340 | 60.228 | 62.070 | 43.180 | 1.00 | 59.91 |
| ATOM | 2533 | CA | ILE | 340 | 59.011 | 61.920 | 43.958 | 1.00 | 61.02 |
| ATOM | 2534 | CB | ILE | 340 | 59.236 | 62.413 | 45.417 | 1.00 | 61.44 |
| ATOM | 2535 | CG2 | ILE | 340 | 59.850 | 63.807 | 45.413 | 1.00 | 60.10 |
| ATOM | 2536 | CG1 | ILE | 340 | 57.918 | 62.368 | 46.196 | 1.00 | 60.95 |
| ATOM | 2537 | CD1 | ILE | 340 | 57.392 | 60.970 | 46.422 | 1.00 | 59.08 |
| ATOM | 2538 | C | ILE | 340 | 57.785 | 62.617 | 43.359 | 1.00 | 61.93 |
| ATOM | 2539 | O | ILE | 340 | 56.683 | 62.056 | 43.362 | 1.00 | 61.85 |
| ATOM | 2540 | N | GLN | 341 | 57.966 | 63.830 | 42.843 | 1.00 | 63.02 |
| ATOM | 2541 | CA | GLN | 341 | 56.847 | 64.575 | 42.268 | 1.00 | 63.53 |
| ATOM | 2542 | CB | GLN | 341 | 57.258 | 66.018 | 41.958 | 1.00 | 65.29 |
| ATOM | 2543 | CG | GLN | 341 | 56.910 | 67.016 | 43.070 | 1.00 | 66.87 |
| ATOM | 2544 | CD | GLN | 341 | 55.411 | 67.055 | 43.386 | 1.00 | 67.11 |
| ATOM | 2545 | OE1 | GLN | 341 | 54.579 | 67.344 | 42.516 | 1.00 | 66.49 |
| ATOM | 2546 | NE2 | GLN | 341 | 55.066 | 66.762 | 44.636 | 1.00 | 65.92 |
| ATOM | 2547 | C | GLN | 341 | 56.238 | 63.944 | 41.025 | 1.00 | 63.04 |
| ATOM | 2548 | O | GLN | 341 | 55.080 | 64.204 | 40.702 | 1.00 | 64.33 |
| ATOM | 2549 | N | GLU | 342 | 57.011 | 63.112 | 40.333 | 1.00 | 60.92 |
| ATOM | 2550 | CA | GLU | 342 | 56.528 | 62.450 | 39.126 | 1.00 | 59.81 |
| ATOM | 2551 | CB | GLU | 342 | 57.655 | 61.627 | 38.490 | 1.00 | 60.69 |
| ATOM | 2552 | CG | GLU | 342 | 58.774 | 62.431 | 37.861 | 1.00 | 61.90 |
| ATOM | 2553 | CD | GLU | 342 | 59.878 | 61.543 | 37.304 | 1.00 | 64.99 |
| ATOM | 2554 | OE1 | GLU | 342 | 59.547 | 60.520 | 36.660 | 1.00 | 65.22 |
| ATOM | 2555 | OE2 | GLU | 342 | 61.073 | 61.871 | 37.498 | 1.00 | 65.92 |
| ATOM | 2556 | C | GLU | 342 | 55.324 | 61.529 | 39.377 | 1.00 | 58.93 |
| ATOM | 2557 | O | GLU | 342 | 54.658 | 61.093 | 38.431 | 1.00 | 59.33 |
| ATOM | 2558 | N | PHE | 343 | 55.031 | 61.237 | 40.641 | 1.00 | 56.77 |
| ATOM | 2559 | CA | PHE | 343 | 53.928 | 60.332 | 40.944 | 1.00 | 54.80 |
| ATOM | 2560 | CB | PHE | 343 | 54.373 | 59.303 | 41.980 | 1.00 | 51.72 |
| ATOM | 2561 | CG | PHE | 343 | 55.591 | 58.538 | 41.565 | 1.00 | 48.42 |
| ATOM | 2562 | CD1 | PHE | 343 | 56.796 | 58.704 | 42.238 | 1.00 | 46.96 |
| ATOM | 2563 | CD2 | PHE | 343 | 55.548 | 57.689 | 40.467 | 1.00 | 47.13 |
| ATOM | 2564 | CE1 | PHE | 343 | 57.940 | 58.040 | 41.824 | 1.00 | 44.73 |
| ATOM | 2565 | CE2 | PHE | 343 | 56.688 | 57.019 | 40.045 | 1.00 | 46.48 |
| ATOM | 2566 | CZ | PHE | 343 | 57.889 | 57.197 | 40.727 | 1.00 | 44.96 |
| ATOM | 2567 | C | PHE | 343 | 52.660 | 61.009 | 41.399 | 1.00 | 54.34 |
| ATOM | 2568 | O | PHE | 343 | 51.655 | 60.344 | 41.662 | 1.00 | 53.92 |
| ATOM | 2569 | N | ALA | 344 | 52.704 | 62.333 | 41.483 | 1.00 | 53.73 |
| ATOM | 2570 | CA | ALA | 344 | 51.541 | 63.107 | 41.894 | 1.00 | 52.84 |
| ATOM | 2571 | CB | ALA | 344 | 51.797 | 64.577 | 41.640 | 1.00 | 52.20 |
| ATOM | 2572 | C | ALA | 344 | 50.286 | 62.658 | 41.140 | 1.00 | 53.05 |
| ATOM | 2573 | O | ALA | 344 | 50.287 | 62.559 | 39.910 | 1.00 | 52.61 |
| ATOM | 2574 | N | GLY | 345 | 49.222 | 62.374 | 41.888 | 1.00 | 53.32 |
| ATOM | 2575 | CA | GLY | 345 | 47.965 | 61.968 | 41.283 | 1.00 | 53.54 |
| ATOM | 2576 | C | GLY | 345 | 47.972 | 60.678 | 40.482 | 1.00 | 55.19 |
| ATOM | 2577 | O | GLY | 345 | 47.063 | 60.422 | 39.682 | 1.00 | 54.90 |
| ATOM | 2578 | N | CYS | 346 | 48.989 | 59.852 | 40.686 | 1.00 | 54.98 |
| ATOM | 2579 | CA | CYS | 346 | 49.064 | 58.591 | 39.969 | 1.00 | 55.73 |
| ATOM | 2580 | C | CYS | 346 | 48.192 | 57.548 | 40.658 | 1.00 | 56.57 |
| ATOM | 2581 | O | CYS | 346 | 48.427 | 57.218 | 41.817 | 1.00 | 58.72 |
| ATOM | 2582 | CB | CYS | 346 | 50.510 | 58.118 | 39.928 | 1.00 | 57.10 |
| ATOM | 2583 | SG | CYS | 346 | 51.587 | 59.170 | 38.905 | 1.00 | 59.00 |
| ATOM | 2584 | N | LYS | 347 | 47.174 | 57.042 | 39.966 | 1.00 | 56.79 |
| ATOM | 2585 | CA | LYS | 347 | 46.305 | 56.025 | 40.559 | 1.00 | 56.60 |
| ATOM | 2586 | CB | LYS | 347 | 44.938 | 55.984 | 39.866 | 1.00 | 56.84 |
| ATOM | 2587 | CG | LYS | 347 | 44.073 | 57.208 | 40.113 | 1.00 | 60.70 |
| ATOM | 2588 | CD | LYS | 347 | 42.609 | 56.931 | 39.810 | 0.01 | 59.47 |
| ATOM | 2589 | CE | LYS | 347 | 41.733 | 58.096 | 40.247 | 0.01 | 59.77 |
| ATOM | 2590 | NZ | LYS | 347 | 40.283 | 57.799 | 40.085 | 0.01 | 59.55 |
| ATOM | 2591 | C | LYS | 347 | 46.965 | 54.672 | 40.391 | 1.00 | 56.47 |
| ATOM | 2592 | O | LYS | 347 | 46.939 | 53.817 | 41.282 | 1.00 | 54.68 |
| ATOM | 2593 | N | LYS | 348 | 47.570 | 54.501 | 39.226 | 1.00 | 55.45 |
| ATOM | 2594 | CA | LYS | 348 | 48.219 | 53.261 | 38.873 | 1.00 | 53.89 |
| ATOM | 2595 | CB | LYS | 348 | 47.436 | 52.625 | 37.722 | 1.00 | 53.77 |
| ATOM | 2596 | CG | LYS | 348 | 47.806 | 51.205 | 37.384 | 1.00 | 57.56 |
| ATOM | 2597 | CD | LYS | 348 | 47.031 | 50.747 | 36.160 | 1.00 | 60.25 |
| ATOM | 2598 | CE | LYS | 348 | 47.394 | 49.329 | 35.769 | 1.00 | 62.70 |
| ATOM | 2599 | NZ | LYS | 348 | 46.633 | 48.881 | 34.569 | 1.00 | 66.48 |
| ATOM | 2600 | C | LYS | 348 | 49.653 | 53.557 | 38.459 | 1.00 | 52.28 |
| ATOM | 2601 | O | LYS | 348 | 49.920 | 54.560 | 37.794 | 1.00 | 53.57 |
| ATOM | 2602 | N | ILE | 349 | 50.582 | 52.700 | 38.872 | 1.00 | 49.65 |
| ATOM | 2603 | CA | ILE | 349 | 51.979 | 52.882 | 38.510 | 1.00 | 47.48 |
| ATOM | 2604 | CB | ILE | 349 | 52.854 | 53.153 | 39.732 | 1.00 | 45.57 |
| ATOM | 2605 | CG2 | ILE | 349 | 54.325 | 52.998 | 39.346 | 1.00 | 46.75 |
| ATOM | 2606 | CG1 | ILE | 349 | 52.575 | 54.561 | 40.259 | 1.00 | 45.22 |
| ATOM | 2607 | CD1 | ILE | 349 | 53.449 | 54.981 | 41.421 | 1.00 | 43.85 |
| ATOM | 2608 | C | ILE | 349 | 52.527 | 51.666 | 37.780 | 1.00 | 46.46 |
| ATOM | 2609 | O | ILE | 349 | 52.541 | 50.560 | 38.313 | 1.00 | 45.46 |
| ATOM | 2610 | N | PHE | 350 | 52.981 | 51.884 | 36.554 | 1.00 | 45.65 |
| ATOM | 2611 | CA | PHE | 350 | 53.528 | 50.809 | 35.740 | 1.00 | 45.55 |
| ATOM | 2612 | CB | PHE | 350 | 53.276 | 51.106 | 34.259 | 1.00 | 46.63 |
| ATOM | 2613 | CG | PHE | 350 | 51.816 | 51.317 | 33.928 | 1.00 | 47.66 |
| ATOM | 2614 | CD1 | PHE | 350 | 51.181 | 52.513 | 34.243 | 1.00 | 48.14 |
| ATOM | 2615 | CD2 | PHE | 350 | 51.072 | 50.309 | 33.325 | 1.00 | 49.55 |
| ATOM | 2616 | CE1 | PHE | 350 | 49.821 | 52.703 | 33.965 | 1.00 | 48.95 |
| ATOM | 2617 | CE2 | PHE | 350 | 49.707 | 50.491 | 33.042 | 1.00 | 50.22 |
| ATOM | 2618 | CZ | PHE | 350 | 49.086 | 51.688 | 33.365 | 1.00 | 49.30 |
| ATOM | 2619 | C | PHE | 350 | 55.014 | 50.693 | 36.039 | 1.00 | 43.17 |
| ATOM | 2620 | O | PHE | 350 | 55.830 | 51.416 | 35.479 | 1.00 | 43.46 |
| ATOM | 2621 | N | GLY | 351 | 55.341 | 49.773 | 36.939 | 1.00 | 42.05 |
| ATOM | 2622 | CA | GLY | 351 | 56.712 | 49.578 | 37.370 | 1.00 | 41.42 |
| ATOM | 2623 | C | GLY | 351 | 56.740 | 49.343 | 38.875 | 1.00 | 41.84 |
| ATOM | 2624 | O | GLY | 351 | 55.744 | 48.905 | 39.452 | 1.00 | 43.39 |
| ATOM | 2625 | N | SER | 352 | 57.857 | 49.658 | 39.523 | 1.00 | 40.45 |
| ATOM | 2626 | CA | SER | 352 | 57.990 | 49.427 | 40.952 | 1.00 | 38.91 |
| ATOM | 2627 | CB | SER | 352 | 59.028 | 48.332 | 41.195 | 1.00 | 39.49 |
| ATOM | 2628 | OG | SER | 352 | 58.712 | 47.165 | 40.452 | 1.00 | 39.72 |
| ATOM | 2629 | C | SER | 352 | 58.372 | 50.649 | 41.771 | 1.00 | 39.34 |
| ATOM | 2630 | O | SER | 352 | 58.798 | 51.663 | 41.238 | 1.00 | 39.29 |
| ATOM | 2631 | N | LEU | 353 | 58.209 | 50.536 | 43.084 | 1.00 | 39.89 |
| ATOM | 2632 | CA | LEU | 353 | 58.556 | 51.608 | 44.005 | 1.00 | 39.25 |
| ATOM | 2633 | CB | LEU | 353 | 57.312 | 52.148 | 44.712 | 1.00 | 38.43 |
| ATOM | 2634 | CG | LEU | 353 | 56.288 | 52.925 | 43.887 | 1.00 | 37.43 |
| ATOM | 2635 | CD1 | LEU | 353 | 55.179 | 53.438 | 44.808 | 1.00 | 35.64 |
| ATOM | 2636 | CD2 | LEU | 353 | 56.980 | 54.089 | 43.190 | 1.00 | 37.71 |
| ATOM | 2637 | C | LEU | 353 | 59.504 | 51.030 | 45.032 | 1.00 | 38.86 |
| ATOM | 2638 | O | LEU | 353 | 59.224 | 49.994 | 45.633 | 1.00 | 42.35 |
| ATOM | 2639 | N | ALA | 354 | 60.636 | 51.685 | 45.227 | 1.00 | 38.83 |
| ATOM | 2640 | CA | ALA | 354 | 61.620 | 51.210 | 46.198 | 1.00 | 39.22 |
| ATOM | 2641 | CB | ALA | 354 | 62.766 | 50.480 | 45.481 | 1.00 | 34.77 |
| ATOM | 2642 | C | ALA | 354 | 62.169 | 52.384 | 47.002 | 1.00 | 39.17 |
| ATOM | 2643 | O | ALA | 354 | 62.535 | 53.425 | 46.444 | 1.00 | 39.80 |
| ATOM | 2644 | N | PHE | 355 | 62.204 | 52.215 | 48.316 | 1.00 | 39.01 |
| ATOM | 2645 | CA | PHE | 355 | 62.708 | 53.248 | 49.198 | 1.00 | 40.18 |
| ATOM | 2646 | CB | PHE | 355 | 61.615 | 53.702 | 50.177 | 1.00 | 39.58 |
| ATOM | 2647 | CG | PHE | 355 | 60.340 | 54.134 | 49.497 | 1.00 | 38.96 |
| ATOM | 2648 | CD1 | PHE | 355 | 59.444 | 53.191 | 49.009 | 1.00 | 37.60 |
| ATOM | 2649 | CD2 | PHE | 355 | 60.060 | 55.485 | 49.304 | 1.00 | 38.55 |
| ATOM | 2650 | CE1 | PHE | 355 | 58.285 | 53.585 | 48.332 | 1.00 | 39.45 |
| ATOM | 2651 | CE2 | PHE | 355 | 58.905 | 55.891 | 48.630 | 1.00 | 38.69 |
| ATOM | 2652 | CZ | PHE | 355 | 58.016 | 54.938 | 48.142 | 1.00 | 38.52 |
| ATOM | 2653 | C | PHE | 355 | 63.880 | 52.665 | 49.949 | 1.00 | 40.41 |
| ATOM | 2654 | O | PHE | 355 | 63.750 | 51.635 | 50.604 | 1.00 | 41.91 |
| ATOM | 2655 | N | LEU | 356 | 65.032 | 53.312 | 49.835 | 1.00 | 41.13 |
| ATOM | 2656 | CA | LEU | 356 | 66.230 | 52.838 | 50.513 | 1.00 | 41.92 |
| ATOM | 2657 | CB | LEU | 356 | 67.298 | 52.463 | 49.479 | 1.00 | 42.83 |
| ATOM | 2658 | CG | LEU | 356 | 67.051 | 51.250 | 48.578 | 1.00 | 45.38 |
| ATOM | 2659 | CD1 | LEU | 356 | 65.821 | 51.477 | 47.695 | 1.00 | 45.56 |
| ATOM | 2660 | CD2 | LEU | 356 | 68.288 | 51.012 | 47.718 | 1.00 | 46.11 |
| ATOM | 2661 | C | LEU | 356 | 66.773 | 53.917 | 51.440 | 1.00 | 41.93 |
| ATOM | 2662 | O | LEU | 356 | 66.251 | 55.026 | 51.481 | 1.00 | 40.02 |
| ATOM | 2663 | N | PRO | 357 | 67.816 | 53.594 | 52.219 | 1.00 | 44.66 |
| ATOM | 2664 | CD | PRO | 357 | 68.397 | 52.253 | 52.435 | 1.00 | 44.52 |
| ATOM | 2665 | CA | PRO | 357 | 68.409 | 54.571 | 53.134 | 1.00 | 46.97 |
| ATOM | 2666 | CB | PRO | 357 | 69.730 | 53.915 | 53.506 | 1.00 | 45.42 |
| ATOM | 2667 | CG | PRO | 357 | 69.333 | 52.476 | 53.622 | 1.00 | 45.44 |
| ATOM | 2668 | C | PRO | 357 | 68.606 | 55.914 | 52.432 | 1.00 | 50.51 |
| ATOM | 2669 | O | PRO | 357 | 68.132 | 56.958 | 52.902 | 1.00 | 51.44 |
| ATOM | 2670 | N | GLU | 358 | 69.295 | 55.872 | 51.295 | 1.00 | 52.17 |
| ATOM | 2671 | CA | GLU | 358 | 69.568 | 57.072 | 50.513 | 1.00 | 53.67 |
| ATOM | 2672 | CB | GLU | 358 | 70.243 | 56.688 | 49.194 | 1.00 | 55.12 |
| ATOM | 2673 | CG | GLU | 358 | 69.738 | 55.382 | 48.617 | 1.00 | 57.84 |
| ATOM | 2674 | CD | GLU | 358 | 70.386 | 55.030 | 47.296 | 1.00 | 58.68 |
| ATOM | 2675 | OE1 | GLU | 358 | 70.171 | 55.782 | 46.315 | 1.00 | 57.40 |
| ATOM | 2676 | OE2 | GLU | 358 | 71.105 | 54.002 | 47.243 | 1.00 | 57.52 |
| ATOM | 2677 | C | GLU | 358 | 68.325 | 57.906 | 50.232 | 1.00 | 53.93 |
| ATOM | 2678 | O | GLU | 358 | 68.398 | 59.137 | 50.187 | 1.00 | 53.80 |
| ATOM | 2679 | N | SER | 359 | 67.188 | 57.240 | 50.044 | 1.00 | 54.11 |
| ATOM | 2680 | CA | SER | 359 | 65.942 | 57.946 | 49.759 | 1.00 | 55.21 |
| ATOM | 2681 | CB | SER | 359 | 64.745 | 56.991 | 49.789 | 1.00 | 53.38 |
| ATOM | 2682 | OG | SER | 359 | 64.804 | 56.030 | 48.755 | 1.00 | 51.19 |
| ATOM | 2683 | C | SER | 359 | 65.707 | 59.049 | 50.776 | 1.00 | 56.90 |
| ATOM | 2684 | O | SER | 359 | 65.442 | 60.197 | 50.415 | 1.00 | 57.15 |
| ATOM | 2685 | N | PHE | 360 | 65.817 | 58.702 | 52.054 | 1.00 | 58.29 |
| ATOM | 2686 | CA | PHE | 360 | 65.583 | 59.682 | 53.102 | 1.00 | 60.53 |
| ATOM | 2687 | CB | PHE | 360 | 64.794 | 59.036 | 54.239 | 1.00 | 55.67 |
| ATOM | 2688 | CG | PHE | 360 | 63.556 | 58.357 | 53.766 | 1.00 | 52.83 |
| ATOM | 2689 | CD1 | PHE | 360 | 63.555 | 56.991 | 53.499 | 1.00 | 50.96 |
| ATOM | 2690 | CD2 | PHE | 360 | 62.418 | 59.099 | 53.471 | 1.00 | 51.66 |
| ATOM | 2691 | CE1 | PHE | 360 | 62.441 | 56.378 | 52.942 | 1.00 | 48.89 |
| ATOM | 2692 | CE2 | PHE | 360 | 61.297 | 58.491 | 52.911 | 1.00 | 49.47 |
| ATOM | 2693 | CZ | PHE | 360 | 61.311 | 57.130 | 52.646 | 1.00 | 47.46 |
| ATOM | 2694 | C | PHE | 360 | 66.851 | 60.347 | 53.607 | 1.00 | 62.88 |
| ATOM | 2695 | O | PHE | 360 | 66.785 | 61.388 | 54.259 | 1.00 | 62.93 |
| ATOM | 2696 | N | ASP | 361 | 68.002 | 59.751 | 53.300 | 1.00 | 65.54 |
| ATOM | 2697 | CA | ASP | 361 | 69.277 | 60.332 | 53.700 | 1.00 | 67.51 |
| ATOM | 2698 | CB | ASP | 361 | 70.425 | 59.325 | 53.556 | 1.00 | 66.61 |
| ATOM | 2699 | CG | ASP | 361 | 70.430 | 58.263 | 54.642 | 1.00 | 66.17 |
| ATOM | 2700 | OD1 | ASP | 361 | 69.825 | 58.483 | 55.716 | 1.00 | 63.22 |
| ATOM | 2701 | OD2 | ASP | 361 | 71.063 | 57.208 | 54.419 | 1.00 | 66.05 |
| ATOM | 2702 | C | ASP | 361 | 69.543 | 61.508 | 52.764 | 1.00 | 69.79 |
| ATOM | 2703 | O | ASP | 361 | 70.107 | 62.531 | 53.167 | 1.00 | 69.60 |
| ATOM | 2704 | N | GLY | 362 | 69.118 | 61.353 | 51.512 | 1.00 | 72.02 |
| ATOM | 2705 | CA | GLY | 362 | 69.335 | 62.388 | 50.518 | 1.00 | 73.73 |
| ATOM | 2706 | C | GLY | 362 | 70.783 | 62.338 | 50.073 | 1.00 | 74.63 |
| ATOM | 2707 | O | GLY | 362 | 71.641 | 61.849 | 50.805 | 1.00 | 73.91 |
| ATOM | 2708 | N | ASP | 363 | 71.064 | 62.819 | 48.869 | 1.00 | 76.81 |
| ATOM | 2709 | CA | ASP | 363 | 72.436 | 62.819 | 48.380 | 1.00 | 79.49 |
| ATOM | 2710 | CB | ASP | 363 | 72.563 | 61.984 | 47.100 | 1.00 | 80.20 |
| ATOM | 2711 | CG | ASP | 363 | 74.010 | 61.796 | 46.665 | 1.00 | 80.95 |
| ATOM | 2712 | OD1 | ASP | 363 | 74.842 | 61.403 | 47.515 | 1.00 | 80.92 |
| ATOM | 2713 | OD2 | ASP | 363 | 74.314 | 62.033 | 45.474 | 1.00 | 81.00 |
| ATOM | 2714 | C | ASP | 363 | 72.883 | 64.252 | 48.116 | 1.00 | 80.85 |
| ATOM | 2715 | O | ASP | 363 | 72.604 | 64.817 | 47.054 | 1.00 | 80.38 |
| ATOM | 2716 | N | PRO | 364 | 73.566 | 64.866 | 49.101 | 1.00 | 81.73 |
| ATOM | 2717 | CD | PRO | 364 | 73.770 | 64.322 | 50.458 | 1.00 | 81.93 |
| ATOM | 2718 | CA | PRO | 364 | 74.070 | 66.240 | 49.015 | 1.00 | 81.54 |
| ATOM | 2719 | CB | PRO | 364 | 74.791 | 66.422 | 50.348 | 1.00 | 81.54 |
| ATOM | 2720 | CG | PRO | 364 | 73.966 | 65.575 | 51.279 | 1.00 | 81.80 |
| ATOM | 2721 | C | PRO | 364 | 74.994 | 66.450 | 47.816 | 1.00 | 81.87 |
| ATOM | 2722 | O | PRO | 364 | 75.011 | 67.530 | 47.220 | 1.00 | 81.95 |
| ATOM | 2723 | N | ALA | 365 | 75.756 | 65.413 | 47.469 | 1.00 | 81.95 |
| ATOM | 2724 | CA | ALA | 365 | 76.676 | 65.471 | 46.334 | 1.00 | 82.33 |
| ATOM | 2725 | CB | ALA | 365 | 77.417 | 64.141 | 46.186 | 1.00 | 81.35 |
| ATOM | 2726 | C | ALA | 365 | 75.911 | 65.797 | 45.050 | 1.00 | 82.82 |
| ATOM | 2727 | O | ALA | 365 | 76.395 | 66.547 | 44.201 | 1.00 | 83.65 |
| ATOM | 2728 | N | SER | 366 | 74.715 | 65.233 | 44.913 | 1.00 | 82.50 |
| ATOM | 2729 | CA | SER | 366 | 73.885 | 65.484 | 43.740 | 1.00 | 82.52 |
| ATOM | 2730 | CB | SER | 366 | 73.148 | 64.206 | 43.329 | 1.00 | 82.26 |
| ATOM | 2731 | OG | SER | 366 | 72.287 | 63.753 | 44.360 | 1.00 | 83.81 |
| ATOM | 2732 | C | SER | 366 | 72.876 | 66.592 | 44.054 | 1.00 | 82.34 |
| ATOM | 2733 | O | SER | 366 | 72.016 | 66.925 | 43.231 | 1.00 | 81.76 |
| ATOM | 2734 | N | ASN | 367 | 73.002 | 67.162 | 45.250 | 1.00 | 82.16 |
| ATOM | 2735 | CA | ASN | 367 | 72.115 | 68.224 | 45.721 | 1.00 | 82.06 |
| ATOM | 2736 | CB | ASN | 367 | 72.221 | 69.465 | 44.830 | 1.00 | 81.56 |
| ATOM | 2737 | CG | ASN | 367 | 71.427 | 70.634 | 45.377 | 1.00 | 80.51 |
| ATOM | 2738 | OD1 | ASN | 367 | 71.597 | 71.027 | 46.531 | 1.00 | 79.99 |
| ATOM | 2739 | ND2 | ASN | 367 | 70.553 | 71.194 | 44.554 | 1.00 | 81.11 |
| ATOM | 2740 | C | ASN | 367 | 70.661 | 67.756 | 45.785 | 1.00 | 81.68 |
| ATOM | 2741 | O | ASN | 367 | 69.723 | 68.565 | 45.747 | 1.00 | 81.41 |
| ATOM | 2742 | N | THR | 368 | 70.486 | 66.439 | 45.872 | 1.00 | 80.67 |
| ATOM | 2743 | CA | THR | 368 | 69.167 | 65.832 | 45.979 | 1.00 | 78.44 |
| ATOM | 2744 | CB | THR | 368 | 69.172 | 64.390 | 45.435 | 1.00 | 78.87 |
| ATOM | 2745 | OG1 | THR | 368 | 70.291 | 63.679 | 45.982 | 1.00 | 78.61 |
| ATOM | 2746 | CG2 | THR | 368 | 69.270 | 64.395 | 43.913 | 1.00 | 78.71 |
| ATOM | 2747 | C | THR | 368 | 68.846 | 65.822 | 47.470 | 1.00 | 77.12 |
| ATOM | 2748 | O | THR | 368 | 69.304 | 64.953 | 48.214 | 1.00 | 76.03 |
| ATOM | 2749 | N | ALA | 369 | 68.077 | 66.816 | 47.901 | 1.00 | 76.38 |
| ATOM | 2750 | CA | ALA | 369 | 67.709 | 66.944 | 49.300 | 1.00 | 76.74 |
| ATOM | 2751 | CB | ALA | 369 | 66.828 | 68.169 | 49.498 | 1.00 | 76.05 |
| ATOM | 2752 | C | ALA | 369 | 66.984 | 65.696 | 49.782 | 1.00 | 77.16 |
| ATOM | 2753 | O | ALA | 369 | 66.275 | 65.042 | 49.016 | 1.00 | 78.07 |
| ATOM | 2754 | N | PRO | 370 | 67.173 | 65.332 | 51.059 | 1.00 | 76.47 |
| ATOM | 2755 | CD | PRO | 370 | 68.080 | 65.903 | 52.075 | 1.00 | 75.55 |
| ATOM | 2756 | CA | PRO | 370 | 66.492 | 64.143 | 51.570 | 1.00 | 74.73 |
| ATOM | 2757 | CB | PRO | 370 | 66.781 | 64.208 | 53.063 | 1.00 | 75.53 |
| ATOM | 2758 | CG | PRO | 370 | 68.175 | 64.779 | 53.089 | 1.00 | 75.54 |
| ATOM | 2759 | C | PRO | 370 | 65.003 | 64.232 | 51.258 | 1.00 | 73.48 |
| ATOM | 2760 | O | PRO | 370 | 64.473 | 65.313 | 50.992 | 1.00 | 72.25 |
| ATOM | 2761 | N | LEU | 371 | 64.331 | 63.092 | 51.267 | 1.00 | 72.66 |
| ATOM | 2762 | CA | LEU | 371 | 62.909 | 63.078 | 50.987 | 1.00 | 70.92 |
| ATOM | 2763 | CB | LEU | 371 | 62.488 | 61.699 | 50.484 | 1.00 | 70.30 |
| ATOM | 2764 | CG | LEU | 371 | 61.186 | 61.639 | 49.689 | 1.00 | 70.82 |
| ATOM | 2765 | CD1 | LEU | 371 | 61.188 | 62.676 | 48.576 | 1.00 | 70.62 |
| ATOM | 2766 | CD2 | LEU | 371 | 61.034 | 60.249 | 49.110 | 1.00 | 71.78 |
| ATOM | 2767 | C | LEU | 371 | 62.231 | 63.419 | 52.304 | 1.00 | 70.26 |
| ATOM | 2768 | O | LEU | 371 | 62.697 | 63.006 | 53.367 | 1.00 | 70.33 |
| ATOM | 2769 | N | GLN | 372 | 61.153 | 64.193 | 52.236 | 1.00 | 69.31 |
| ATOM | 2770 | CA | GLN | 372 | 60.439 | 64.604 | 53.437 | 1.00 | 68.71 |
| ATOM | 2771 | CB | GLN | 372 | 60.097 | 66.098 | 53.371 | 1.00 | 69.99 |
| ATOM | 2772 | CG | GLN | 372 | 61.321 | 66.998 | 53.286 | 1.00 | 71.40 |
| ATOM | 2773 | CD | GLN | 372 | 62.246 | 66.847 | 54.484 | 1.00 | 71.80 |
| ATOM | 2774 | OE1 | GLN | 372 | 63.432 | 67.164 | 54.405 | 1.00 | 71.93 |
| ATOM | 2775 | NE2 | GLN | 372 | 61.703 | 66.368 | 55.601 | 1.00 | 71.67 |
| ATOM | 2776 | C | GLN | 372 | 59.170 | 63.805 | 53.646 | 1.00 | 67.36 |
| ATOM | 2777 | O | GLN | 372 | 58.424 | 63.546 | 52.703 | 1.00 | 67.20 |
| ATOM | 2778 | N | PRO | 373 | 58.907 | 63.408 | 54.897 | 1.00 | 65.94 |
| ATOM | 2779 | CD | PRO | 373 | 59.725 | 63.676 | 56.094 | 1.00 | 64.60 |
| ATOM | 2780 | CA | PRO | 373 | 57.716 | 62.631 | 55.241 | 1.00 | 66.02 |
| ATOM | 2781 | CB | PRO | 373 | 57.665 | 62.753 | 56.759 | 1.00 | 65.24 |
| ATOM | 2782 | CG | PRO | 373 | 59.117 | 62.742 | 57.119 | 1.00 | 64.73 |
| ATOM | 2783 | C | PRO | 373 | 56.442 | 63.140 | 54.565 | 1.00 | 66.69 |
| ATOM | 2784 | O | PRO | 373 | 55.578 | 62.349 | 54.191 | 1.00 | 66.11 |
| ATOM | 2785 | N | GLU | 374 | 56.336 | 64.458 | 54.402 | 1.00 | 67.78 |
| ATOM | 2786 | CA | GLU | 374 | 55.155 | 65.063 | 53.783 | 1.00 | 67.96 |
| ATOM | 2787 | CB | GLU | 374 | 55.102 | 66.571 | 54.062 | 1.00 | 71.04 |
| ATOM | 2788 | CG | GLU | 374 | 55.353 | 66.977 | 55.511 | 1.00 | 76.32 |
| ATOM | 2789 | CD | GLU | 374 | 56.834 | 67.069 | 55.836 | 1.00 | 79.67 |
| ATOM | 2790 | OE1 | GLU | 374 | 57.560 | 67.749 | 55.073 | 1.00 | 82.11 |
| ATOM | 2791 | OE2 | GLU | 374 | 57.269 | 66.474 | 56.851 | 1.00 | 80.79 |
| ATOM | 2792 | C | GLU | 374 | 55.130 | 64.839 | 52.276 | 1.00 | 65.88 |
| ATOM | 2793 | O | GLU | 374 | 54.068 | 64.852 | 51.652 | 1.00 | 64.80 |
| ATOM | 2794 | N | GLN | 375 | 56.306 | 64.650 | 51.690 | 1.00 | 64.56 |
| ATOM | 2795 | CA | GLN | 375 | 56.406 | 64.422 | 50.255 | 1.00 | 63.02 |
| ATOM | 2796 | CB | GLN | 375 | 57.873 | 64.533 | 49.816 | 1.00 | 63.52 |
| ATOM | 2797 | CG | GLN | 375 | 58.463 | 65.934 | 50.022 | 1.00 | 64.59 |
| ATOM | 2798 | CD | GLN | 375 | 59.898 | 66.082 | 49.511 | 1.00 | 65.39 |
| ATOM | 2799 | OE1 | GLN | 375 | 60.853 | 65.602 | 50.131 | 1.00 | 64.63 |
| ATOM | 2800 | NE2 | GLN | 375 | 60.048 | 66.750 | 48.370 | 1.00 | 64.25 |
| ATOM | 2801 | C | GLN | 375 | 55.819 | 63.048 | 49.894 | 1.00 | 61.79 |
| ATOM | 2802 | O | GLN | 375 | 55.289 | 62.852 | 48.796 | 1.00 | 59.93 |
| ATOM | 2803 | N | LEU | 376 | 55.893 | 62.111 | 50.836 | 1.00 | 60.12 |
| ATOM | 2804 | CA | LEU | 376 | 55.370 | 60.767 | 50.628 | 1.00 | 58.67 |
| ATOM | 2805 | CB | LEU | 376 | 55.747 | 59.865 | 51.807 | 1.00 | 56.26 |
| ATOM | 2806 | CG | LEU | 376 | 57.233 | 59.609 | 52.064 | 1.00 | 54.81 |
| ATOM | 2807 | CD1 | LEU | 376 | 57.400 | 58.770 | 53.323 | 1.00 | 52.63 |
| ATOM | 2808 | CD2 | LEU | 376 | 57.847 | 58.909 | 50.870 | 1.00 | 52.72 |
| ATOM | 2809 | C | LEU | 376 | 53.848 | 60.757 | 50.452 | 1.00 | 58.95 |
| ATOM | 2810 | O | LEU | 376 | 53.275 | 59.771 | 49.996 | 1.00 | 57.95 |
| ATOM | 2811 | N | GLN | 377 | 53.195 | 61.855 | 50.812 | 1.00 | 60.33 |
| ATOM | 2812 | CA | GLN | 377 | 51.740 | 61.936 | 50.694 | 1.00 | 62.04 |
| ATOM | 2813 | CB | GLN | 377 | 51.225 | 63.212 | 51.374 | 1.00 | 64.78 |
| ATOM | 2814 | CG | GLN | 377 | 51.792 | 63.448 | 52.782 | 1.00 | 69.22 |
| ATOM | 2815 | CD | GLN | 377 | 51.407 | 62.363 | 53.783 | 1.00 | 72.43 |
| ATOM | 2816 | OE1 | GLN | 377 | 51.981 | 62.273 | 54.881 | 1.00 | 73.12 |
| ATOM | 2817 | NE2 | GLN | 377 | 50.426 | 61.541 | 53.415 | 1.00 | 72.94 |
| ATOM | 2818 | C | GLN | 377 | 51.332 | 61.910 | 49.221 | 1.00 | 60.41 |
| ATOM | 2819 | O | GLN | 377 | 50.155 | 62.000 | 48.879 | 1.00 | 59.09 |
| ATOM | 2820 | N | VAL | 378 | 52.324 | 61.784 | 48.353 | 1.00 | 59.46 |
| ATOM | 2821 | CA | VAL | 378 | 52.086 | 61.732 | 46.922 | 1.00 | 59.66 |
| ATOM | 2822 | CB | VAL | 378 | 53.421 | 61.887 | 46.152 | 1.00 | 59.85 |
| ATOM | 2823 | CG1 | VAL | 378 | 53.229 | 61.598 | 44.679 | 1.00 | 61.73 |
| ATOM | 2824 | CG2 | VAL | 378 | 53.948 | 63.297 | 46.326 | 1.00 | 61.51 |
| ATOM | 2825 | C | VAL | 378 | 51.411 | 60.416 | 46.511 | 1.00 | 59.34 |
| ATOM | 2826 | O | VAL | 378 | 50.664 | 60.369 | 45.531 | 1.00 | 59.84 |
| ATOM | 2827 | N | PHE | 379 | 51.651 | 59.353 | 47.268 | 1.00 | 57.35 |
| ATOM | 2828 | CA | PHE | 379 | 51.077 | 58.060 | 46.922 | 1.00 | 57.45 |
| ATOM | 2829 | CB | PHE | 379 | 52.025 | 56.942 | 47.348 | 1.00 | 54.33 |
| ATOM | 2830 | CG | PHE | 379 | 53.397 | 57.072 | 46.773 | 1.00 | 51.15 |
| ATOM | 2831 | CD1 | PHE | 379 | 54.446 | 57.542 | 47.547 | 1.00 | 49.57 |
| ATOM | 2832 | CD2 | PHE | 379 | 53.635 | 56.758 | 45.445 | 1.00 | 49.60 |
| ATOM | 2833 | CE1 | PHE | 379 | 55.716 | 57.700 | 47.009 | 1.00 | 47.98 |
| ATOM | 2834 | CE2 | PHE | 379 | 54.906 | 56.914 | 44.899 | 1.00 | 49.85 |
| ATOM | 2835 | CZ | PHE | 379 | 55.946 | 57.386 | 45.687 | 1.00 | 46.39 |
| ATOM | 2836 | C | PHE | 379 | 49.699 | 57.779 | 47.491 | 1.00 | 59.16 |
| ATOM | 2837 | O | PHE | 379 | 49.169 | 56.675 | 47.323 | 1.00 | 59.83 |
| ATOM | 2838 | N | GLU | 380 | 49.107 | 58.773 | 48.141 | 1.00 | 60.36 |
| ATOM | 2839 | CA | GLU | 380 | 47.800 | 58.580 | 48.750 | 1.00 | 60.34 |
| ATOM | 2840 | CB | GLU | 380 | 47.431 | 59.804 | 49.595 | 1.00 | 64.04 |
| ATOM | 2841 | CG | GLU | 380 | 48.559 | 60.216 | 50.561 | 1.00 | 69.08 |
| ATOM | 2842 | CD | GLU | 380 | 48.062 | 60.875 | 51.841 | 1.00 | 71.31 |
| ATOM | 2843 | OE1 | GLU | 380 | 47.450 | 60.167 | 52.670 | 1.00 | 72.79 |
| ATOM | 2844 | OE2 | GLU | 380 | 48.286 | 62.096 | 52.018 | 1.00 | 73.08 |
| ATOM | 2845 | C | GLU | 380 | 46.705 | 58.257 | 47.746 | 1.00 | 58.30 |
| ATOM | 2846 | O | GLU | 380 | 45.672 | 57.700 | 48.110 | 1.00 | 58.43 |
| ATOM | 2847 | N | THR | 381 | 46.927 | 58.583 | 46.479 | 1.00 | 55.99 |
| ATOM | 2848 | CA | THR | 381 | 45.924 | 58.284 | 45.461 | 1.00 | 55.42 |
| ATOM | 2849 | CB | THR | 381 | 45.879 | 59.387 | 44.369 | 1.00 | 57.10 |
| ATOM | 2850 | OG1 | THR | 381 | 47.178 | 59.545 | 43.787 | 1.00 | 58.07 |
| ATOM | 2851 | CG2 | THR | 381 | 45.442 | 60.718 | 44.967 | 1.00 | 55.87 |
| ATOM | 2852 | C | THR | 381 | 46.219 | 56.936 | 44.798 | 1.00 | 54.20 |
| ATOM | 2853 | O | THR | 381 | 45.359 | 56.357 | 44.125 | 1.00 | 53.97 |
| ATOM | 2854 | N | LEU | 382 | 47.440 | 56.442 | 45.013 | 1.00 | 52.85 |
| ATOM | 2855 | CA | LEU | 382 | 47.907 | 55.179 | 44.444 | 1.00 | 49.26 |
| ATOM | 2856 | CB | LEU | 382 | 49.377 | 54.959 | 44.820 | 1.00 | 47.79 |
| ATOM | 2857 | CG | LEU | 382 | 50.073 | 53.734 | 44.210 | 1.00 | 46.53 |
| ATOM | 2858 | CD1 | LEU | 382 | 50.071 | 53.838 | 42.693 | 1.00 | 42.81 |
| ATOM | 2859 | CD2 | LEU | 382 | 51.493 | 53.635 | 44.736 | 1.00 | 46.51 |
| ATOM | 2860 | C | LEU | 382 | 47.078 | 53.956 | 44.850 | 1.00 | 48.27 |
| ATOM | 2861 | O | LEU | 382 | 46.925 | 53.656 | 46.031 | 1.00 | 47.89 |
| ATOM | 2862 | N | GLU | 383 | 46.564 | 53.252 | 43.848 | 1.00 | 46.86 |
| ATOM | 2863 | CA | GLU | 383 | 45.742 | 52.067 | 44.047 | 1.00 | 48.66 |
| ATOM | 2864 | CB | GLU | 383 | 44.392 | 52.251 | 43.340 | 1.00 | 49.79 |
| ATOM | 2865 | CG | GLU | 383 | 43.444 | 53.219 | 44.030 | 1.00 | 54.40 |
| ATOM | 2866 | CD | GLU | 383 | 42.353 | 53.762 | 43.110 | 1.00 | 54.51 |
| ATOM | 2867 | OE1 | GLU | 383 | 41.654 | 52.965 | 42.440 | 1.00 | 52.52 |
| ATOM | 2868 | OE2 | GLU | 383 | 42.194 | 55.002 | 43.071 | 1.00 | 57.33 |
| ATOM | 2869 | C | GLU | 383 | 46.398 | 50.792 | 43.511 | 1.00 | 48.27 |
| ATOM | 2870 | O | GLU | 383 | 46.054 | 49.681 | 43.941 | 1.00 | 48.13 |
| ATOM | 2871 | N | GLU | 384 | 47.338 | 50.956 | 42.579 | 1.00 | 46.27 |
| ATOM | 2872 | CA | GLU | 384 | 47.998 | 49.818 | 41.948 | 1.00 | 43.47 |
| ATOM | 2873 | CB | GLU | 384 | 47.182 | 49.377 | 40.727 | 1.00 | 43.66 |
| ATOM | 2874 | CG | GLU | 384 | 47.716 | 48.150 | 39.991 | 1.00 | 44.19 |
| ATOM | 2875 | CD | GLU | 384 | 46.854 | 47.776 | 38.783 | 1.00 | 46.00 |
| ATOM | 2876 | OE1 | GLU | 384 | 45.688 | 48.226 | 38.712 | 1.00 | 49.83 |
| ATOM | 2877 | OE2 | GLU | 384 | 47.331 | 47.027 | 37.908 | 1.00 | 45.41 |
| ATOM | 2878 | C | GLU | 384 | 49.444 | 50.030 | 41.510 | 1.00 | 41.83 |
| ATOM | 2879 | O | GLU | 384 | 49.822 | 51.096 | 41.028 | 1.00 | 38.67 |
| ATOM | 2880 | N | ILE | 385 | 50.232 | 48.973 | 41.684 | 1.00 | 40.65 |
| ATOM | 2881 | CA | ILE | 385 | 51.636 | 48.925 | 41.301 | 1.00 | 37.49 |
| ATOM | 2882 | CB | ILE | 385 | 52.569 | 48.874 | 42.535 | 1.00 | 36.30 |
| ATOM | 2883 | CG2 | ILE | 385 | 53.981 | 48.449 | 42.110 | 1.00 | 35.11 |
| ATOM | 2884 | CG1 | ILE | 385 | 52.581 | 50.238 | 43.239 | 1.00 | 35.95 |
| ATOM | 2885 | CD1 | ILE | 385 | 53.340 | 50.264 | 44.568 | 1.00 | 29.52 |
| ATOM | 2886 | C | ILE | 385 | 51.766 | 47.616 | 40.532 | 1.00 | 36.95 |
| ATOM | 2887 | O | ILE | 385 | 51.464 | 46.556 | 41.068 | 1.00 | 35.48 |
| ATOM | 2888 | N | THR | 386 | 52.196 | 47.684 | 39.277 | 1.00 | 37.82 |
| ATOM | 2889 | CA | THR | 386 | 52.341 | 46.475 | 38.474 | 1.00 | 37.30 |
| ATOM | 2890 | CB | THR | 386 | 52.386 | 46.786 | 36.949 | 1.00 | 39.40 |
| ATOM | 2891 | OG1 | THR | 386 | 53.414 | 47.751 | 36.670 | 1.00 | 37.23 |
| ATOM | 2892 | CG2 | THR | 386 | 51.027 | 47.308 | 36.477 | 1.00 | 38.44 |
| ATOM | 2893 | C | THR | 386 | 53.615 | 45.746 | 38.861 | 1.00 | 38.20 |
| ATOM | 2894 | O | THR | 386 | 53.722 | 44.521 | 38.684 | 1.00 | 38.03 |
| ATOM | 2895 | N | GLY | 387 | 54.574 | 46.497 | 39.398 | 1.00 | 36.02 |
| ATOM | 2896 | CA | GLY | 387 | 55.834 | 45.896 | 39.805 | 1.00 | 36.07 |
| ATOM | 2897 | C | GLY | 387 | 55.839 | 45.377 | 41.231 | 1.00 | 35.54 |
| ATOM | 2898 | O | GLY | 387 | 55.028 | 44.527 | 41.601 | 1.00 | 37.73 |
| ATOM | 2899 | N | TYR | 388 | 56.759 | 45.877 | 42.041 | 1.00 | 33.72 |
| ATOM | 2900 | CA | TYR | 388 | 56.845 | 45.438 | 43.426 | 1.00 | 34.18 |
| ATOM | 2901 | CB | TYR | 388 | 58.033 | 44.481 | 43.603 | 1.00 | 33.76 |
| ATOM | 2902 | CG | TYR | 388 | 59.384 | 45.087 | 43.268 | 1.00 | 33.25 |
| ATOM | 2903 | CD1 | TYR | 388 | 60.077 | 45.871 | 44.198 | 1.00 | 35.21 |
| ATOM | 2904 | CE1 | TYR | 388 | 61.317 | 46.446 | 43.886 | 1.00 | 33.69 |
| ATOM | 2905 | CD2 | TYR | 388 | 59.963 | 44.895 | 42.013 | 1.00 | 33.97 |
| ATOM | 2906 | CE2 | TYR | 388 | 61.199 | 45.470 | 41.685 | 1.00 | 33.20 |
| ATOM | 2907 | CZ | TYR | 388 | 61.870 | 46.238 | 42.626 | 1.00 | 34.87 |
| ATOM | 2908 | OH | TYR | 388 | 63.097 | 46.782 | 42.308 | 1.00 | 34.96 |
| ATOM | 2909 | C | TYR | 388 | 57.013 | 46.651 | 44.322 | 1.00 | 34.08 |
| ATOM | 2910 | O | TYR | 388 | 57.157 | 47.776 | 43.842 | 1.00 | 36.49 |
| ATOM | 2911 | N | LEU | 389 | 56.967 | 46.432 | 45.624 | 1.00 | 32.82 |
| ATOM | 2912 | CA | LEU | 389 | 57.163 | 47.522 | 46.559 | 1.00 | 33.35 |
| ATOM | 2913 | CB | LEU | 389 | 55.872 | 47.816 | 47.334 | 1.00 | 33.62 |
| ATOM | 2914 | CG | LEU | 389 | 55.963 | 48.815 | 48.497 | 1.00 | 34.53 |
| ATOM | 2915 | CD1 | LEU | 389 | 56.529 | 50.133 | 47.997 | 1.00 | 34.74 |
| ATOM | 2916 | CD2 | LEU | 389 | 54.583 | 49.036 | 49.132 | 1.00 | 33.40 |
| ATOM | 2917 | C | LEU | 389 | 58.269 | 47.031 | 47.480 | 1.00 | 34.94 |
| ATOM | 2918 | O | LEU | 389 | 58.185 | 45.934 | 48.050 | 1.00 | 34.97 |
| ATOM | 2919 | N | TYR | 390 | 59.322 | 47.832 | 47.594 | 1.00 | 35.52 |
| ATOM | 2920 | CA | TYR | 390 | 60.472 | 47.490 | 48.426 | 1.00 | 36.11 |
| ATOM | 2921 | CB | TYR | 390 | 61.687 | 47.241 | 47.515 | 1.00 | 36.83 |
| ATOM | 2922 | CG | TYR | 390 | 62.984 | 46.903 | 48.215 | 1.00 | 36.84 |
| ATOM | 2923 | CD1 | TYR | 390 | 63.449 | 45.588 | 48.260 | 1.00 | 39.20 |
| ATOM | 2924 | CE1 | TYR | 390 | 64.675 | 45.269 | 48.865 | 1.00 | 39.01 |
| ATOM | 2925 | CD2 | TYR | 390 | 63.770 | 47.902 | 48.800 | 1.00 | 36.30 |
| ATOM | 2926 | CE2 | TYR | 390 | 64.997 | 47.594 | 49.415 | 1.00 | 37.61 |
| ATOM | 2927 | CZ | TYR | 390 | 65.441 | 46.278 | 49.440 | 1.00 | 38.56 |
| ATOM | 2928 | OH | TYR | 390 | 66.642 | 45.965 | 50.034 | 1.00 | 40.00 |
| ATOM | 2929 | C | TYR | 390 | 60.722 | 48.675 | 49.359 | 1.00 | 36.57 |
| ATOM | 2930 | O | TYR | 390 | 60.818 | 49.812 | 48.902 | 1.00 | 39.11 |
| ATOM | 2931 | N | ILE | 391 | 60.826 | 48.411 | 50.656 | 1.00 | 34.84 |
| ATOM | 2932 | CA | ILE | 391 | 61.047 | 49.461 | 51.634 | 1.00 | 36.11 |
| ATOM | 2933 | CB | ILE | 391 | 59.742 | 49.799 | 52.392 | 1.00 | 38.59 |
| ATOM | 2934 | CG2 | ILE | 391 | 59.975 | 50.992 | 53.304 | 1.00 | 33.31 |
| ATOM | 2935 | CG1 | ILE | 391 | 58.606 | 50.080 | 51.394 | 1.00 | 37.52 |
| ATOM | 2936 | CD1 | ILE | 391 | 57.306 | 50.498 | 52.045 | 1.00 | 36.81 |
| ATOM | 2937 | C | ILE | 391 | 62.082 | 49.024 | 52.657 | 1.00 | 37.80 |
| ATOM | 2938 | O | ILE | 391 | 61.784 | 48.240 | 53.558 | 1.00 | 39.48 |
| ATOM | 2939 | N | SER | 392 | 63.296 | 49.541 | 52.524 | 1.00 | 38.87 |
| ATOM | 2940 | CA | SER | 392 | 64.376 | 49.186 | 53.438 | 1.00 | 39.77 |
| ATOM | 2941 | CB | SER | 392 | 65.609 | 48.743 | 52.652 | 1.00 | 40.15 |
| ATOM | 2942 | OG | SER | 392 | 66.009 | 49.756 | 51.739 | 1.00 | 42.76 |
| ATOM | 2943 | C | SER | 392 | 64.722 | 50.370 | 54.320 | 1.00 | 38.92 |
| ATOM | 2944 | O | SER | 392 | 65.679 | 50.325 | 55.090 | 1.00 | 38.90 |
| ATOM | 2945 | N | ALA | 393 | 63.941 | 51.435 | 54.185 | 1.00 | 38.10 |
| ATOM | 2946 | CA | ALA | 393 | 64.117 | 52.637 | 54.987 | 1.00 | 39.21 |
| ATOM | 2947 | CB | ALA | 393 | 65.262 | 53.492 | 54.444 | 1.00 | 38.87 |
| ATOM | 2948 | C | ALA | 393 | 62.809 | 53.424 | 54.978 | 1.00 | 40.68 |
| ATOM | 2949 | O | ALA | 393 | 62.103 | 53.488 | 53.963 | 1.00 | 38.40 |
| ATOM | 2950 | N | TRP | 394 | 62.488 | 54.019 | 56.120 | 1.00 | 41.96 |
| ATOM | 2951 | CA | TRP | 394 | 61.252 | 54.775 | 56.264 | 1.00 | 43.86 |
| ATOM | 2952 | CB | TRP | 394 | 60.110 | 53.794 | 56.583 | 1.00 | 42.73 |
| ATOM | 2953 | CG | TRP | 394 | 58.729 | 54.325 | 56.387 | 1.00 | 42.06 |
| ATOM | 2954 | CD2 | TRP | 394 | 58.072 | 54.578 | 55.139 | 1.00 | 41.48 |
| ATOM | 2955 | CE2 | TRP | 394 | 56.780 | 55.068 | 55.438 | 1.00 | 40.33 |
| ATOM | 2956 | CE3 | TRP | 394 | 58.448 | 54.439 | 53.797 | 1.00 | 41.31 |
| ATOM | 2957 | CD1 | TRP | 394 | 57.839 | 54.661 | 57.361 | 1.00 | 43.59 |
| ATOM | 2958 | NE1 | TRP | 394 | 56.664 | 55.107 | 56.801 | 1.00 | 43.49 |
| ATOM | 2959 | CZ2 | TRP | 394 | 55.862 | 55.421 | 54.447 | 1.00 | 39.55 |
| ATOM | 2960 | CZ3 | TRP | 394 | 57.528 | 54.793 | 52.803 | 1.00 | 41.64 |
| ATOM | 2961 | CH2 | TRP | 394 | 56.249 | 55.278 | 53.140 | 1.00 | 40.34 |
| ATOM | 2962 | C | TRP | 394 | 61.508 | 55.748 | 57.415 | 1.00 | 44.64 |
| ATOM | 2963 | O | TRP | 394 | 62.259 | 55.427 | 58.335 | 1.00 | 42.87 |
| ATOM | 2964 | N | PRO | 395 | 60.907 | 56.954 | 57.366 | 1.00 | 46.80 |
| ATOM | 2965 | CD | PRO | 395 | 59.942 | 57.432 | 56.356 | 1.00 | 47.29 |
| ATOM | 2966 | CA | PRO | 395 | 61.096 | 57.963 | 58.420 | 1.00 | 48.66 |
| ATOM | 2967 | CB | PRO | 395 | 60.358 | 59.184 | 57.859 | 1.00 | 48.97 |
| ATOM | 2968 | CG | PRO | 395 | 59.242 | 58.576 | 57.079 | 1.00 | 47.43 |
| ATOM | 2969 | C | PRO | 395 | 60.565 | 57.543 | 59.785 | 1.00 | 49.74 |
| ATOM | 2970 | O | PRO | 395 | 59.376 | 57.256 | 59.917 | 1.00 | 50.66 |
| ATOM | 2971 | N | ASP | 396 | 61.438 | 57.512 | 60.795 | 1.00 | 51.99 |
| ATOM | 2972 | CA | ASP | 396 | 61.033 | 57.121 | 62.153 | 1.00 | 54.46 |
| ATOM | 2973 | CB | ASP | 396 | 62.155 | 57.360 | 63.164 | 1.00 | 56.07 |
| ATOM | 2974 | CG | ASP | 396 | 63.283 | 56.373 | 63.022 | 1.00 | 61.08 |
| ATOM | 2975 | OD1 | ASP | 396 | 62.993 | 55.169 | 62.829 | 1.00 | 62.53 |
| ATOM | 2976 | OD2 | ASP | 396 | 64.458 | 56.799 | 63.115 | 1.00 | 63.88 |
| ATOM | 2977 | C | ASP | 396 | 59.790 | 57.850 | 62.645 | 1.00 | 54.39 |
| ATOM | 2978 | O | ASP | 396 | 58.986 | 57.289 | 63.390 | 1.00 | 54.40 |
| ATOM | 2979 | N | SER | 397 | 59.635 | 59.103 | 62.238 | 1.00 | 53.50 |
| ATOM | 2980 | CA | SER | 397 | 58.475 | 59.870 | 62.653 | 1.00 | 54.04 |
| ATOM | 2981 | CB | SER | 397 | 58.551 | 61.287 | 62.085 | 1.00 | 53.63 |
| ATOM | 2982 | OG | SER | 397 | 58.623 | 61.262 | 60.673 | 1.00 | 56.20 |
| ATOM | 2983 | C | SER | 397 | 57.161 | 59.199 | 62.229 | 1.00 | 53.65 |
| ATOM | 2984 | O | SER | 397 | 56.164 | 59.288 | 62.951 | 1.00 | 56.24 |
| ATOM | 2985 | N | LEU | 398 | 57.150 | 58.532 | 61.074 | 1.00 | 50.45 |
| ATOM | 2986 | CA | LEU | 398 | 55.936 | 57.858 | 60.606 | 1.00 | 49.41 |
| ATOM | 2987 | CB | LEU | 398 | 55.917 | 57.766 | 59.075 | 1.00 | 47.89 |
| ATOM | 2988 | CG | LEU | 398 | 55.848 | 59.089 | 58.296 | 1.00 | 48.30 |
| ATOM | 2989 | CD1 | LEU | 398 | 55.639 | 58.783 | 56.834 | 1.00 | 47.77 |
| ATOM | 2990 | CD2 | LEU | 398 | 54.706 | 59.967 | 58.798 | 1.00 | 46.68 |
| ATOM | 2991 | C | LEU | 398 | 55.801 | 56.457 | 61.220 | 1.00 | 47.95 |
| ATOM | 2992 | O | LEU | 398 | 56.700 | 55.627 | 61.100 | 1.00 | 47.18 |
| ATOM | 2993 | N | PRO | 399 | 54.662 | 56.182 | 61.885 | 1.00 | 45.97 |
| ATOM | 2994 | CD | PRO | 399 | 53.594 | 57.154 | 62.195 | 1.00 | 42.77 |
| ATOM | 2995 | CA | PRO | 399 | 54.393 | 54.892 | 62.533 | 1.00 | 45.25 |
| ATOM | 2996 | CB | PRO | 399 | 53.327 | 55.254 | 63.566 | 1.00 | 44.03 |
| ATOM | 2997 | CG | PRO | 399 | 52.528 | 56.285 | 62.842 | 1.00 | 43.17 |
| ATOM | 2998 | C | PRO | 399 | 53.940 | 53.753 | 61.618 | 1.00 | 45.16 |
| ATOM | 2999 | O | PRO | 399 | 54.021 | 52.575 | 61.991 | 1.00 | 46.16 |
| ATOM | 3000 | N | ASP | 400 | 53.451 | 54.093 | 60.434 | 1.00 | 42.35 |
| ATOM | 3001 | CA | ASP | 400 | 52.987 | 53.069 | 59.512 | 1.00 | 41.88 |
| ATOM | 3002 | CB | ASP | 400 | 51.493 | 52.790 | 59.736 | 1.00 | 42.58 |
| ATOM | 3003 | CG | ASP | 400 | 50.617 | 54.024 | 59.516 | 1.00 | 44.56 |
| ATOM | 3004 | OD1 | ASP | 400 | 51.096 | 55.030 | 58.943 | 1.00 | 44.51 |
| ATOM | 3005 | OD2 | ASP | 400 | 49.434 | 53.983 | 59.912 | 1.00 | 45.18 |
| ATOM | 3006 | C | ASP | 400 | 53.229 | 53.461 | 58.062 | 1.00 | 41.12 |
| ATOM | 3007 | O | ASP | 400 | 53.945 | 54.425 | 57.779 | 1.00 | 41.52 |
| ATOM | 3008 | N | LEU | 401 | 52.630 | 52.711 | 57.144 | 1.00 | 39.96 |
| ATOM | 3009 | CA | LEU | 401 | 52.787 | 52.985 | 55.718 | 1.00 | 40.52 |
| ATOM | 3010 | CB | LEU | 401 | 53.207 | 51.704 | 54.986 | 1.00 | 37.20 |
| ATOM | 3011 | CG | LEU | 401 | 54.389 | 50.918 | 55.572 | 1.00 | 34.86 |
| ATOM | 3012 | CD1 | LEU | 401 | 54.417 | 49.516 | 54.978 | 1.00 | 29.03 |
| ATOM | 3013 | CD2 | LEU | 401 | 55.697 | 51.668 | 55.317 | 1.00 | 32.51 |
| ATOM | 3014 | C | LEU | 401 | 51.445 | 53.481 | 55.178 | 1.00 | 42.33 |
| ATOM | 3015 | O | LEU | 401 | 51.102 | 53.249 | 54.010 | 1.00 | 42.00 |
| ATOM | 3016 | N | SER | 402 | 50.691 | 54.174 | 56.029 | 1.00 | 43.15 |
| ATOM | 3017 | CA | SER | 402 | 49.376 | 54.659 | 55.635 | 1.00 | 45.17 |
| ATOM | 3018 | CB | SER | 402 | 48.704 | 55.422 | 56.790 | 1.00 | 46.37 |
| ATOM | 3019 | OG | SER | 402 | 49.390 | 56.621 | 57.108 | 1.00 | 50.42 |
| ATOM | 3020 | C | SER | 402 | 49.379 | 55.505 | 54.360 | 1.00 | 45.55 |
| ATOM | 3021 | O | SER | 402 | 48.332 | 55.649 | 53.718 | 1.00 | 44.73 |
| ATOM | 3022 | N | VAL | 403 | 50.531 | 56.059 | 53.977 | 1.00 | 43.17 |
| ATOM | 3023 | CA | VAL | 403 | 50.562 | 56.833 | 52.746 | 1.00 | 44.06 |
| ATOM | 3024 | CB | VAL | 403 | 51.995 | 57.324 | 52.361 | 1.00 | 46.39 |
| ATOM | 3025 | CG1 | VAL | 403 | 52.570 | 58.199 | 53.450 | 1.00 | 45.24 |
| ATOM | 3026 | CG2 | VAL | 403 | 52.908 | 56.131 | 52.075 | 1.00 | 48.94 |
| ATOM | 3027 | C | VAL | 403 | 50.056 | 55.922 | 51.625 | 1.00 | 43.40 |
| ATOM | 3028 | O | VAL | 403 | 49.513 | 56.397 | 50.624 | 1.00 | 44.33 |
| ATOM | 3029 | N | PHE | 404 | 50.238 | 54.614 | 51.797 | 1.00 | 40.95 |
| ATOM | 3030 | CA | PHE | 404 | 49.795 | 53.636 | 50.804 | 1.00 | 41.83 |
| ATOM | 3031 | CB | PHE | 404 | 50.849 | 52.528 | 50.636 | 1.00 | 42.48 |
| ATOM | 3032 | CG | PHE | 404 | 52.148 | 53.000 | 50.065 | 1.00 | 39.30 |
| ATOM | 3033 | CD1 | PHE | 404 | 52.217 | 53.489 | 48.770 | 1.00 | 38.75 |
| ATOM | 3034 | CD2 | PHE | 404 | 53.306 | 52.953 | 50.825 | 1.00 | 38.89 |
| ATOM | 3035 | CE1 | PHE | 404 | 53.426 | 53.921 | 48.245 | 1.00 | 38.45 |
| ATOM | 3036 | CE2 | PHE | 404 | 54.519 | 53.383 | 50.307 | 1.00 | 37.33 |
| ATOM | 3037 | CZ | PHE | 404 | 54.577 | 53.866 | 49.017 | 1.00 | 37.18 |
| ATOM | 3038 | C | PHE | 404 | 48.472 | 52.990 | 51.220 | 1.00 | 42.45 |
| ATOM | 3039 | O | PHE | 404 | 48.150 | 51.877 | 50.792 | 1.00 | 39.55 |
| ATOM | 3040 | N | GLN | 405 | 47.699 | 53.687 | 52.045 | 1.00 | 44.33 |
| ATOM | 3041 | CA | GLN | 405 | 46.442 | 53.130 | 52.520 | 1.00 | 45.10 |
| ATOM | 3042 | CB | GLN | 405 | 45.816 | 54.054 | 53.564 | 1.00 | 45.82 |
| ATOM | 3043 | CG | GLN | 405 | 45.431 | 55.426 | 53.053 | 1.00 | 51.73 |
| ATOM | 3044 | CD | GLN | 405 | 44.871 | 56.304 | 54.156 | 1.00 | 53.97 |
| ATOM | 3045 | OE1 | GLN | 405 | 43.860 | 55.960 | 54.778 | 1.00 | 54.78 |
| ATOM | 3046 | NE2 | GLN | 405 | 45.527 | 57.443 | 54.411 | 1.00 | 53.84 |
| ATOM | 3047 | C | GLN | 405 | 45.426 | 52.790 | 51.429 | 1.00 | 44.71 |
| ATOM | 3048 | O | GLN | 405 | 44.518 | 51.996 | 51.667 | 1.00 | 45.50 |
| ATOM | 3049 | N | ASN | 406 | 45.565 | 53.368 | 50.239 | 1.00 | 43.57 |
| ATOM | 3050 | CA | ASN | 406 | 44.624 | 53.056 | 49.160 | 1.00 | 44.72 |
| ATOM | 3051 | CB | ASN | 406 | 44.169 | 54.326 | 48.421 | 1.00 | 46.41 |
| ATOM | 3052 | CG | ASN | 406 | 43.366 | 55.270 | 49.299 | 1.00 | 47.55 |
| ATOM | 3053 | OD1 | ASN | 406 | 42.443 | 54.858 | 50.011 | 1.00 | 45.86 |
| ATOM | 3054 | ND2 | ASN | 406 | 43.705 | 56.555 | 49.236 | 1.00 | 46.55 |
| ATOM | 3055 | C | ASN | 406 | 45.223 | 52.094 | 48.137 | 1.00 | 44.32 |
| ATOM | 3056 | O | ASN | 406 | 44.677 | 51.917 | 47.044 | 1.00 | 45.85 |
| ATOM | 3057 | N | LEU | 407 | 46.355 | 51.492 | 48.482 | 1.00 | 42.87 |
| ATOM | 3058 | CA | LEU | 407 | 47.011 | 50.546 | 47.595 | 1.00 | 41.65 |
| ATOM | 3059 | CB | LEU | 407 | 48.438 | 50.296 | 48.078 | 1.00 | 39.43 |
| ATOM | 3060 | CG | LEU | 407 | 49.305 | 49.362 | 47.244 | 1.00 | 38.68 |
| ATOM | 3061 | CD1 | LEU | 407 | 49.279 | 49.793 | 45.796 | 1.00 | 37.29 |
| ATOM | 3062 | CD2 | LEU | 407 | 50.718 | 49.377 | 47.794 | 1.00 | 39.44 |
| ATOM | 3063 | C | LEU | 407 | 46.191 | 49.257 | 47.627 | 1.00 | 42.87 |
| ATOM | 3064 | O | LEU | 407 | 46.066 | 48.618 | 48.668 | 1.00 | 41.20 |
| ATOM | 3065 | N | GLN | 408 | 45.626 | 48.880 | 46.487 | 1.00 | 44.64 |
| ATOM | 3066 | CA | GLN | 408 | 44.789 | 47.686 | 46.422 | 1.00 | 46.93 |
| ATOM | 3067 | CB | GLN | 408 | 43.540 | 47.979 | 45.599 | 1.00 | 46.58 |
| ATOM | 3068 | CG | GLN | 408 | 42.557 | 48.874 | 46.317 | 1.00 | 51.50 |
| ATOM | 3069 | CD | GLN | 408 | 41.587 | 49.554 | 45.374 | 1.00 | 52.88 |
| ATOM | 3070 | OE1 | GLN | 408 | 40.933 | 48.907 | 44.545 | 1.00 | 52.20 |
| ATOM | 3071 | NE2 | GLN | 408 | 41.486 | 50.872 | 45.497 | 1.00 | 55.91 |
| ATOM | 3072 | C | GLN | 408 | 45.464 | 46.458 | 45.852 | 1.00 | 47.02 |
| ATOM | 3073 | O | GLN | 408 | 45.213 | 45.334 | 46.300 | 1.00 | 47.65 |
| ATOM | 3074 | N | VAL | 409 | 46.317 | 46.672 | 44.861 | 1.00 | 44.93 |
| ATOM | 3075 | CA | VAL | 409 | 46.984 | 45.562 | 44.227 | 1.00 | 44.05 |
| ATOM | 3076 | CB | VAL | 409 | 46.259 | 45.169 | 42.920 | 1.00 | 45.18 |
| ATOM | 3077 | CG1 | VAL | 409 | 47.031 | 44.075 | 42.194 | 1.00 | 45.20 |
| ATOM | 3078 | CG2 | VAL | 409 | 44.850 | 44.698 | 43.231 | 1.00 | 47.18 |
| ATOM | 3079 | C | VAL | 409 | 48.434 | 45.835 | 43.890 | 1.00 | 43.61 |
| ATOM | 3080 | O | VAL | 409 | 48.813 | 46.949 | 43.528 | 1.00 | 42.67 |
| ATOM | 3081 | N | ILE | 410 | 49.239 | 44.791 | 44.038 | 1.00 | 41.59 |
| ATOM | 3082 | CA | ILE | 410 | 50.644 | 44.823 | 43.693 | 1.00 | 38.73 |
| ATOM | 3083 | CB | ILE | 410 | 51.557 | 44.752 | 44.937 | 1.00 | 34.96 |
| ATOM | 3084 | CG2 | ILE | 410 | 53.034 | 44.643 | 44.501 | 1.00 | 30.70 |
| ATOM | 3085 | CG1 | ILE | 410 | 51.347 | 46.003 | 45.793 | 1.00 | 30.25 |
| ATOM | 3086 | CD1 | ILE | 410 | 52.241 | 46.107 | 47.012 | 1.00 | 25.93 |
| ATOM | 3087 | C | ILE | 410 | 50.752 | 43.546 | 42.877 | 1.00 | 39.73 |
| ATOM | 3088 | O | ILE | 410 | 50.887 | 42.461 | 43.435 | 1.00 | 40.26 |
| ATOM | 3089 | N | ARG | 411 | 50.630 | 43.680 | 41.556 | 1.00 | 41.18 |
| ATOM | 3090 | CA | ARG | 411 | 50.694 | 42.536 | 40.651 | 1.00 | 41.50 |
| ATOM | 3091 | CB | ARG | 411 | 50.614 | 42.987 | 39.193 | 1.00 | 44.32 |
| ATOM | 3092 | CG | ARG | 411 | 49.216 | 43.133 | 38.611 | 1.00 | 50.54 |
| ATOM | 3093 | CD | ARG | 411 | 48.556 | 44.418 | 39.037 | 1.00 | 54.13 |
| ATOM | 3094 | NE | ARG | 411 | 47.302 | 44.679 | 38.322 | 1.00 | 60.27 |
| ATOM | 3095 | CZ | ARG | 411 | 46.201 | 43.930 | 38.392 | 1.00 | 61.55 |
| ATOM | 3096 | NH1 | ARG | 411 | 46.162 | 42.836 | 39.144 | 1.00 | 59.64 |
| ATOM | 3097 | NH2 | ARG | 411 | 45.118 | 44.299 | 37.719 | 1.00 | 63.09 |
| ATOM | 3098 | C | ARG | 411 | 51.961 | 41.720 | 40.819 | 1.00 | 41.76 |
| ATOM | 3099 | O | ARG | 411 | 51.927 | 40.494 | 40.760 | 1.00 | 43.44 |
| ATOM | 3100 | N | GLY | 412 | 53.086 | 42.392 | 41.018 | 1.00 | 40.54 |
| ATOM | 3101 | CA | GLY | 412 | 54.327 | 41.659 | 41.154 | 1.00 | 41.36 |
| ATOM | 3102 | C | GLY | 412 | 54.784 | 41.094 | 39.815 | 1.00 | 41.71 |
| ATOM | 3103 | O | GLY | 412 | 55.437 | 40.055 | 39.771 | 1.00 | 39.26 |
| ATOM | 3104 | N | ARG | 413 | 54.436 | 41.770 | 38.717 | 1.00 | 43.65 |
| ATOM | 3105 | CA | ARG | 413 | 54.857 | 41.316 | 37.391 | 1.00 | 44.63 |
| ATOM | 3106 | CB | ARG | 413 | 54.234 | 42.165 | 36.287 | 1.00 | 45.78 |
| ATOM | 3107 | CG | ARG | 413 | 52.743 | 41.912 | 36.102 | 1.00 | 49.28 |
| ATOM | 3108 | CD | ARG | 413 | 52.314 | 42.174 | 34.661 | 1.00 | 50.29 |
| ATOM | 3109 | NE | ARG | 413 | 51.307 | 43.227 | 34.552 | 1.00 | 53.86 |
| ATOM | 3110 | CZ | ARG | 413 | 50.043 | 43.113 | 34.950 | 1.00 | 51.79 |
| ATOM | 3111 | NH1 | ARG | 413 | 49.608 | 41.978 | 35.493 | 1.00 | 50.37 |
| ATOM | 3112 | NH2 | ARG | 413 | 49.217 | 44.142 | 34.799 | 1.00 | 48.50 |
| ATOM | 3113 | C | ARG | 413 | 56.373 | 41.398 | 37.331 | 1.00 | 44.29 |
| ATOM | 3114 | O | ARG | 413 | 57.008 | 40.727 | 36.524 | 1.00 | 46.07 |
| ATOM | 3115 | N | ILE | 414 | 56.938 | 42.242 | 38.186 | 1.00 | 42.36 |
| ATOM | 3116 | CA | ILE | 414 | 58.380 | 42.380 | 38.311 | 1.00 | 41.85 |
| ATOM | 3117 | CB | ILE | 414 | 58.868 | 43.768 | 37.862 | 1.00 | 41.95 |
| ATOM | 3118 | CG2 | ILE | 414 | 60.374 | 43.869 | 38.024 | 1.00 | 38.29 |
| ATOM | 3119 | CG1 | ILE | 414 | 58.504 | 44.006 | 36.401 | 1.00 | 42.75 |
| ATOM | 3120 | CD1 | ILE | 414 | 58.947 | 45.376 | 35.886 | 1.00 | 45.50 |
| ATOM | 3121 | C | ILE | 414 | 58.632 | 42.209 | 39.820 | 1.00 | 42.04 |
| ATOM | 3122 | O | ILE | 414 | 58.010 | 42.896 | 40.634 | 1.00 | 42.22 |
| ATOM | 3123 | N | LEU | 415 | 59.525 | 41.293 | 40.194 | 1.00 | 40.03 |
| ATOM | 3124 | CA | LEU | 415 | 59.807 | 41.040 | 41.608 | 1.00 | 39.68 |
| ATOM | 3125 | CB | LEU | 415 | 59.630 | 39.553 | 41.915 | 1.00 | 37.15 |
| ATOM | 3126 | CG | LEU | 415 | 58.331 | 38.933 | 41.382 | 1.00 | 37.95 |
| ATOM | 3127 | CD1 | LEU | 415 | 58.434 | 37.428 | 41.472 | 1.00 | 34.92 |
| ATOM | 3128 | CD2 | LEU | 415 | 57.118 | 39.454 | 42.150 | 1.00 | 36.11 |
| ATOM | 3129 | C | LEU | 415 | 61.202 | 41.475 | 42.029 | 1.00 | 38.94 |
| ATOM | 3130 | O | LEU | 415 | 62.129 | 41.464 | 41.229 | 1.00 | 40.10 |
| ATOM | 3131 | N | HIS | 416 | 61.351 | 41.870 | 43.286 | 1.00 | 37.91 |
| ATOM | 3132 | CA | HIS | 416 | 62.661 | 42.277 | 43.765 | 1.00 | 37.95 |
| ATOM | 3133 | CB | HIS | 416 | 62.549 | 42.958 | 45.117 | 1.00 | 35.21 |
| ATOM | 3134 | CG | HIS | 416 | 63.853 | 43.482 | 45.611 | 1.00 | 37.67 |
| ATOM | 3135 | CD2 | HIS | 416 | 64.475 | 44.666 | 45.396 | 1.00 | 36.84 |
| ATOM | 3136 | ND1 | HIS | 416 | 64.735 | 42.708 | 46.333 | 1.00 | 37.22 |
| ATOM | 3137 | CE1 | HIS | 416 | 65.846 | 43.394 | 46.540 | 1.00 | 38.59 |
| ATOM | 3138 | NE2 | HIS | 416 | 65.714 | 44.584 | 45.981 | 1.00 | 36.63 |
| ATOM | 3139 | C | HIS | 416 | 63.520 | 41.011 | 43.849 | 1.00 | 38.20 |
| ATOM | 3140 | O | HIS | 416 | 63.062 | 39.973 | 44.355 | 1.00 | 37.39 |
| ATOM | 3141 | N | ASN | 417 | 64.760 | 41.092 | 43.359 | 1.00 | 36.97 |
| ATOM | 3142 | CA | ASN | 417 | 65.634 | 39.913 | 43.298 | 1.00 | 35.90 |
| ATOM | 3143 | CB | ASN | 417 | 66.035 | 39.399 | 44.683 | 1.00 | 38.02 |
| ATOM | 3144 | CG | ASN | 417 | 67.226 | 40.139 | 45.260 | 1.00 | 40.21 |
| ATOM | 3145 | OD1 | ASN | 417 | 68.074 | 40.625 | 44.525 | 1.00 | 44.23 |
| ATOM | 3146 | ND2 | ASN | 417 | 67.304 | 40.210 | 46.582 | 1.00 | 39.54 |
| ATOM | 3147 | C | ASN | 417 | 64.845 | 38.823 | 42.562 | 1.00 | 36.30 |
| ATOM | 3148 | O | ASN | 417 | 65.093 | 37.631 | 42.738 | 1.00 | 35.72 |
| ATOM | 3149 | N | GLY | 418 | 63.883 | 39.252 | 41.747 | 1.00 | 33.78 |
| ATOM | 3150 | CA | GLY | 418 | 63.071 | 38.327 | 40.989 | 1.00 | 34.34 |
| ATOM | 3151 | C | GLY | 418 | 62.245 | 37.374 | 41.831 | 1.00 | 35.85 |
| ATOM | 3152 | O | GLY | 418 | 61.760 | 36.363 | 41.323 | 1.00 | 37.04 |
| ATOM | 3153 | N | ALA | 419 | 62.055 | 37.690 | 43.108 | 1.00 | 35.17 |
| ATOM | 3154 | CA | ALA | 419 | 61.296 | 36.793 | 43.972 | 1.00 | 34.41 |
| ATOM | 3155 | CB | ALA | 419 | 62.275 | 35.987 | 44.854 | 1.00 | 30.62 |
| ATOM | 3156 | C | ALA | 419 | 60.226 | 37.444 | 44.855 | 1.00 | 33.91 |
| ATOM | 3157 | O | ALA | 419 | 59.134 | 36.893 | 45.021 | 1.00 | 33.84 |
| ATOM | 3158 | N | TYR | 420 | 60.547 | 38.611 | 45.405 | 1.00 | 33.11 |
| ATOM | 3159 | CA | TYR | 420 | 59.672 | 39.325 | 46.327 | 1.00 | 31.95 |
| ATOM | 3160 | CB | TYR | 420 | 60.514 | 39.806 | 47.505 | 1.00 | 33.26 |
| ATOM | 3161 | CG | TYR | 420 | 61.335 | 38.713 | 48.139 | 1.00 | 34.39 |
| ATOM | 3162 | CD1 | TYR | 420 | 62.715 | 38.640 | 47.936 | 1.00 | 37.18 |
| ATOM | 3163 | CE1 | TYR | 420 | 63.487 | 37.626 | 48.541 | 1.00 | 38.42 |
| ATOM | 3164 | CD2 | TYR | 420 | 60.733 | 37.749 | 48.958 | 1.00 | 35.52 |
| ATOM | 3165 | CE2 | TYR | 420 | 61.486 | 36.738 | 49.562 | 1.00 | 37.85 |
| ATOM | 3166 | CZ | TYR | 420 | 62.862 | 36.681 | 49.351 | 1.00 | 38.60 |
| ATOM | 3167 | OH | TYR | 420 | 63.595 | 35.676 | 49.942 | 1.00 | 39.21 |
| ATOM | 3168 | C | TYR | 420 | 58.879 | 40.497 | 45.764 | 1.00 | 31.18 |
| ATOM | 3169 | O | TYR | 420 | 59.442 | 41.404 | 45.164 | 1.00 | 32.88 |
| ATOM | 3170 | N | SER | 421 | 57.571 | 40.488 | 45.991 | 1.00 | 31.19 |
| ATOM | 3171 | CA | SER | 421 | 56.697 | 41.549 | 45.499 | 1.00 | 30.32 |
| ATOM | 3172 | CB | SER | 421 | 55.345 | 40.970 | 45.077 | 1.00 | 32.75 |
| ATOM | 3173 | OG | SER | 421 | 54.882 | 40.006 | 46.012 | 1.00 | 36.22 |
| ATOM | 3174 | C | SER | 421 | 56.497 | 42.603 | 46.559 | 1.00 | 30.53 |
| ATOM | 3175 | O | SER | 421 | 56.132 | 43.744 | 46.264 | 1.00 | 31.77 |
| ATOM | 3176 | N | LEU | 422 | 56.738 | 42.221 | 47.805 | 1.00 | 30.40 |
| ATOM | 3177 | CA | LEU | 422 | 56.604 | 43.151 | 48.917 | 1.00 | 29.35 |
| ATOM | 3178 | CB | LEU | 422 | 55.254 | 42.986 | 49.622 | 1.00 | 30.25 |
| ATOM | 3179 | CG | LEU | 422 | 55.121 | 43.842 | 50.888 | 1.00 | 29.90 |
| ATOM | 3180 | CD1 | LEU | 422 | 55.036 | 45.319 | 50.512 | 1.00 | 28.81 |
| ATOM | 3181 | CD2 | LEU | 422 | 53.893 | 43.415 | 51.663 | 1.00 | 31.47 |
| ATOM | 3182 | C | LEU | 422 | 57.706 | 42.893 | 49.920 | 1.00 | 29.76 |
| ATOM | 3183 | O | LEU | 422 | 57.782 | 41.808 | 50.501 | 1.00 | 29.18 |
| ATOM | 3184 | N | THR | 423 | 58.551 | 43.896 | 50.137 | 1.00 | 29.22 |
| ATOM | 3185 | CA | THR | 423 | 59.640 | 43.755 | 51.080 | 1.00 | 29.16 |
| ATOM | 3186 | CB | THR | 423 | 60.979 | 43.717 | 50.349 | 1.00 | 30.73 |
| ATOM | 3187 | OG1 | THR | 423 | 60.952 | 42.685 | 49.357 | 1.00 | 26.80 |
| ATOM | 3188 | CG2 | THR | 423 | 62.103 | 43.462 | 51.326 | 1.00 | 29.57 |
| ATOM | 3189 | C | THR | 423 | 59.671 | 44.904 | 52.076 | 1.00 | 32.07 |
| ATOM | 3190 | O | THR | 423 | 59.665 | 46.070 | 51.701 | 1.00 | 34.06 |
| ATOM | 3191 | N | LEU | 424 | 59.697 | 44.572 | 53.356 | 1.00 | 33.32 |
| ATOM | 3192 | CA | LEU | 424 | 59.757 | 45.588 | 54.386 | 1.00 | 32.23 |
| ATOM | 3193 | CB | LEU | 424 | 58.432 | 45.680 | 55.131 | 1.00 | 31.70 |
| ATOM | 3194 | CG | LEU | 424 | 57.199 | 45.898 | 54.266 | 1.00 | 31.60 |
| ATOM | 3195 | CD1 | LEU | 424 | 56.001 | 46.055 | 55.196 | 1.00 | 32.03 |
| ATOM | 3196 | CD2 | LEU | 424 | 57.374 | 47.131 | 53.384 | 1.00 | 30.01 |
| ATOM | 3197 | C | LEU | 424 | 60.830 | 45.109 | 55.321 | 1.00 | 32.73 |
| ATOM | 3198 | O | LEU | 424 | 60.655 | 44.093 | 55.990 | 1.00 | 33.11 |
| ATOM | 3199 | N | GLN | 425 | 61.941 | 45.834 | 55.367 | 1.00 | 35.85 |
| ATOM | 3200 | CA | GLN | 425 | 63.054 | 45.448 | 56.225 | 1.00 | 38.67 |
| ATOM | 3201 | CB | GLN | 425 | 64.051 | 44.596 | 55.427 | 1.00 | 39.34 |
| ATOM | 3202 | CG | GLN | 425 | 64.565 | 45.265 | 54.172 | 1.00 | 41.63 |
| ATOM | 3203 | CD | GLN | 425 | 65.575 | 44.410 | 53.435 | 1.00 | 44.59 |
| ATOM | 3204 | OE1 | GLN | 425 | 65.325 | 43.229 | 53.160 | 1.00 | 45.28 |
| ATOM | 3205 | NE2 | GLN | 425 | 66.723 | 44.999 | 53.105 | 1.00 | 42.97 |
| ATOM | 3206 | C | GLN | 425 | 63.794 | 46.613 | 56.891 | 1.00 | 39.68 |
| ATOM | 3207 | O | GLN | 425 | 63.935 | 47.704 | 56.320 | 1.00 | 40.17 |
| ATOM | 3208 | N | GLY | 426 | 64.258 | 46.348 | 58.112 | 1.00 | 40.10 |
| ATOM | 3209 | CA | GLY | 426 | 64.992 | 47.323 | 58.899 | 1.00 | 39.31 |
| ATOM | 3210 | C | GLY | 426 | 64.228 | 48.585 | 59.233 | 1.00 | 39.60 |
| ATOM | 3211 | O | GLY | 426 | 64.839 | 49.576 | 59.615 | 1.00 | 41.24 |
| ATOM | 3212 | N | LEU | 427 | 62.906 | 48.562 | 59.114 | 1.00 | 38.98 |
| ATOM | 3213 | CA | LEU | 427 | 62.117 | 49.759 | 59.390 | 1.00 | 39.49 |
| ATOM | 3214 | CB | LEU | 427 | 60.808 | 49.704 | 58.613 | 1.00 | 39.56 |
| ATOM | 3215 | CG | LEU | 427 | 60.970 | 49.266 | 57.157 | 1.00 | 39.73 |
| ATOM | 3216 | CD1 | LEU | 427 | 59.601 | 49.006 | 56.567 | 1.00 | 39.64 |
| ATOM | 3217 | CD2 | LEU | 427 | 61.711 | 50.331 | 56.365 | 1.00 | 38.74 |
| ATOM | 3218 | C | LEU | 427 | 61.817 | 49.990 | 60.866 | 1.00 | 40.51 |
| ATOM | 3219 | O | LEU | 427 | 62.114 | 49.149 | 61.727 | 1.00 | 40.55 |
| ATOM | 3220 | N | GLY | 428 | 61.228 | 51.148 | 61.149 | 1.00 | 40.84 |
| ATOM | 3221 | CA | GLY | 428 | 60.884 | 51.493 | 62.514 | 1.00 | 38.84 |
| ATOM | 3222 | C | GLY | 428 | 59.381 | 51.565 | 62.697 | 1.00 | 40.06 |
| ATOM | 3223 | O | GLY | 428 | 58.897 | 51.957 | 63.761 | 1.00 | 40.32 |
| ATOM | 3224 | N | ILE | 429 | 58.630 | 51.179 | 61.669 | 1.00 | 39.38 |
| ATOM | 3225 | CA | ILE | 429 | 57.178 | 51.230 | 61.759 | 1.00 | 38.33 |
| ATOM | 3226 | CB | ILE | 429 | 56.518 | 50.853 | 60.428 | 1.00 | 37.87 |
| ATOM | 3227 | CG2 | ILE | 429 | 56.897 | 51.876 | 59.371 | 1.00 | 38.44 |
| ATOM | 3228 | CG1 | ILE | 429 | 56.932 | 49.444 | 60.002 | 1.00 | 37.36 |
| ATOM | 3229 | CD1 | ILE | 429 | 56.211 | 48.957 | 58.741 | 1.00 | 34.27 |
| ATOM | 3230 | C | ILE | 429 | 56.615 | 50.343 | 62.865 | 1.00 | 38.00 |
| ATOM | 3231 | O | ILE | 429 | 57.205 | 49.313 | 63.219 | 1.00 | 35.51 |
| ATOM | 3232 | N | SER | 430 | 55.472 | 50.758 | 63.412 | 1.00 | 37.06 |
| ATOM | 3233 | CA | SER | 430 | 54.826 | 50.013 | 64.480 | 1.00 | 36.12 |
| ATOM | 3234 | CB | SER | 430 | 54.293 | 50.968 | 65.531 | 1.00 | 37.07 |
| ATOM | 3235 | OG | SER | 430 | 55.382 | 51.538 | 66.235 | 1.00 | 37.62 |
| ATOM | 3236 | C | SER | 430 | 53.729 | 49.107 | 63.960 | 1.00 | 35.48 |
| ATOM | 3237 | O | SER | 430 | 53.370 | 48.132 | 64.612 | 1.00 | 35.19 |
| ATOM | 3238 | N | TRP | 431 | 53.204 | 49.426 | 62.781 | 1.00 | 36.17 |
| ATOM | 3239 | CA | TRP | 431 | 52.190 | 48.588 | 62.132 | 1.00 | 37.40 |
| ATOM | 3240 | CB | TRP | 431 | 50.798 | 48.784 | 62.763 | 1.00 | 38.03 |
| ATOM | 3241 | CG | TRP | 431 | 50.242 | 50.167 | 62.666 | 1.00 | 38.99 |
| ATOM | 3242 | CD2 | TRP | 431 | 50.377 | 51.200 | 63.643 | 1.00 | 37.91 |
| ATOM | 3243 | CE2 | TRP | 431 | 49.793 | 52.370 | 63.099 | 1.00 | 36.90 |
| ATOM | 3244 | CE3 | TRP | 431 | 50.944 | 51.255 | 64.922 | 1.00 | 37.49 |
| ATOM | 3245 | CD1 | TRP | 431 | 49.588 | 50.726 | 61.600 | 1.00 | 39.59 |
| ATOM | 3246 | NE1 | TRP | 431 | 49.318 | 52.054 | 61.854 | 1.00 | 38.21 |
| ATOM | 3247 | CZ2 | TRP | 431 | 49.762 | 53.581 | 63.790 | 1.00 | 36.66 |
| ATOM | 3248 | CZ3 | TRP | 431 | 50.913 | 52.463 | 65.610 | 1.00 | 40.07 |
| ATOM | 3249 | CH2 | TRP | 431 | 50.325 | 53.612 | 65.040 | 1.00 | 37.72 |
| ATOM | 3250 | C | TRP | 431 | 52.177 | 48.900 | 60.636 | 1.00 | 38.01 |
| ATOM | 3251 | O | TRP | 431 | 52.692 | 49.925 | 60.205 | 1.00 | 38.72 |
| ATOM | 3252 | N | LEU | 432 | 51.605 | 48.014 | 59.835 | 1.00 | 38.11 |
| ATOM | 3253 | CA | LEU | 432 | 51.596 | 48.239 | 58.402 | 1.00 | 37.62 |
| ATOM | 3254 | CB | LEU | 432 | 51.281 | 46.929 | 57.692 | 1.00 | 37.35 |
| ATOM | 3255 | CG | LEU | 432 | 52.243 | 45.818 | 58.122 | 1.00 | 34.41 |
| ATOM | 3256 | CD1 | LEU | 432 | 51.976 | 44.598 | 57.284 | 1.00 | 37.13 |
| ATOM | 3257 | CD2 | LEU | 432 | 53.680 | 46.269 | 57.959 | 1.00 | 32.05 |
| ATOM | 3258 | C | LEU | 432 | 50.657 | 49.344 | 57.940 | 1.00 | 38.77 |
| ATOM | 3259 | O | LEU | 432 | 51.105 | 50.356 | 57.384 | 1.00 | 39.52 |
| ATOM | 3260 | N | GLY | 433 | 49.359 | 49.154 | 58.144 | 1.00 | 38.86 |
| ATOM | 3261 | CA | GLY | 433 | 48.411 | 50.180 | 57.742 | 1.00 | 38.46 |
| ATOM | 3262 | C | GLY | 433 | 48.067 | 50.179 | 56.267 | 1.00 | 40.43 |
| ATOM | 3263 | O | GLY | 433 | 47.531 | 51.159 | 55.742 | 1.00 | 39.11 |
| ATOM | 3264 | N | LEU | 434 | 48.393 | 49.083 | 55.588 | 1.00 | 41.72 |
| ATOM | 3265 | CA | LEU | 434 | 48.091 | 48.942 | 54.166 | 1.00 | 44.07 |
| ATOM | 3266 | CB | LEU | 434 | 49.052 | 47.922 | 53.552 | 1.00 | 41.25 |
| ATOM | 3267 | CG | LEU | 434 | 50.539 | 48.289 | 53.655 | 1.00 | 41.54 |
| ATOM | 3268 | CD1 | LEU | 434 | 51.399 | 47.068 | 53.381 | 1.00 | 39.57 |
| ATOM | 3269 | CD2 | LEU | 434 | 50.872 | 49.397 | 52.668 | 1.00 | 40.90 |
| ATOM | 3270 | C | LEU | 434 | 46.636 | 48.443 | 54.092 | 1.00 | 46.14 |
| ATOM | 3271 | O | LEU | 434 | 46.341 | 47.409 | 53.483 | 1.00 | 45.75 |
| ATOM | 3272 | N | ARG | 435 | 45.734 | 49.201 | 54.716 | 1.00 | 47.92 |
| ATOM | 3273 | CA | ARG | 435 | 44.319 | 48.834 | 54.812 | 1.00 | 50.23 |
| ATOM | 3274 | CB | ARG | 435 | 43.540 | 49.923 | 55.549 | 1.00 | 54.76 |
| ATOM | 3275 | CG | ARG | 435 | 43.562 | 51.270 | 54.874 | 1.00 | 59.93 |
| ATOM | 3276 | CD | ARG | 435 | 42.595 | 52.231 | 55.537 | 1.00 | 64.22 |
| ATOM | 3277 | NE | ARG | 435 | 42.370 | 53.382 | 54.672 | 1.00 | 71.06 |
| ATOM | 3278 | CZ | ARG | 435 | 41.303 | 54.170 | 54.722 | 1.00 | 73.22 |
| ATOM | 3279 | NH1 | ARG | 435 | 40.338 | 53.940 | 55.611 | 1.00 | 74.09 |
| ATOM | 3280 | NH2 | ARG | 435 | 41.196 | 55.180 | 53.864 | 1.00 | 74.84 |
| ATOM | 3281 | C | ARG | 435 | 43.550 | 48.452 | 53.559 | 1.00 | 49.26 |
| ATOM | 3282 | O | ARG | 435 | 42.602 | 47.682 | 53.649 | 1.00 | 49.02 |
| ATOM | 3283 | N | SER | 436 | 43.937 | 48.970 | 52.398 | 1.00 | 48.80 |
| ATOM | 3284 | CA | SER | 436 | 43.236 | 48.633 | 51.161 | 1.00 | 47.08 |
| ATOM | 3285 | CB | SER | 436 | 43.154 | 49.855 | 50.251 | 1.00 | 49.11 |
| ATOM | 3286 | OG | SER | 436 | 42.243 | 50.807 | 50.760 | 1.00 | 51.09 |
| ATOM | 3287 | C | SER | 436 | 43.851 | 47.481 | 50.374 | 1.00 | 46.81 |
| ATOM | 3288 | O | SER | 436 | 43.266 | 47.009 | 49.396 | 1.00 | 48.99 |
| ATOM | 3289 | N | LEU | 437 | 45.019 | 47.014 | 50.801 | 1.00 | 46.11 |
| ATOM | 3290 | CA | LEU | 437 | 45.710 | 45.940 | 50.096 | 1.00 | 44.52 |
| ATOM | 3291 | CB | LEU | 437 | 47.107 | 45.758 | 50.674 | 1.00 | 42.22 |
| ATOM | 3292 | CG | LEU | 437 | 47.982 | 44.747 | 49.937 | 1.00 | 40.49 |
| ATOM | 3293 | CD1 | LEU | 437 | 48.241 | 45.250 | 48.519 | 1.00 | 36.99 |
| ATOM | 3294 | CD2 | LEU | 437 | 49.290 | 44.552 | 50.698 | 1.00 | 39.32 |
| ATOM | 3295 | C | LEU | 437 | 44.976 | 44.610 | 50.132 | 1.00 | 45.78 |
| ATOM | 3296 | O | LEU | 437 | 44.915 | 43.955 | 51.174 | 1.00 | 45.88 |
| ATOM | 3297 | N | ARG | 438 | 44.436 | 44.194 | 48.993 | 1.00 | 46.68 |
| ATOM | 3298 | CA | ARG | 438 | 43.715 | 42.928 | 48.949 | 1.00 | 49.57 |
| ATOM | 3299 | CB | ARG | 438 | 42.283 | 43.137 | 48.435 | 1.00 | 51.68 |
| ATOM | 3300 | CG | ARG | 438 | 42.228 | 43.703 | 47.025 | 1.00 | 57.84 |
| ATOM | 3301 | CD | ARG | 438 | 41.034 | 43.180 | 46.234 | 1.00 | 62.85 |
| ATOM | 3302 | NE | ARG | 438 | 41.190 | 43.425 | 44.800 | 1.00 | 64.06 |
| ATOM | 3303 | CZ | ARG | 438 | 41.115 | 44.625 | 44.233 | 1.00 | 65.00 |
| ATOM | 3304 | NH1 | ARG | 438 | 40.874 | 45.700 | 44.978 | 1.00 | 63.57 |
| ATOM | 3305 | NH2 | ARG | 438 | 41.305 | 44.753 | 42.925 | 1.00 | 65.53 |
| ATOM | 3306 | C | ARG | 438 | 44.404 | 41.880 | 48.081 | 1.00 | 48.81 |
| ATOM | 3307 | O | ARG | 438 | 43.934 | 40.743 | 47.998 | 1.00 | 49.66 |
| ATOM | 3308 | N | GLU | 439 | 45.504 | 42.245 | 47.425 | 1.00 | 47.06 |
| ATOM | 3309 | CA | GLU | 439 | 46.194 | 41.273 | 46.577 | 1.00 | 46.12 |
| ATOM | 3310 | CB | GLU | 439 | 45.407 | 41.026 | 45.280 | 1.00 | 45.20 |
| ATOM | 3311 | CG | GLU | 439 | 46.269 | 40.369 | 44.188 | 1.00 | 48.25 |
| ATOM | 3312 | CD | GLU | 439 | 45.546 | 40.096 | 42.873 | 1.00 | 49.06 |
| ATOM | 3313 | OE1 | GLU | 439 | 44.639 | 40.867 | 42.494 | 1.00 | 52.99 |
| ATOM | 3314 | OE2 | GLU | 439 | 45.910 | 39.112 | 42.199 | 1.00 | 49.12 |
| ATOM | 3315 | C | GLU | 439 | 47.649 | 41.519 | 46.180 | 1.00 | 44.21 |
| ATOM | 3316 | O | GLU | 439 | 48.061 | 42.634 | 45.860 | 1.00 | 43.68 |
| ATOM | 3317 | N | LEU | 440 | 48.403 | 40.429 | 46.192 | 1.00 | 41.98 |
| ATOM | 3318 | CA | LEU | 440 | 49.789 | 40.407 | 45.768 | 1.00 | 40.35 |
| ATOM | 3319 | CB | LEU | 440 | 50.687 | 39.888 | 46.885 | 1.00 | 38.01 |
| ATOM | 3320 | CG | LEU | 440 | 50.834 | 40.836 | 48.079 | 1.00 | 36.14 |
| ATOM | 3321 | CD1 | LEU | 440 | 51.618 | 40.156 | 49.172 | 1.00 | 36.28 |
| ATOM | 3322 | CD2 | LEU | 440 | 51.549 | 42.102 | 47.643 | 1.00 | 37.02 |
| ATOM | 3323 | C | LEU | 440 | 49.724 | 39.412 | 44.618 | 1.00 | 40.73 |
| ATOM | 3324 | O | LEU | 440 | 49.757 | 38.204 | 44.830 | 1.00 | 42.71 |
| ATOM | 3325 | N | GLY | 441 | 49.590 | 39.932 | 43.402 | 1.00 | 41.24 |
| ATOM | 3326 | CA | GLY | 441 | 49.472 | 39.088 | 42.223 | 1.00 | 41.63 |
| ATOM | 3327 | C | GLY | 441 | 50.400 | 37.892 | 42.136 | 1.00 | 42.55 |
| ATOM | 3328 | O | GLY | 441 | 50.009 | 36.821 | 41.668 | 1.00 | 43.43 |
| ATOM | 3329 | N | SER | 442 | 51.641 | 38.076 | 42.563 | 1.00 | 41.02 |
| ATOM | 3330 | CA | SER | 442 | 52.613 | 37.002 | 42.531 | 1.00 | 40.92 |
| ATOM | 3331 | CB | SER | 442 | 53.030 | 36.683 | 41.092 | 1.00 | 41.27 |
| ATOM | 3332 | OG | SER | 442 | 53.645 | 37.802 | 40.492 | 1.00 | 45.36 |
| ATOM | 3333 | C | SER | 442 | 53.808 | 37.447 | 43.338 | 1.00 | 40.20 |
| ATOM | 3334 | O | SER | 442 | 53.890 | 38.613 | 43.742 | 1.00 | 40.30 |
| ATOM | 3335 | N | GLY | 443 | 54.728 | 36.518 | 43.582 | 1.00 | 38.65 |
| ATOM | 3336 | CA | GLY | 443 | 55.901 | 36.834 | 44.369 | 1.00 | 37.83 |
| ATOM | 3337 | C | GLY | 443 | 55.631 | 36.603 | 45.844 | 1.00 | 39.08 |
| ATOM | 3338 | O | GLY | 443 | 54.475 | 36.493 | 46.268 | 1.00 | 39.98 |
| ATOM | 3339 | N | LEU | 444 | 56.696 | 36.525 | 46.631 | 1.00 | 37.91 |
| ATOM | 3340 | CA | LEU | 444 | 56.567 | 36.310 | 48.053 | 1.00 | 37.00 |
| ATOM | 3341 | CB | LEU | 444 | 57.694 | 35.415 | 48.538 | 1.00 | 38.41 |
| ATOM | 3342 | CG | LEU | 444 | 57.537 | 33.963 | 48.088 | 1.00 | 42.05 |
| ATOM | 3343 | CD1 | LEU | 444 | 58.864 | 33.394 | 47.609 | 1.00 | 45.89 |
| ATOM | 3344 | CD2 | LEU | 444 | 56.983 | 33.161 | 49.241 | 1.00 | 42.56 |
| ATOM | 3345 | C | LEU | 444 | 56.630 | 37.640 | 48.764 | 1.00 | 38.87 |
| ATOM | 3346 | O | LEU | 444 | 56.975 | 38.656 | 48.162 | 1.00 | 42.31 |
| ATOM | 3347 | N | ALA | 445 | 56.276 | 37.632 | 50.044 | 1.00 | 37.35 |
| ATOM | 3348 | CA | ALA | 445 | 56.310 | 38.821 | 50.868 | 1.00 | 34.33 |
| ATOM | 3349 | CB | ALA | 445 | 54.996 | 39.004 | 51.575 | 1.00 | 34.87 |
| ATOM | 3350 | C | ALA | 445 | 57.413 | 38.595 | 51.881 | 1.00 | 34.94 |
| ATOM | 3351 | O | ALA | 445 | 57.487 | 37.534 | 52.506 | 1.00 | 34.95 |
| ATOM | 3352 | N | LEU | 446 | 58.282 | 39.582 | 52.040 | 1.00 | 33.98 |
| ATOM | 3353 | CA | LEU | 446 | 59.366 | 39.457 | 52.990 | 1.00 | 32.14 |
| ATOM | 3354 | CB | LEU | 446 | 60.705 | 39.427 | 52.259 | 1.00 | 31.79 |
| ATOM | 3355 | CG | LEU | 446 | 61.968 | 39.387 | 53.128 | 1.00 | 35.26 |
| ATOM | 3356 | CD1 | LEU | 446 | 61.878 | 38.262 | 54.154 | 1.00 | 35.34 |
| ATOM | 3357 | CD2 | LEU | 446 | 63.183 | 39.202 | 52.237 | 1.00 | 35.63 |
| ATOM | 3358 | C | LEU | 446 | 59.302 | 40.627 | 53.946 | 1.00 | 34.42 |
| ATOM | 3359 | O | LEU | 446 | 59.372 | 41.787 | 53.526 | 1.00 | 36.09 |
| ATOM | 3360 | N | ILE | 447 | 59.148 | 40.316 | 55.233 | 1.00 | 34.59 |
| ATOM | 3361 | CA | ILE | 447 | 59.059 | 41.326 | 56.279 | 1.00 | 33.79 |
| ATOM | 3362 | CB | ILE | 447 | 57.617 | 41.456 | 56.770 | 1.00 | 33.38 |
| ATOM | 3363 | CG2 | ILE | 447 | 57.528 | 42.521 | 57.846 | 1.00 | 34.06 |
| ATOM | 3364 | CG1 | ILE | 447 | 56.699 | 41.770 | 55.588 | 1.00 | 32.75 |
| ATOM | 3365 | CD1 | ILE | 447 | 55.245 | 42.062 | 55.994 | 1.00 | 34.78 |
| ATOM | 3366 | C | ILE | 447 | 59.944 | 40.889 | 57.438 | 1.00 | 35.09 |
| ATOM | 3367 | O | ILE | 447 | 59.622 | 39.931 | 58.147 | 1.00 | 36.39 |
| ATOM | 3368 | N | HIS | 448 | 61.061 | 41.574 | 57.643 | 1.00 | 34.59 |
| ATOM | 3369 | CA | HIS | 448 | 61.945 | 41.156 | 58.716 | 1.00 | 37.11 |
| ATOM | 3370 | CB | HIS | 448 | 62.853 | 40.028 | 58.223 | 1.00 | 39.27 |
| ATOM | 3371 | CG | HIS | 448 | 63.904 | 40.486 | 57.264 | 1.00 | 38.82 |
| ATOM | 3372 | CD2 | HIS | 448 | 63.852 | 40.725 | 55.933 | 1.00 | 39.04 |
| ATOM | 3373 | ND1 | HIS | 448 | 65.177 | 40.825 | 57.667 | 1.00 | 39.32 |
| ATOM | 3374 | CE1 | HIS | 448 | 65.864 | 41.257 | 56.624 | 1.00 | 40.51 |
| ATOM | 3375 | NE2 | HIS | 448 | 65.083 | 41.207 | 55.560 | 1.00 | 41.38 |
| ATOM | 3376 | C | HIS | 448 | 62.792 | 42.277 | 59.285 | 1.00 | 39.00 |
| ATOM | 3377 | O | HIS | 448 | 63.011 | 43.306 | 58.637 | 1.00 | 39.77 |
| ATOM | 3378 | N | HIS | 449 | 63.276 | 42.046 | 60.502 | 1.00 | 39.63 |
| ATOM | 3379 | CA | HIS | 449 | 64.091 | 43.008 | 61.222 | 1.00 | 40.27 |
| ATOM | 3380 | CB | HIS | 449 | 65.477 | 43.120 | 60.594 | 1.00 | 40.23 |
| ATOM | 3381 | CG | HIS | 449 | 66.346 | 41.940 | 60.892 | 1.00 | 45.12 |
| ATOM | 3382 | CD2 | HIS | 449 | 67.248 | 41.725 | 61.879 | 1.00 | 45.97 |
| ATOM | 3383 | ND1 | HIS | 449 | 66.260 | 40.757 | 60.188 | 1.00 | 46.84 |
| ATOM | 3384 | CE1 | HIS | 449 | 67.071 | 39.864 | 60.731 | 1.00 | 47.26 |
| ATOM | 3385 | NE2 | HIS | 449 | 67.682 | 40.426 | 61.761 | 1.00 | 46.02 |
| ATOM | 3386 | C | HIS | 449 | 63.439 | 44.370 | 61.362 | 1.00 | 39.67 |
| ATOM | 3387 | O | HIS | 449 | 64.067 | 45.410 | 61.157 | 1.00 | 39.10 |
| ATOM | 3388 | N | ASN | 450 | 62.157 | 44.339 | 61.711 | 1.00 | 39.45 |
| ATOM | 3389 | CA | ASN | 450 | 61.371 | 45.544 | 61.965 | 1.00 | 38.40 |
| ATOM | 3390 | CB | ASN | 450 | 60.119 | 45.525 | 61.110 | 1.00 | 37.35 |
| ATOM | 3391 | CG | ASN | 450 | 60.442 | 45.445 | 59.639 | 1.00 | 35.81 |
| ATOM | 3392 | OD1 | ASN | 450 | 61.142 | 46.313 | 59.106 | 1.00 | 35.66 |
| ATOM | 3393 | ND2 | ASN | 450 | 59.942 | 44.407 | 58.971 | 1.00 | 32.14 |
| ATOM | 3394 | C | ASN | 450 | 61.034 | 45.410 | 63.445 | 1.00 | 38.45 |
| ATOM | 3395 | O | ASN | 450 | 60.003 | 44.846 | 63.816 | 1.00 | 38.05 |
| ATOM | 3396 | N | THR | 451 | 61.948 | 45.908 | 64.274 | 1.00 | 38.23 |
| ATOM | 3397 | CA | THR | 451 | 61.881 | 45.839 | 65.729 | 1.00 | 39.20 |
| ATOM | 3398 | CB | THR | 451 | 62.996 | 46.689 | 66.323 | 1.00 | 40.04 |
| ATOM | 3399 | OG1 | THR | 451 | 64.247 | 46.203 | 65.835 | 1.00 | 44.58 |
| ATOM | 3400 | CG2 | THR | 451 | 62.984 | 46.622 | 67.840 | 1.00 | 38.85 |
| ATOM | 3401 | C | THR | 451 | 60.590 | 46.192 | 66.463 | 1.00 | 40.85 |
| ATOM | 3402 | O | THR | 451 | 60.184 | 45.491 | 67.400 | 1.00 | 40.47 |
| ATOM | 3403 | N | HIS | 452 | 59.955 | 47.281 | 66.058 | 1.00 | 40.04 |
| ATOM | 3404 | CA | HIS | 452 | 58.745 | 47.724 | 66.721 | 1.00 | 40.12 |
| ATOM | 3405 | CB | HIS | 452 | 58.771 | 49.249 | 66.842 | 1.00 | 41.64 |
| ATOM | 3406 | CG | HIS | 452 | 60.048 | 49.797 | 67.401 | 1.00 | 42.19 |
| ATOM | 3407 | CD2 | HIS | 452 | 61.034 | 50.523 | 66.823 | 1.00 | 42.10 |
| ATOM | 3408 | ND1 | HIS | 452 | 60.404 | 49.655 | 68.726 | 1.00 | 41.64 |
| ATOM | 3409 | CE1 | HIS | 452 | 61.551 | 50.277 | 68.941 | 1.00 | 42.19 |
| ATOM | 3410 | NE2 | HIS | 452 | 61.955 | 50.813 | 67.803 | 1.00 | 41.89 |
| ATOM | 3411 | C | HIS | 452 | 57.504 | 47.316 | 65.951 | 1.00 | 40.45 |
| ATOM | 3412 | O | HIS | 452 | 56.407 | 47.801 | 66.231 | 1.00 | 41.14 |
| ATOM | 3413 | N | LEU | 453 | 57.660 | 46.424 | 64.984 | 1.00 | 40.04 |
| ATOM | 3414 | CA | LEU | 453 | 56.521 | 46.049 | 64.167 | 1.00 | 40.19 |
| ATOM | 3415 | CB | LEU | 453 | 57.001 | 45.606 | 62.782 | 1.00 | 37.00 |
| ATOM | 3416 | CG | LEU | 453 | 55.893 | 45.347 | 61.759 | 1.00 | 37.34 |
| ATOM | 3417 | CD1 | LEU | 453 | 55.194 | 46.659 | 61.397 | 1.00 | 32.28 |
| ATOM | 3418 | CD2 | LEU | 453 | 56.490 | 44.686 | 60.516 | 1.00 | 35.69 |
| ATOM | 3419 | C | LEU | 453 | 55.597 | 44.993 | 64.746 | 1.00 | 40.85 |
| ATOM | 3420 | O | LEU | 453 | 56.018 | 43.883 | 65.038 | 1.00 | 41.66 |
| ATOM | 3421 | N | CYS | 454 | 54.333 | 45.360 | 64.925 | 1.00 | 43.03 |
| ATOM | 3422 | CA | CYS | 454 | 53.324 | 44.426 | 65.404 | 1.00 | 45.80 |
| ATOM | 3423 | C | CYS | 454 | 52.311 | 44.337 | 64.277 | 1.00 | 47.23 |
| ATOM | 3424 | O | CYS | 454 | 52.589 | 44.766 | 63.160 | 1.00 | 50.06 |
| ATOM | 3425 | CB | CYS | 454 | 52.647 | 44.928 | 66.682 | 1.00 | 48.08 |
| ATOM | 3426 | SG | CYS | 454 | 53.699 | 44.807 | 68.162 | 1.00 | 52.71 |
| ATOM | 3427 | N | PHE | 455 | 51.144 | 43.776 | 64.554 | 1.00 | 46.70 |
| ATOM | 3428 | CA | PHE | 455 | 50.107 | 43.660 | 63.537 | 1.00 | 46.11 |
| ATOM | 3429 | CB | PHE | 455 | 49.487 | 45.025 | 63.255 | 1.00 | 44.91 |
| ATOM | 3430 | CG | PHE | 455 | 48.892 | 45.645 | 64.460 | 1.00 | 45.40 |
| ATOM | 3431 | CD1 | PHE | 455 | 49.692 | 46.348 | 65.358 | 1.00 | 46.60 |
| ATOM | 3432 | CD2 | PHE | 455 | 47.555 | 45.440 | 64.763 | 1.00 | 45.00 |
| ATOM | 3433 | CE1 | PHE | 455 | 49.169 | 46.831 | 66.545 | 1.00 | 48.01 |
| ATOM | 3434 | CE2 | PHE | 455 | 47.015 | 45.916 | 65.947 | 1.00 | 46.34 |
| ATOM | 3435 | CZ | PHE | 455 | 47.819 | 46.613 | 66.844 | 1.00 | 47.91 |
| ATOM | 3436 | C | PHE | 455 | 50.564 | 43.033 | 62.244 | 1.00 | 45.88 |
| ATOM | 3437 | O | PHE | 455 | 50.200 | 43.484 | 61.168 | 1.00 | 46.84 |
| ATOM | 3438 | N | VAL | 456 | 51.367 | 41.989 | 62.355 | 1.00 | 47.26 |
| ATOM | 3439 | CA | VAL | 456 | 51.833 | 41.263 | 61.191 | 1.00 | 49.14 |
| ATOM | 3440 | CB | VAL | 456 | 53.332 | 40.892 | 61.315 | 1.00 | 50.57 |
| ATOM | 3441 | CG1 | VAL | 456 | 53.768 | 40.038 | 60.121 | 1.00 | 48.77 |
| ATOM | 3442 | CG2 | VAL | 456 | 54.174 | 42.159 | 61.412 | 1.00 | 49.21 |
| ATOM | 3443 | C | VAL | 456 | 50.998 | 39.988 | 61.183 | 1.00 | 51.32 |
| ATOM | 3444 | O | VAL | 456 | 50.487 | 39.569 | 60.137 | 1.00 | 51.84 |
| ATOM | 3445 | N | HIS | 457 | 50.844 | 39.387 | 62.365 | 1.00 | 52.85 |
| ATOM | 3446 | CA | HIS | 457 | 50.080 | 38.149 | 62.499 | 1.00 | 54.27 |
| ATOM | 3447 | CB | HIS | 457 | 50.398 | 37.435 | 63.830 | 1.00 | 57.53 |
| ATOM | 3448 | CG | HIS | 457 | 49.938 | 38.172 | 65.056 | 1.00 | 62.48 |
| ATOM | 3449 | CD2 | HIS | 457 | 48.920 | 37.921 | 65.917 | 1.00 | 63.06 |
| ATOM | 3450 | ND1 | HIS | 457 | 50.562 | 39.310 | 65.527 | 1.00 | 63.53 |
| ATOM | 3451 | CE1 | HIS | 457 | 49.950 | 39.726 | 66.622 | 1.00 | 63.21 |
| ATOM | 3452 | NE2 | HIS | 457 | 48.951 | 38.903 | 66.880 | 1.00 | 63.61 |
| ATOM | 3453 | C | HIS | 457 | 48.583 | 38.390 | 62.389 | 1.00 | 52.92 |
| ATOM | 3454 | O | HIS | 457 | 47.819 | 37.448 | 62.190 | 1.00 | 52.93 |
| ATOM | 3455 | N | THR | 458 | 48.173 | 39.652 | 62.505 | 1.00 | 51.57 |
| ATOM | 3456 | CA | THR | 458 | 46.760 | 40.016 | 62.422 | 1.00 | 49.96 |
| ATOM | 3457 | CB | THR | 458 | 46.488 | 41.361 | 63.099 | 1.00 | 50.57 |
| ATOM | 3458 | OG1 | THR | 458 | 47.275 | 42.382 | 62.468 | 1.00 | 48.76 |
| ATOM | 3459 | CG2 | THR | 458 | 46.835 | 41.290 | 64.576 | 1.00 | 50.84 |
| ATOM | 3460 | C | THR | 458 | 46.275 | 40.127 | 60.984 | 1.00 | 49.72 |
| ATOM | 3461 | O | THR | 458 | 45.078 | 40.284 | 60.742 | 1.00 | 49.69 |
| ATOM | 3462 | N | VAL | 459 | 47.204 | 40.055 | 60.037 | 1.00 | 47.78 |
| ATOM | 3463 | CA | VAL | 459 | 46.862 | 40.152 | 58.626 | 1.00 | 46.18 |
| ATOM | 3464 | CB | VAL | 459 | 47.970 | 40.901 | 57.836 | 1.00 | 45.27 |
| ATOM | 3465 | CG1 | VAL | 459 | 47.655 | 40.896 | 56.357 | 1.00 | 41.77 |
| ATOM | 3466 | CG2 | VAL | 459 | 48.088 | 42.327 | 58.340 | 1.00 | 42.75 |
| ATOM | 3467 | C | VAL | 459 | 46.649 | 38.768 | 58.008 | 1.00 | 47.65 |
| ATOM | 3468 | O | VAL | 459 | 47.468 | 37.862 | 58.174 | 1.00 | 47.15 |
| ATOM | 3469 | N | PRO | 460 | 45.532 | 38.590 | 57.281 | 1.00 | 48.28 |
| ATOM | 3470 | CD | PRO | 460 | 44.465 | 39.590 | 57.079 | 1.00 | 47.27 |
| ATOM | 3471 | CA | PRO | 460 | 45.188 | 37.323 | 56.626 | 1.00 | 47.47 |
| ATOM | 3472 | CB | PRO | 460 | 43.693 | 37.475 | 56.388 | 1.00 | 47.62 |
| ATOM | 3473 | CG | PRO | 460 | 43.589 | 38.932 | 56.022 | 1.00 | 46.72 |
| ATOM | 3474 | C | PRO | 460 | 45.965 | 37.157 | 55.318 | 1.00 | 47.25 |
| ATOM | 3475 | O | PRO | 460 | 45.376 | 37.114 | 54.234 | 1.00 | 47.13 |
| ATOM | 3476 | N | TRP | 461 | 47.285 | 37.052 | 55.427 | 1.00 | 45.76 |
| ATOM | 3477 | CA | TRP | 461 | 48.151 | 36.934 | 54.258 | 1.00 | 44.54 |
| ATOM | 3478 | CB | TRP | 461 | 49.569 | 36.609 | 54.717 | 1.00 | 40.95 |
| ATOM | 3479 | CG | TRP | 461 | 50.152 | 37.709 | 55.539 | 1.00 | 40.30 |
| ATOM | 3480 | CD2 | TRP | 461 | 50.621 | 38.978 | 55.060 | 1.00 | 40.35 |
| ATOM | 3481 | CE2 | TRP | 461 | 51.053 | 39.716 | 56.185 | 1.00 | 39.87 |
| ATOM | 3482 | CE3 | TRP | 461 | 50.718 | 39.562 | 53.789 | 1.00 | 39.46 |
| ATOM | 3483 | CD1 | TRP | 461 | 50.310 | 37.731 | 56.897 | 1.00 | 38.03 |
| ATOM | 3484 | NE1 | TRP | 461 | 50.850 | 38.934 | 57.293 | 1.00 | 38.31 |
| ATOM | 3485 | CZ2 | TRP | 461 | 51.576 | 41.008 | 56.075 | 1.00 | 39.81 |
| ATOM | 3486 | CZ3 | TRP | 461 | 51.237 | 40.847 | 53.681 | 1.00 | 38.47 |
| ATOM | 3487 | CH2 | TRP | 461 | 51.660 | 41.555 | 54.818 | 1.00 | 40.35 |
| ATOM | 3488 | C | TRP | 461 | 47.707 | 35.960 | 53.166 | 1.00 | 45.92 |
| ATOM | 3489 | O | TRP | 461 | 47.841 | 36.254 | 51.973 | 1.00 | 42.98 |
| ATOM | 3490 | N | ASP | 462 | 47.172 | 34.810 | 53.563 | 1.00 | 48.29 |
| ATOM | 3491 | CA | ASP | 462 | 46.713 | 33.817 | 52.593 | 1.00 | 50.04 |
| ATOM | 3492 | CB | ASP | 462 | 46.136 | 32.615 | 53.327 | 1.00 | 54.39 |
| ATOM | 3493 | CG | ASP | 462 | 47.196 | 31.833 | 54.068 | 1.00 | 60.56 |
| ATOM | 3494 | OD1 | ASP | 462 | 46.845 | 31.154 | 55.064 | 1.00 | 63.99 |
| ATOM | 3495 | OD2 | ASP | 462 | 48.380 | 31.890 | 53.648 | 1.00 | 62.32 |
| ATOM | 3496 | C | ASP | 462 | 45.676 | 34.388 | 51.631 | 1.00 | 47.95 |
| ATOM | 3497 | O | ASP | 462 | 45.593 | 33.979 | 50.482 | 1.00 | 47.15 |
| ATOM | 3498 | N | GLN | 463 | 44.879 | 35.327 | 52.112 | 1.00 | 47.85 |
| ATOM | 3499 | CA | GLN | 463 | 43.868 | 35.951 | 51.279 | 1.00 | 49.93 |
| ATOM | 3500 | CB | GLN | 463 | 42.964 | 36.862 | 52.132 | 1.00 | 52.77 |
| ATOM | 3501 | CG | GLN | 463 | 41.948 | 36.126 | 53.015 | 1.00 | 57.25 |
| ATOM | 3502 | CD | GLN | 463 | 41.022 | 37.074 | 53.790 | 1.00 | 60.95 |
| ATOM | 3503 | OE1 | GLN | 463 | 40.433 | 38.005 | 53.219 | 1.00 | 62.40 |
| ATOM | 3504 | NE2 | GLN | 463 | 40.880 | 36.830 | 55.095 | 1.00 | 62.71 |
| ATOM | 3505 | C | GLN | 463 | 44.504 | 36.766 | 50.138 | 1.00 | 49.27 |
| ATOM | 3506 | O | GLN | 463 | 43.979 | 36.789 | 49.026 | 1.00 | 49.51 |
| ATOM | 3507 | N | LEU | 464 | 45.636 | 37.418 | 50.405 | 1.00 | 47.31 |
| ATOM | 3508 | CA | LEU | 464 | 46.304 | 38.237 | 49.394 | 1.00 | 46.23 |
| ATOM | 3509 | CB | LEU | 464 | 47.310 | 39.169 | 50.068 | 1.00 | 46.42 |
| ATOM | 3510 | CG | LEU | 464 | 46.803 | 39.973 | 51.266 | 1.00 | 47.92 |
| ATOM | 3511 | CD1 | LEU | 464 | 47.859 | 40.972 | 51.695 | 1.00 | 46.98 |
| ATOM | 3512 | CD2 | LEU | 464 | 45.526 | 40.685 | 50.896 | 1.00 | 49.82 |
| ATOM | 3513 | C | LEU | 464 | 47.012 | 37.480 | 48.257 | 1.00 | 45.85 |
| ATOM | 3514 | O | LEU | 464 | 47.095 | 37.977 | 47.129 | 1.00 | 45.77 |
| ATOM | 3515 | N | PHE | 465 | 47.524 | 36.290 | 48.542 | 1.00 | 44.21 |
| ATOM | 3516 | CA | PHE | 465 | 48.229 | 35.514 | 47.532 | 1.00 | 45.10 |
| ATOM | 3517 | CB | PHE | 465 | 49.029 | 34.400 | 48.203 | 1.00 | 42.93 |
| ATOM | 3518 | CG | PHE | 465 | 50.001 | 34.895 | 49.240 | 1.00 | 42.74 |
| ATOM | 3519 | CD1 | PHE | 465 | 50.800 | 36.010 | 48.987 | 1.00 | 41.77 |
| ATOM | 3520 | CD2 | PHE | 465 | 50.131 | 34.243 | 50.463 | 1.00 | 41.86 |
| ATOM | 3521 | CE1 | PHE | 465 | 51.713 | 36.467 | 49.935 | 1.00 | 41.10 |
| ATOM | 3522 | CE2 | PHE | 465 | 51.041 | 34.688 | 51.421 | 1.00 | 40.99 |
| ATOM | 3523 | CZ | PHE | 465 | 51.833 | 35.801 | 51.160 | 1.00 | 42.60 |
| ATOM | 3524 | C | PHE | 465 | 47.279 | 34.935 | 46.486 | 1.00 | 47.59 |
| ATOM | 3525 | O | PHE | 465 | 46.080 | 34.816 | 46.735 | 1.00 | 49.78 |
| ATOM | 3526 | N | ARG | 466 | 47.823 | 34.570 | 45.323 | 1.00 | 47.40 |
| ATOM | 3527 | CA | ARG | 466 | 47.036 | 34.028 | 44.221 | 1.00 | 45.90 |
| ATOM | 3528 | CB | ARG | 466 | 46.903 | 35.084 | 43.130 | 1.00 | 45.38 |
| ATOM | 3529 | CG | ARG | 466 | 46.126 | 36.321 | 43.557 | 1.00 | 46.15 |
| ATOM | 3530 | CD | ARG | 466 | 44.642 | 36.023 | 43.736 | 1.00 | 43.17 |
| ATOM | 3531 | NE | ARG | 466 | 43.879 | 37.221 | 44.084 | 1.00 | 42.43 |
| ATOM | 3532 | CZ | ARG | 466 | 43.744 | 37.693 | 45.321 | 1.00 | 41.90 |
| ATOM | 3533 | NH1 | ARG | 466 | 44.318 | 37.068 | 46.337 | 1.00 | 38.17 |
| ATOM | 3534 | NH2 | ARG | 466 | 43.035 | 38.795 | 45.544 | 1.00 | 41.69 |
| ATOM | 3535 | C | ARG | 466 | 47.551 | 32.724 | 43.590 | 1.00 | 47.53 |
| ATOM | 3536 | O | ARG | 466 | 46.932 | 32.200 | 42.660 | 1.00 | 48.19 |
| ATOM | 3537 | N | ASN | 467 | 48.682 | 32.209 | 44.064 | 1.00 | 47.38 |
| ATOM | 3538 | CA | ASN | 467 | 49.220 | 30.955 | 43.535 | 1.00 | 47.50 |
| ATOM | 3539 | CB | ASN | 467 | 50.154 | 31.209 | 42.334 | 1.00 | 48.16 |
| ATOM | 3540 | CG | ASN | 467 | 51.524 | 31.725 | 42.736 | 1.00 | 50.64 |
| ATOM | 3541 | OD1 | ASN | 467 | 52.339 | 30.991 | 43.311 | 1.00 | 52.47 |
| ATOM | 3542 | ND2 | ASN | 467 | 51.793 | 32.990 | 42.425 | 1.00 | 46.93 |
| ATOM | 3543 | C | ASN | 467 | 49.924 | 30.199 | 44.668 | 1.00 | 46.88 |
| ATOM | 3544 | O | ASN | 467 | 50.341 | 30.802 | 45.659 | 1.00 | 46.18 |
| ATOM | 3545 | N | PRO | 468 | 50.063 | 28.869 | 44.534 | 1.00 | 47.46 |
| ATOM | 3546 | CD | PRO | 468 | 49.835 | 28.116 | 43.291 | 1.00 | 46.51 |
| ATOM | 3547 | CA | PRO | 468 | 50.693 | 28.003 | 45.542 | 1.00 | 48.63 |
| ATOM | 3548 | CB | PRO | 468 | 50.617 | 26.610 | 44.914 | 1.00 | 48.24 |
| ATOM | 3549 | CG | PRO | 468 | 49.584 | 26.747 | 43.814 | 1.00 | 47.11 |
| ATOM | 3550 | C | PRO | 468 | 52.116 | 28.344 | 45.930 | 1.00 | 49.84 |
| ATOM | 3551 | O | PRO | 468 | 52.572 | 27.957 | 46.999 | 1.00 | 52.80 |
| ATOM | 3552 | N | HIS | 469 | 52.814 | 29.066 | 45.065 | 1.00 | 50.04 |
| ATOM | 3553 | CA | HIS | 469 | 54.199 | 29.409 | 45.318 | 1.00 | 49.71 |
| ATOM | 3554 | CB | HIS | 469 | 54.940 | 29.548 | 43.988 | 1.00 | 53.11 |
| ATOM | 3555 | CG | HIS | 469 | 55.012 | 28.276 | 43.201 | 1.00 | 54.91 |
| ATOM | 3556 | CD2 | HIS | 469 | 54.448 | 27.924 | 42.021 | 1.00 | 54.62 |
| ATOM | 3557 | ND1 | HIS | 469 | 55.737 | 27.181 | 43.622 | 1.00 | 56.43 |
| ATOM | 3558 | CE1 | HIS | 469 | 55.620 | 26.211 | 42.733 | 1.00 | 56.70 |
| ATOM | 3559 | NE2 | HIS | 469 | 54.843 | 26.636 | 41.752 | 1.00 | 55.63 |
| ATOM | 3560 | C | HIS | 469 | 54.423 | 30.653 | 46.154 | 1.00 | 48.30 |
| ATOM | 3561 | O | HIS | 469 | 55.567 | 31.030 | 46.394 | 1.00 | 48.74 |
| ATOM | 3562 | N | GLN | 470 | 53.349 | 31.287 | 46.610 | 1.00 | 47.55 |
| ATOM | 3563 | CA | GLN | 470 | 53.479 | 32.506 | 47.414 | 1.00 | 46.16 |
| ATOM | 3564 | CB | GLN | 470 | 52.433 | 33.535 | 46.993 | 1.00 | 45.94 |
| ATOM | 3565 | CG | GLN | 470 | 52.547 | 34.014 | 45.560 | 1.00 | 47.79 |
| ATOM | 3566 | CD | GLN | 470 | 51.472 | 35.034 | 45.218 | 1.00 | 48.54 |
| ATOM | 3567 | OE1 | GLN | 470 | 50.320 | 34.677 | 44.971 | 1.00 | 48.75 |
| ATOM | 3568 | NE2 | GLN | 470 | 51.840 | 36.309 | 45.223 | 1.00 | 44.96 |
| ATOM | 3569 | C | GLN | 470 | 53.331 | 32.255 | 48.909 | 1.00 | 46.20 |
| ATOM | 3570 | O | GLN | 470 | 52.631 | 31.331 | 49.333 | 1.00 | 47.30 |
| ATOM | 3571 | N | ALA | 471 | 53.972 | 33.107 | 49.702 | 1.00 | 44.42 |
| ATOM | 3572 | CA | ALA | 471 | 53.923 | 33.000 | 51.150 | 1.00 | 43.97 |
| ATOM | 3573 | CB | ALA | 471 | 54.650 | 31.745 | 51.607 | 1.00 | 43.76 |
| ATOM | 3574 | C | ALA | 471 | 54.588 | 34.219 | 51.763 | 1.00 | 44.57 |
| ATOM | 3575 | O | ALA | 471 | 55.245 | 35.000 | 51.071 | 1.00 | 42.63 |
| ATOM | 3576 | N | LEU | 472 | 54.417 | 34.377 | 53.069 | 1.00 | 42.98 |
| ATOM | 3577 | CA | LEU | 472 | 55.031 | 35.484 | 53.767 | 1.00 | 41.66 |
| ATOM | 3578 | CB | LEU | 472 | 54.049 | 36.128 | 54.746 | 1.00 | 41.67 |
| ATOM | 3579 | CG | LEU | 472 | 54.740 | 37.049 | 55.766 | 1.00 | 42.01 |
| ATOM | 3580 | CD1 | LEU | 472 | 55.350 | 38.268 | 55.051 | 1.00 | 40.33 |
| ATOM | 3581 | CD2 | LEU | 472 | 53.745 | 37.477 | 56.824 | 1.00 | 40.11 |
| ATOM | 3582 | C | LEU | 472 | 56.215 | 34.965 | 54.549 | 1.00 | 41.03 |
| ATOM | 3583 | O | LEU | 472 | 56.069 | 34.034 | 55.330 | 1.00 | 42.94 |
| ATOM | 3584 | N | LEU | 473 | 57.388 | 35.553 | 54.336 | 1.00 | 41.80 |
| ATOM | 3585 | CA | LEU | 473 | 58.582 | 35.154 | 55.075 | 1.00 | 40.99 |
| ATOM | 3586 | CB | LEU | 473 | 59.789 | 35.020 | 54.144 | 1.00 | 39.87 |
| ATOM | 3587 | CG | LEU | 473 | 59.554 | 34.118 | 52.927 | 1.00 | 40.65 |
| ATOM | 3588 | CD1 | LEU | 473 | 60.896 | 33.717 | 52.334 | 1.00 | 40.09 |
| ATOM | 3589 | CD2 | LEU | 473 | 58.754 | 32.884 | 53.324 | 1.00 | 38.13 |
| ATOM | 3590 | C | LEU | 473 | 58.792 | 36.271 | 56.078 | 1.00 | 41.31 |
| ATOM | 3591 | O | LEU | 473 | 58.828 | 37.441 | 55.712 | 1.00 | 41.51 |
| ATOM | 3592 | N | HIS | 474 | 58.929 | 35.910 | 57.349 | 1.00 | 43.35 |
| ATOM | 3593 | CA | HIS | 474 | 59.061 | 36.909 | 58.402 | 1.00 | 42.81 |
| ATOM | 3594 | CB | HIS | 474 | 57.668 | 37.264 | 58.888 | 1.00 | 42.99 |
| ATOM | 3595 | CG | HIS | 474 | 56.926 | 36.090 | 59.442 | 1.00 | 43.37 |
| ATOM | 3596 | CD2 | HIS | 474 | 56.177 | 35.143 | 58.830 | 1.00 | 43.37 |
| ATOM | 3597 | ND1 | HIS | 474 | 56.986 | 35.732 | 60.772 | 1.00 | 44.24 |
| ATOM | 3598 | CE1 | HIS | 474 | 56.307 | 34.614 | 60.954 | 1.00 | 44.58 |
| ATOM | 3599 | NE2 | HIS | 474 | 55.806 | 34.235 | 59.791 | 1.00 | 44.84 |
| ATOM | 3600 | C | HIS | 474 | 59.884 | 36.411 | 59.581 | 1.00 | 43.18 |
| ATOM | 3601 | O | HIS | 474 | 59.763 | 35.261 | 60.004 | 1.00 | 44.46 |
| ATOM | 3602 | N | THR | 475 | 60.703 | 37.292 | 60.132 | 1.00 | 42.59 |
| ATOM | 3603 | CA | THR | 475 | 61.533 | 36.920 | 61.257 | 1.00 | 41.62 |
| ATOM | 3604 | CB | THR | 475 | 62.673 | 35.973 | 60.802 | 1.00 | 42.51 |
| ATOM | 3605 | OG1 | THR | 475 | 63.340 | 35.423 | 61.948 | 1.00 | 47.00 |
| ATOM | 3606 | CG2 | THR | 475 | 63.685 | 36.724 | 59.953 | 1.00 | 42.92 |
| ATOM | 3607 | C | THR | 475 | 62.118 | 38.195 | 61.826 | 1.00 | 40.91 |
| ATOM | 3608 | O | THR | 475 | 62.245 | 39.188 | 61.117 | 1.00 | 41.61 |
| ATOM | 3609 | N | ALA | 476 | 62.458 | 38.171 | 63.110 | 1.00 | 41.19 |
| ATOM | 3610 | CA | ALA | 476 | 63.057 | 39.324 | 63.778 | 1.00 | 39.52 |
| ATOM | 3611 | CB | ALA | 476 | 64.442 | 39.612 | 63.178 | 1.00 | 37.39 |
| ATOM | 3612 | C | ALA | 476 | 62.221 | 40.615 | 63.814 | 1.00 | 40.28 |
| ATOM | 3613 | O | ALA | 476 | 62.774 | 41.719 | 63.790 | 1.00 | 41.36 |
| ATOM | 3614 | N | ASN | 477 | 60.897 | 40.493 | 63.846 | 1.00 | 39.57 |
| ATOM | 3615 | CA | ASN | 477 | 60.068 | 41.683 | 63.981 | 1.00 | 39.64 |
| ATOM | 3616 | CB | ASN | 477 | 58.836 | 41.609 | 63.084 | 1.00 | 40.20 |
| ATOM | 3617 | CG | ASN | 477 | 59.198 | 41.577 | 61.593 | 1.00 | 42.24 |
| ATOM | 3618 | OD1 | ASN | 477 | 59.810 | 42.514 | 61.062 | 1.00 | 40.34 |
| ATOM | 3619 | ND2 | ASN | 477 | 58.818 | 40.497 | 60.918 | 1.00 | 40.44 |
| ATOM | 3620 | C | ASN | 477 | 59.686 | 41.674 | 65.465 | 1.00 | 40.57 |
| ATOM | 3621 | O | ASN | 477 | 60.159 | 40.809 | 66.214 | 1.00 | 38.44 |
| ATOM | 3622 | N | ARG | 478 | 58.858 | 42.614 | 65.914 | 1.00 | 41.50 |
| ATOM | 3623 | CA | ARG | 478 | 58.506 | 42.630 | 67.329 | 1.00 | 40.80 |
| ATOM | 3624 | CB | ARG | 478 | 57.610 | 43.816 | 67.665 | 1.00 | 41.88 |
| ATOM | 3625 | CG | ARG | 478 | 57.488 | 44.006 | 69.161 | 1.00 | 42.92 |
| ATOM | 3626 | CD | ARG | 478 | 56.713 | 45.238 | 69.585 | 1.00 | 42.69 |
| ATOM | 3627 | NE | ARG | 478 | 56.840 | 45.392 | 71.035 | 1.00 | 46.73 |
| ATOM | 3628 | CZ | ARG | 478 | 56.010 | 46.080 | 71.814 | 1.00 | 44.48 |
| ATOM | 3629 | NH1 | ARG | 478 | 54.959 | 46.702 | 71.302 | 1.00 | 45.76 |
| ATOM | 3630 | NH2 | ARG | 478 | 56.239 | 46.144 | 73.115 | 1.00 | 45.84 |
| ATOM | 3631 | C | ARG | 478 | 57.796 | 41.341 | 67.724 | 1.00 | 42.21 |
| ATOM | 3632 | O | ARG | 478 | 56.818 | 40.941 | 67.094 | 1.00 | 42.14 |
| ATOM | 3633 | N | PRO | 479 | 58.276 | 40.672 | 68.783 | 1.00 | 43.41 |
| ATOM | 3634 | CD | PRO | 479 | 59.428 | 41.039 | 69.629 | 1.00 | 41.40 |
| ATOM | 3635 | CA | PRO | 479 | 57.658 | 39.415 | 69.238 | 1.00 | 43.35 |
| ATOM | 3636 | CB | PRO | 479 | 58.435 | 39.087 | 70.510 | 1.00 | 40.98 |
| ATOM | 3637 | CG | PRO | 479 | 59.794 | 39.717 | 70.254 | 1.00 | 41.03 |
| ATOM | 3638 | C | PRO | 479 | 56.155 | 39.544 | 69.494 | 1.00 | 45.50 |
| ATOM | 3639 | O | PRO | 479 | 55.686 | 40.533 | 70.060 | 1.00 | 45.59 |
| ATOM | 3640 | N | GLU | 480 | 55.401 | 38.539 | 69.072 | 1.00 | 49.19 |
| ATOM | 3641 | CA | GLU | 480 | 53.955 | 38.546 | 69.266 | 1.00 | 53.07 |
| ATOM | 3642 | CB | GLU | 480 | 53.330 | 37.306 | 68.617 | 1.00 | 54.18 |
| ATOM | 3643 | CG | GLU | 480 | 53.173 | 37.452 | 67.107 | 1.00 | 58.24 |
| ATOM | 3644 | CD | GLU | 480 | 52.862 | 36.146 | 66.416 | 1.00 | 61.65 |
| ATOM | 3645 | OE1 | GLU | 480 | 51.961 | 35.426 | 66.897 | 1.00 | 63.71 |
| ATOM | 3646 | OE2 | GLU | 480 | 53.514 | 35.845 | 65.387 | 1.00 | 63.95 |
| ATOM | 3647 | C | GLU | 480 | 53.551 | 38.642 | 70.734 | 1.00 | 53.99 |
| ATOM | 3648 | O | GLU | 480 | 52.522 | 39.223 | 71.054 | 1.00 | 55.14 |
| ATOM | 3649 | N | ASP | 481 | 54.361 | 38.086 | 71.627 | 1.00 | 55.81 |
| ATOM | 3650 | CA | ASP | 481 | 54.046 | 38.146 | 73.048 | 1.00 | 57.69 |
| ATOM | 3651 | CB | ASP | 481 | 54.943 | 37.196 | 73.850 | 1.00 | 59.27 |
| ATOM | 3652 | CG | ASP | 481 | 54.607 | 35.732 | 73.611 | 1.00 | 61.89 |
| ATOM | 3653 | OD1 | ASP | 481 | 53.412 | 35.367 | 73.706 | 1.00 | 62.97 |
| ATOM | 3654 | OD2 | ASP | 481 | 55.540 | 34.947 | 73.337 | 1.00 | 63.73 |
| ATOM | 3655 | C | ASP | 481 | 54.194 | 39.564 | 73.586 | 1.00 | 58.87 |
| ATOM | 3656 | O | ASP | 481 | 53.484 | 39.956 | 74.517 | 1.00 | 59.57 |
| ATOM | 3657 | N | GLU | 482 | 55.111 | 40.335 | 73.005 | 1.00 | 58.07 |
| ATOM | 3658 | CA | GLU | 482 | 55.322 | 41.707 | 73.442 | 1.00 | 57.66 |
| ATOM | 3659 | CB | GLU | 482 | 56.653 | 42.231 | 72.922 | 1.00 | 58.24 |
| ATOM | 3660 | CG | GLU | 482 | 57.814 | 41.616 | 73.634 | 1.00 | 61.21 |
| ATOM | 3661 | CD | GLU | 482 | 57.684 | 41.785 | 75.130 | 1.00 | 64.17 |
| ATOM | 3662 | OE1 | GLU | 482 | 57.895 | 42.916 | 75.630 | 1.00 | 65.63 |
| ATOM | 3663 | OE2 | GLU | 482 | 57.350 | 40.787 | 75.803 | 1.00 | 65.01 |
| ATOM | 3664 | C | GLU | 482 | 54.188 | 42.584 | 72.951 | 1.00 | 58.20 |
| ATOM | 3665 | O | GLU | 482 | 53.732 | 43.485 | 73.655 | 1.00 | 58.45 |
| ATOM | 3666 | N | CYS | 483 | 53.736 | 42.318 | 71.732 | 1.00 | 57.89 |
| ATOM | 3667 | CA | CYS | 483 | 52.638 | 43.082 | 71.167 | 1.00 | 58.21 |
| ATOM | 3668 | C | CYS | 483 | 51.434 | 42.873 | 72.084 | 1.00 | 59.50 |
| ATOM | 3669 | O | CYS | 483 | 50.901 | 43.828 | 72.657 | 1.00 | 58.80 |
| ATOM | 3670 | CB | CYS | 483 | 52.327 | 42.595 | 69.747 | 1.00 | 56.50 |
| ATOM | 3671 | SG | CYS | 483 | 53.718 | 42.813 | 68.586 | 1.00 | 54.37 |
| ATOM | 3672 | N | VAL | 484 | 51.028 | 41.614 | 72.231 | 1.00 | 59.47 |
| ATOM | 3673 | CA | VAL | 484 | 49.901 | 41.264 | 73.084 | 1.00 | 59.07 |
| ATOM | 3674 | CB | VAL | 484 | 49.638 | 39.742 | 73.037 | 1.00 | 60.26 |
| ATOM | 3675 | CG1 | VAL | 484 | 48.555 | 39.356 | 74.038 | 1.00 | 61.90 |
| ATOM | 3676 | CG2 | VAL | 484 | 49.218 | 39.339 | 71.626 | 1.00 | 58.82 |
| ATOM | 3677 | C | VAL | 484 | 50.190 | 41.710 | 74.518 | 1.00 | 58.07 |
| ATOM | 3678 | O | VAL | 484 | 49.300 | 42.180 | 75.228 | 1.00 | 56.65 |
| ATOM | 3679 | N | GLY | 485 | 51.446 | 41.580 | 74.928 | 1.00 | 57.91 |
| ATOM | 3680 | CA | GLY | 485 | 51.831 | 41.992 | 76.267 | 1.00 | 59.03 |
| ATOM | 3681 | C | GLY | 485 | 51.524 | 43.460 | 76.507 | 1.00 | 59.68 |
| ATOM | 3682 | O | GLY | 485 | 51.071 | 43.840 | 77.586 | 1.00 | 60.34 |
| ATOM | 3683 | N | GLU | 486 | 51.768 | 44.288 | 75.498 | 1.00 | 60.09 |
| ATOM | 3684 | CA | GLU | 486 | 51.502 | 45.717 | 75.602 | 1.00 | 60.59 |
| ATOM | 3685 | CB | GLU | 486 | 52.252 | 46.477 | 74.502 | 1.00 | 61.23 |
| ATOM | 3686 | CG | GLU | 486 | 52.540 | 47.925 | 74.838 | 1.00 | 62.83 |
| ATOM | 3687 | CD | GLU | 486 | 53.215 | 48.666 | 73.701 | 1.00 | 65.64 |
| ATOM | 3688 | OE1 | GLU | 486 | 52.522 | 49.010 | 72.721 | 1.00 | 66.77 |
| ATOM | 3689 | OE2 | GLU | 486 | 54.440 | 48.901 | 73.782 | 1.00 | 67.03 |
| ATOM | 3690 | C | GLU | 486 | 49.999 | 45.935 | 75.446 | 1.00 | 60.37 |
| ATOM | 3691 | O | GLU | 486 | 49.507 | 47.061 | 75.539 | 1.00 | 59.61 |
| ATOM | 3692 | N | GLY | 487 | 49.276 | 44.843 | 75.197 | 1.00 | 59.61 |
| ATOM | 3693 | CA | GLY | 487 | 47.835 | 44.927 | 75.028 | 1.00 | 58.40 |
| ATOM | 3694 | C | GLY | 487 | 47.369 | 45.243 | 73.614 | 1.00 | 57.71 |
| ATOM | 3695 | O | GLY | 487 | 46.177 | 45.471 | 73.392 | 1.00 | 58.87 |
| ATOM | 3696 | N | LEU | 488 | 48.292 | 45.248 | 72.655 | 1.00 | 56.03 |
| ATOM | 3697 | CA | LEU | 488 | 47.952 | 45.546 | 71.268 | 1.00 | 54.46 |
| ATOM | 3698 | CB | LEU | 488 | 49.218 | 45.866 | 70.473 | 1.00 | 52.69 |
| ATOM | 3699 | CG | LEU | 488 | 50.005 | 47.084 | 70.965 | 1.00 | 52.11 |
| ATOM | 3700 | CD1 | LEU | 488 | 51.361 | 47.177 | 70.274 | 1.00 | 50.51 |
| ATOM | 3701 | CD2 | LEU | 488 | 49.186 | 48.324 | 70.703 | 1.00 | 54.20 |
| ATOM | 3702 | C | LEU | 488 | 47.206 | 44.404 | 70.591 | 1.00 | 56.04 |
| ATOM | 3703 | O | LEU | 488 | 47.516 | 43.226 | 70.797 | 1.00 | 56.18 |
| ATOM | 3704 | N | ALA | 489 | 46.220 | 44.766 | 69.775 | 1.00 | 56.93 |
| ATOM | 3705 | CA | ALA | 489 | 45.414 | 43.791 | 69.045 | 1.00 | 57.23 |
| ATOM | 3706 | CB | ALA | 489 | 44.704 | 42.861 | 70.015 | 1.00 | 56.66 |
| ATOM | 3707 | C | ALA | 489 | 44.396 | 44.510 | 68.177 | 1.00 | 57.16 |
| ATOM | 3708 | O | ALA | 489 | 44.236 | 45.722 | 68.272 | 1.00 | 55.01 |
| ATOM | 3709 | N | CYS | 490 | 43.703 | 43.754 | 67.335 | 1.00 | 59.68 |
| ATOM | 3710 | CA | CYS | 490 | 42.704 | 44.331 | 66.446 | 1.00 | 61.86 |
| ATOM | 3711 | C | CYS | 490 | 41.547 | 44.932 | 67.230 | 1.00 | 65.58 |
| ATOM | 3712 | O | CYS | 490 | 41.165 | 44.415 | 68.284 | 1.00 | 66.33 |
| ATOM | 3713 | CB | CYS | 490 | 42.174 | 43.264 | 65.482 | 1.00 | 61.11 |
| ATOM | 3714 | SG | CYS | 490 | 43.394 | 42.650 | 64.269 | 1.00 | 60.72 |
| ATOM | 3715 | N | HIS | 491 | 40.994 | 46.026 | 66.712 | 1.00 | 68.46 |
| ATOM | 3716 | CA | HIS | 491 | 39.872 | 46.701 | 67.356 | 1.00 | 70.45 |
| ATOM | 3717 | CB | HIS | 491 | 39.443 | 47.912 | 66.529 | 1.00 | 71.52 |
| ATOM | 3718 | CG | HIS | 491 | 38.432 | 48.782 | 67.208 | 1.00 | 73.85 |
| ATOM | 3719 | CD2 | HIS | 491 | 38.477 | 50.088 | 67.564 | 1.00 | 73.89 |
| ATOM | 3720 | ND1 | HIS | 491 | 37.183 | 48.328 | 67.576 | 1.00 | 74.38 |
| ATOM | 3721 | CE1 | HIS | 491 | 36.502 | 49.318 | 68.125 | 1.00 | 74.29 |
| ATOM | 3722 | NE2 | HIS | 491 | 37.264 | 50.397 | 68.128 | 1.00 | 74.01 |
| ATOM | 3723 | C | HIS | 491 | 38.700 | 45.730 | 67.517 | 1.00 | 72.75 |
| ATOM | 3724 | O | HIS | 491 | 38.435 | 44.897 | 66.639 | 1.00 | 71.43 |
| ATOM | 3725 | N | GLN | 492 | 38.007 | 45.846 | 68.651 | 1.00 | 75.11 |
| ATOM | 3726 | CA | GLN | 492 | 36.876 | 44.978 | 68.979 | 1.00 | 76.23 |
| ATOM | 3727 | CB | GLN | 492 | 36.141 | 45.524 | 70.213 | 1.00 | 79.06 |
| ATOM | 3728 | CG | GLN | 492 | 35.318 | 44.487 | 70.987 | 1.00 | 82.25 |
| ATOM | 3729 | CD | GLN | 492 | 34.127 | 43.951 | 70.201 | 1.00 | 84.79 |
| ATOM | 3730 | OE1 | GLN | 492 | 33.217 | 44.702 | 69.841 | 1.00 | 85.69 |
| ATOM | 3731 | NE2 | GLN | 492 | 34.129 | 42.646 | 69.932 | 1.00 | 84.27 |
| ATOM | 3732 | C | GLN | 492 | 35.903 | 44.845 | 67.812 | 1.00 | 75.18 |
| ATOM | 3733 | O | GLN | 492 | 35.266 | 43.802 | 67.633 | 1.00 | 74.06 |
| ATOM | 3734 | N | LEU | 493 | 35.803 | 45.898 | 67.009 | 1.00 | 73.83 |
| ATOM | 3735 | CA | LEU | 493 | 34.895 | 45.884 | 65.878 | 1.00 | 74.17 |
| ATOM | 3736 | CB | LEU | 493 | 34.506 | 47.318 | 65.515 | 1.00 | 74.20 |
| ATOM | 3737 | CG | LEU | 493 | 33.341 | 47.339 | 64.712 | 1.00 | 74.76 |
| ATOM | 3738 | C | LEU | 493 | 35.456 | 45.156 | 64.647 | 1.00 | 74.56 |
| ATOM | 3739 | O | LEU | 493 | 34.791 | 45.088 | 63.613 | 1.00 | 74.80 |
| ATOM | 3740 | N | CYS | 494 | 36.674 | 44.620 | 64.745 | 1.00 | 73.90 |
| ATOM | 3741 | CA | CYS | 494 | 37.259 | 43.895 | 63.617 | 1.00 | 73.92 |
| ATOM | 3742 | C | CYS | 494 | 36.675 | 42.491 | 63.604 | 1.00 | 74.79 |
| ATOM | 3743 | O | CYS | 494 | 37.077 | 41.628 | 64.382 | 1.00 | 74.58 |
| ATOM | 3744 | CB | CYS | 494 | 38.787 | 43.817 | 63.727 | 1.00 | 73.15 |
| ATOM | 3745 | SG | CYS | 494 | 39.687 | 45.373 | 63.407 | 1.00 | 70.37 |
| ATOM | 3746 | N | ALA | 495 | 35.724 | 42.283 | 62.702 | 1.00 | 76.40 |
| ATOM | 3747 | CA | ALA | 495 | 35.006 | 41.021 | 62.551 | 1.00 | 77.39 |
| ATOM | 3748 | CB | ALA | 495 | 34.438 | 40.932 | 61.149 | 1.00 | 78.27 |
| ATOM | 3749 | C | ALA | 495 | 35.730 | 39.719 | 62.886 | 1.00 | 77.47 |
| ATOM | 3750 | O | ALA | 495 | 35.413 | 39.077 | 63.891 | 1.00 | 77.55 |
| ATOM | 3751 | N | ARG | 496 | 36.686 | 39.319 | 62.049 | 1.00 | 76.19 |
| ATOM | 3752 | CA | ARG | 496 | 37.391 | 38.060 | 62.274 | 1.00 | 75.09 |
| ATOM | 3753 | CB | ARG | 496 | 37.472 | 37.270 | 60.963 | 1.00 | 77.48 |
| ATOM | 3754 | CG | ARG | 496 | 36.656 | 35.975 | 60.967 | 1.00 | 79.11 |
| ATOM | 3755 | CD | ARG | 496 | 36.916 | 35.131 | 59.720 | 1.00 | 80.25 |
| ATOM | 3756 | NE | ARG | 496 | 36.382 | 35.803 | 58.462 | 1.00 | 82.56 |
| ATOM | 3757 | CZ | ARG | 496 | 34.891 | 35.920 | 58.474 | 1.00 | 83.50 |
| ATOM | 3758 | C | ARG | 496 | 38.783 | 38.171 | 62.888 | 1.00 | 73.32 |
| ATOM | 3759 | O | ARG | 496 | 39.644 | 37.330 | 62.636 | 1.00 | 73.14 |
| ATOM | 3760 | N | GLY | 497 | 38.995 | 39.196 | 63.707 | 1.00 | 71.64 |
| ATOM | 3761 | CA | GLY | 497 | 40.287 | 39.387 | 64.345 | 1.00 | 68.82 |
| ATOM | 3762 | C | GLY | 497 | 41.390 | 39.845 | 63.405 | 1.00 | 66.99 |
| ATOM | 3763 | O | GLY | 497 | 42.553 | 39.886 | 63.790 | 1.00 | 67.23 |
| ATOM | 3764 | N | HIS | 498 | 41.023 | 40.201 | 62.177 | 1.00 | 65.61 |
| ATOM | 3765 | CA | HIS | 498 | 41.987 | 40.646 | 61.171 | 1.00 | 64.32 |
| ATOM | 3766 | CB | HIS | 498 | 41.646 | 40.025 | 59.816 | 1.00 | 66.38 |
| ATOM | 3767 | CG | HIS | 498 | 41.612 | 38.531 | 59.832 | 1.00 | 67.04 |
| ATOM | 3768 | CD2 | HIS | 498 | 42.128 | 37.639 | 60.709 | 1.00 | 66.05 |
| ATOM | 3769 | ND1 | HIS | 498 | 40.993 | 37.791 | 58.846 | 1.00 | 68.48 |
| ATOM | 3770 | CE1 | HIS | 498 | 41.131 | 36.506 | 59.115 | 1.00 | 69.68 |
| ATOM | 3771 | NE2 | HIS | 498 | 41.815 | 36.386 | 60.240 | 1.00 | 69.46 |
| ATOM | 3772 | C | HIS | 498 | 42.037 | 42.158 | 61.010 | 1.00 | 62.27 |
| ATOM | 3773 | O | HIS | 498 | 41.003 | 42.812 | 60.867 | 1.00 | 62.26 |
| ATOM | 3774 | N | CYS | 499 | 43.250 | 42.704 | 61.009 | 1.00 | 59.39 |
| ATOM | 3775 | CA | CYS | 499 | 43.442 | 44.139 | 60.856 | 1.00 | 56.07 |
| ATOM | 3776 | C | CYS | 499 | 44.878 | 44.451 | 60.436 | 1.00 | 54.88 |
| ATOM | 3777 | O | CYS | 499 | 45.765 | 43.611 | 60.576 | 1.00 | 54.18 |
| ATOM | 3778 | CB | CYS | 499 | 43.121 | 44.842 | 62.171 | 1.00 | 53.82 |
| ATOM | 3779 | SG | CYS | 499 | 44.212 | 44.370 | 63.550 | 1.00 | 53.18 |
| ATOM | 3780 | N | TRP | 500 | 45.096 | 45.660 | 59.922 | 1.00 | 54.46 |
| ATOM | 3781 | CA | TRP | 500 | 46.422 | 46.096 | 59.483 | 1.00 | 54.35 |
| ATOM | 3782 | CB | TRP | 500 | 46.345 | 46.877 | 58.169 | 1.00 | 52.68 |
| ATOM | 3783 | CG | TRP | 500 | 45.967 | 46.062 | 56.991 | 1.00 | 52.13 |
| ATOM | 3784 | CD2 | TRP | 500 | 46.855 | 45.330 | 56.134 | 1.00 | 52.51 |
| ATOM | 3785 | CE2 | TRP | 500 | 46.059 | 44.678 | 55.170 | 1.00 | 52.41 |
| ATOM | 3786 | CE3 | TRP | 500 | 48.246 | 45.163 | 56.090 | 1.00 | 52.09 |
| ATOM | 3787 | CD1 | TRP | 500 | 44.710 | 45.834 | 56.525 | 1.00 | 51.98 |
| ATOM | 3788 | NE1 | TRP | 500 | 44.754 | 45.002 | 55.432 | 1.00 | 53.67 |
| ATOM | 3789 | CZ2 | TRP | 500 | 46.606 | 43.870 | 54.171 | 1.00 | 51.30 |
| ATOM | 3790 | CZ3 | TRP | 500 | 48.792 | 44.360 | 55.094 | 1.00 | 51.18 |
| ATOM | 3791 | CH2 | TRP | 500 | 47.970 | 43.724 | 54.148 | 1.00 | 51.05 |
| ATOM | 3792 | C | TRP | 500 | 47.048 | 47.002 | 60.519 | 1.00 | 55.36 |
| ATOM | 3793 | O | TRP | 500 | 48.159 | 47.500 | 60.328 | 1.00 | 55.37 |
| ATOM | 3794 | N | GLY | 501 | 46.324 | 47.234 | 61.607 | 1.00 | 56.86 |
| ATOM | 3795 | CA | GLY | 501 | 46.834 | 48.106 | 62.643 | 1.00 | 58.32 |
| ATOM | 3796 | C | GLY | 501 | 45.906 | 48.224 | 63.834 | 1.00 | 60.22 |
| ATOM | 3797 | O | GLY | 501 | 44.953 | 47.457 | 63.963 | 1.00 | 59.76 |
| ATOM | 3798 | N | PRO | 502 | 46.159 | 49.199 | 64.719 | 1.00 | 61.74 |
| ATOM | 3799 | CD | PRO | 502 | 47.338 | 50.076 | 64.639 | 1.00 | 61.18 |
| ATOM | 3800 | CA | PRO | 502 | 45.395 | 49.479 | 65.939 | 1.00 | 62.96 |
| ATOM | 3801 | CB | PRO | 502 | 46.227 | 50.565 | 66.626 | 1.00 | 62.80 |
| ATOM | 3802 | CG | PRO | 502 | 47.607 | 50.353 | 66.085 | 1.00 | 62.29 |
| ATOM | 3803 | C | PRO | 502 | 43.933 | 49.904 | 65.799 | 1.00 | 64.19 |
| ATOM | 3804 | O | PRO | 502 | 43.023 | 49.154 | 66.155 | 1.00 | 63.69 |
| ATOM | 3805 | N | GLY | 503 | 43.720 | 51.115 | 65.294 | 1.00 | 65.19 |
| ATOM | 3806 | CA | GLY | 503 | 42.376 | 51.657 | 65.168 | 1.00 | 67.81 |
| ATOM | 3807 | C | GLY | 503 | 41.284 | 50.832 | 64.511 | 1.00 | 69.48 |
| ATOM | 3808 | O | GLY | 503 | 41.498 | 49.675 | 64.149 | 1.00 | 69.49 |
| ATOM | 3809 | N | PRO | 504 | 40.078 | 51.412 | 64.359 | 1.00 | 70.50 |
| ATOM | 3810 | CD | PRO | 504 | 39.672 | 52.684 | 64.974 | 1.00 | 70.99 |
| ATOM | 3811 | CA | PRO | 504 | 38.918 | 50.755 | 63.742 | 1.00 | 70.28 |
| ATOM | 3812 | CB | PRO | 504 | 37.727 | 51.582 | 64.244 | 1.00 | 70.32 |
| ATOM | 3813 | CG | PRO | 504 | 38.272 | 52.375 | 65.398 | 1.00 | 70.99 |
| ATOM | 3814 | C | PRO | 504 | 39.036 | 50.827 | 62.225 | 1.00 | 69.41 |
| ATOM | 3815 | O | PRO | 504 | 38.374 | 50.083 | 61.499 | 1.00 | 69.42 |
| ATOM | 3816 | N | THR | 505 | 39.884 | 51.742 | 61.763 | 1.00 | 68.13 |
| ATOM | 3817 | CA | THR | 505 | 40.119 | 51.950 | 60.340 | 1.00 | 67.47 |
| ATOM | 3818 | CB | THR | 505 | 40.878 | 53.259 | 60.102 | 1.00 | 67.81 |
| ATOM | 3819 | OG1 | THR | 505 | 40.220 | 54.325 | 60.796 | 1.00 | 67.46 |
| ATOM | 3820 | CG2 | THR | 505 | 40.936 | 53.574 | 58.611 | 1.00 | 68.41 |
| ATOM | 3821 | C | THR | 505 | 40.947 | 50.834 | 59.714 | 1.00 | 67.11 |
| ATOM | 3822 | O | THR | 505 | 40.949 | 50.669 | 58.496 | 1.00 | 66.67 |
| ATOM | 3823 | N | GLN | 506 | 41.641 | 50.067 | 60.550 | 1.00 | 67.09 |
| ATOM | 3824 | CA | GLN | 506 | 42.513 | 49.000 | 60.074 | 1.00 | 66.20 |
| ATOM | 3825 | CB | GLN | 506 | 43.757 | 48.940 | 60.963 | 1.00 | 65.97 |
| ATOM | 3826 | CG | GLN | 506 | 44.438 | 50.292 | 61.156 | 1.00 | 65.10 |
| ATOM | 3827 | CD | GLN | 506 | 44.931 | 50.897 | 59.851 | 1.00 | 64.76 |
| ATOM | 3828 | OE1 | GLN | 506 | 45.477 | 52.001 | 59.833 | 1.00 | 63.49 |
| ATOM | 3829 | NE2 | GLN | 506 | 44.747 | 50.174 | 58.754 | 1.00 | 64.72 |
| ATOM | 3830 | C | GLN | 506 | 41.928 | 47.598 | 59.937 | 1.00 | 66.13 |
| ATOM | 3831 | O | GLN | 506 | 42.640 | 46.681 | 59.535 | 1.00 | 65.92 |
| ATOM | 3832 | N | CYS | 507 | 40.651 | 47.418 | 60.259 | 1.00 | 66.51 |
| ATOM | 3833 | CA | CYS | 507 | 40.043 | 46.094 | 60.147 | 1.00 | 67.92 |
| ATOM | 3834 | C | CYS | 507 | 40.079 | 45.584 | 58.711 | 1.00 | 69.10 |
| ATOM | 3835 | O | CYS | 507 | 40.336 | 46.351 | 57.786 | 1.00 | 68.98 |
| ATOM | 3836 | CB | CYS | 507 | 38.594 | 46.107 | 60.642 | 1.00 | 68.38 |
| ATOM | 3837 | SG | CYS | 507 | 38.351 | 46.532 | 62.400 | 1.00 | 67.08 |
| ATOM | 3838 | N | VAL | 508 | 39.804 | 44.293 | 58.534 | 1.00 | 71.60 |
| ATOM | 3839 | CA | VAL | 508 | 39.824 | 43.665 | 57.214 | 1.00 | 74.36 |
| ATOM | 3840 | CB | VAL | 508 | 41.094 | 42.802 | 57.046 | 1.00 | 74.35 |
| ATOM | 3841 | CG1 | VAL | 508 | 41.169 | 42.249 | 55.637 | 1.00 | 73.86 |
| ATOM | 3842 | CG2 | VAL | 508 | 42.325 | 43.620 | 57.372 | 1.00 | 74.61 |
| ATOM | 3843 | C | VAL | 508 | 38.618 | 42.760 | 56.926 | 1.00 | 76.71 |
| ATOM | 3844 | O | VAL | 508 | 37.993 | 42.232 | 57.846 | 1.00 | 76.96 |
| ATOM | 3845 | N | ASN | 509 | 38.320 | 42.583 | 55.637 | 1.00 | 78.89 |
| ATOM | 3846 | CA | ASN | 509 | 37.231 | 41.729 | 55.161 | 0.01 | 79.46 |
| ATOM | 3847 | CB | ASN | 509 | 37.748 | 40.301 | 54.959 | 0.01 | 79.66 |
| ATOM | 3848 | CG | ASN | 509 | 36.706 | 39.434 | 54.544 | 0.01 | 79.80 |
| ATOM | 3849 | C | ASN | 509 | 35.994 | 41.690 | 56.048 | 0.01 | 80.03 |
| ATOM | 3850 | O | ASN | 509 | 35.706 | 40.669 | 56.672 | 0.01 | 80.13 |
| ATOM | 3851 | N | ASP | 510 | 35.256 | 42.796 | 56.081 | 0.01 | 80.55 |
| ATOM | 3852 | CA | ASP | 510 | 34.042 | 42.894 | 56.885 | 0.01 | 81.01 |
| ATOM | 3853 | CB | ASP | 510 | 32.979 | 41.945 | 56.341 | 0.01 | 81.12 |
| ATOM | 3854 | C | ASP | 510 | 34.333 | 42.570 | 58.343 | 0.01 | 81.22 |
| ATOM | 3855 | O | ASP | 510 | 34.135 | 43.457 | 59.200 | 0.01 | 81.38 |
| ATOM | 3856 | OXT | ASP | 510 | 34.756 | 41.429 | 58.608 | 0.01 | 81.41 |
| ATOM | 3857 | OH2 | WAT | 601 | 66.524 | 30.665 | 42.494 | 1.00 | 28.61 |
| ATOM | 3858 | OH2 | WAT | 602 | 48.735 | 23.280 | 22.419 | 1.00 | 71.18 |
| ATOM | 3859 | OH2 | WAT | 603 | 65.388 | 13.447 | 32.927 | 1.00 | 57.63 |
| ATOM | 3860 | OH2 | WAT | 604 | 41.973 | 47.301 | 64.542 | 1.00 | 35.83 |
| ATOM | 3861 | OH2 | WAT | 605 | 69.684 | 31.298 | 45.975 | 1.00 | 26.09 |
| ATOM | 3862 | OH2 | WAT | 606 | 63.686 | 41.502 | 49.027 | 1.00 | 33.31 |
| ATOM | 3863 | OH2 | WAT | 607 | 59.703 | 43.909 | 47.356 | 1.00 | 39.87 |
| ATOM | 3864 | OH2 | WAT | 608 | 57.621 | 15.652 | 21.616 | 1.00 | 48.33 |
| ATOM | 3865 | OH2 | WAT | 609 | 72.561 | 35.414 | 45.496 | 1.00 | 41.23 |
| ATOM | 3866 | OH2 | WAT | 610 | 60.694 | 29.164 | 22.844 | 1.00 | 40.92 |
| ATOM | 3867 | OH2 | WAT | 611 | 57.286 | 37.916 | 62.350 | 1.00 | 30.64 |
| ATOM | 3868 | OH2 | WAT | 612 | 46.597 | 49.739 | 51.334 | 1.00 | 23.74 |
| ATOM | 3869 | OH2 | WAT | 613 | 66.260 | 35.222 | 49.508 | 1.00 | 30.53 |
| ATOM | 3870 | OH2 | WAT | 614 | 65.596 | 48.769 | 43.073 | 1.00 | 47.24 |
| ATOM | 3871 | OH2 | WAT | 615 | 50.445 | 34.432 | 40.487 | 1.00 | 49.48 |
| ATOM | 3872 | OH2 | WAT | 616 | 64.437 | 47.570 | 62.680 | 1.00 | 39.37 |
| ATOM | 3873 | OH2 | WAT | 617 | 64.474 | 34.466 | 36.845 | 1.00 | 49.08 |
| ATOM | 3874 | OH2 | WAT | 618 | 44.017 | 32.385 | 41.614 | 1.00 | 53.85 |
| ATOM | 3875 | OH2 | WAT | 619 | 49.339 | 30.195 | 12.563 | 1.00 | 42.42 |
| ATOM | 3876 | OH2 | WAT | 620 | 54.537 | 33.867 | 42.583 | 1.00 | 37.72 |
| ATOM | 3877 | OH2 | WAT | 621 | 64.364 | 53.511 | 57.998 | 1.00 | 38.10 |
| ATOM | 3878 | OH2 | WAT | 622 | 76.463 | 9.761 | 42.429 | 1.00 | 57.34 |
| ATOM | 3879 | OH2 | WAT | 623 | 70.186 | 16.762 | 34.786 | 1.00 | 43.49 |
| ATOM | 3880 | OH2 | WAT | 624 | 79.053 | 23.302 | 19.458 | 1.00 | 59.95 |
| ATOM | 3881 | OH2 | WAT | 625 | 47.646 | 54.357 | 48.883 | 1.00 | 46.72 |
| ATOM | 3882 | OH2 | WAT | 626 | 56.831 | 53.403 | 65.052 | 1.00 | 46.20 |
| ATOM | 3883 | OH2 | WAT | 627 | 59.570 | 19.307 | 44.131 | 1.00 | 34.57 |
| ATOM | 3884 | OH2 | WAT | 628 | 59.681 | 48.082 | 63.468 | 1.00 | 38.16 |
| ATOM | 3885 | OH2 | WAT | 629 | 66.403 | 51.834 | 57.456 | 1.00 | 37.88 |
| ATOM | 3886 | OH2 | WAT | 630 | 66.042 | 43.477 | 41.897 | 1.00 | 47.27 |
| ATOM | 3887 | OH2 | WAT | 631 | 59.733 | 22.694 | 19.076 | 1.00 | 39.36 |
| ATOM | 3888 | OH2 | WAT | 632 | 71.933 | 32.168 | 53.584 | 1.00 | 51.96 |
| ATOM | 3889 | OH2 | WAT | 633 | 67.238 | 29.817 | 55.382 | 1.00 | 39.02 |
| ATOM | 3890 | OH2 | WAT | 634 | 59.851 | 33.676 | 40.899 | 1.00 | 45.95 |
| ATOM | 3891 | OH2 | WAT | 635 | 83.739 | 22.332 | 45.520 | 1.00 | 50.48 |
| ATOM | 3892 | OH2 | WAT | 636 | 61.181 | 20.281 | 41.584 | 1.00 | 42.52 |
| ATOM | 3893 | OH2 | WAT | 637 | 61.537 | 40.611 | 38.196 | 1.00 | 40.98 |
| ATOM | 3894 | OH2 | WAT | 638 | 61.615 | 29.853 | 19.905 | 1.00 | 52.46 |
| ATOM | 3895 | OH2 | WAT | 639 | 59.028 | 54.089 | 61.722 | 1.00 | 40.76 |
| ATOM | 3896 | OH2 | WAT | 640 | 43.735 | 38.307 | −3.516 | 1.00 | 60.92 |
| ATOM | 3897 | OH2 | WAT | 641 | 55.950 | 37.085 | 38.131 | 1.00 | 48.00 |
| ATOM | 3898 | OH2 | WAT | 642 | 55.110 | 41.451 | 65.214 | 1.00 | 32.81 |
| ATOM | 3899 | OH2 | WAT | 643 | 61.727 | 25.862 | 29.188 | 1.00 | 46.24 |
| ATOM | 3900 | OH2 | WAT | 644 | 65.928 | 18.150 | 38.925 | 1.00 | 40.13 |
| ATOM | 3901 | OH2 | WAT | 645 | 55.177 | 34.966 | 24.691 | 1.00 | 46.33 |
| ATOM | 3902 | OH2 | WAT | 646 | 41.487 | 51.896 | 47.974 | 1.00 | 65.65 |
| ATOM | 3903 | OH2 | WAT | 647 | 55.395 | 15.492 | 23.549 | 1.00 | 56.95 |
| ATOM | 3904 | OH2 | WAT | 648 | 59.898 | 21.613 | 26.137 | 1.00 | 33.49 |
| ATOM | 3905 | OH2 | WAT | 649 | 58.699 | 47.923 | 70.573 | 1.00 | 54.29 |
| ATOM | 3906 | OH2 | WAT | 650 | 64.003 | 58.536 | 60.177 | 1.00 | 49.67 |
| ATOM | 3907 | OH2 | WAT | 651 | 68.010 | 11.136 | 22.922 | 1.00 | 46.58 |
| ATOM | 3908 | OH2 | WAT | 652 | 55.678 | 50.674 | 68.586 | 1.00 | 46.85 |
| ATOM | 3909 | OH2 | WAT | 653 | 45.897 | 25.870 | 27.002 | 1.00 | 57.96 |
| ATOM | 3910 | OH2 | WAT | 654 | 54.359 | 26.790 | 48.758 | 1.00 | 66.55 |
| ATOM | 3911 | OH2 | WAT | 655 | 80.363 | 16.850 | 38.325 | 1.00 | 34.34 |
| ATOM | 3912 | OH2 | WAT | 656 | 61.431 | 53.904 | 60.446 | 1.00 | 58.46 |
| ATOM | 3913 | OH2 | WAT | 657 | 64.395 | 35.461 | 52.723 | 1.00 | 35.60 |
| ATOM | 3914 | OH2 | WAT | 658 | 52.526 | 40.800 | 64.728 | 1.00 | 50.80 |
| ATOM | 3915 | OH2 | WAT | 659 | 56.877 | 31.825 | 56.618 | 1.00 | 45.68 |
| ATOM | 3916 | OH2 | WAT | 660 | 55.868 | 27.878 | 30.041 | 1.00 | 53.59 |
| ATOM | 3917 | OH2 | WAT | 661 | 77.776 | 15.905 | 45.073 | 1.00 | 59.14 |
| ATOM | 3918 | OH2 | WAT | 662 | 63.754 | 25.151 | 14.194 | 1.00 | 46.52 |
| ATOM | 3919 | OH2 | WAT | 663 | 41.878 | 53.249 | 63.058 | 1.00 | 44.80 |
| ATOM | 3920 | OH2 | WAT | 664 | 80.591 | 16.000 | 36.009 | 1.00 | 51.39 |
| ATOM | 3921 | OH2 | WAT | 665 | 59.393 | 45.412 | 72.211 | 1.00 | 77.96 |
| ATOM | 3922 | OH2 | WAT | 666 | 51.497 | 25.366 | 16.022 | 1.00 | 38.80 |
| ATOM | 3923 | OH2 | WAT | 667 | 61.863 | 12.713 | 17.304 | 1.00 | 43.79 |
| ATOM | 3924 | OH2 | WAT | 668 | 63.503 | 14.941 | 33.560 | 1.00 | 42.89 |
| ATOM | 3925 | OH2 | WAT | 669 | 61.973 | 36.087 | 65.135 | 1.00 | 50.06 |
| ATOM | 3926 | OH2 | WAT | 670 | 53.296 | 56.350 | 56.050 | 1.00 | 44.60 |
| ATOM | 3927 | OH2 | WAT | 671 | 59.130 | 10.280 | 24.662 | 1.00 | 48.60 |
| ATOM | 3928 | OH2 | WAT | 672 | 78.580 | 23.713 | 47.588 | 1.00 | 55.99 |
| ATOM | 3929 | OH2 | WAT | 673 | 56.562 | 19.517 | 13.895 | 1.00 | 44.27 |
| ATOM | 3930 | OH2 | WAT | 674 | 65.541 | 48.376 | 65.243 | 1.00 | 65.80 |
| ATOM | 3931 | OH2 | WAT | 675 | 68.517 | 47.817 | 50.926 | 1.00 | 51.64 |
| ATOM | 3932 | OH2 | WAT | 676 | 52.940 | 45.320 | 33.774 | 1.00 | 66.09 |
| ATOM | 3933 | OH2 | WAT | 677 | 75.668 | 19.068 | 52.383 | 1.00 | 57.66 |
| ATOM | 3934 | OH2 | WAT | 678 | 70.723 | 37.503 | 60.123 | 1.00 | 46.72 |
| ATOM | 3935 | OH2 | WAT | 679 | 50.335 | 33.290 | 18.844 | 1.00 | 45.69 |
| ATOM | 3936 | OH2 | WAT | 680 | 52.500 | 32.843 | 54.796 | 1.00 | 53.72 |
| ATOM | 3937 | OH2 | WAT | 681 | 57.066 | 23.070 | 13.074 | 1.00 | 50.65 |
| ATOM | 3938 | OH2 | WAT | 682 | 61.915 | 33.379 | 63.218 | 1.00 | 48.04 |
| ATOM | 3939 | OH2 | WAT | 683 | 67.948 | 43.379 | 49.661 | 1.00 | 44.42 |
| ATOM | 3940 | OH2 | WAT | 684 | 57.359 | 22.912 | 26.139 | 1.00 | 41.68 |
| ATOM | 3941 | OH2 | WAT | 685 | 79.814 | 28.506 | 19.445 | 1.00 | 49.19 |
| ATOM | 3942 | OH2 | WAT | 686 | 74.126 | 12.870 | 30.657 | 1.00 | 42.03 |
| ATOM | 3943 | OH2 | WAT | 687 | 49.421 | 21.379 | 14.426 | 1.00 | 61.74 |
| ATOM | 3944 | OH2 | WAT | 688 | 66.525 | 36.888 | 51.906 | 1.00 | 42.72 |
| ATOM | 3945 | OH2 | WAT | 689 | 82.488 | 11.904 | 44.349 | 1.00 | 51.00 |
| ATOM | 3946 | OH2 | WAT | 690 | 73.678 | 42.567 | 41.135 | 1.00 | 56.59 |
| ATOM | 3947 | OH2 | WAT | 691 | 55.539 | 33.853 | 27.113 | 1.00 | 58.89 |
| ATOM | 3948 | OH2 | WAT | 692 | 44.960 | 47.684 | 36.358 | 1.00 | 58.30 |
| ATOM | 3949 | OH2 | WAT | 693 | 63.736 | 54.302 | 65.440 | 1.00 | 63.02 |
| ATOM | 3950 | OH2 | WAT | 694 | 48.109 | 58.899 | 55.284 | 1.00 | 55.29 |
| ATOM | 3951 | OH2 | WAT | 695 | 45.345 | 35.120 | −1.208 | 1.00 | 60.11 |
| ATOM | 3952 | OH2 | WAT | 696 | 58.248 | 26.866 | 28.828 | 1.00 | 46.10 |
| ATOM | 3953 | OH2 | WAT | 697 | 66.972 | 48.406 | 56.023 | 1.00 | 38.88 |
| ATOM | 3954 | OH2 | WAT | 698 | 67.380 | 47.152 | 45.433 | 1.00 | 46.93 |
| ATOM | 3955 | OH2 | WAT | 699 | 54.190 | 24.235 | 12.345 | 1.00 | 43.07 |
| ATOM | 3956 | OH2 | WAT | 700 | 82.014 | 37.287 | 31.826 | 1.00 | 49.62 |
| ATOM | 3957 | OH2 | WAT | 701 | 72.612 | 9.107 | 14.973 | 1.00 | 63.48 |
| ATOM | 3958 | OH2 | WAT | 702 | 60.555 | 29.814 | 25.951 | 1.00 | 49.47 |
| ATOM | 3959 | OH2 | WAT | 703 | 67.063 | 39.237 | 50.605 | 1.00 | 64.66 |
| ATOM | 3960 | OH2 | WAT | 704 | 84.336 | 13.624 | 49.262 | 1.00 | 78.36 |
| ATOM | 3961 | OH2 | WAT | 705 | 48.431 | 55.687 | 34.738 | 1.00 | 57.72 |
| ATOM | 3962 | OH2 | WAT | 706 | 71.691 | 37.538 | 29.164 | 1.00 | 48.64 |
| ATOM | 3963 | OH2 | WAT | 707 | 60.252 | 65.177 | 41.763 | 1.00 | 57.87 |
| ATOM | 3964 | OH2 | WAT | 708 | 61.977 | 18.461 | 29.063 | 1.00 | 53.49 |
| ATOM | 3965 | OH2 | WAT | 709 | 77.177 | 25.254 | 19.532 | 1.00 | 84.59 |
| ATOM | 3966 | OH2 | WAT | 710 | 52.321 | 48.027 | 67.553 | 1.00 | 44.06 |
| ATOM | 3967 | OH2 | WAT | 711 | 55.147 | 47.193 | 34.020 | 1.00 | 62.19 |
| ATOM | 3968 | OH2 | WAT | 712 | 47.265 | 40.316 | 39.544 | 1.00 | 53.27 |
| ATOM | 3969 | OH2 | WAT | 713 | 64.134 | 61.597 | 36.364 | 1.00 | 61.55 |
| ATOM | 3970 | OH2 | WAT | 714 | 45.208 | 24.605 | 18.377 | 1.00 | 48.53 |
| ATOM | 3971 | OH2 | WAT | 715 | 48.252 | 61.583 | 44.775 | 1.00 | 59.25 |
| ATOM | 3972 | OH2 | WAT | 716 | 74.802 | 15.285 | 29.771 | 1.00 | 50.29 |
| ATOM | 3973 | OH2 | WAT | 717 | 55.024 | 47.905 | 68.582 | 1.00 | 45.87 |
| ATOM | 3974 | OH2 | WAT | 718 | 67.997 | 16.025 | 14.692 | 1.00 | 57.23 |
| ATOM | 3975 | OH2 | WAT | 719 | 52.457 | 50.945 | 71.009 | 1.00 | 53.06 |
| ATOM | 3976 | OH2 | WAT | 720 | 47.846 | 40.861 | 69.090 | 1.00 | 67.31 |
| ATOM | 3977 | OH2 | WAT | 721 | 60.706 | 31.257 | 56.916 | 1.00 | 60.88 |
| ATOM | 3978 | OH2 | WAT | 722 | 65.455 | 41.843 | 50.751 | 1.00 | 40.91 |
| ATOM | 3979 | OH2 | WAT | 723 | 57.205 | 29.454 | 54.969 | 1.00 | 47.67 |
| ATOM | 3980 | OH2 | WAT | 724 | 67.371 | 61.279 | 47.906 | 1.00 | 61.63 |
| ATOM | 3981 | OH2 | WAT | 725 | 42.132 | 44.861 | 54.036 | 1.00 | 72.42 |
| ATOM | 3982 | OH2 | WAT | 726 | 49.901 | 45.638 | 59.964 | 1.00 | 58.08 |
| ATOM | 3983 | OH2 | WAT | 727 | 78.431 | 30.042 | 52.188 | 1.00 | 54.52 |
| ATOM | 3984 | OH2 | WAT | 728 | 67.232 | 20.133 | 17.586 | 1.00 | 48.39 |
| ATOM | 3985 | OH2 | WAT | 729 | 56.846 | 21.629 | 28.220 | 1.00 | 66.25 |
| ATOM | 3986 | OH2 | WAT | 731 | 66.550 | 67.628 | 45.297 | 1.00 | 61.99 |
| ATOM | 3987 | OH2 | WAT | 732 | 42.679 | 47.745 | 68.650 | 1.00 | 59.22 |
| ATOM | 3988 | OH2 | WAT | 733 | 59.753 | 54.875 | 64.155 | 1.00 | 46.18 |
| ATOM | 3989 | OH2 | WAT | 734 | 69.441 | 41.606 | 48.205 | 1.00 | 56.13 |
| ATOM | 3990 | OH2 | WAT | 735 | 54.860 | 31.772 | 59.102 | 1.00 | 52.80 |
| ATOM | 3991 | C1 | NAG | 911 | 72.132 | 24.580 | 59.897 | 1.00 | 88.45 |
| ATOM | 3992 | C2 | NAG | 911 | 71.432 | 23.893 | 61.092 | 1.00 | 90.78 |
| ATOM | 3993 | N2 | NAG | 911 | 70.376 | 23.011 | 60.624 | 1.00 | 92.23 |
| ATOM | 3994 | C7 | NAG | 911 | 70.502 | 21.689 | 60.728 | 1.00 | 93.50 |
| ATOM | 3995 | O7 | NAG | 911 | 71.152 | 21.141 | 61.623 | 1.00 | 93.42 |
| ATOM | 3996 | C8 | NAG | 911 | 69.801 | 20.845 | 59.672 | 1.00 | 93.63 |
| ATOM | 3997 | C3 | NAG | 911 | 70.831 | 24.939 | 62.047 | 1.00 | 91.07 |
| ATOM | 3998 | O3 | NAG | 911 | 70.344 | 24.295 | 63.218 | 1.00 | 90.85 |
| ATOM | 3999 | C4 | NAG | 911 | 71.879 | 25.985 | 62.436 | 1.00 | 91.28 |
| ATOM | 4000 | O4 | NAG | 911 | 71.274 | 27.004 | 63.221 | 1.00 | 91.82 |
| ATOM | 4001 | C5 | NAG | 911 | 72.494 | 26.594 | 61.170 | 1.00 | 91.20 |
| ATOM | 4002 | O5 | NAG | 911 | 73.089 | 25.551 | 60.360 | 1.00 | 90.01 |
| ATOM | 4003 | C6 | NAG | 911 | 73.588 | 27.607 | 61.477 | 1.00 | 91.15 |
| ATOM | 4004 | O6 | NAG | 911 | 73.105 | 28.667 | 62.292 | 1.00 | 89.63 |
| ATOM | 4005 | C1 | NAG | 941 | 94.155 | 27.124 | 39.668 | 1.00 | 83.74 |
| ATOM | 4006 | C2 | NAG | 941 | 93.682 | 28.571 | 39.467 | 1.00 | 85.78 |
| ATOM | 4007 | N2 | NAG | 941 | 92.491 | 28.591 | 38.639 | 1.00 | 87.29 |
| ATOM | 4008 | C7 | NAG | 941 | 91.434 | 29.308 | 39.013 | 1.00 | 87.84 |
| ATOM | 4009 | O7 | NAG | 941 | 90.910 | 29.191 | 40.121 | 1.00 | 88.39 |
| ATOM | 4010 | C8 | NAG | 941 | 90.882 | 30.316 | 38.016 | 1.00 | 87.53 |
| ATOM | 4011 | C3 | NAG | 941 | 94.793 | 29.413 | 38.819 | 1.00 | 87.28 |
| ATOM | 4012 | O3 | NAG | 941 | 94.397 | 30.779 | 38.775 | 1.00 | 87.44 |
| ATOM | 4013 | C4 | NAG | 941 | 96.097 | 29.280 | 39.622 | 1.00 | 88.25 |
| ATOM | 4014 | O4 | NAG | 941 | 97.156 | 29.948 | 38.945 | 1.00 | 88.64 |
| ATOM | 4015 | C5 | NAG | 941 | 96.450 | 27.795 | 39.806 | 1.00 | 88.22 |
| ATOM | 4016 | O5 | NAG | 941 | 95.356 | 27.100 | 40.454 | 1.00 | 85.86 |
| ATOM | 4017 | C6 | NAG | 941 | 97.695 | 27.577 | 40.652 | 1.00 | 88.36 |
| ATOM | 4018 | O6 | NAG | 941 | 97.384 | 27.551 | 42.038 | 1.00 | 88.02 |
| ATOM | 4019 | C1 | NAG | 951 | 54.055 | 18.637 | 25.913 | 1.00 | 63.88 |
| ATOM | 4020 | C2 | NAG | 951 | 55.035 | 17.683 | 26.604 | 1.00 | 64.92 |
| ATOM | 4021 | N2 | NAG | 951 | 56.133 | 17.389 | 25.708 | 1.00 | 62.42 |
| ATOM | 4022 | C7 | NAG | 951 | 57.321 | 17.936 | 25.923 | 1.00 | 61.61 |
| ATOM | 4023 | O7 | NAG | 951 | 57.488 | 18.897 | 26.673 | 1.00 | 61.97 |
| ATOM | 4024 | C8 | NAG | 951 | 58.507 | 17.323 | 25.201 | 1.00 | 62.25 |
| ATOM | 4025 | C3 | NAG | 951 | 54.326 | 16.379 | 27.003 | 1.00 | 68.63 |
| ATOM | 4026 | O3 | NAG | 951 | 55.206 | 15.566 | 27.768 | 1.00 | 66.57 |
| ATOM | 4027 | C4 | NAG | 951 | 53.057 | 16.687 | 27.814 | 1.00 | 71.99 |
| ATOM | 4028 | O4 | NAG | 951 | 52.327 | 15.468 | 28.079 | 1.00 | 78.42 |
| ATOM | 4029 | C5 | NAG | 951 | 52.178 | 17.671 | 27.024 | 1.00 | 70.23 |
| ATOM | 4030 | O5 | NAG | 951 | 52.923 | 18.877 | 26.751 | 1.00 | 66.53 |
| ATOM | 4031 | C6 | NAG | 951 | 50.911 | 18.086 | 27.746 | 1.00 | 69.73 |
| ATOM | 4032 | O6 | NAG | 951 | 49.903 | 18.488 | 26.828 | 1.00 | 68.69 |
| ATOM | 4033 | C1 | NAG | 952 | 52.203 | 15.123 | 29.418 | 1.00 | 84.50 |
| ATOM | 4034 | C2 | NAG | 952 | 50.843 | 14.467 | 29.674 | 1.00 | 87.78 |
| ATOM | 4035 | N2 | NAG | 952 | 49.773 | 15.386 | 29.332 | 1.00 | 88.99 |
| ATOM | 4036 | C7 | NAG | 952 | 49.117 | 15.239 | 28.184 | 1.00 | 90.08 |
| ATOM | 4037 | O7 | NAG | 952 | 49.630 | 14.740 | 27.176 | 1.00 | 90.89 |
| ATOM | 4038 | C8 | NAG | 952 | 47.670 | 15.708 | 28.144 | 1.00 | 90.00 |
| ATOM | 4039 | C3 | NAG | 952 | 50.744 | 14.054 | 31.149 | 1.00 | 89.39 |
| ATOM | 4040 | O3 | NAG | 952 | 49.530 | 13.345 | 31.371 | 1.00 | 90.01 |
| ATOM | 4041 | C4 | NAG | 952 | 51.934 | 13.163 | 31.527 | 1.00 | 90.10 |
| ATOM | 4042 | O4 | NAG | 952 | 51.910 | 12.898 | 32.925 | 1.00 | 92.36 |
| ATOM | 4043 | C5 | NAG | 952 | 53.256 | 13.853 | 31.148 | 1.00 | 89.28 |
| ATOM | 4044 | O5 | NAG | 952 | 53.253 | 14.204 | 29.745 | 1.00 | 86.82 |
| ATOM | 4045 | C6 | NAG | 952 | 54.470 | 12.971 | 31.390 | 1.00 | 88.91 |
| ATOM | 4046 | O6 | NAG | 952 | 54.846 | 12.275 | 30.211 | 1.00 | 89.44 |
| END | | | | | | | | | |

1-46. (canceled)

47. A method for identifying a potential modulator compound for ErbB2 which method comprises:
(a) designing or selecting a compound which binds to the structure formed by amino acids 1 to 509 of an ErbB2 polypeptide having the atomic coordinates shown in Appendix I, or atomic coordinates having a root mean square deviation of backbone atoms of not more than 1.5 Å when superimposed on the corresponding backbone atoms having the atomic coordinates shown in Appendix I, wherein binding of the compound to the structure is favoured energetically; and
(b) testing the compound designed or selected in (a) for its ability to interact with and/or modulate the activity of ErbB2.

48. A method as claimed in claim 47, wherein the compound binds to a subset of amino acids selected from at least one of the CR1 domain, the potential CR1 loop docking site between the L1, CR1 and L2 domains, the CR1-L2 hinge region, the regions of the L1 and L2 domains that contact each other in a closed conformation.

49. A method as claimed in claim 47, wherein the subset of amino acids defines at least a part of the heterodimerisation surface with another member of the EGF receptor family.

50. A method as claimed in claim 49, wherein the member of the EGF receptor family is selected from the group consisting of ErbB1 (EGF receptor), ErbB3 and ErbB4.

51. A method as claimed in claim 49, wherein the heterodimerisation surface includes at least one of (i) the N-terminal end of the CR1 domain, (ii) the CR1 domain dimerisation loop and adjacent residues and (iii) the C-terminal end of the CR1 domain.

52. A method according to claim 51, wherein the surface comprises at least one of residues selected from 200-203, 210-213, 216-218, 225-230, 247-268, 244-246, 285-289) and 294-319.

53. A method as claimed in claim 48, wherein the subset defines the CR1 loop docking site.

54. A method as claimed in claim 53, wherein the docking site comprises at least one of the following ErbB2 residues: Gln 36, Gln 60, Arg 82, Thr 84, Gln 85, Phe 237, Thr 269, Phe 270, Gly 271, Ala 272, Tyr 282, Thr 285, Gly 288, Ser 289, Cys 290, Thr 291, Leu 292, Val 293, Cys 294, Pro 295 and Cys 310.

55. A method as claimed in claim 47 wherein the method is performed in silico.

56. A method as claimed in claim 55, wherein the candidate compound is selected from a real compound, a virtual compound or a combination thereof.

57. A method as claimed in claim 56, wherein the compound is in a library with at least one other candidate compound.

58. A method as claimed in claim 56, wherein the method is used for targeted screening.

59. A method as claimed in claim 57, wherein the library comprises an array of maximally diverse compounds.

60. A crystal of ErbB2 polypeptide.

61. A crystal of ErbB2 polypeptide having a space group P2₁2₁2₁ with unit cell dimensions of a=75.96 Å, b=82.24 Å, and c=110.06 Å, with up to about 1% variation in any cell dimension

62. A crystalline composition comprising a crystal of ErbB2.

63. An antibody that binds to ErbB2, the antibody being directed against at least one of the N-terminal end of the CR1 domain, the CR1 domain dimerisation loop and adjacent residues and the C-terminal end of the CR1 domain.

64. An antibody as claimed in claim 63, the antibody being directed against a structure defined by (i) ErbB2 amino acid residues 200-203, (ii) ErbB2 amino acid residues 210-213, (iii) ErbB2 amino acid residues 216-218, (iv) ErbB2 amino acid residues 225-230, (v) ErbB2 amino acid residues 247-268 or a subset thereof; (vi) ErbB2 amino acid residues 244-246, (vii) ErbB2 amino acid residues 285-289, or (viii) ErbB2 amino acid residues 294-319 or a subset thereof.
